(19) United States
(12) Patent Application Publication
(10) Pub. No.: US 2025/0327138 A1
(43) Pub. Date: Oct. 23, 2025

(54) METHODS AND COMPOSITIONS FOR DETECTING GUANITOXIN PRODUCING BACTERIA

(71) Applicants: THE REGENTS OF THE UNIVERSITY OF CALIFORNIA, Oakland, CA (US); UNIVERSITY OF SÃO PAULO, Piracicaba, SP (GB)

(72) Inventors: Bradley S. MOORE, La Jolla, CA (US); Timothy R. FALLON, La Jolla, CA (US); Jonathan R. CHEKAN, La Jolla, CA (US); Shaun M. K. MCKINNIE, La Jolla, CA (US); Stella T. LIMA, Piracicaba, SP (BR); Marli F. FIORE, Piracicaba, SP (BR)

(21) Appl. No.: 18/837,154

(22) PCT Filed: Feb. 10, 2023
(86) PCT No.: PCT/US2023/062430

§ 371 Date: Aug. 8, 2024

Related U.S. Application Data
(60) Provisional application No. 63/267,862, filed on Feb. 11, 2022.

(51) Int. Cl. C12Q 1/689; C12Q 1/6811

(52) U.S. Cl. CPC C12Q 1/689; C12Q 1/6811

(57) ABSTRACT

Provided herein, inter alia, are compositions and methods for detecting guanitoxin producing bacteria in an aqueous liquid. The methods provided herein include detecting one or more more guanitoxin biosynthetic genes in the aqueous liquid. Compositions provided herein include one or more nucleic acids at least partially complementary to a guanitoxin biosynthetic gene.

### CROSS-REFERENCE TO RELATED APPLICATIONS

This application claims priority to U.S. Provisional Application No. 63/267,862, filed Feb. 11, 2022, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under ES032056, awarded by the National Institutes of Health (NIH). The government has certain rights in the invention.

### REFERENCE TO A “SEQUENCE LISTING,” A TABLE, OR A COMPUTER PROGRAM LISTING APPENDIX SUBMITTED AS AN ASCII FILE

The contents of the electronic sequence listing (048537-651001WO_ST26.xml; Size 137,677 bytes; and Date of Creation: Feb. 9, 2023) is hereby incorporated by reference in its entirety.

### BACKGROUND OF THE INVENTION

Freshwater is essential for drinking and agriculture, yet potable watersheds are increasingly impacted by the undesirable high-density growth of algae and/or cyanobacteria.^{1, 3} Harmful algal blooms (HABs) are symptomatic of ecosystem imbalance, often caused by the varied environmental changes that demonstrate human interference and climate change. HABs are a major issue in marine, brackish, and freshwater systems worldwide. HABs are hazardous and sometimes fatal to human and animal populations, either through toxicity, or by creating ecological conditions, such as oxygen depletion, which can kill fish and other economically or ecologically important organisms. Understanding, monitoring, and remediating harmful algal/cyanobacterial blooms (HABs/cyanoHABs) and their associated toxins is essential to reducing their societal impact.⁴ Recent scientific and technological advances continue to improve an environmental cyanoHAB detection and prediction.^{5, 7} However, the vast cyanotoxin structural chemodiversity creates challenges in their comprehensive detection and quantification using standard analytical chemistry assays. In contrast, quantitative molecular biological detection of biosynthetic genes via polymerase chain reaction (PCR) provides a multiplexable and cost-effective monitoring strategy to identify the toxic potential of harmful algal blooms (HABs) independent of active toxin synthesis.⁸

The biosynthetic gene clusters (BGCs) for important freshwater cyanotoxins like a microcystin,⁹ a cylindrospermopsin,¹⁰ a saxitoxin,¹¹ and an anatoxin-a¹² have been defined and applied towards detection over the past decades. However, the biosynthetic pathway and genes for a guanitoxin (1), the only known natural organophosphate neurotoxin, have yet to be described. Previously known as the anatoxin-a(s),¹³ a guanitoxin is an irreversible inhibitor of acetylcholinesterase,¹⁴ sharing an identical mechanism of action with organophosphates like the synthetic chemical warfare agent sarin and the banned pesticide parathion (FIG. 1A). The guanitoxin induces an acute neurological toxicity that may lead to rapid death, showing comparable lethality (LD₅₀=20 μg/kg i.p.)¹⁵ to the saxitoxin, the most potent known cyanotoxin. Sporadic detection in the Americas,¹⁵ Europe,¹⁶ and Middle East¹⁷ coupled with harmful algal bloom-related animal deaths consistent with exposure suggests the guanitoxin might be an under-recognized threat in global watersheds.

While its unique pharmacology²¹ and chemical structure²² have been known for decades, the guanitoxin remains largely unmonitored in the environment due to its incompatibility with commonly used analytical detection methods and chemical instability.²³ Although previous L-arginine derived metabolites have been isolated from the guanitoxin producing cyanobacteria and incorporated in vivo via stable isotope labeling experiments (FIG. 1B),^{24,25} a lack of knowledge regarding the guanitoxin biosynthesis and accessibility to its stable metabolites as standards has hampered an understanding of its environmental significance.

Disclosed herein, inter alia, are solutions to these and other problems in the art.

### BRIEF SUMMARY OF THE INVENTION

Provided herein, inter alia, are methods and compositions for detecting guanitoxin producing bacteria in an aqueous liquid. In embodiments, the methods include detecting one or more guanitoxin biosynthetic genes in the aqueous liquid, wherein the one or more guanitoxin biosynthetic gene is GntB, GntC, GntD, GntG, GntE, GntF, GntA, GntI, GntJ, GntT, or a combination thereof. In embodiments, the methods and compositions include one or more nucleic acid each at least partially complementary to a portion of a guanitoxin biosynthetic gene.

In an aspect is provided a method of detecting guanitoxin-producing bacteria in an aqueous liquid, the method including detecting one or more guanitoxin biosynthetic genes in the aqueous liquid, wherein the one or more guanitoxin biosynthetic genes are GntA, GntB, GntC, GntD, GntE, GntF, GntG, GntH, GntI, GntJ, GntT, or a combination thereof.

In another aspect is provided a kit for detecting guanitoxin-producing bacteria in an aqueous liquid, the kit including one or more nucleic acids each at least partially complementary to a portion of one or more guanitoxin biosynthetic genes, wherein the one or more guanitoxin biosynthetic genes are GntA, GntB, GntC, GntD, GntE, GntF, GntG, GntH, GntI, GntJ, GntT, or a combination thereof.

In an aspect is provided a composition including one or more nucleic acids each independently including a sequence having at least 80% identity to any one of SEQ ID NO:1 to SEQ ID NO:22, wherein each nucleic acid of the one or more nucleic acids is different.

In an aspect is provided a method for determining cyanobacterial toxin contamination in a freshwater sample by the detection of a guanitoxin biosynthetic gene sequence in said sample. In embodiments, the guanitoxin biosynthetic gene is GntB, GntC, GntD, GntG, GntE, GntF, GntA, GntI, or GntJ.

### BRIEF DESCRIPTION OF THE DRAWINGS

FIG. 1A shows guanitoxin (1) as a potent cyanobacterial organophosphate neurotoxin with an anticholinesterase mechanism of action comparable to organophosphate pesticide parathion and chemical warfare agent sarin.

FIG. 1B shows retrobiosynthetic proposal to produce the guanitoxin (1) from a L-arginine via previously isolated cyanobacterial metabolites (S)-4-hydroxy-L-arginine (2) and L-enduracididine (3).

FIGS. 2A-2C show discovery of a candidate gnt biosynthetic gene cluster (BGC) via sequencing a guanitoxin producing cyanobacterium. FIG. 2A shows annotation of the assembled 5.24 Mbp Sphaerospermopsis torques-reginae ITEP-024 genome using antiSMASH v6.0¹⁸ detected 11 candidate BGCs, while the single candidate guanitoxin BGC was identified via colocalization of relevant candidate enzyme activities. Figure produced with Circos v0.69-8.¹⁹ FIG. 2B illustrates organization of the guanitoxin BGC. gnt locus figure designed from National Center for Biotechnology Information (NCBI) accession CP080598.1, using Clinker v0.0.21.²⁰. FIG. 2C shows the guanitoxin biosynthetic pathway in Sphaerospermopsis torques-reginae ITEP-024.

FIG. 3A shows GntF N,N-dimethylates primary amine substrate 6 to form tertiary amine 7 in vitro.

FIG. 3B shows relative intensities of positive mode extracted ion chromatograms were extracted from Hydrophilic interaction liquid chromatography-Mass Spectrometry (HILIC-MS) traces for both GntF substrate 6 and product 7 masses as appropriate ([M+H]+ (115.0978, 143.1291)±0.0100 m/z, respectively). Asterisks indicate that the MS intensities are increased ten-fold relative to other traces for improved visualization of compounds with variable ionization efficiencies.

FIG. 4A shows characterization of GntC/GntD/GntG/GntE biosynthetic enzymes with intermediate 2.

FIG. 4B shows relative intensities of positive mode extracted ion chromatograms were extracted from Ultra Performance Liquid Chromatography-Mass Spectrometry (UPLC-MS) traces following 1-fluoro-2,4-dinitrophenyl-5-L-alanine amide (L-FDAA; Marfey's reagent) derivatization of primary amine-containing intermediates 2, 3, 4, 6, and glycine ([M+H]+ (443.16; 425.15; 441.15; 367.15; 328.09)±0.20 m/z, respectively) after incubation of 2 with GntC, GntC/GntD, or GntC/GntD/GntG/GntE and all necessary cofactors and cosubstrates.

FIG. 5A shows GntA, GntI, and GntJ construct the anticholinesterase organophosphate pharmacophore of guanitoxin.

FIG. 5B shows relative intensities of positive mode extracted ion chromatograms were extracted from HILIC-MS for guanitoxin ([M+H]+ 253.1060±0.0100 m/z) following extraction from Sphaerospermopsis torques-reginae ITEP-024 and in vitro incubation of intermediate 7 with GntA/GntI/GntJ and all necessary cofactors and cosubstrates.

FIG. 5C shows Acetylcholinesterase (AChE) inhibition as assessed via the Bio Vision Acetylcholinesterase Inhibitor Screening Kit (Colorimetric). The inhibition of AChE is coupled to the decreased formation of the yellow TNB chromophore (412 nanometre (nm) absorbance) via the scheme depicted. GntA/GntI/GntJ reactions were carried out as previously described, diluted between 10-200×, and analyzed for AChE inhibition. In situ-generated 8 and 9 showed negligible AChE inhibition at all dilutions tested compared to the reversible inhibitor donepezil positive control. However, potent AChE inhibition was observed following the addition of GntJ (in situ guanitoxin) and showed a decreasing inhibitory effect at a higher reaction dilution, highlighting the significance of O-methylation for biological activity.

FIGS. 6A-6B show environmental detection of guanitoxin biosynthetic capability through metagenomic and metatranscriptomic sequencing. FIG. 6A shows geographic sites with literature reports of guanitoxin or detection of the gnt BGC through environmental sequencing datasets. FIG. 6B illustrates the gnt BGC gene structure from metagenomic and metatranscriptomic de novo assemblies of environmental samples. The two metagenomic samples were successfully linked to their respective taxon of origin via a metagenomic assembled genome (MAG) approach.

FIG. 7 shows the geographic location of guanitoxin-producing Sphaerospermopsis torques-reginae ITEP-024 cyanoHAB bloom. Sphaerospermopsis torques-reginae ITEP-024 bloom localization during the end of summer and beginning of fall 2002, Tapacurá Reservoir, Recife, PE—Brazil (8.037° S 35.169° W)⁸⁷. Photo of the Tapacurá Reservoir used with permission from Dr. Cihelio Alves Amorim (Department of Biological Sciences, Middle East Technical University, Ankara, Turkey).

FIG. 8 shows genome phylogeny for Sphaerospermopsis torques-reginae ITEP-024. The genome tree is inferred using GTDB-Tk⁸¹ by approximately-maximum-likelihood phylogenetic analysis from an aligned concatenated set of 120 single copy marker proteins for Bacteria using Genome Taxonomy Database (GTDB)⁸². The robustness of the phylogenetic tree was estimated via bootstrap analysis using 1000 replications. Bar: 0.1 changes per nucleotide position.

FIGS. 9A-9B show an identification of guanitoxin biosynthetic intermediates 3, 7, and 8 in Sphaerospermopsis torques-reginae ITEP-024 methanolic culture extracts. Positive mode HILIC-MS chromatograms identify the presence of 3, 7, and 8 in the cyanobacteria culture extract as compared with synthetic standards 3 and 7, as well as in situ enzyme-generated intermediates 7 and 8. FIG. 9A shows the positive mode extracted ion chromatogram (EIC±0.0010 m/z) comparison for 3 ([M+H]⁺ 173.1033 m/z) from the synthetic standard and cyanobacterial culture extracts. FIG. 9B shows the positive mode extracted ion chromatogram (EIC±0.0100 m/z) comparisons for 7 and 8 ([M+H]⁺ 143.1291, 159.1240 m/z respectively) from synthetic standard 7, enzyme-generated 7 and 8, and cyanobacterial culture extracts.

FIGS. 10A-10B show Sphaerospermopsis torques-reginae ITEP-024 genome and guanitoxin biosynthetic gene cluster information. FIG. 10A is the genome representation with 5.2 Mbp of length and guanitoxin biosynthetic gene cluster position in the genome. FIG. 10B is the genome data of Sphaerospermopsis torques-reginae ITEP-024 cyanobacterium.

FIG. 11 shows a sodium dodecyl-sulfate polyacrylamide gel electrophoresis (SDS-PAGE) analysis of GntA/C/D/E/F/G/I/J proteins. 4-15% Mini-PROTEAN® TGX™ Precast Protein Gels (Bio-Rad) loaded with Precision Plus Protein Dual Color Standards (Bio-Rad) and purified soluble Gnt pathway proteins (2 μg).

FIGS. 12A-12B show guanitoxin pyridoxal 5′-phosphate (PLP)-dependent enzymes in a possible reaction with an L-arginine substrate. FIG. 12A illustrates possible products for GntC/GntG/GntE reactions. The reactions were set up as previously described for 5 hours at room temperature. Half of each assay was methanol-quenched, while the other half was derivatized with Marfey's reagent for optimized retention times and diastereomer separation prior to UPLC-MS analysis. FIG. 12B shows relative intensities of positive mode extracted ion chromatograms were extracted from UPLC-MS traces (EIC±0.30 m/z) for non-derivatized arginine ([M+H]⁺ 175.12 m/z) and derivatized arginine ([M+H]⁺ 427.14 m/z), or all putative non-derivatized products ([M+H]⁺ 191.11, 174.08, 190.00, 173.10, 172.10 m/z) or and all putative derivatized products ([M+H]⁺ 443.16, 425.15 m/z).

FIG. 13A shows GntD does not hydroxylate L-arginine. GntD reactions were set up as previously described and incubated for 4 hours at room temperature. Reactions were derivatized via Marfey's analysis for optimized retention times prior to UPLC-MS analysis.

FIG. 13B illustrates relative intensities of positive mode extracted ion chromatograms from UPLC-MS traces (EIC±0.30 m/z) for either derivatized L-arginine ([M+H]⁺ 427.14 m/z) or all putative derivatized products ([M+H]⁺ 443.16, 459.15, 475.15, 425.15, 441.14 m/z).

FIGS. 14A-14D show GntC substrate specificity, time course, and PLP-dependence experiments. GntC assays were set up as previously described and incubated at room temperature and aliquots were taken at the time points listed on the figures (panels of FIGS. 14A-14B: 25 h; panel of FIG. 14C: 15 min, 6 h, 25 h; panel of FIG. 14D: 20 h). Reactions were derivatized with Marfey's reagent for optimized retention times and diastereomer separation prior to UPLC-MS analysis. Relative intensities of positive mode extracted ion chromatograms were extracted from UPLC-MS traces (EIC±0.30 m/z) for Marfey derivatized starting material 2 and product 3 ([M+H]⁺ 443.16, 425.15 m/z respectively) in all traces unless otherwise listed (ie the bottom traces in FIGS. 14A-14B). FIG. 14A illustrates that GntC catalyzes the cyclodehydration of 2 in vitro to produce 3. FIG. 14B shows that GntC shows negligible activity towards epimerized substrate SI-12 and does not produce epimer SI-14. FIG. 14C shows data illustrating that GntC shows a time-dependent increase in activity over the course of the 25 h assay, and FIG. 14D shows that exogenous PLP is not needed for catalysis due to co-purifying with this cofactor.

FIG. 15 shows gntB-gntC-pCOLADuet-1 vector map. pCOLADuet-1 vector assembled with gntB and gntC genes from the guanitoxin pathway for enduracididine production. The vector is designed for the coexpression of two target genes from a single plasmid, which encodes two multiple cloning sites (MCS) each of which is preceded by a T7 promoter, lac operon and ribosome binding site. The vector has the COLA replicon from ColA ori and kanamycin resistance gene.

FIG. 16A shows GntBC produces L-enduracididine (3) in vivo in E. coli. The GntBC in vivo assay was set up as previously described and incubated at 18° C. for five days. A 100 μM internal standard of synthetic 3 was added to pET28a cell lysate to correct for variations in retention time based on media components. In vivo reactions were derivatized with Marfey's reagent prior to UPLC-MS analysis.

FIG. 16B, relative intensities of positive mode extracted ion chromatograms were extracted from UPLC-MS traces (EIC±0.30 m/z) for Marfey-derivatized GntC product 3 ([M+H]⁺ 425.15 m/z). The in vivo production of 3 was dependent on the presence of both gntB and gntC genes but was not observed in the gntB-pET28a or empty vector pET28a incubations.

FIGS. 17A-17D show divergent cyclic arginine amino acid biosyntheses that use PLP-dependent enzymology. Comparison of guanitoxin and previously characterized actinobacterial biosynthetic pathways. FIG. 17A illustrates a portion of the guanitoxin biosynthesis pathway using Sphaerospermopsis torques-reginae (cyanobacteria). FIG. 17B illustrates the mannopeptimycin biosynthesis using Streptomycin hygroscopicus (actinobacteria) shown in studies of Han et. al., Biochemistry 2015, 54, 7092; Han et al., Biochemistry 2018, 57, 3252; Burroughs et al., Biochemistry, 2013, 52, 4492 and Haltli et al., Chem. Biol., 2005, 12, 1163. FIG. 17C illustrates the viomycin biosynthesis using Streptomyces punices and other sp. (actinobacteria) shown in studies of Yin et al., ChemBioChem, 2004, 5, 1274; Ju et al., ChemBioChem, 2004, 5, 1281; Yin et al., ChemBioChem, 2004, 5, 1278; Fei et al., J. Nat. Prod., 2007, 70, 618 and Barkei et al., ChemBioChem, 2009, 10, 366. FIG. 17D illustrates the steptolidine biosynthesis using Streptomyces lavendulae (actinobacteria) shown in studies of Chang et al., Angew. Chem. Int. Ed., 2014, 53, 1943.

FIGS. 18A-18C show GntD substrate specificity, time course and dependence experiments. GntD assays were set up as previously described at room temperature, and aliquots were taken at the time points listed on the figures (panels of FIGS. 18A-18B: 15 min., 90 min., 5 h; panel of FIG. 18C: 2 h, 20 h). Reactions were derivatized with Marfey's reagent for optimized retention times and diastereomer separation prior to UPLC-MS analysis. Relative intensities of positive mode extracted ion chromatograms were extracted from UPLC-MS traces (EIC±0.50 m/z) for both Marfey derivatized substrate 3 and product 4 ([M+H]⁺ 424.15, 441.14 m/z, respectively) in all traces. FIG. 18A shows that GntD rapidly hydroxylates substrate 3 in vitro but FIG. 18B shows negligible activity towards epimer SI-14. FIG. 18C illustrates results from a GntD dependence assay.

FIG. 19A shows the GntE and GntG forward aldol assay. GntE and GntG in vitro aldol reaction dependence assays were set up as previously described and incubated at room temperature for 18 hours. Reactions were derivatized with Marfey's reagent for optimized retention times prior to UPLC-MS analysis.

FIG. 19B shows relative intensities of positive mode extracted ion chromatograms were extracted from UPLC-MS traces (EIC±0.50 m/z) for Marfey derivatized 4 ([M+H]⁺ 441.14 m/z). The enzymatically isolated GntD reaction product 4 was compared to the incubation of 6 with one or both GntE/G enzymes, and a no enzyme control. The inclusion of only GntE (−GntG trace) showed production of 4, indicating that GntE may be capable of performing both aldolase and transamination chemistries. In contrast, the presence of GntG only (−GntE trace) shows no 4 production, indicating that this functional promiscuity is limited to GntE.

FIG. 20A shows GntCDGE in vitro one pot dependence assay. Enzyme assays were set up as previously described and incubated at room temperature for 18 hours. One condition included all enzymes, while other conditions omitted one or more enzymes. Reactions were derivatized with Marfey's reagent prior to UPLC-MS analysis for improved retention times.

FIG. 20B illustrates relative intensities of positive mode extracted ion chromatograms from UPLC-MS traces (EIC±0.20 m/z) for all potential products 2, 3, 4, glycine, and 6 for all traces ([M+H]⁺ 443.16, 425.15, 441.14, 328.08, and 367.14 m/z respectively). The reaction progression from 2 to 6 was halted depending on the omission of particular enzymes that corresponded to their native biosynthetic roles. Analogous to the results obtained in FIG. 16A-16B, the omission of aldolase GntG (−GntG trace) showed no significant deviation from the full assay (all component trace) due to the dual functionality of GntE as both an aldolase and transaminase. However, the omission of aminotransferase GntE (−GntE trace) supports the aldolase activity of GntG via conversion of 4 into glycine and 5 (not detected).

FIG. 21A shows GntA hydroxylates cyclic guanidine substrate 7. GntA reactions were set up as previously described and incubated overnight at 27° C. Reactions were quenched with acetonitrile and subjected to HILIC-MS analysis.

FIG. 21B illustrates relative intensities of positive mode extracted ion chromatograms from HILIC-MS traces (EIC±0.0100 m/z) for the GntA product 8 and substrate 7 masses as appropriate ([M+H]⁺ 159.1240 and 143.1291 m/z respectively).

FIG. 22A shows GntAIJ produce guanitoxin in situ from synthetic substrate 7. The GntA, GntI, and GntJ reactions were set up as previously described beginning with synthetic substrate 7. Reactions were quenched with acetonitrile and subjected to LC-MS analysis.

FIG. 22B shows GntA and GntI reactions analyzed using the HILIC Method, and relative intensities of positive mode extracted ion chromatograms extracted from HILIC-MS traces (EIC±0.0100 m/z) for substrate 7, GntA product 8, and GntI product 9 as appropriate ([M+H]+ 143.1291, 159.1240, and 239.0904 m/z respectively).

FIG. 22C shows the GntAIJ coupled reaction, no substrate control, and Sphaerospermopsis torques-reginae ITEP-024 extract analyzed using the reverse phase (RP) method. Relative intensities of positive mode extracted ion chromatograms were extracted from reversed phase-liquid chromatography-mass spectrometry (RP-LC-MS) traces (EIC±0.0100 m/z) for the guanitoxin (1) mass ([M+H]⁺ 253.1060 m/z). Asterisks indicate that the MS intensities are increased 25-fold relative to other traces for improved visualization.

FIG. 23A shows guanitoxin biosynthetic intermediates from Sphaerospermopsis torques-reginae ITEP-024.

FIG. 23B-23D illustrates mass spectrometry-mass spectrometry analyses of guanitoxin biosynthetic intermediates. Intermediates 7 (FIG. 23B), 8 (FIG. 23C), and guanitoxin (1) (FIG. 23D) showed diagnostic fragment A (58.0652 m/z) following HILIC-MS/MS analyses.

FIG. 24 shows phylogenomic tree for taxonomic classification of MAGs based on Genome Taxonomy Database (GTDB)⁸². Cuspidothrix bin 5 belongs to Amazon River and Aphanizomenon bin 35 belongs to Lake Mendota. The genome tree is generated using GTDB-Tk⁸¹ by the identification and alignment of 120 bacterial single-copy conserved marker genes, then inferred the phylogeny of the concatenated sequences with the WAG+GAMMA models and maximum likelihood algorithm. The robustness of the phylogenetic tree was estimated via bootstrap analysis using 1000 replications. Bar: 0.1 changes per nucleotide position.

FIG. 25 shows genome similarity matrix of MAG-assembled gnt-containing cyanobacteria. Similarity between Lake Mendota bin 35 (Aphanizomenon) and all Aphanizomenon available genomes, and Amazon River bin 5 (Cuspidothrix) with the only available Cuspidothrix genome. Average nucleotide identities were calculated with OrthoANI v1.4⁸⁶.

FIG. 26 shows the synthetic scheme of primary amine intermediate 6.

FIG. 27 shows the synthetic scheme of dimethylamine intermediate 7.

FIG. 28 shows the synthetic scheme of γ-hydroxy-L-arginine diastereomers SI-7 and SI-8.

FIG. 29 shows the synthetic scheme of (S)-γ-hydroxy-L-arginine 2.

FIG. 30 shows the synthetic scheme of L-enduracididine (3).

FIG. 31 shows the synthetic scheme of (R)-γ-hydroxy-L-arginine (SI-12).

FIG. 32 shows the synthetic scheme of L-allo-enduracididine (SI-14).

### DETAILED DESCRIPTION OF THE INVENTION

Unless specifically defined otherwise, all technical and scientific terms used herein shall be taken to have the same meaning as commonly understood by one of ordinary skill in the art (e.g., in oncology, cell culture, molecular genetics, epigenetics, and biochemistry).

As used herein, the term “about” in the context of a numerical value or range means±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” discloses 0.2 mg, 0.3 mg, 0.4 mg, 0.5 mg, 0.6 mg etc. up to and including 5.0 mg. Additionally, where two values for a parameter are disclosed, then a range of all values between and including those two values is also disclosed. For example, “1, 2, and 3” discloses, e.g., 1-2, 1-3, and 2-3.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene (e.g. GntA protein, GntB protein, GntC protein, GntD protein, GntE protein, GntF protein, GntG protein, GntH protein, GntI protein, GntJ protein, or GntT protein). In embodiments, a gene includes a coding sequence, a promoter region sequence, a terminator region sequence, or an intergene region sequence.

The term “promoter” or “promoter region sequence” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene. The promoter comprises a DNA regulatory sequence, wherein the regulatory sequence is derived from a gene, which is capable of binding RNA polymerase and initiating transcription of a downstream (3′-direction) coding sequence.

The term “terminator” or “terminator region sequence” refers to a nucleic acid sequence that determines the end of a gene during the transcription process. The terminator may include a sequence that directly or indirectly releases the transcript RNA from the transcriptional complex. For example, the terminator region sequence may include the sequence that determines the detachment of RNA polymerase from the DNA template strand.

The term “coding sequence” (CDS), or “coding region” refers to the portion of a gene that codes for protein. For example, the coding sequence may be the DNA or RNA sequence that determines the sequence of amino acids in a protein.

The term “intergenic region” or “intergenic region sequence” refers to the nucleic acid sequence between genes. An intergenic region sequence in bacteria may be a non-protein coding sequence. For example, an intergenic region sequence may comprise a part of a bacterial genome located between the last nucleotide of a coding region and the first nucleotide of a subsequent coding region.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. In embodiments, the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment in length within sequences for comparison are well-known in the art. Optimal alignment in length within sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “heme dependent pre-guanitoxin N-hydrolase” or “heme dependent pre-guanitoxin N-hydrolase protein” as referred to herein includes any of the recombinant or naturally-occurring forms of heme dependent pre-guanitoxin N-hydrolase (GntA protein) or variants or homologs thereof that maintain heme dependent pre-guanitoxin N-hydrolase activity (e.g. within at least 50%, 80%, 90%, 95%, 96%, 97%, 98%, 99% or 100% activity compared to heme dependent pre-guanitoxin N-hydrolase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring heme dependent pre-guanitoxin N-hydrolase protein (e.g. SEQ ID NO:76). In embodiments, the heme dependent pre-guanitoxin N-hydrolase includes the sequence of SEQ ID NO:76. In embodiments, the heme dependent pre-guanitoxin N-hydrolase is encoded by the sequence of SEQ ID NO:23. In embodiments, the heme dependent pre-guanitoxin N-hydrolase is encoded by the sequence of SEQ ID NO:24. In embodiments, the heme dependent pre-guanitoxin N-hydrolase is encoded by the sequence of SEQ ID NO:25. In embodiments, the heme dependent pre-guanitoxin N-hydrolase is encoded by the sequence of SEQ ID NO:26. In embodiments, the heme dependent pre-guanitoxin N-hydrolase is encoded by the sequence of SEQ ID NO:27.

A “L-arginine gamma (S) hydroxylase” or “L-arginine gamma (S) hydroxylase protein” as referred to herein includes any of the recombinant or naturally-occurring forms of L-arginine gamma (S) hydroxylase (GntB protein) or variants or homologs thereof that maintain L-arginine gamma (S) hydroxylase activity (e.g. within at least 50%, 80%, 90%, 95%, 96%, 97%, 98%, 99% or 100% activity compared to L-arginine gamma (S) hydroxylase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring L-arginine gamma (S) hydroxylase protein (e.g. SEQ ID NO:77). In embodiments, the L-arginine gamma (S) hydroxylase includes the sequence of SEQ ID NO:77. In embodiments, the L-arginine gamma (S) hydroxylase is encoded by the sequence of SEQ ID NO:28. In embodiments, the L-arginine gamma (S) hydroxylase is encoded by the sequence of SEQ ID NO:29. In embodiments, the L-arginine gamma (S) hydroxylase is encoded by the sequence of SEQ ID NO:30. In embodiments, the L-arginine gamma (S) hydroxylase is encoded by the sequence of SEQ ID NO:31. In embodiments, the L-arginine gamma (S) hydroxylase is encoded by the sequence of SEQ ID NO:32.

A “PLP-dependent (S)-gamma-hydroxy-L-arginine cyclodehydratase” or “PLP-dependent (S)-gamma-hydroxy-L-arginine cyclodehydratase protein” as referred to herein includes any of the recombinant or naturally-occurring forms of PLP-dependent (S)-gamma-hydroxy-L-arginine cyclodehydratase (GntC protein) or variants or homologs thereof that maintain PLP-dependent (S)-gamma-hydroxy-L-arginine cyclodehydratase (e.g. within at least 50%, 80%, 90%, 95%, 96%, 97%, 98%, 99% or 100% activity compared to PLP-dependent (S)-gamma-hydroxy-L-arginine cyclodehydratase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PLP-dependent (S)-gamma-hydroxy-L-arginine cyclodehydratase protein (e.g. SEQ ID NO:78). In embodiments, the PLP-dependent (S)-gamma-hydroxy-L-arginine cyclodehydratase includes the sequence of SEQ ID NO:78. In embodiments, the PLP-dependent (S)-gamma-hydroxy-L-arginine cyclodehydratase is encoded by the sequence of SEQ ID NO:33. In embodiments, the PLP-dependent (S)-gamma-hydroxy-L-arginine cyclodehydratase is encoded by the sequence of SEQ ID NO:34. In embodiments, the PLP-dependent (S)-gamma-hydroxy-L-arginine cyclodehydratase is encoded by the sequence of SEQ ID NO:35. In embodiments, the PLP-dependent (S)-gamma-hydroxy-L-arginine cyclodehydratase is encoded by the sequence of SEQ ID NO:36. In embodiments, the PLP-dependent (S)-gamma-hydroxy-L-arginine cyclodehydratase is encoded by the sequence of SEQ ID NO:37.

A “L-enduracididine beta-hydroxylase” or “L-enduracididine beta-hydroxylase protein” as referred to herein includes any of the recombinant or naturally-occurring forms of L-enduracididine beta-hydroxylase (GntD protein) or variants or homologs thereof that maintain L-enduracididine beta-hydroxylase (e.g. within at least 50%, 80%, 90%, 95%, 96%, 97%, 98%, 99% or 100% activity compared to L-enduracididine beta-hydroxylase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring L-enduracididine beta-hydroxylase protein (e.g. SEQ ID NO:79). In embodiments, the L-enduracididine beta-hydroxylase includes the sequence of SEQ ID NO:79. In embodiments, the L-enduracididine beta-hydroxylase is encoded by the sequence of SEQ ID NO:38. In embodiments, the L-enduracididine beta-hydroxylase is encoded by the sequence of SEQ ID NO:39. In embodiments, the L-enduracididine beta-hydroxylase is encoded by the sequence of SEQ ID NO:40. In embodiments, the L-enduracididine beta-hydroxylase is encoded by the sequence of SEQ ID NO:41. In embodiments, the L-enduracididine beta-hydroxylase is encoded by the sequence of SEQ ID NO:42.

A “PLP-dependent transaminase” or “PLP-dependent transaminase protein” as referred to herein includes any of the recombinant or naturally-occurring forms of PLP-dependent transaminase (GntE protein) or variants or homologs thereof that maintain PLP-dependent transaminase (e.g. within at least 50%, 80%, 90%, 95%, 96%, 97%, 98%, 99% or 100% activity compared to PLP-dependent transaminase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PLP-dependent transaminase protein (e.g. SEQ ID NO:80). In embodiments, the PLP-dependent transaminase includes the sequence of SEQ ID NO:80. In embodiments, the PLP-dependent transaminase is encoded by the sequence of SEQ ID NO:43. In embodiments, the PLP-dependent transaminase is encoded by the sequence of SEQ ID NO:44. In embodiments, the PLP-dependent transaminase is encoded by the sequence of SEQ ID NO:45. In embodiments, the PLP-dependent transaminase is encoded by the sequence of SEQ ID NO:46. In embodiments, the PLP-dependent transaminase is encoded by the sequence of SEQ ID NO:47.

A “pre-guanitoxin forming N-methyltransferase” or “pre-guanitoxin forming N-methyltransferase protein” as referred to herein includes any of the recombinant or naturally-occurring forms of pre-guanitoxin forming N-methyltransferase (GntF protein) or variants or homologs thereof that maintain pre-guanitoxin forming N-methyltransferase (e.g. within at least 50%, 80%, 90%, 95%, 96%, 97%, 98%, 99% or 100% activity compared to pre-guanitoxin forming N-methyltransferase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring pre-guanitoxin forming N-methyltransferase protein (e.g. SEQ ID NO:81). In embodiments, the pre-guanitoxin forming N-methyltransferase includes the sequence of SEQ ID NO:81. In embodiments, the pre-guanitoxin forming N-methyltransferase is encoded by the sequence of SEQ ID NO:48. In embodiments, the pre-guanitoxin forming N-methyltransferase is encoded by the sequence of SEQ ID NO:49. In embodiments, the pre-guanitoxin forming N-methyltransferase is encoded by the sequence of SEQ ID NO:50. In embodiments, the pre-guanitoxin forming N-methyltransferase is encoded by the sequence of SEQ ID NO:51. In embodiments, the pre-guanitoxin forming N-methyltransferase is encoded by the sequence of SEQ ID NO:52.

A “PLP-dependent aldolase” or “PLP-dependent aldolase protein” as referred to herein includes any of the recombinant or naturally-occurring forms of PLP-dependent aldolase (GntG protein) or variants or homologs thereof that maintain PLP-dependent aldolase (e.g. within at least 50%, 80%, 90%, 95%, 96%, 97%, 98%, 99% or 100% activity compared to PLP-dependent aldolase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PLP-dependent aldolase protein (e.g. SEQ ID NO:82). In embodiments, the PLP-dependent aldolase includes the sequence of SEQ ID NO:82. In embodiments, the PLP-dependent aldolase is encoded by the sequence of SEQ ID NO:53. In embodiments, the PLP-dependent aldolase is encoded by the sequence of SEQ ID NO:54. In embodiments, the PLP-dependent aldolase is encoded by the sequence of SEQ ID NO:55. In embodiments, the PLP-dependent aldolase is encoded by the sequence of SEQ ID NO:56. In embodiments, the PLP-dependent aldolase is encoded by the sequence of SEQ ID NO:57.

A “MBL fold metallo-hydrolase” or “MBL fold metallo-hydrolase protein” as referred to herein includes any of the recombinant or naturally-occurring forms of MBL fold metallo-hydrolase (GntH protein) or variants or homologs thereof that maintain MBL fold metallo-hydrolase (e.g. within at least 50%, 80%, 90%, 95%, 96%, 97%, 98%, 99% or 100% activity compared to MBL fold metallo-hydrolase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MBL fold metallo-hydrolase protein (e.g. SEQ ID NO:83). In embodiments, the MBL fold metallo-hydrolase includes the sequence of SEQ ID NO:83. In embodiments, the MBL fold metallo-hydrolase is encoded by the sequence of SEQ ID NO:56. In embodiments, the MBL fold metallo-hydrolase is encoded by the sequence of SEQ ID NO:57. In embodiments, the MBL fold metallo-hydrolase is encoded by the sequence of SEQ ID NO:58. In embodiments, the MBL fold metallo-hydrolase is encoded by the sequence of SEQ ID NO:59. In embodiments, the MBL fold metallo-hydrolase is encoded by the sequence of SEQ ID NO:60. In embodiments, the MBL fold metallo-hydrolase is encoded by the sequence of SEQ ID NO:61. In embodiments, the MBL fold metallo-hydrolase is encoded by the sequence of SEQ ID NO:62.

A “pre-guanitoxin N-oxide kinase” or “pre-guanitoxin N-oxide kinase protein” as referred to herein includes any of the recombinant or naturally-occurring forms of pre-guanitoxin N-oxide kinase (GntI protein) or variants or homologs thereof that maintain pre-guanitoxin N-oxide kinase (e.g. within at least 50%, 80%, 90%, 95%, 96%, 97%, 98%, 99% or 100% activity compared to pre-guanitoxin N-oxide kinase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring pre-guanitoxin N-oxide kinase protein (e.g. SEQ ID NO:84). In embodiments, the pre-guanitoxin N-oxide kinase includes the sequence of SEQ ID NO:84. In embodiments, the pre-guanitoxin N-oxide kinase is encoded by the sequence of SEQ ID NO:63. In embodiments, the pre-guanitoxin N-oxide kinase is encoded by the sequence of SEQ ID NO:64. In embodiments, the pre-guanitoxin N-oxide kinase is encoded by the sequence of SEQ ID NO:65. In embodiments, the pre-guanitoxin N-oxide kinase is encoded by the sequence of SEQ ID NO:66. In embodiments, the pre-guanitoxin N-oxide kinase is encoded by the sequence of SEQ ID NO:67.

A “guanitoxin forming phosphate O-methyltransferase” or “guanitoxin forming phosphate O-methyltransferase protein” as referred to herein includes any of the recombinant or naturally-occurring forms of guanitoxin forming phosphate O-methyltransferase (GntJ protein) or variants or homologs thereof that maintain guanitoxin forming phosphate O-methyltransferase (e.g. within at least 50%, 80%, 90%, 95%, 96%, 97%, 98%, 99% or 100% activity compared to guanitoxin forming phosphate O-methyltransferase). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring guanitoxin forming phosphate O-methyltransferase protein (e.g. SEQ ID NO:85). In embodiments, the guanitoxin forming phosphate O-methyltransferase includes the sequence of SEQ ID NO:85. In embodiments, the guanitoxin forming phosphate O-methyltransferase is encoded by the sequence of SEQ ID NO:68. In embodiments, the guanitoxin forming phosphate O-methyltransferase is encoded by the sequence of SEQ ID NO:69. In embodiments, the guanitoxin forming phosphate O-methyltransferase is encoded by the sequence of SEQ ID NO:70. In embodiments, the guanitoxin forming phosphate O-methyltransferase is encoded by the sequence of SEQ ID NO:71. In embodiments, the guanitoxin forming phosphate O-methyltransferase is encoded by the sequence of SEQ ID NO:72.

A “MATE family efflux transporter” or “MATE family efflux transporter protein” as referred to herein includes any of the recombinant or naturally-occurring forms of g MATE family efflux transporter (GntT protein) or variants or homologs thereof that maintain MATE family efflux transporter (e.g. within at least 50%, 80%, 90%, 95%, 96%, 97%, 98%, 99% or 100% activity compared to MATE family efflux transporter). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MATE family efflux transporter protein (e.g. SEQ ID NO:86). In embodiments, the MATE family efflux transporter includes the sequence of SEQ ID NO:86. In embodiments, the MATE family efflux transporter is encoded by the sequence of SEQ ID NO:73. In embodiments, the MATE family efflux transporter is encoded by the sequence of SEQ ID NO:74. In embodiments, the MATE family efflux transporter is encoded by the sequence of SEQ ID NO:75.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

A “label”, “detectable label” or “detectable moiety” are used interchangeably and refer to a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a nucleic acid. Any appropriate method known in the art for conjugating a nucleic acid to the label may be employed, e.g., using methods described in Hermanson, Bioconjugate Techniques 1996, Academic Press, Inc., San Diego. In embodiments, the label is a dye that binds to double-stranded DNA. In embodiments, the label is a fluorescent label. In embodiments, the label is FAM, SUN, 3, Texas Red-X, or Cy5. In embodiments, the label includes a plurality of fluorescent labels wherein each fluorescent label of the plurality has a different emission wavelength (e.g. for multiplex PCR methods (e.g. qPCR, RT qPCR, etc.)).

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells. In embodiments, the cell is a bacteria cell. In embodiments, the cell is a cyanobacteria cell. In embodiments, the cell is a guanitoxin producing bacteria cell.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject, patient, or a liquid (e.g. a lake, river pond; a private water system, a public water system). A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. A sample may include a volume of liquid (e.g. an aqueous liquid) taken from a lake, river pond, private water system, or public water system. The sample may include bacteria, including guanitoxin producing bacteria.

A “control” or “standard control” refers to a sample, measurement, or value that serves as a reference, usually a known reference, for comparison to a test sample, measurement, or value. For example, a test sample (e.g. aqueous liquid) can be taken from a lake, pond, river, public water system, or private water system suspected of having guanitoxin producing bacteria. The test sample (e.g. aqueous liquid) can be compared to a known sample including one or more guanitoxin biosynthetic genes (e.g. a standard control). For example, a control DNA can be one or more guanitoxin biosynthetic genes (e.g. GntA, GntB, GntC, GntD, GntE, GntF, GntG, GntH, GntI, GntJ, GntT, or a combination thereof). The control DNA may be a positive control in a PCR or isothermal amplification method, for example, in qPCR. In another example, a standard control value can also be obtained from a lake, pond, river, public water system, or private water system prior to contamination with guanitoxin producing bacteria. In another example, a standard control can be obtained by excluding one or more reagent from a test, assay, or method. For example, a negative control for a PCR method (e.g. pPCR, RT-aPCR) may include performing the PCR method without one or more reagents (e.g. polymerase).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition (e.g. guanitoxin toxicity, symptom of guanitoxin toxicity) that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The term “associated” or “associated with” in the context of a substance or substance activity or function associated with a disease or condition (e.g. guanitoxin associated toxicity, guanitoxin producing bacteria associated toxicity) means that the disease or condition is caused by (in whole or in part), or a symptom of the disease is caused by (in whole or in part) the substance (guanitoxin, guanitoxin producing bacteria) or substance activity or function. As used herein, what is described as being associated with a disease, if a causative agent, could be a target for treatment of the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as guanitoxin toxicity (e.g. guanitoxin induced toxicity). In embodiments, the therapeutic agent is a muscle relaxant, benzodiazepine, or barbiturate. In embodiments, the therapeutic agent is atropine. In embodiments, the treat therapeutic agent is glycopyrrolate. In embodiments, the therapeutic agent is physostigmine. In embodiments, the therapeutic agent is 2-PAM. In embodiments, the therapeutic is an agent identified herein having utility in treating symptoms (e.g. seizure, tremors, etc) of guanitoxin toxicity.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

### METHODS

Provided herein are, inter alia, methods for detecting guanitoxin-producing bacteria in aqueous liquids. The methods described herein provide sensitive and accurate detection of guanitoxin producing bacteria by detecting one or more guanitoxin biosynthetic genes in the aqueous liquid, wherein the one or more guanitoxin biosynthetic genes are GntA, GntB, GntC, GntD, GntE, GntF, GntG, GntH, GntI, GntJ, GntT, or a combination thereof. The methods provided herein including embodiments thereof are contemplated to be effective for diagnosing guanitoxin contamination in an aqueous liquid (e.g. derived from a pond, lake, or river; derived from a public water system or private water system) by detecting guanitoxin-producing bacteria in the aqueous liquid. The methods provided herein including embodiments thereof are further contemplated to be useful for treating guanitoxin toxicity in a subject in need thereof. Thus, in an aspect is provided a method of detecting guanitoxin-producing bacteria in an aqueous liquid, the method including detecting one or more guanitoxin biosynthetic genes in the aqueous liquid, wherein the one or more guanitoxin biosynthetic genes are GntA, GntB, GntC, GntD, GntE, GntF, GntG, GntH, GntI, GntJ, GntT, or a combination thereof.

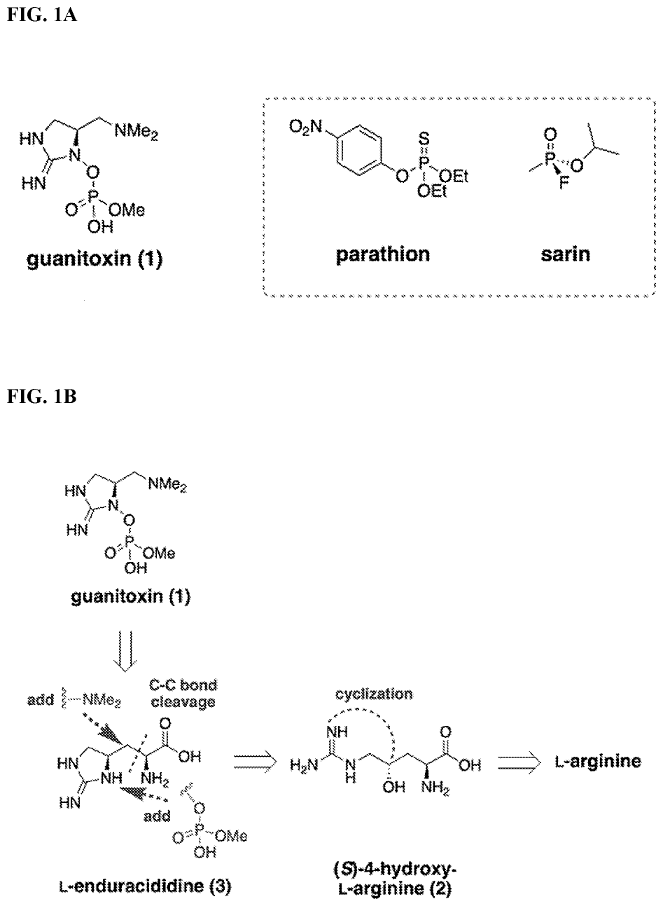

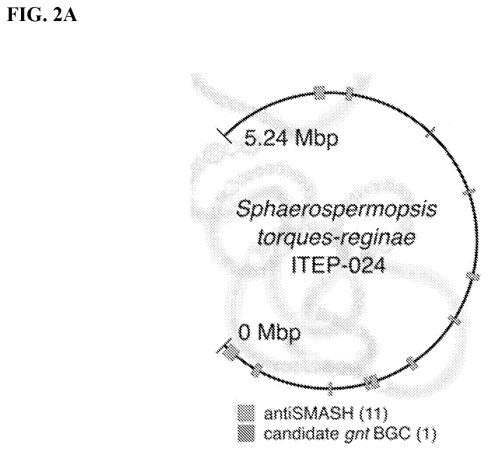

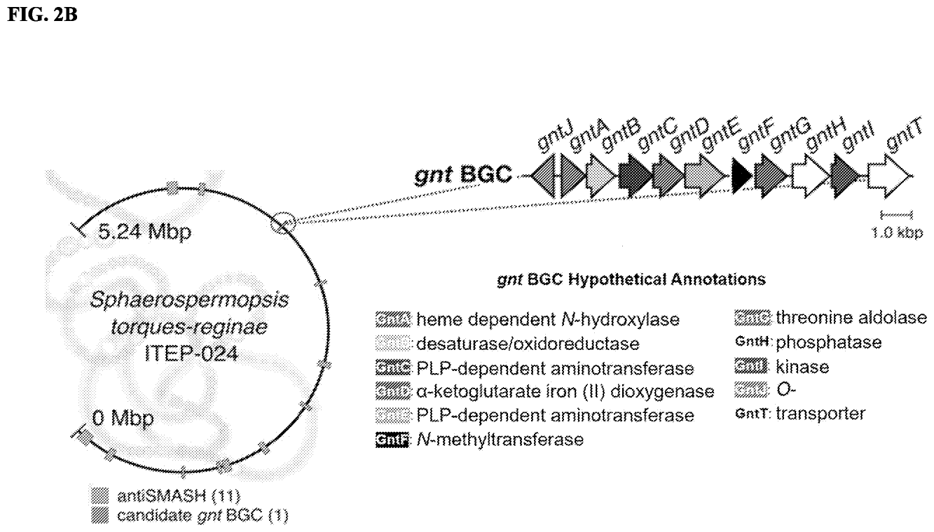

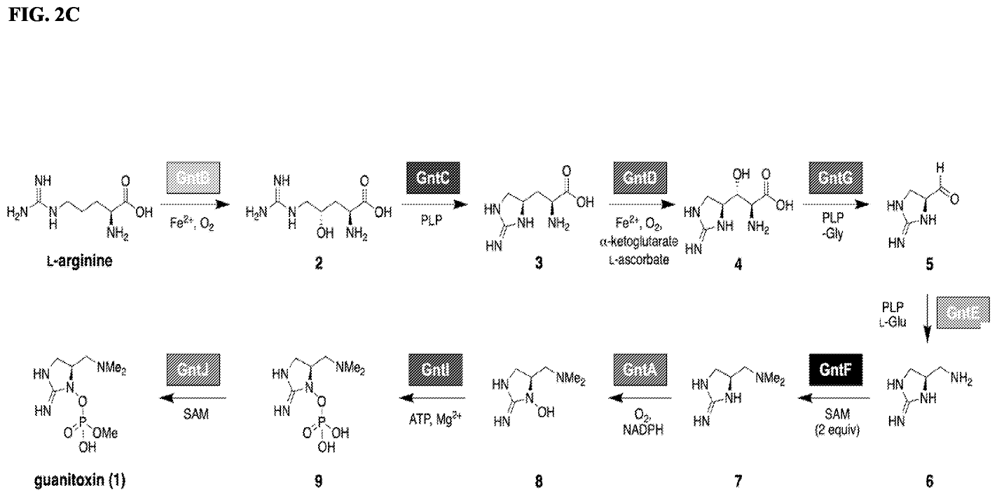

The term “guanitoxin”, also referred to as “anatoxin-a(S)”, is used in accordance to its ordinary meaning in the art, and refers to a compound having the structure shown in FIG. 1A (left panel). Guanitoxin may be produced by cyanobacteria (e.g. guanitoxin producing bacteria). Guanitoxin may irreversibly inhibit the active site of the enzyme acetylcholinesterase, thereby causing toxicity in a subject who has ingested, inhaled, or come in contact with an aqueous liquid including guanitoxin producing bacteria. Thus, the term “guanitoxin producing bacteria” refers to freshwater bacteria (e.g. cyanobacteria) that are capable of guanitoxin biosynthesis. Guanitoxin producing bacteria may produce guanitoxin or an intermediate compound in the biosynthesis of guanitoxin. In embodiments, an intermediate compound in guanitoxin biosynthesis includes any one of the structures shown in FIG. 2C (compounds 2-9).

“Detecting” or “assaying” means using a procedure (e.g. a PCR method, an isothermal amplification method, a sequencing method, etc.) to qualitatively assess or quantitatively measure the presence or amount of the guanitoxin biosynthetic genes as described herein such as, for example, detecting the presence of one or more of GntA, GntB, GntC, GntD, GntE, GntF, GntG, GntH, GntI, GntJ, GntT, or any combination thereof, using a method (such as qPCR, RT-PCR, sequencing, etc.) to qualitatively assess or quantitatively measure the presence or amount of the selected guanitoxin biosynthetic gene.

As used herein, the term “guanitoxin biosynthetic gene” refers to a gene that encodes a protein involved in producing guanitoxin or an intermediate compound in the biosynthesis of guanitoxin. In embodiments, guanitoxin biosynthetic gene is GntA, GntB, GntC, GntD, GntE, GntF, GntG, GntH, GntI, GntJ, or GntT or a fragment thereof. In embodiments, the guanitoxin biosynthetic gene is GntA or a fragment thereof. In embodiments, the guanitoxin biosynthetic gene is GntB or a fragment thereof. In embodiments, the guanitoxin biosynthetic gene is GntC or a fragment thereof. In embodiments, the guanitoxin biosynthetic gene is GntD or a fragment thereof. In embodiments, the guanitoxin biosynthetic gene is GntE or a fragment thereof. In embodiments, the guanitoxin biosynthetic gene is GntF or a fragment thereof. In embodiments, the guanitoxin biosynthetic gene is GntG or a fragment thereof. In embodiments, the guanitoxin biosynthetic gene is GntH or a fragment thereof. In embodiments, the guanitoxin biosynthetic gene is GntI or a fragment thereof. In embodiments, the guanitoxin biosynthetic gene is GntJ or a fragment thereof. In embodiments, the guanitoxin biosynthetic gene is GntT or a fragment thereof.

The term “GntA gene” or “GntA” as used herein refer to the any of the recombinant or naturally-occurring forms of the GntA gene or variants or homologs thereof. In embodiments, the GntA gene codes for a GntA polypeptide capable of maintaining the activity of the GntA polypeptide (e.g., within at least 50%, 80%, 90%, 95%, 96%, 97%, 98%, 99% or 100% activity compared to GntA polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntA gene (e.g. SEQ ID NO:23-27).

In embodiments, the GntA gene is substantially identical to the nucleic acid sequence of SEQ ID NO:23 or a variant or homolog having substantial identity thereto. In embodiments, the GntA gene includes the nucleic acid sequence of SEQ ID NO:23. In embodiments, the GntA gene is the nucleic acid sequence of SEQ ID NO:23. In embodiments, the GntA gene is a portion of SEQ ID NO:23.

In embodiments, the GntA gene is substantially identical to the nucleic acid sequence of SEQ ID NO:24 or a variant or homolog having substantial identity thereto. In embodiments, the GntA gene includes the nucleic acid sequence of SEQ ID NO:24. In embodiments, the GntA gene is the nucleic acid sequence of SEQ ID NO:24. In embodiments, the GntA gene is a portion of SEQ ID NO:24.

In embodiments, the GntA gene is substantially identical to the nucleic acid sequence of SEQ ID NO:25 or a variant or homolog having substantial identity thereto. In embodiments, the GntA gene includes the nucleic acid sequence of SEQ ID NO:25. In embodiments, the GntA gene is the nucleic acid sequence of SEQ ID NO:25. In embodiments, the GntA gene is a portion of SEQ ID NO:25.

In embodiments, the GntA gene is substantially identical to the nucleic acid sequence of SEQ ID NO:26 or a variant or homolog having substantial identity thereto. In embodiments, the GntA gene includes the nucleic acid sequence of SEQ ID NO:26. In embodiments, the GntA gene is the nucleic acid sequence of SEQ ID NO:26. In embodiments, the GntA gene is a portion of SEQ ID NO:26.

In embodiments, the GntA gene is substantially identical to the nucleic acid sequence of SEQ ID NO:27 or a variant or homolog having substantial identity thereto. In embodiments, the GntA gene includes the nucleic acid sequence of SEQ ID NO:27. In embodiments, the GntA gene is the nucleic acid sequence of SEQ ID NO:27. In embodiments, the GntA gene is a portion of SEQ ID NO:27.

In embodiments, the GntA gene is about 50 nt to about 800 nt in length. In embodiments, the GntA gene is about 100 nt to about 800 nt in length. In embodiments, the GntA gene is about 150 nt to about 800 nt in length. In embodiments, the GntA gene is about 200 nt to about 800 nt in length. In embodiments, the GntA gene is about 250 nt to about 800 nt in length. In embodiments, the GntA gene is about 300 nt to about 800 nt in length. In embodiments, the GntA gene is about 350 nt to about 800 nt in length. In embodiments, the GntA gene is about 400 nt to about 800 nt in length. In embodiments, the GntA gene is about 450 nt to about 800 nt in length. In embodiments, the GntA gene is about 500 nt to about 800 nt in length. In embodiments, the GntA gene is about 550 nt to about 800 nt in length. In embodiments, the GntA gene is about 600 nt to about 800 nt in length. In embodiments, the GntA gene is about 650 nt to about 800 nt in length. In embodiments, the GntA gene is about 700 nt to about 800 nt in length. In embodiments, the GntA gene is about 750 nt to about 800 nt in length.

In embodiments, the GntA gene is about 50 nt to about 750 nt in length. In embodiments, the GntA gene is about 50 nt to about 700 nt in length. In embodiments, the GntA gene is about 50 nt to about 650 nt in length. In embodiments, the GntA gene is about 50 nt to about 600 nt in length. In embodiments, the GntA gene is about 50 nt to about 550 nt in length. In embodiments, the GntA gene is about 50 nt to about 500 nt in length. In embodiments, the GntA gene is about 50 nt to about 450 nt in length. In embodiments, the GntA gene is about 50 nt to about 400 nt in length. In embodiments, the GntA gene is about 50 nt to about 350 nt in length. In embodiments, the GntA gene is about 50 nt to about 300 nt in length. In embodiments, the GntA gene is about 50 nt to about 250 nt in length. In embodiments, the GntA gene is about 50 nt to about 200 nt in length. In embodiments, the GntA gene is about 50 nt to about 150 nt in length. In embodiments, the GntA gene is about 50 nt to about 100 nt in length. In embodiments, the GntA gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, or 800 nt in length. In embodiments, the GntA gene is about 804 nt in length. In embodiments, the GntA gene is 804 nt in length. In embodiments, the sequence lengths described herein include a fragment or a portion of the nucleic acid sequence of SEQ ID NO:23. In embodiments, the sequence lengths described herein include a fragment or a portion of the nucleic acid sequence of SEQ ID NO:24. In embodiments, the sequence lengths described herein include a fragment or a portion of the nucleic acid sequence of SEQ ID NO:25. In embodiments, the sequence lengths described herein include a fragment or a portion of the nucleic acid sequence of SEQ ID NO:26. In embodiments, the sequence lengths described herein include a fragment or a portion of the nucleic acid sequence of SEQ ID NO:27.

The term “GntB gene” or “GntB” as used herein refer to the any of the recombinant or naturally-occurring forms of the GntB gene or variants or homologs thereof. In embodiments, the GntB gene codes for a GntB polypeptide capable of maintaining the activity of the GntB polypeptide (e.g., within at least 50%, 80%, 90%, 95%, 96%, 97%, 98%, 99% or 100% activity compared to GntB polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntB gene (SEQ ID NO:28-32).

In embodiments, the GntB gene is substantially identical to the nucleic acid sequence of SEQ ID NO:28 or a variant or homolog having substantial identity thereto. In embodiments, the GntB gene includes the nucleic acid sequence of SEQ ID NO:28. In embodiments, the GntB gene is the nucleic acid sequence of SEQ ID NO:28. In embodiments, the GntB gene is a portion of SEQ ID NO:28.

In embodiments, the GntB gene is substantially identical to the nucleic acid sequence of SEQ ID NO:29 or a variant or homolog having substantial identity thereto. In embodiments, the GntB gene includes the nucleic acid sequence of SEQ ID NO:29. In embodiments, the GntB gene is the nucleic acid sequence of SEQ ID NO:29. In embodiments, the GntB gene is a portion of SEQ ID NO:29.

In embodiments, the GntB gene is substantially identical to the nucleic acid sequence of SEQ ID NO:30 or a variant or homolog having substantial identity thereto. In embodiments, the GntB gene includes the nucleic acid sequence of SEQ ID NO:30. In embodiments, the GntB gene is the nucleic acid sequence of SEQ ID NO:30. In embodiments, the GntB gene is a portion of SEQ ID NO:30.

In embodiments, the GntB gene is substantially identical to the nucleic acid sequence of SEQ ID NO:31 or a variant or homolog having substantial identity thereto. In embodiments, the GntB gene includes the nucleic acid sequence of SEQ ID NO:31. In embodiments, the GntB gene is the nucleic acid sequence of SEQ ID NO:31. In embodiments, the GntB gene is a portion of SEQ ID NO:31.

In embodiments, the GntB gene is substantially identical to the nucleic acid sequence of SEQ ID NO:32 or a variant or homolog having substantial identity thereto. In embodiments, the GntB gene includes the nucleic acid sequence of SEQ ID NO:32. In embodiments, the GntB gene is the nucleic acid sequence of SEQ ID NO:32. In embodiments, the GntB gene is a portion of SEQ ID NO:32.

In embodiments, the GntB gene is about 50 nt to about 1000 nt in length. In embodiments, the GntB gene is about 100 nt to about 1000 nt in length. In embodiments, the GntB gene is about 150 nt to about 1000 nt in length. In embodiments, the GntB gene is about 200 nt to about 1000 nt in length. In embodiments, the GntB gene is about 250 nt to about 1000 nt in length. In embodiments, the GntB gene is about 300 nt to about 1000 nt in length. In embodiments, the GntB gene is about 350 nt to about 1000 nt in length. In embodiments, the GntB gene is about 400 nt to about 1000 nt in length. In embodiments, the GntB gene is about 450 nt to about 1000 nt in length. In embodiments, the GntB gene is about 500 nt to about 1000 nt in length. In embodiments, the GntB gene is about 550 nt to about 1000 nt in length. In embodiments, the GntB gene is about 600 nt to about 1000 nt in length. In embodiments, the GntB gene is about 650 nt to about 1000 nt in length. In embodiments, the GntB gene is about 700 nt to about 1000 nt in length. In embodiments, the GntB gene is about 750 nt to about 1000 nt in length. In embodiments, the GntB gene is about 800 nt to about 1000 nt in length. In embodiments, the GntB gene is about 750 nt to about 1000 nt in length. In embodiments, the GntB gene is about 850 nt to about 1000 nt in length. In embodiments, the GntB gene is about 750 nt to about 1000 nt in length. In embodiments, the GntB gene is about 900 nt to about 1000 nt in length. In embodiments, the GntB gene is about 950 nt to about 1000 nt in length.

In embodiments, the GntB gene is about 50 nt to about 950 nt in length. In embodiments, the GntB gene is about 50 nt to about 900 nt in length. In embodiments, the GntB gene is about 50 nt to about 850 nt in length. In embodiments, the GntB gene is about 50 nt to about 800 nt in length. In embodiments, the GntB gene is about 50 nt to about 750 nt in length. In embodiments, the GntB gene is about 50 nt to about 700 nt in length. In embodiments, the GntB gene is about 50 nt to about 650 nt in length. In embodiments, the GntB gene is about 50 nt to about 600 nt in length. In embodiments, the GntB gene is about 50 nt to about 550 nt in length. In embodiments, the GntB gene is about 50 nt to about 500 nt in length. In embodiments, the GntB gene is about 50 nt to about 450 nt in length. In embodiments, the GntB gene is about 50 nt to about 400 nt in length. In embodiments, the GntB gene is about 50 nt to about 350 nt in length. In embodiments, the GntB gene is about 50 nt to about 300 nt in length. In embodiments, the GntB gene is about 50 nt to about 250 nt in length. In embodiments, the GntB gene is about 50 nt to about 200 nt in length. In embodiments, the GntB gene is about 50 nt to about 150 nt in length. In embodiments, the GntB gene is about 50 nt to about 100 nt in length. In embodiments, the GntB gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, or 1000 nt in length. In embodiments, the GntB gene is about 957 nt in length. In embodiments, the GntB gene is 957 nt in length. In embodiments, the sequence lengths described herein include a fragment or a portion of the nucleic acid sequence of SEQ ID NO:28. In embodiments, the sequence lengths described herein include a fragment or a portion of the nucleic acid sequence of SEQ ID NO:29. In embodiments, the sequence lengths described herein include a fragment or a portion of the nucleic acid sequence of SEQ ID NO:30. In embodiments, the sequence lengths described herein include a fragment or a portion of the nucleic acid sequence of SEQ ID NO:31. In embodiments, the sequence lengths described herein include a fragment or a portion of the nucleic acid sequence of SEQ ID NO:32.

The term “GntC gene” or “GntC” as used herein refer to the any of the recombinant or naturally-occurring forms of the GntC gene or variants or homologs thereof. In embodiments, the GntC gene codes for a GntC polypeptide capable of maintaining the activity of the GntC polypeptide (e.g., within at least 50%, 80%, 90%, 95%, 96%, 97%, 98%, 99% or 100% activity compared to GntC polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntC gene (e.g. SEQ ID NO:33-37).

In embodiments, the GntC gene is substantially identical to the nucleic acid sequence of SEQ ID NO:33 or a variant or homolog having substantial identity thereto. In embodiments, the GntC gene includes the nucleic acid sequence of SEQ ID NO:33. In embodiments, the GntC gene is the nucleic acid sequence of SEQ ID NO:33. In embodiments, the GntC gene is a portion of SEQ ID NO:33.

In embodiments, the GntC gene is substantially identical to the nucleic acid sequence of SEQ ID NO:34 or a variant or homolog having substantial identity thereto. In embodiments, the GntC gene includes the nucleic acid sequence of SEQ ID NO:34. In embodiments, the GntC gene is the nucleic acid sequence of SEQ ID NO:34. In embodiments, the GntC gene is a portion of SEQ ID NO:34.

In embodiments, the GntC gene is substantially identical to the nucleic acid sequence of SEQ ID NO:35 or a variant or homolog having substantial identity thereto. In embodiments, the GntC gene includes the nucleic acid sequence of SEQ ID NO:35. In embodiments, the GntC gene is the nucleic acid sequence of SEQ ID NO:35. In embodiments, the GntC gene is a portion of SEQ ID NO:35.

In embodiments, the GntC gene is substantially identical to the nucleic acid sequence of SEQ ID NO:36 or a variant or homolog having substantial identity thereto. In embodiments, the GntC gene includes the nucleic acid sequence of SEQ ID NO:36. In embodiments, the GntC gene is the nucleic acid sequence of SEQ ID NO:36. In embodiments, the GntC gene is a portion of SEQ ID NO:36.

In embodiments, the GntC gene is substantially identical to the nucleic acid sequence of SEQ ID NO:37 or a variant or homolog having substantial identity thereto. In embodiments, the GntC gene includes the nucleic acid sequence of SEQ ID NO:37. In embodiments, the GntC gene is the nucleic acid sequence of SEQ ID NO:37. In embodiments, the GntC gene is a portion of SEQ ID NO:37.

In embodiments, the GntC gene is about 50 nt to about 1100 nt in length. In embodiments, the GntC gene is about 100 nt to about 1100 nt in length. In embodiments, the GntC gene is about 150 nt to about 1100 nt in length. In embodiments, the GntC gene is about 200 nt to about 1100 nt in length. In embodiments, the GntC gene is about 250 nt to about 1100 nt in length. In embodiments, the GntC gene is about 300 nt to about 1100 nt in length. In embodiments, the GntC gene is about 350 nt to about 1100 nt in length. In embodiments, the GntC gene is about 400 nt to about 1100 nt in length. In embodiments, the GntC gene is about 450 nt to about 1100 nt in length. In embodiments, the GntC gene is about 500 nt to about 1100 nt in length. In embodiments, the GntC gene is about 550 nt to about 1100 nt in length. In embodiments, the GntC gene is about 600 nt to about 1100 nt in length. In embodiments, the GntC gene is about 650 nt to about 1100 nt in length. In embodiments, the GntC gene is about 700 nt to about 1100 nt in length. In embodiments, the GntC gene is about 750 nt to about 1100 nt in length. In embodiments, the GntC gene is about 800 nt to about 1100 nt in length. In embodiments, the GntC gene is about 750 nt to about 1100 nt in length. In embodiments, the GntC gene is about 850 nt to about 1100 nt in length. In embodiments, the GntC gene is about 750 nt to about 1100 nt in length. In embodiments, the GntC gene is about 900 nt to about 1100 nt in length. In embodiments, the GntC gene is about 950 nt to about 1100 nt in length. In embodiments, the GntC gene is about 1000 nt to about 1100 nt in length. In embodiments, the GntC gene is about 1050 nt to about 1100 nt in length.

In embodiments, the GntC gene is about 50 nt to about 1050 nt in length. In embodiments, the GntC gene is about 50 nt to about 1000 nt in length. In embodiments, the GntC gene is about 50 nt to about 950 nt in length. In embodiments, the GntC gene is about 50 nt to about 900 nt in length. In embodiments, the GntC gene is about 50 nt to about 850 nt in length. In embodiments, the GntC gene is about 50 nt to about 800 nt in length. In embodiments, the GntC gene is about 50 nt to about 750 nt in length. In embodiments, the GntC gene is about 50 nt to about 700 nt in length. In embodiments, the GntC gene is about 50 nt to about 650 nt in length. In embodiments, the GntC gene is about 50 nt to about 600 nt in length. In embodiments, the GntC gene is about 50 nt to about 550 nt in length. In embodiments, the GntC gene is about 50 nt to about 500 nt in length. In embodiments, the GntC gene is about 50 nt to about 450 nt in length. In embodiments, the GntC gene is about 50 nt to about 400 nt in length. In embodiments, the GntC gene is about 50 nt to about 350 nt in length. In embodiments, the GntC gene is about 50 nt to about 300 nt in length. In embodiments, the GntC gene is about 50 nt to about 250 nt in length. In embodiments, the GntC gene is about 50 nt to about 200 nt in length. In embodiments, the GntC gene is about 50 nt to about 150 nt in length. In embodiments, the GntC gene is about 50 nt to about 100 nt in length. In embodiments, the GntC gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1000 nt, 1050 nt, or 1100 nt in length. In embodiments, the GntC gene is about 1113 nt in length. In embodiments, the GntC gene is 1113 nt in length. In embodiments, the sequence lengths described herein are within nucleic acid sequence of SEQ ID NO:33. In embodiments, the sequence lengths described herein are within nucleic acid sequence of SEQ ID NO:34. In embodiments, the sequence lengths described herein are within nucleic acid sequence of SEQ ID NO:35. In embodiments, the sequence lengths described herein are within nucleic acid sequence of SEQ ID NO:36. In embodiments, the sequence lengths described herein are within nucleic acid sequence of SEQ ID NO:37.

The term “GntD gene” or “GntD” as used herein refer to the any of the recombinant or naturally-occurring forms of the GntD gene or variants or homologs thereof. In embodiments, the GntD gene codes for a GntD polypeptide capable of maintaining the activity of the GntD polypeptide (e.g., within at least 50%, 80%, 90%, 95%, 96%, 97%, 98%, 99% or 100% activity compared to GntD polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntD gene (e.g. SEQ ID NO:38-42).

In embodiments, the GntD gene is substantially identical to the nucleic acid sequence of SEQ ID NO:38 or a variant or homolog having substantial identity thereto. In embodiments, the GntD gene includes the nucleic acid sequence of SEQ ID NO:38. In embodiments, the GntD gene is the nucleic acid sequence of SEQ ID NO:38. In embodiments, the GntD gene is a portion of SEQ ID NO:38.

In embodiments, the GntD gene is substantially identical to the nucleic acid sequence of SEQ ID NO:39 or a variant or homolog having substantial identity thereto. In embodiments, the GntD gene includes the nucleic acid sequence of SEQ ID NO:39. In embodiments, the GntD gene is the nucleic acid sequence of SEQ ID NO:38. In embodiments, the GntD gene is a portion of SEQ ID NO:39.

In embodiments, the GntD gene is substantially identical to the nucleic acid sequence of SEQ ID NO:40 or a variant or homolog having substantial identity thereto. In embodiments, the GntD gene includes the nucleic acid sequence of SEQ ID NO:40. In embodiments, the GntD gene is the nucleic acid sequence of SEQ ID NO:40. In embodiments, the GntD gene is a portion of SEQ ID NO:40.

In embodiments, the GntD gene is substantially identical to the nucleic acid sequence of SEQ ID NO:41 or a variant or homolog having substantial identity thereto. In embodiments, the GntD gene includes the nucleic acid sequence of SEQ ID NO:41. In embodiments, the GntD gene is the nucleic acid sequence of SEQ ID NO:38. In embodiments, the GntD gene is a portion of SEQ ID NO:41.

In embodiments, the GntD gene is substantially identical to the nucleic acid sequence of SEQ ID NO:42 or a variant or homolog having substantial identity thereto. In embodiments, the GntD gene includes the nucleic acid sequence of SEQ ID NO:42. In embodiments, the GntD gene is the nucleic acid sequence of SEQ ID NO:42. In embodiments, the GntD gene is a portion of SEQ ID NO:42.

In embodiments, the GntD gene is about 50 nt to about 1050 nt in length. In embodiments, the GntD gene is about 100 nt to about 1050 nt in length. In embodiments, the GntD gene is about 150 nt to about 1050 nt in length. In embodiments, the GntD gene is about 200 nt to about 1050 nt in length. In embodiments, the GntD gene is about 250 nt to about 1050 nt in length. In embodiments, the GntD gene is about 300 nt to about 1050 nt in length. In embodiments, the GntD gene is about 350 nt to about 1050 nt in length. In embodiments, the GntD gene is about 400 nt to about 1050 nt in length. In embodiments, the GntD gene is about 450 nt to about 1050 nt in length. In embodiments, the GntD gene is about 500 nt to about 1050 nt in length. In embodiments, the GntD gene is about 550 nt to about 1050 nt in length. In embodiments, the GntD gene is about 600 nt to about 1050 nt in length. In embodiments, the GntD gene is about 650 nt to about 1050 nt in length. In embodiments, the GntD gene is about 700 nt to about 1050 nt in length. In embodiments, the GntD gene is about 750 nt to about 1050 nt in length. In embodiments, the GntD gene is about 800 nt to about 1050 nt in length. In embodiments, the GntD gene is about 750 nt to about 1050 nt in length. In embodiments, the GntD gene is about 850 nt to about 1050 nt in length. In embodiments, the GntD gene is about 750 nt to about 1050 nt in length. In embodiments, the GntD gene is about 900 nt to about 1050 nt in length. In embodiments, the GntD gene is about 950 nt to about 1050 nt in length. In embodiments, the GntD gene is about 1000 nt to about 1050 nt in length.

In embodiments, the GntD gene is about 50 nt to about 1000 nt in length. In embodiments, the GntD gene is about 50 nt to about 950 nt in length. In embodiments, the GntD gene is about 50 nt to about 900 nt in length. In embodiments, the GntD gene is about 50 nt to about 850 nt in length. In embodiments, the GntD gene is about 50 nt to about 800 nt in length. In embodiments, the GntD gene is about 50 nt to about 750 nt in length. In embodiments, the GntD gene is about 50 nt to about 700 nt in length. In embodiments, the GntD gene is about 50 nt to about 650 nt in length. In embodiments, the GntD gene is about 50 nt to about 600 nt in length. In embodiments, the GntD gene is about 50 nt to about 550 nt in length. In embodiments, the GntD gene is about 50 nt to about 500 nt in length. In embodiments, the GntD gene is about 50 nt to about 450 nt in length. In embodiments, the GntD gene is about 50 nt to about 400 nt in length. In embodiments, the GntD gene is about 50 nt to about 350 nt in length. In embodiments, the GntD gene is about 50 nt to about 300 nt in length. In embodiments, the GntD gene is about 50 nt to about 250 nt in length. In embodiments, the GntD gene is about 50 nt to about 200 nt in length. In embodiments, the GntD gene is about 50 nt to about 150 nt in length. In embodiments, the GntD gene is about 50 nt to about 100 nt in length. In embodiments, the GntD gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1000 nt, or 1050 nt in length. In embodiments, the GntD gene is about 1044 nt in length. In embodiments, the GntD gene is 1044 nt in length.

In embodiments, the sequence lengths described herein are within nucleic acid sequence of SEQ ID NO:38. In embodiments, the sequence lengths described herein are within nucleic acid sequence of SEQ ID NO:39. In embodiments, the sequence lengths described herein are within nucleic acid sequence of SEQ ID NO:40. In embodiments, the sequence lengths described herein are within nucleic acid sequence of SEQ ID NO:41. In embodiments, the sequence lengths described herein are within nucleic acid sequence of SEQ ID NO:42.

The term “GntE gene” or “GntE” as used herein refer to the any of the recombinant or naturally-occurring forms of the GntE gene or variants or homologs thereof. In embodiments, the GntE gene codes for a GntE polypeptide capable of maintaining the activity of the GntE polypeptide (e.g., within at least 50%, 80%, 90%, 95%, 96%, 97%, 98%, 99% or 100% activity compared to GntE polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntE gene (e.g. SEQ ID NO:43-47).

In embodiments, the GntE gene is substantially identical to the nucleic acid sequence of SEQ ID NO:43 or a variant or homolog having substantial identity thereto. In embodiments, the GntE gene includes the nucleic acid sequence of SEQ ID NO:43. In embodiments, the GntE gene is the nucleic acid sequence of SEQ ID NO:43. In embodiments, the GntE gene is a portion of SEQ ID NO:43.

In embodiments, the GntE gene is substantially identical to the nucleic acid sequence of SEQ ID NO:44 or a variant or homolog having substantial identity thereto. In embodiments, the GntE gene includes the nucleic acid sequence of SEQ ID NO:44. In embodiments, the GntE gene is the nucleic acid sequence of SEQ ID NO:44. In embodiments, the GntE gene is a portion of SEQ ID NO:44.

In embodiments, the GntE gene is substantially identical to the nucleic acid sequence of SEQ ID NO:45 or a variant or homolog having substantial identity thereto. In embodiments, the GntE gene includes the nucleic acid sequence of SEQ ID NO:45. In embodiments, the GntE gene is the nucleic acid sequence of SEQ ID NO:45. In embodiments, the GntE gene is a portion of SEQ ID NO:45.

In embodiments, the GntE gene is substantially identical to the nucleic acid sequence of SEQ ID NO:46 or a variant or homolog having substantial identity thereto. In embodiments, the GntE gene includes the nucleic acid sequence of SEQ ID NO:46. In embodiments, the GntE gene is the nucleic acid sequence of SEQ ID NO:46. In embodiments, the GntE gene is a portion of SEQ ID NO:46.

In embodiments, the GntE gene is substantially identical to the nucleic acid sequence of SEQ ID NO:47 or a variant or homolog having substantial identity thereto. In embodiments, the GntE gene includes the nucleic acid sequence of SEQ ID NO:47. In embodiments, the GntE gene is the nucleic acid sequence of SEQ ID NO:47. In embodiments, the GntE gene is a portion of SEQ ID NO:47.

In embodiments, the GntE gene is about 50 nt to about 1300 nt in length. In embodiments, the GntE gene is about 100 nt to about 1300 nt in length. In embodiments, the GntE gene is about 150 nt to about 1300 nt in length. In embodiments, the GntE gene is about 200 nt to about 1300 nt in length. In embodiments, the GntE gene is about 250 nt to about 1300 nt in length. In embodiments, the GntE gene is about 300 nt to about 1300 nt in length.

In embodiments, the GntE gene is about 350 nt to about 1300 nt in length. In embodiments, the GntE gene is about 400 nt to about 1300 nt in length. In embodiments, the GntE gene is about 450 nt to about 1300 nt in length. In embodiments, the GntE gene is about 500 nt to about 1300 nt in length. In embodiments, the GntE gene is about 550 nt to about 1300 nt in length. In embodiments, the GntE gene is about 600 nt to about 1300 nt in length. In embodiments, the GntE gene is about 650 nt to about 1300 nt in length. In embodiments, the GntE gene is about 700 nt to about 1300 nt in length. In embodiments, the GntE gene is about 750 nt to about 1300 nt in length. In embodiments, the GntE gene is about 800 nt to about 1300 nt in length. In embodiments, the GntE gene is about 750 nt to about 1300 nt in length. In embodiments, the GntE gene is about 850 nt to about 1300 nt in length. In embodiments, the GntE gene is about 750 nt to about 1300 nt in length. In embodiments, the GntE gene is about 900 nt to about 1300 nt in length. In embodiments, the GntE gene is about 950 nt to about 1300 nt in length. In embodiments, the GntE gene is about 1000 nt to about 1300 nt in length. In embodiments, the GntE gene is about 1050 nt to about 1300 nt in length. In embodiments, the GntE gene is about 1100 nt to about 1300 nt in length. In embodiments, the GntE gene is about 1150 nt to about 1300 nt in length. In embodiments, the GntE gene is about 1200 nt to about 1300 nt in length. In embodiments, the GntE gene is about 1250 nt to about 1300 nt in length.

In embodiments, the GntE gene is about 50 nt to about 1250 nt in length. In embodiments, the GntE gene is about 50 nt to about 1200 nt in length. In embodiments, the GntE gene is about 50 nt to about 1150 nt in length. In embodiments, the GntE gene is about 50 nt to about 1100 nt in length. In embodiments, the GntE gene is about 50 nt to about 1050 nt in length. In embodiments, the GntE gene is about 50 nt to about 1000 nt in length. In embodiments, the GntE gene is about 50 nt to about 950 nt in length. In embodiments, the GntE gene is about 50 nt to about 900 nt in length. In embodiments, the GntE gene is about 50 nt to about 850 nt in length. In embodiments, the GntE gene is about 50 nt to about 800 nt in length. In embodiments, the GntE gene is about 50 nt to about 750 nt in length. In embodiments, the GntE gene is about 50 nt to about 700 nt in length. In embodiments, the GntE gene is about 50 nt to about 650 nt in length. In embodiments, the GntE gene is about 50 nt to about 600 nt in length. In embodiments, the GntE gene is about 50 nt to about 550 nt in length. In embodiments, the GntE gene is about 50 nt to about 500 nt in length. In embodiments, the GntE gene is about 50 nt to about 450 nt in length. In embodiments, the GntE gene is about 50 nt to about 400 nt in length. In embodiments, the GntE gene is about 50 nt to about 350 nt in length. In embodiments, the GntE gene is about 50 nt to about 300 nt in length. In embodiments, the GntE gene is about 50 nt to about 250 nt in length. In embodiments, the GntE gene is about 50 nt to about 200 nt in length. In embodiments, the GntE gene is about 50 nt to about 150 nt in length. In embodiments, the GntE gene is about 50 nt to about 100 nt in length. In embodiments, the GntE gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1000 nt, 1050 nt, 1100 nt, 1150 nt, 1200 nt, 1250 nt, or 1300 nt in length. In embodiments, the GntE gene is about 1311 nt in length. In embodiments, the GntE gene is 1311 nt in length. In embodiments, the sequence lengths described herein are within nucleic acid sequence of SEQ ID NO:43. In embodiments, the sequence lengths described herein are within nucleic acid sequence of SEQ ID NO:44. In embodiments, the sequence lengths described herein are within nucleic acid sequence of SEQ ID NO:45. In embodiments, the sequence lengths described herein are within nucleic acid sequence of SEQ ID NO:46. In embodiments, the sequence lengths described herein are within nucleic acid sequence of SEQ ID NO:47.

The term “GntF gene” or “GntF” as used herein refer to the any of the recombinant or naturally-occurring forms of the GntF gene or variants or homologs thereof. In embodiments, the GntF gene codes for a GntF polypeptide capable of maintaining the activity of the GntF polypeptide (e.g., within at least 50%, 80%, 90%, 95%, 96%, 97%, 98%, 99% or 100% activity compared to GntF polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntF gene (e.g. SEQ ID NO:48-52).

In embodiments, the GntF gene is substantially identical to the nucleic acid sequence of SEQ ID NO:48 or a variant or homolog having substantial identity thereto. In embodiments, the GntF gene includes the nucleic acid sequence of SEQ ID NO:48. In embodiments, the GntF gene is the nucleic acid sequence of SEQ ID NO:48. In embodiments, the GntF gene is a portion of SEQ ID NO:48.

In embodiments, the GntF gene is substantially identical to the nucleic acid sequence of SEQ ID NO:49 or a variant or homolog having substantial identity thereto. In embodiments, the GntF gene includes the nucleic acid sequence of SEQ ID NO:49. In embodiments, the GntF gene is the nucleic acid sequence of SEQ ID NO:49. In embodiments, the GntF gene is a portion of SEQ ID NO:49.

In embodiments, the GntF gene is substantially identical to the nucleic acid sequence of SEQ ID NO:50 or a variant or homolog having substantial identity thereto. In embodiments, the GntF gene includes the nucleic acid sequence of SEQ ID NO:50. In embodiments, the GntF gene is the nucleic acid sequence of SEQ ID NO:50. In embodiments, the GntF gene is a portion of SEQ ID NO:50.

In embodiments, the GntF gene is substantially identical to the nucleic acid sequence of SEQ ID NO:51 or a variant or homolog having substantial identity thereto. In embodiments, the GntF gene includes the nucleic acid sequence of SEQ ID NO:51. In embodiments, the GntF gene is the nucleic acid sequence of SEQ ID NO:51. In embodiments, the GntF gene is a portion of SEQ ID NO:51.

In embodiments, the GntF gene is substantially identical to the nucleic acid sequence of SEQ ID NO:52 or a variant or homolog having substantial identity thereto. In embodiments, the GntF gene includes the nucleic acid sequence of SEQ ID NO:52. In embodiments, the GntF gene is the nucleic acid sequence of SEQ ID NO:52. In embodiments, the GntF gene is a portion of SEQ ID NO:52.

In embodiments, the GntF gene is about 50 nt to about 800 nt in length. In embodiments, the GntF gene is about 100 nt to about 800 nt in length. In embodiments, the GntF gene is about 150 nt to about 800 nt in length. In embodiments, the GntF gene is about 200 nt to about 800 nt in length. In embodiments, the GntF gene is about 250 nt to about 800 nt in length. In embodiments, the GntF gene is about 330 nt to about 800 nt in length. In embodiments, the GntF gene is about 350 nt to about 800 nt in length. In embodiments, the GntF gene is about 400 nt to about 800 nt in length. In embodiments, the GntF gene is about 450 nt to about 800 nt in length. In embodiments, the GntF gene is about 500 nt to about 800 nt in length. In embodiments, the GntF gene is about 550 nt to about 800 nt in length. In embodiments, the GntF gene is about 600 nt to about 800 nt in length. In embodiments, the GntF gene is about 650 nt to about 800 nt in length. In embodiments, the GntF gene is about 700 nt to about 800 nt in length. In embodiments, the GntF gene is about 750 nt to about 800 nt in length.

In embodiments, the GntF gene is about 50 nt to about 750 nt in length. In embodiments, the GntF gene is about 50 nt to about 700 nt in length. In embodiments, the GntF gene is about 50 nt to about 650 nt in length. In embodiments, the GntF gene is about 50 nt to about 600 nt in length. In embodiments, the GntF gene is about 50 nt to about 550 nt in length. In embodiments, the GntF gene is about 50 nt to about 500 nt in length. In embodiments, the GntF gene is about 50 nt to about 450 nt in length. In embodiments, the GntF gene is about 50 nt to about 400 nt in length. In embodiments, the GntF gene is about 50 nt to about 350 nt in length. In embodiments, the GntF gene is about 50 nt to about 330 nt in length. In embodiments, the GntF gene is about 50 nt to about 250 nt in length. In embodiments, the GntF gene is about 50 nt to about 200 nt in length. In embodiments, the GntF gene is about 50 nt to about 150 nt in length. In embodiments, the GntF gene is about 50 nt to about 100 nt in length. In embodiments, the GntF gene is about 50 nt, 100 nt, 150 nt, 200 nt, 250 nt, 330 nt, 350 nt, 400 nt, 450 nt, 500 nt, 550 nt, 600 nt, 650 nt, 700 nt, 750 nt, or 800 nt in length. In embodiments, the GntF gene is about 825 nt in length. In embodiments, the GntF gene is 825 nt in length. In embodiments, the sequence lengths described herein are within nucleic acid sequence of SEQ ID NO:48. In embodiments, the sequence lengths described herein are within nucleic acid sequence of SEQ ID NO:49. In embodiments, the sequence lengths described herein are within nucleic acid sequence of SEQ ID NO:50. In embodiments, the sequence lengths described herein are within nucleic acid sequence of SEQ ID NO:51. In embodiments, the sequence lengths described herein are within nucleic acid sequence of SEQ ID NO:52.

The term “GntG gene” or “GntG” as used herein refer to the any of the recombinant or naturally-occurring forms of the GntG gene or variants or homologs thereof. In embodiments, the GntG gene codes for a GntG polypeptide capable of maintaining the activity of the GntG polypeptide (e.g., within at least 50%, 80%, 90%, 95%, 96%, 97%, 98%, 99% or 100% activity compared to GntG polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntG gene (e.g. SEQ ID NO:53-57).

In embodiments, the GntG gene is substantially identical to the nucleic acid sequence of SEQ ID NO:53 or a variant or homolog having substantial identity thereto. In embodiments, the GntG gene includes the nucleic acid sequence of SEQ ID NO:53. In embodiments, the GntG gene is the nucleic acid sequence of SEQ ID NO:53. In embodiments, the GntG gene is a portion of SEQ ID NO:53.

In embodiments, the GntG gene is substantially identical to the nucleic acid sequence of SEQ ID NO:54 or a variant or homolog having substantial identity thereto. In embodiments, the GntG gene includes the nucleic acid sequence of SEQ ID NO:54. In embodiments, the GntG gene is the nucleic acid sequence of SEQ ID NO:54. In embodiments, the GntG gene is a portion of SEQ ID NO:54.

In embodiments, the GntG gene is substantially identical to the nucleic acid sequence of SEQ ID NO:55 or a variant or homolog having substantial identity thereto. In embodiments, the GntG gene includes the nucleic acid sequence of SEQ ID NO:55. In embodiments, the GntG gene is the nucleic acid sequence of SEQ ID NO:55. In embodiments, the GntG gene is a portion of SEQ ID NO:55.

In embodiments, the GntG gene is substantially identical to the nucleic acid sequence of SEQ ID NO:56 or a variant or homolog having substantial identity thereto. In embodiments, the GntG gene includes the nucleic acid sequence of SEQ ID NO:56. In embodiments, the GntG gene is the nucleic acid sequence of SEQ ID NO:56. In embodiments, the GntG gene is a portion of SEQ ID NO:56.

In embodiments, the GntG gene is substantially identical to the nucleic acid sequence of SEQ ID NO:57 or a variant or homolog having substantial identity thereto. In embodiments, the GntG gene includes the nucleic acid sequence of SEQ ID NO:57. In embodiments, the GntG gene is the nucleic acid sequence of SEQ ID NO:57. In embodiments, the GntG gene is a portion of SEQ ID NO:57.

In embodiments, the GntG gene is about 50 nt to about 1000 nt in length. In embodiments, the GntG gene is about 100 nt to about 1000 nt in length. In embodiments, the GntG gene is about 150 nt to about 1000 nt in length. In embodiments, the GntG gene is about 200 nt to about 1000 nt in length. In embodiments, the GntG gene is about 250 nt to about 1000 nt in length. In embodiments, the GntG gene is about 300 nt to about 1000 nt in length. In embodiments, the GntG gene is about 350 nt to about 1000 nt in length. In embodiments, the GntG gene is about 400 nt to about 1000 nt in length. In embodiments, the GntG gene is about 450 nt to about 1000 nt in length. In embodiments, the GntG gene is about 500 nt to about 1000 nt in length. In embodiments, the GntG gene is about 550 nt to about 1000 nt in length. In embodiments, the GntG gene is about 600 nt to about 1000 nt in length. In embodiments, the GntG gene is about 650 nt to about 1000 nt in length. In embodiments, the GntG gene is about 700 nt to about 1000 nt in length. In embodiments, the GntG gene is about 750 nt to about 1000 nt in length. In embodiments, the GntG gene is about 800 nt to about 1000 nt in length. In embodiments, the GntG gene is about 750 nt to about 1000 nt in length. In embodiments, the GntG gene is about 850 nt to about 1000 nt in length. In embodiments, the GntG gene is about 750 nt to about 1000 nt in length. In embodiments, the GntG gene is about 900 nt to about 1000 nt in length. In embodiments, the GntG gene is about 950 nt to about 1000 nt in length.

In embodiments, the GntG gene is about 50 nt to about 950 nt in length. In embodiments, the GntG gene is about 50 nt to about 900 nt in length. In embodiments, the GntG gene is about 50 nt to about 850 nt in length. In embodiments, the GntG gene is about 50 nt to about 800 nt in length. In embodiments, the GntG gene is about 50 nt to about 750 nt in length. In embodiments, the GntG gene is about 50 nt to about 700 nt in length. In embodiments, the GntG gene is about 50 nt to about 650 nt in length. In embodiments, the GntG gene is about 50 nt to about 600 nt in length. In embodiments, the GntG gene is about 50 nt to about 550 nt in length. In embodiments, the GntG gene is about 50 nt to about 500 nt in length. In embodiments, the GntG gene is about 50 nt to about 450 nt in length. In embodiments, the GntG gene is about 50 nt to about 400 nt in length. In embodiments, the GntG gene is about 50 nt to about 350 nt in length. In embodiments, the GntG gene is about 50 nt to about 300 nt in length. In embodiments, the GntG gene is about 50 nt to about 250 nt in length. In embodiments, the GntG gene is about 50 nt to about 200 nt in length. In embodiments, the GntG gene is about 50 nt to about 150 nt in length. In embodiments, the GntG gene is about 50 nt to about 100 nt in length. In embodiments, the GntG gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, or 1000 nt in length. In embodiments, the GntG gene is about 1035 nt in length. In embodiments, the GntG gene is 1035 nt in length. In embodiments, the sequence lengths described herein are within nucleic acid sequence of SEQ ID NO:53. In embodiments, the sequence lengths described herein are within nucleic acid sequence of SEQ ID NO:54. In embodiments, the sequence lengths described herein are within nucleic acid sequence of SEQ ID NO:55. In embodiments, the sequence lengths described herein are within nucleic acid sequence of SEQ ID NO:56. In embodiments, the sequence lengths described herein are within nucleic acid sequence of SEQ ID NO:57.

The term “GntH gene” or “GntH” as used herein refer to the any of the recombinant or naturally-occurring forms of the GntH gene or variants or homologs thereof. In embodiments, the GntH gene codes for a GntH polypeptide capable of maintaining the activity of the GntH polypeptide (e.g., within at least 50%, 80%, 90%, 95%, 96%, 97%, 98%, 99% or 100% activity compared to GntH polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntH gene (e.g. SEQ ID NO:58-62).

In embodiments, the GntH gene is substantially identical to the nucleic acid sequence of SEQ ID NO:58 or a variant or homolog having substantial identity thereto. In embodiments, the GntH gene includes the nucleic acid sequence of SEQ ID NO:58. In embodiments, the GntH gene is the nucleic acid sequence of SEQ ID NO:58. In embodiments, the GntH gene is a portion of SEQ ID NO:58.

In embodiments, the GntH gene is substantially identical to the nucleic acid sequence of SEQ ID NO:59 or a variant or homolog having substantial identity thereto. In embodiments, the GntH gene includes the nucleic acid sequence of SEQ ID NO:59. In embodiments, the GntH gene is the nucleic acid sequence of SEQ ID NO:59. In embodiments, the GntH gene is a portion of SEQ ID NO:59.

In embodiments, the GntH gene is substantially identical to the nucleic acid sequence of SEQ ID NO:60 or a variant or homolog having substantial identity thereto. In embodiments, the GntH gene includes the nucleic acid sequence of SEQ ID NO:60. In embodiments, the GntH gene is the nucleic acid sequence of SEQ ID NO:60. In embodiments, the GntH gene is a portion of SEQ ID NO:60.

In embodiments, the GntH gene is substantially identical to the nucleic acid sequence of SEQ ID NO:61 or a variant or homolog having substantial identity thereto. In embodiments, the GntH gene includes the nucleic acid sequence of SEQ ID NO:61. In embodiments, the GntH gene is the nucleic acid sequence of SEQ ID NO:61. In embodiments, the GntH gene is a portion of SEQ ID NO:61.

In embodiments, the GntH gene is substantially identical to the nucleic acid sequence of SEQ ID NO:62 or a variant or homolog having substantial identity thereto. In embodiments, the GntH gene includes the nucleic acid sequence of SEQ ID NO:62. In embodiments, the GntH gene is the nucleic acid sequence of SEQ ID NO:62. In embodiments, the GntH gene is a portion of SEQ ID NO:62.

In embodiments, the GntH gene is about 50 nt to about 1200 nt in length. In embodiments, the GntH gene is about 100 nt to about 1200 nt in length. In embodiments, the GntH gene is about 150 nt to about 1200 nt in length. In embodiments, the GntH gene is about 200 nt to about 1200 nt in length. In embodiments, the GntH gene is about 250 nt to about 1200 nt in length. In embodiments, the GntH gene is about 300 nt to about 1200 nt in length. In embodiments, the GntH gene is about 350 nt to about 1200 nt in length. In embodiments, the GntH gene is about 400 nt to about 1200 nt in length. In embodiments, the GntH gene is about 450 nt to about 1200 nt in length. In embodiments, the GntH gene is about 500 nt to about 1200 nt in length. In embodiments, the GntH gene is about 550 nt to about 1200 nt in length. In embodiments, the GntH gene is about 600 nt to about 1200 nt in length. In embodiments, the GntH gene is about 650 nt to about 1200 nt in length. In embodiments, the GntH gene is about 700 nt to about 1200 nt in length. In embodiments, the GntH gene is about 750 nt to about 1200 nt in length. In embodiments, the GntH gene is about 800 nt to about 1200 nt in length. In embodiments, the GntH gene is about 750 nt to about 1200 nt in length. In embodiments, the GntH gene is about 850 nt to about 1200 nt in length. In embodiments, the GntH gene is about 750 nt to about 1200 nt in length. In embodiments, the GntH gene is about 900 nt to about 1200 nt in length. In embodiments, the GntH gene is about 950 nt to about 1200 nt in length. In embodiments, the GntH gene is about 1000 nt to about 1200 nt in length. In embodiments, the GntH gene is about 1050 nt to about 1200 nt in length. In embodiments, the GntH gene is about 1100 nt to about 1200 nt in length. In embodiments, the GntH gene is about 1150 nt to about 1200 nt in length.

In embodiments, the GntH gene is about 50 nt to about 1150 nt in length. In embodiments, the GntH gene is about 50 nt to about 1100 nt in length. In embodiments, the GntH gene is about 50 nt to about 1050 nt in length. In embodiments, the GntH gene is about 50 nt to about 1000 nt in length. In embodiments, the GntH gene is about 50 nt to about 950 nt in length. In embodiments, the GntH gene is about 50 nt to about 900 nt in length. In embodiments, the GntH gene is about 50 nt to about 850 nt in length. In embodiments, the GntH gene is about 50 nt to about 800 nt in length. In embodiments, the GntH gene is about 50 nt to about 750 nt in length. In embodiments, the GntH gene is about 50 nt to about 700 nt in length. In embodiments, the GntH gene is about 50 nt to about 650 nt in length. In embodiments, the GntH gene is about 50 nt to about 600 nt in length. In embodiments, the GntH gene is about 50 nt to about 550 nt in length. In embodiments, the GntH gene is about 50 nt to about 500 nt in length. In embodiments, the GntH gene is about 50 nt to about 450 nt in length. In embodiments, the GntH gene is about 50 nt to about 400 nt in length. In embodiments, the GntH gene is about 50 nt to about 350 nt in length. In embodiments, the GntH gene is about 50 nt to about 300 nt in length. In embodiments, the GntH gene is about 50 nt to about 250 nt in length. In embodiments, the GntH gene is about 50 nt to about 200 nt in length. In embodiments, the GntH gene is about 50 nt to about 150 nt in length. In embodiments, the GntH gene is about 50 nt to about 100 nt in length. In embodiments, the GntH gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1000 nt, 1050 nt, 1100 nt, 1150 nt, 1200 nt in length. In embodiments, the GntH gene is about 1242 nt in length. In embodiments, the GntH gene is 1242 nt in length. In embodiments, the sequence lengths described herein are within nucleic acid sequence of SEQ ID NO:58. In embodiments, the sequence lengths described herein are within nucleic acid sequence of SEQ ID NO:59. In embodiments, the sequence lengths described herein are within nucleic acid sequence of SEQ ID NO:60. In embodiments, the sequence lengths described herein are within nucleic acid sequence of SEQ ID NO:61. In embodiments, the sequence lengths described herein are within nucleic acid sequence of SEQ ID NO:62.

The term “GntI gene” or “GntI” as used herein refer to the any of the recombinant or naturally-occurring forms of the GntI gene or variants or homologs thereof. In embodiments, the GntI gene codes for a GntI polypeptide capable of maintaining the activity of the GntI polypeptide (e.g., within at least 50%, 80%, 90%, 95%, 96%, 97%, 98%, 99% or 100% activity compared to GntI polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntI gene (e.g. SEQ ID NO:63-67).

In embodiments, the GntI gene is substantially identical to the nucleic acid sequence of SEQ ID NO:63 or a variant or homolog having substantial identity thereto. In embodiments, the GntI gene includes the nucleic acid sequence of SEQ ID NO:63. In embodiments, the GntI gene is the nucleic acid sequence of SEQ ID NO:63. In embodiments, the GntI gene is a portion of SEQ ID NO:63.

In embodiments, the GntI gene is substantially identical to the nucleic acid sequence of SEQ ID NO:64 or a variant or homolog having substantial identity thereto. In embodiments, the GntI gene includes the nucleic acid sequence of SEQ ID NO:64. In embodiments, the GntI gene is the nucleic acid sequence of SEQ ID NO:64. In embodiments, the GntI gene is a portion of SEQ ID NO:64.

In embodiments, the GntI gene is substantially identical to the nucleic acid sequence of SEQ ID NO:65 or a variant or homolog having substantial identity thereto. In embodiments, the GntI gene includes the nucleic acid sequence of SEQ ID NO:65. In embodiments, the GntI gene is the nucleic acid sequence of SEQ ID NO:65. In embodiments, the GntI gene is a portion of SEQ ID NO:65.

In embodiments, the GntI gene is substantially identical to the nucleic acid sequence of SEQ ID NO:66 or a variant or homolog having substantial identity thereto. In embodiments, the GntI gene includes the nucleic acid sequence of SEQ ID NO:66. In embodiments, the GntI gene is the nucleic acid sequence of SEQ ID NO:66. In embodiments, the GntI gene is a portion of SEQ ID NO:66.

In embodiments, the GntI gene is substantially identical to the nucleic acid sequence of SEQ ID NO:67 or a variant or homolog having substantial identity thereto. In embodiments, the GntI gene includes the nucleic acid sequence of SEQ ID NO:67. In embodiments, the GntI gene is the nucleic acid sequence of SEQ ID NO:67. In embodiments, the GntI gene is a portion of SEQ ID NO:67.

In embodiments, the GntI gene is about 50 nt to about 900 nt in length. In embodiments, the GntI gene is about 100 nt to about 900 nt in length. In embodiments, the GntI gene is about 150 nt to about 900 nt in length. In embodiments, the GntI gene is about 200 nt to about 900 nt in length. In embodiments, the GntI gene is about 250 nt to about 900 nt in length. In embodiments, the GntI gene is about 300 nt to about 900 nt in length. In embodiments, the GntI gene is about 350 nt to about 900 nt in length. In embodiments, the GntI gene is about 400 nt to about 900 nt in length. In embodiments, the GntI gene is about 450 nt to about 900 nt in length. In embodiments, the GntI gene is about 500 nt to about 900 nt in length. In embodiments, the GntI gene is about 550 nt to about 900 nt in length. In embodiments, the GntI gene is about 600 nt to about 900 nt in length. In embodiments, the GntI gene is about 650 nt to about 900 nt in length. In embodiments, the GntI gene is about 700 nt to about 900 nt in length. In embodiments, the GntI gene is about 750 nt to about 900 nt in length. In embodiments, the GntI gene is about 800 nt to about 900 nt in length. In embodiments, the GntI gene is about 850 nt to about 900 nt in length.

In embodiments, the GntI gene is about 50 nt to about 850 nt in length. In embodiments, the GntI gene is about 50 nt to about 800 nt in length. In embodiments, the GntI gene is about 50 nt to about 750 nt in length. In embodiments, the GntI gene is about 50 nt to about 700 nt in length. In embodiments, the GntI gene is about 50 nt to about 650 nt in length. In embodiments, the GntI gene is about 50 nt to about 600 nt in length. In embodiments, the GntI gene is about 50 nt to about 550 nt in length. In embodiments, the GntI gene is about 50 nt to about 500 nt in length. In embodiments, the GntI gene is about 50 nt to about 450 nt in length. In embodiments, the GntI gene is about 50 nt to about 400 nt in length. In embodiments, the GntI gene is about 50 nt to about 350 nt in length. In embodiments, the GntI gene is about 50 nt to about 300 nt in length. In embodiments, the GntI gene is about 50 nt to about 250 nt in length. In embodiments, the GntI gene is about 50 nt to about 200 nt in length. In embodiments, the GntI gene is about 50 nt to about 150 nt in length. In embodiments, the GntI gene is about 50 nt to about 100 nt in length. In embodiments, the GntI gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, or 900 nt in length. In embodiments, the GntI gene is about 918 nt in length. In embodiments, the GntI gene is 918 nt in length. In embodiments, the sequence lengths described herein are within nucleic acid sequence of SEQ ID NO:63. In embodiments, the sequence lengths described herein are within nucleic acid sequence of SEQ ID NO:64. In embodiments, the sequence lengths described herein are within nucleic acid sequence of SEQ ID NO:65. In embodiments, the sequence lengths described herein are within nucleic acid sequence of SEQ ID NO:66. In embodiments, the sequence lengths described herein are within nucleic acid sequence of SEQ ID NO:67.

The term “GntJ gene” or “GntJ” as used herein refer to the any of the recombinant or naturally-occurring forms of the GntJ gene or variants or homologs thereof. In embodiments, the GntJ gene codes for a GntJ polypeptide capable of maintaining the activity of the GntJ polypeptide (e.g., within at least 50%, 80%, 90%, 95%, 96%, 97%, 98%, 99% or 100% activity compared to GntJ polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntJ gene (e.g. SEQ ID NO:68-72).

In embodiments, the GntJ gene is substantially identical to the nucleic acid sequence of SEQ ID NO:68 or a variant or homolog having substantial identity thereto. In embodiments, the GntJ gene includes the nucleic acid sequence of SEQ ID NO:68. In embodiments, the GntJ gene is the nucleic acid sequence of SEQ ID NO:68. In embodiments, the GntJ gene is a portion of SEQ ID NO:68.

In embodiments, the GntJ gene is substantially identical to the nucleic acid sequence of SEQ ID NO:69 or a variant or homolog having substantial identity thereto. In embodiments, the GntJ gene includes the nucleic acid sequence of SEQ ID NO:69. In embodiments, the GntJ gene is the nucleic acid sequence of SEQ ID NO:69. In embodiments, the GntJ gene is a portion of SEQ ID NO:69.

In embodiments, the GntJ gene is substantially identical to the nucleic acid sequence of SEQ ID NO:70 or a variant or homolog having substantial identity thereto. In embodiments, the GntJ gene includes the nucleic acid sequence of SEQ ID NO:70. In embodiments, the GntJ gene is the nucleic acid sequence of SEQ ID NO:70. In embodiments, the GntJ gene is a portion of SEQ ID NO:70.

In embodiments, the GntJ gene is substantially identical to the nucleic acid sequence of SEQ ID NO:71 or a variant or homolog having substantial identity thereto. In embodiments, the GntJ gene includes the nucleic acid sequence of SEQ ID NO:71. In embodiments, the GntJ gene is the nucleic acid sequence of SEQ ID NO:71. In embodiments, the GntJ gene is a portion of SEQ ID NO:71.

In embodiments, the GntJ gene is substantially identical to the nucleic acid sequence of SEQ ID NO:72 or a variant or homolog having substantial identity thereto. In embodiments, the GntJ gene includes the nucleic acid sequence of SEQ ID NO:72. In embodiments, the GntJ gene is the nucleic acid sequence of SEQ ID NO:72. In embodiments, the GntJ gene is a portion of SEQ ID NO:72.

In embodiments, the GntJ gene is about 50 nt to about 750 nt in length. In embodiments, the GntJ gene is about 100 nt to about 750 nt in length. In embodiments, the GntJ gene is about 150 nt to about 750 nt in length. In embodiments, the GntJ gene is about 200 nt to about 750 nt in length. In embodiments, the GntJ gene is about 250 nt to about 750 nt in length. In embodiments, the GntJ gene is about 330 nt to about 750 nt in length. In embodiments, the GntJ gene is about 350 nt to about 750 nt in length. In embodiments, the GntJ gene is about 400 nt to about 750 nt in length. In embodiments, the GntJ gene is about 450 nt to about 750 nt in length. In embodiments, the GntJ gene is about 500 nt to about 750 nt in length. In embodiments, the GntJ gene is about 550 nt to about 750 nt in length. In embodiments, the GntJ gene is about 600 nt to about 750 nt in length. In embodiments, the GntJ gene is about 650 nt to about 750 nt in length. In embodiments, the GntJ gene is about 700 nt to about 750 nt in length.

In embodiments, the GntJ gene is about 50 nt to about 700 nt in length. In embodiments, the GntJ gene is about 50 nt to about 650 nt in length. In embodiments, the GntJ gene is about 50 nt to about 600 nt in length. In embodiments, the GntJ gene is about 50 nt to about 550 nt in length. In embodiments, the GntJ gene is about 50 nt to about 500 nt in length. In embodiments, the GntJ gene is about 50 nt to about 450 nt in length. In embodiments, the GntJ gene is about 50 nt to about 400 nt in length. In embodiments, the GntJ gene is about 50 nt to about 350 nt in length. In embodiments, the GntJ gene is about 50 nt to about 330 nt in length. In embodiments, the GntJ gene is about 50 nt to about 250 nt in length. In embodiments, the GntJ gene is about 50 nt to about 200 nt in length. In embodiments, the GntJ gene is about 50 nt to about 150 nt in length. In embodiments, the GntJ gene is about 50 nt to about 100 nt in length. In embodiments, the GntJ gene is about 50 nt, 100 nt, 150 nt, 200 nt, 250 nt, 330 nt, 350 nt, 400 nt, 450 nt, 500 nt, 550 nt, 600 nt, 650 nt, 700 nt, or 750 nt in length. In embodiments, the GntJ gene is about 750 nt in length. In embodiments, the GntJ gene is 750 nt in length. In embodiments, the sequence lengths described herein are within nucleic acid sequence of SEQ ID NO:68. In embodiments, the sequence lengths described herein are within nucleic acid sequence of SEQ ID NO:69. In embodiments, the sequence lengths described herein are within nucleic acid sequence of SEQ ID NO:70. In embodiments, the sequence lengths described herein are within nucleic acid sequence of SEQ ID NO:71. In embodiments, the sequence lengths described herein are within nucleic acid sequence of SEQ ID NO:72.

The term “GntT gene” or “GntT” as used herein refer to the any of the recombinant or naturally-occurring forms of the GntT gene or variants or homologs thereof. In embodiments, the GntT gene codes for a GntT polypeptide capable of maintaining the activity of the GntT polypeptide (e.g., within at least 50%, 80%, 90%, 95%, 96%, 97%, 98%, 99% or 100% activity compared to GntT polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring GntT gene (e.g. SEQ ID NO:73-75).

In embodiments, the GntT gene is substantially identical to the nucleic acid sequence of SEQ ID NO:73 or a variant or homolog having substantial identity thereto. In embodiments, the GntT gene includes the nucleic acid sequence of SEQ ID NO:73. In embodiments, the GntT gene is the nucleic acid sequence of SEQ ID NO:73. In embodiments, the GntT gene is a portion of SEQ ID NO:73.

In embodiments, the GntT gene is substantially identical to the nucleic acid sequence of SEQ ID NO:74 or a variant or homolog having substantial identity thereto. In embodiments, the GntT gene includes the nucleic acid sequence of SEQ ID NO:74. In embodiments, the GntT gene is the nucleic acid sequence of SEQ ID NO:74. In embodiments, the GntT gene is a portion of SEQ ID NO:74.

In embodiments, the GntT gene is substantially identical to the nucleic acid sequence of SEQ ID NO:75 or a variant or homolog having substantial identity thereto. In embodiments, the GntT gene includes the nucleic acid sequence of SEQ ID NO:75. In embodiments, the GntT gene is the nucleic acid sequence of SEQ ID NO:75. In embodiments, the GntT gene is a portion of SEQ ID NO:75.

In embodiments, the GntT gene is about 50 nt to about 450 nt in length. In embodiments, the GntT gene is about 100 nt to about 450 nt in length. In embodiments, the GntT gene is about 150 nt to about 450 nt in length. In embodiments, the GntT gene is about 200 nt to about 450 nt in length. In embodiments, the GntT gene is about 250 nt to about 450 nt in length. In embodiments, the GntT gene is about 300 nt to about 450 nt in length. In embodiments, the GntT gene is about 350 nt to about 450 nt in length. In embodiments, the GntT gene is about 400 nt to about 450 nt in length.

In embodiments, the GntT gene is about 50 nt to about 400 nt in length. In embodiments, the GntT gene is about 50 nt to about 350 nt in length. In embodiments, the GntT gene is about 50 nt to about 300 nt in length. In embodiments, the GntT gene is about 50 nt to about 250 nt in length. In embodiments, the GntT gene is about 50 nt to about 200 nt in length. In embodiments, the GntT gene is about 50 nt to about 150 nt in length. In embodiments, the GntT gene is about 50 nt to about 100 nt in length. In embodiments, the GntT gene is about 50 nt, 100 nt, 150 nt, 200 nt, 250 nt, 330 nt, 350 nt, 400 nt, or 450 nt in length. In embodiments, the GntT gene is about 473 nt in length. In embodiments, the GntT gene is 473 nt in length. In embodiments, the sequence lengths described herein are within nucleic acid sequence of SEQ ID NO:73. In embodiments, the sequence lengths described herein are within nucleic acid sequence of SEQ ID NO:74. In embodiments, the sequence lengths described herein are within nucleic acid sequence of SEQ ID NO:75.

In embodiments, the GntT gene is about 50 nt to about 1300 nt in length. In embodiments, the GntT gene is about 100 nt to about 1300 nt in length. In embodiments, the GntT gene is about 150 nt to about 1300 nt in length. In embodiments, the GntT gene is about 200 nt to about 1300 nt in length. In embodiments, the GntT gene is about 250 nt to about 1300 nt in length. In embodiments, the GntT gene is about 300 nt to about 1300 nt in length. In embodiments, the GntT gene is about 350 nt to about 1300 nt in length. In embodiments, the GntT gene is about 400 nt to about 1300 nt in length. In embodiments, the GntT gene is about 450 nt to about 1300 nt in length. In embodiments, the GntT gene is about 500 nt to about 1300 nt in length. In embodiments, the GntT gene is about 550 nt to about 1300 nt in length. In embodiments, the GntT gene is about 600 nt to about 1300 nt in length. In embodiments, the GntT gene is about 650 nt to about 1300 nt in length. In embodiments, the GntT gene is about 700 nt to about 1300 nt in length. In embodiments, the GntT gene is about 750 nt to about 1300 nt in length. In embodiments, the GntT gene is about 800 nt to about 1300 nt in length. In embodiments, the GntT gene is about 750 nt to about 1300 nt in length. In embodiments, the GntT gene is about 850 nt to about 1300 nt in length. In embodiments, the GntT gene is about 750 nt to about 1300 nt in length. In embodiments, the GntT gene is about 900 nt to about 1300 nt in length. In embodiments, the GntT gene is about 950 nt to about 1300 nt in length. In embodiments, the GntT gene is about 1000 nt to about 1300 nt in length. In embodiments, the GntT gene is about 1050 nt to about 1300 nt in length. In embodiments, the GntT gene is about 1100 nt to about 1300 nt in length. In embodiments, the GntT gene is about 1150 nt to about 1300 nt in length. In embodiments, the GntT gene is about 1200 nt to about 1300 nt in length. In embodiments, the GntT gene is about 1250 nt to about 1300 nt in length.

In embodiments, the GntT gene is about 50 nt to about 1250 nt in length. In embodiments, the GntT gene is about 50 nt to about 1200 nt in length. In embodiments, the GntT gene is about 50 nt to about 1150 nt in length. In embodiments, the GntT gene is about 50 nt to about 1100 nt in length. In embodiments, the GntT gene is about 50 nt to about 1050 nt in length. In embodiments, the GntT gene is about 50 nt to about 1000 nt in length. In embodiments, the GntT gene is about 50 nt to about 950 nt in length. In embodiments, the GntT gene is about 50 nt to about 900 nt in length. In embodiments, the GntT gene is about 50 nt to about 850 nt in length. In embodiments, the GntT gene is about 50 nt to about 800 nt in length. In embodiments, the GntT gene is about 50 nt to about 750 nt in length. In embodiments, the GntT gene is about 50 nt to about 700 nt in length. In embodiments, the GntT gene is about 50 nt to about 650 nt in length. In embodiments, the GntT gene is about 50 nt to about 600 nt in length. In embodiments, the GntT gene is about 50 nt to about 550 nt in length. In embodiments, the GntT gene is about 50 nt to about 500 nt in length. In embodiments, the GntT gene is about 50 nt to about 450 nt in length. In embodiments, the GntT gene is about 50 nt to about 400 nt in length. In embodiments, the GntT gene is about 50 nt to about 350 nt in length. In embodiments, the GntT gene is about 50 nt to about 300 nt in length. In embodiments, the GntT gene is about 50 nt to about 250 nt in length. In embodiments, the GntT gene is about 50 nt to about 200 nt in length. In embodiments, the GntT gene is about 50 nt to about 150 nt in length. In embodiments, the GntT gene is about 50 nt to about 100 nt in length. In embodiments, the GntT gene is about 50 nt, 100 nt, 150 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1000 nt, 1050 nt, 1100 nt, 1150 nt, 1200 nt, 1250 nt, or 1300 nt in length. In embodiments, the GntT gene is about 1359 nt in length. In embodiments, the GntT gene is 1359 nt in length. In embodiments, the sequence lengths described herein are within nucleic acid sequence of SEQ ID NO:73. In embodiments, the sequence lengths described herein are within nucleic acid sequence of SEQ ID NO:74. In embodiments, the sequence lengths described herein are within nucleic acid sequence of SEQ ID NO:75.

In embodiments, the one or more guanitoxin biosynthetic genes include any combination of genes selected from GntA, GntB, GntC, GntD, GntE, GntF, GntG, GntH, GntI, GntJ, and GntT. In embodiments, the one or more guanitoxin biosynthetic genes include GntA, GntJ, GntC, or a combination thereof. In embodiments, the one or more guanitoxin biosynthetic genes includes GntA. In embodiments, the one or more guanitoxin biosynthetic genes includes GntJ. In embodiments, the one or more guanitoxin biosynthetic genes includes GntC. In embodiments, the one or more guanitoxin biosynthetic genes is GntA. In embodiments, the one or more guanitoxin biosynthetic genes is GntJ. In embodiments, the one or more guanitoxin biosynthetic genes is GntC. In embodiments, the one or more guanitoxin biosynthetic genes include GntA and at least one gene selected from GntB, GntC, GntD, GntE, GntF, GntG, GntH, GntI, GntJ, and GntT. In embodiments, the one or more guanitoxin biosynthetic genes include GntJ and at least one gene selected from GntA, GntB, GntC, GntD, GntE, GntF, GntG, GntH, GntI, and GntT. In embodiments, the one or more guanitoxin biosynthetic genes include GntC and at least one gene selected from GntA, GntB, GntD, GntE, GntF, GntG, GntH, GntI, GntJ, and GntT. In embodiments, the one or more guanitoxin biosynthetic genes include GntB and at least one gene selected from GntA, GntC, GntD, GntE, GntF, GntG, GntH, GntI, GntJ, and GntT. In embodiments, the one or more guanitoxin biosynthetic genes include GntD and at least one gene selected from GntA, GntB, GntC, GntE, GntF, GntG, GntH, GntI, GntJ, and GntT. In embodiments, the one or more guanitoxin biosynthetic genes include GntE and at least one gene selected from GntA, GntB, GntC, GntD, GntF, GntG, GntH, GntI, GntJ, and GntT. In embodiments, the one or more guanitoxin biosynthetic genes include GntF and at least one gene selected from GntA, GntB, GntC, GntD, GntE, GntG, GntH, GntI, GntJ, and GntT. In embodiments, the one or more guanitoxin biosynthetic genes include GntG and at least one gene selected from GntA, GntB, GntC, GntD, GntE, GntF, GntH, GntI, GntJ, and GntT. In embodiments, the one or more guanitoxin biosynthetic genes include GntH and at least one gene selected from GntA, GntB, GntC, GntD, GntE, GntF, GntG, GntI, GntJ, and GntT. In embodiments, the one or more guanitoxin biosynthetic genes include GntI and at least one gene selected from GntA, GntB, GntC, GntD, GntE, GntF, GntG, GntH, GntJ, and GntT. In embodiments, the one or more guanitoxin biosynthetic genes include GntT and at least one gene selected from GntA, GntB, GntC, GntD, GntE, GntF, GntG, GntH, GntI, and GntJ.

In embodiments, the one or more guanitoxin biosynthetic genes is one guanitoxin biosynthetic gene. In embodiments, the one guanitoxin biosynthetic gene is GntA, GntB, GntC, GntD, GntE, GntF, GntG, GntH, GntI, GntJ, or GntT. In embodiments, the guanitoxin biosynthetic gene is GntA and no other guanitoxin biosynthetic gene. In embodiments, the guanitoxin biosynthetic gene is GntB and no other guanitoxin biosynthetic gene. In embodiments, the guanitoxin biosynthetic gene is GntC and no other guanitoxin biosynthetic gene. In embodiments, the guanitoxin biosynthetic gene is GntD and no other guanitoxin biosynthetic gene. In embodiments, the guanitoxin biosynthetic gene is GntE and no other guanitoxin biosynthetic gene. In embodiments, the guanitoxin biosynthetic gene is GntF and no other guanitoxin biosynthetic gene. In embodiments, the guanitoxin biosynthetic gene is GntG and no other guanitoxin biosynthetic gene. In embodiments, the guanitoxin biosynthetic gene is GntH and no other guanitoxin biosynthetic gene. In embodiments, the guanitoxin biosynthetic gene is GntI and no other guanitoxin biosynthetic gene. In embodiments, the guanitoxin biosynthetic gene is GntT and no other guanitoxin biosynthetic gene.

In embodiments, the method includes concentrating bacterial cells or particulate matter in the aqueous liquid. One of skill in the art would recognize that a number of techniques may be used for concentrating cells or particulate matter, for example use of filters with pores quantitatively designed to capture the particulate matter and/or bacterial cells, including STERIVEX™ filters, glass fiber filters, etc. In embodiments, the method includes concentrating bacterial cells using a filter, wherein the filter has a pore size of less than about 1 μm. In embodiments, the method further includes extracting the guanitoxin biosynthetic gene from the concentrated cells or particulate matter. In embodiments, the method further includes purifying the guanitoxin biosynthetic gene from the concentrated cells or particulate matter. In embodiments, the guanitoxin biosynthetic gene is DNA or RNA. In embodiments, the guanitoxin biosynthetic gene is DNA. In embodiments, the guanitoxin biosynthetic gene is RNA. One of skill in the art would recognize that a variety of methods can be used for extracting and/or purifying the guanitoxin biosynthetic gene, including use of a number of commercially available kits (e.g. DNeasy® PowerWater® Kit, etc.). Methods for contracting cells or particulate matter, and extracting and/or purifying nucleic acids from aqueous liquid are described in more detail in Kurmayer, R. et al. (2017). Molecular Tools for the Detection and Quantification of Toxigenic Cyanobacteria. John Wiley and Sons Ltd.

In embodiments, the method includes contacting the aqueous liquid with one or more nucleic acids, wherein each of the one or more nucleic acids are at least partially complementary to a portion of the one or more guanitoxin biosynthetic genes. In embodiments, the one or more nucleic acids is a primer. In embodiments, the one or more nucleic acids is a probe. A “primer” refers to a short, single-stranded DNA sequence used in polymerase chain reaction (PCR) methods and isothermal amplification methods. In embodiments, a pair of primers (e.g. a first nucleic and a second nucleic acid) is used to hybridize with DNA (e.g. guanitoxin biosynthetic gene) in a sample (e.g. aqueous liquid) and define the region of the DNA that will be amplified. A “probe” is a single-stranded DNA sequence used to detect DNA or RNA (e.g. guanitoxin biosynthetic gene) in a sample (e.g. aqueous liquid). In embodiments, a probe hybridizes with the DNA or RNA, and hybridization is detected, thereby allowing detection of the DNA or RNA. In embodiments, the probe is coupled to a detectable label (e.g. a fluorescent or radioactive compound). In embodiments, the method further includes contacting the aqueous liquid with an enzyme. In embodiments, the enzyme is a polymerase. In embodiments, the polymerase is Taq polymerase.

In embodiments, the method further includes performing reverse transcription, thereby producing a cDNA of the one or more guanitoxin biosynthetic gene. Thus, in embodiments, the method further includes contacting the aqueous liquid with an enzyme. In embodiments, the enzyme is a reverse transcriptase.

For the method provided herein, in embodiments, the guanitoxin biosynthetic gene is amplified, thereby producing amplicons. Thus, in embodiments, the method includes performing a PCR method, an isothermal amplification method, or a combination thereof. In embodiments, the method includes performing a PCR method. In embodiments, the PCR method is reverse transcription PCR (RT-PCR), quantitative PCR (qPCR), or reverse transcription quantitative PCR (RT-qPCR). In embodiments, the PCR method is RT-PCR. In embodiments, the PCR method is qPCR. In embodiments, the PCR method is RT-qPCR. In embodiments, the method includes performing an isothermal amplification method. In embodiments, the isothermal amplification method is loop-mediated isothermal amplification (LAMP). In embodiments, the method further includes detecting amplicons. In embodiments, detecting amplicons includes a colorimetric method, a fluorometric method, a luminometric method, an ionic method, or an electrical detection method. In embodiments, the method includes performing a sequencing method. For example, the guanitoxin biosynthetic gene or the amplicon may be sequenced. In embodiments, the sequencing method includes next generation sequencing. Thus, in embodiments, the method includes performing a PCR method, an isothermal amplification method, a sequencing method, or a combination thereof. In embodiments, the PCR method, isothermal amplification method, or sequencing method is a multiplex method. For example, in embodiments, the PCR method, isothermal amplification method, or sequencing method allows for detection of multiple guanitoxin biosynthetic genes simultaneously. In embodiments, 2, 3, 4, 5, 6, 7, 8, 9, 10, or more guanitoxin biosynthetic genes are detected simultaneously.

For the method provided herein, in embodiments, the portion of the one or more guanitoxin biosynthetic genes includes a coding sequence, a promoter region sequence, a terminator region sequence, or an intergene region sequence. In embodiments, the portion of the one or more guanitoxin biosynthetic genes includes a coding sequence. In embodiments, the portion of the one or more guanitoxin biosynthetic genes includes a promoter region sequence. In embodiments, the portion of the one or more guanitoxin biosynthetic genes includes a terminator region sequence. In embodiments, the portion of the one or more guanitoxin biosynthetic genes includes an intergene region sequence.

For the method provided herein, in embodiments, the one or more nucleic acids each independently includes a sequence having at least 80%, 85%, 90%, 91%, 92%, 93%, 94%, 95%, 96%, 97%, 98%, or 99% identity to any one of SEQ ID NO:1 to SEQ ID NO:22, wherein each nucleic acid of the one or more nucleic acids is different. In embodiments, the one or more nucleic acids each independently includes the sequence of one of SEQ ID NO:1 to SEQ ID NO:22, wherein each nucleic acid of the one or more nucleic acids is different.

In embodiments, the one or more nucleic acids includes a sequence having at least 80% identity to SEQ ID NO:1. In embodiments, the one or more nucleic acids includes a sequence having at least 85% identity to SEQ ID NO:1. In embodiments, the one or more nucleic acids includes a sequence having at least 90% identity to SEQ ID NO:1. In embodiments, the one or more nucleic acids includes a sequence having at least 95% identity to SEQ ID NO:1. In embodiments, the one or more nucleic acids includes a sequence having at least 98% identity to SEQ ID NO:1. In embodiments, the one or more nucleic acids includes the sequence of SEQ ID NO:1. In embodiments, the one or more nucleic acids includes SEQ ID NO:1. In embodiments, the one or more nucleic acids is SEQ ID NO:1.

In embodiments, the one or more nucleic acids includes a sequence having at least 80% identity to SEQ ID NO:2. In embodiments, the one or more nucleic acids includes a sequence having at least 85% identity to SEQ ID NO:2. In embodiments, the one or more nucleic acids includes a sequence having at least 90% identity to SEQ ID NO:2. In embodiments, the one or more nucleic acids includes a sequence having at least 95% identity to SEQ ID NO:2. In embodiments, the one or more nucleic acids includes a sequence having at least 98% identity to SEQ ID NO:2. In embodiments, the one or more nucleic acids includes the sequence of SEQ ID NO:2. In embodiments, the one or more nucleic acids includes SEQ ID NO:2. In embodiments, the one or more nucleic acids is SEQ ID NO:2.

In embodiments, the one or more nucleic acids includes a sequence having at least 80% identity to SEQ ID NO:3. In embodiments, the one or more nucleic acids includes a sequence having at least 85% identity to SEQ ID NO:3. In embodiments, the one or more nucleic acids includes a sequence having at least 90% identity to SEQ ID NO:3. In embodiments, the one or more nucleic acids includes a sequence having at least 95% identity to SEQ ID NO:3. In embodiments, the one or more nucleic acids includes a sequence having at least 98% identity to SEQ ID NO:3. In embodiments, the one or more nucleic acids includes the sequence of SEQ ID NO:3. In embodiments, the one or more nucleic acids includes SEQ ID NO:3. In embodiments, the one or more nucleic acids is SEQ ID NO:3.

In embodiments, the one or more nucleic acids includes a sequence having at least 80% identity to SEQ ID NO:4. In embodiments, the one or more nucleic acids includes a sequence having at least 85% identity to SEQ ID NO:4. In embodiments, the one or more nucleic acids includes a sequence having at least 90% identity to SEQ ID NO:4. In embodiments, the one or more nucleic acids includes a sequence having at least 95% identity to SEQ ID NO:4. In embodiments, the one or more nucleic acids includes a sequence having at least 98% identity to SEQ ID NO:4. In embodiments, the one or more nucleic acids includes the sequence of SEQ ID NO:4. In embodiments, the one or more nucleic acids includes SEQ ID NO:4. In embodiments, the one or more nucleic acids is SEQ ID NO:4.

In embodiments, the one or more nucleic acids includes a sequence having at least 80% identity to SEQ ID NO:5. In embodiments, the one or more nucleic acids includes a sequence having at least 85% identity to SEQ ID NO:5. In embodiments, the one or more nucleic acids includes a sequence having at least 90% identity to SEQ ID NO:5. In embodiments, the one or more nucleic acids includes a sequence having at least 95% identity to SEQ ID NO:5. In embodiments, the one or more nucleic acids includes a sequence having at least 98% identity to SEQ ID NO:5. In embodiments, the one or more nucleic acids includes the sequence of SEQ ID NO:5. In embodiments, the one or more nucleic acids includes SEQ ID NO:5. In embodiments, the one or more nucleic acids is SEQ ID NO:5.

In embodiments, the one or more nucleic acids includes a sequence having at least 80% identity to SEQ ID NO:6. In embodiments, the one or more nucleic acids includes a sequence having at least 85% identity to SEQ ID NO:6. In embodiments, the one or more nucleic acids includes a sequence having at least 90% identity to SEQ ID NO:6. In embodiments, the one or more nucleic acids includes a sequence having at least 95% identity to SEQ ID NO:6. In embodiments, the one or more nucleic acids includes a sequence having at least 98% identity to SEQ ID NO:6. In embodiments, the one or more nucleic acids includes the sequence of SEQ ID NO:6. In embodiments, the one or more nucleic acids includes SEQ ID NO:6. In embodiments, the one or more nucleic acids is SEQ ID NO:6.

In embodiments, the one or more nucleic acids includes a sequence having at least 80% identity to SEQ ID NO:7. In embodiments, the one or more nucleic acids includes a sequence having at least 85% identity to SEQ ID NO:7. In embodiments, the one or more nucleic acids includes a sequence having at least 90% identity to SEQ ID NO:7. In embodiments, the one or more nucleic acids includes a sequence having at least 95% identity to SEQ ID NO:7. In embodiments, the one or more nucleic acids includes a sequence having at least 98% identity to SEQ ID NO:7. In embodiments, the one or more nucleic acids includes the sequence of SEQ ID NO:7. In embodiments, the one or more nucleic acids includes SEQ ID NO:7. In embodiments, the one or more nucleic acids is SEQ ID NO:7.

In embodiments, the one or more nucleic acids includes a sequence having at least 80% identity to SEQ ID NO:8. In embodiments, the one or more nucleic acids includes a sequence having at least 85% identity to SEQ ID NO:8. In embodiments, the one or more nucleic acids includes a sequence having at least 90% identity to SEQ ID NO:8. In embodiments, the one or more nucleic acids includes a sequence having at least 95% identity to SEQ ID NO:8. In embodiments, the one or more nucleic acids includes a sequence having at least 98% identity to SEQ ID NO:8. In embodiments, the one or more nucleic acids includes the sequence of SEQ ID NO:8. In embodiments, the one or more nucleic acids includes SEQ ID NO:8. In embodiments, the one or more nucleic acids is SEQ ID NO:8.

In embodiments, the one or more nucleic acids includes a sequence having at least 80% identity to SEQ ID NO:9. In embodiments, the one or more nucleic acids includes a sequence having at least 85% identity to SEQ ID NO:9. In embodiments, the one or more nucleic acids includes a sequence having at least 90% identity to SEQ ID NO:9. In embodiments, the one or more nucleic acids includes a sequence having at least 95% identity to SEQ ID NO:9. In embodiments, the one or more nucleic acids includes a sequence having at least 98% identity to SEQ ID NO:9. In embodiments, the one or more nucleic acids includes the sequence of SEQ ID NO:9. In embodiments, the one or more nucleic acids includes SEQ ID NO:9. In embodiments, the one or more nucleic acids is SEQ ID NO:9.

In embodiments, the one or more nucleic acids includes a sequence having at least 80% identity to SEQ ID NO:10. In embodiments, the one or more nucleic acids includes a sequence having at least 85% identity to SEQ ID NO:10. In embodiments, the one or more nucleic acids includes a sequence having at least 90% identity to SEQ ID NO:10. In embodiments, the one or more nucleic acids includes a sequence having at least 95% identity to SEQ ID NO:10. In embodiments, the one or more nucleic acids includes a sequence having at least 98% identity to SEQ ID NO:10. In embodiments, the one or more nucleic acids includes the sequence of SEQ ID NO:10. In embodiments, the one or more nucleic acids includes SEQ ID NO:10. In embodiments, the one or more nucleic acids is SEQ ID NO:10.

In embodiments, the one or more nucleic acids includes a sequence having at least 80% identity to SEQ ID NO:11. In embodiments, the one or more nucleic acids includes a sequence having at least 85% identity to SEQ ID NO:11. In embodiments, the one or more nucleic acids includes a sequence having at least 90% identity to SEQ ID NO:11. In embodiments, the one or more nucleic acids includes a sequence having at least 95% identity to SEQ ID NO:11. In embodiments, the one or more nucleic acids includes a sequence having at least 98% identity to SEQ ID NO:11. In embodiments, the one or more nucleic acids includes the sequence of SEQ ID NO:11. In embodiments, the one or more nucleic acids includes SEQ ID NO:11. In embodiments, the one or more nucleic acids is SEQ ID NO:11.

In embodiments, the one or more nucleic acids includes a sequence having at least 80% identity to SEQ ID NO:12. In embodiments, the one or more nucleic acids includes a sequence having at least 85% identity to SEQ ID NO:12. In embodiments, the one or more nucleic acids includes a sequence having at least 90% identity to SEQ ID NO:12. In embodiments, the one or more nucleic acids includes a sequence having at least 95% identity to SEQ ID NO:12. In embodiments, the one or more nucleic acids includes a sequence having at least 98% identity to SEQ ID NO:12. In embodiments, the one or more nucleic acids includes the sequence of SEQ ID NO:12. In embodiments, the one or more nucleic acids includes SEQ ID NO:12. In embodiments, the one or more nucleic acids is SEQ ID NO:12.

In embodiments, the one or more nucleic acids includes a sequence having at least 80% identity to SEQ ID NO:13. In embodiments, the one or more nucleic acids includes a sequence having at least 85% identity to SEQ ID NO:13. In embodiments, the one or more nucleic acids includes a sequence having at least 90% identity to SEQ ID NO:13. In embodiments, the one or more nucleic acids includes a sequence having at least 95% identity to SEQ ID NO:13. In embodiments, the one or more nucleic acids includes a sequence having at least 98% identity to SEQ ID NO:13. In embodiments, the one or more nucleic acids includes the sequence of SEQ ID NO:13. In embodiments, the one or more nucleic acids includes SEQ ID NO:13. In embodiments, the one or more nucleic acids is SEQ ID NO:13.

In embodiments, the one or more nucleic acids includes a sequence having at least 80% identity to SEQ ID NO:14. In embodiments, the one or more nucleic acids includes a sequence having at least 85% identity to SEQ ID NO:14. In embodiments, the one or more nucleic acids includes a sequence having at least 90% identity to SEQ ID NO:14. In embodiments, the one or more nucleic acids includes a sequence having at least 95% identity to SEQ ID NO:14. In embodiments, the one or more nucleic acids includes a sequence having at least 98% identity to SEQ ID NO:14. In embodiments, the one or more nucleic acids includes the sequence of SEQ ID NO:14. In embodiments, the one or more nucleic acids includes SEQ ID NO:14. In embodiments, the one or more nucleic acids is SEQ ID NO:14.

In embodiments, the one or more nucleic acids includes a sequence having at least 80% identity to SEQ ID NO:15. In embodiments, the one or more nucleic acids includes a sequence having at least 85% identity to SEQ ID NO:15. In embodiments, the one or more nucleic acids includes a sequence having at least 90% identity to SEQ ID NO:15. In embodiments, the one or more nucleic acids includes a sequence having at least 95% identity to SEQ ID NO:15. In embodiments, the one or more nucleic acids includes a sequence having at least 98% identity to SEQ ID NO:15. In embodiments, the one or more nucleic acids includes the sequence of SEQ ID NO:15. In embodiments, the one or more nucleic acids includes SEQ ID NO:15. In embodiments, the one or more nucleic acids is SEQ ID NO:15.

In embodiments, the one or more nucleic acids includes a sequence having at least 80% identity to SEQ ID NO:16. In embodiments, the one or more nucleic acids includes a sequence having at least 85% identity to SEQ ID NO:16. In embodiments, the one or more nucleic acids includes a sequence having at least 90% identity to SEQ ID NO:16. In embodiments, the one or more nucleic acids includes a sequence having at least 95% identity to SEQ ID NO:16. In embodiments, the one or more nucleic acids includes a sequence having at least 98% identity to SEQ ID NO:16. In embodiments, the one or more nucleic acids includes the sequence of SEQ ID NO:16. In embodiments, the one or more nucleic acids includes SEQ ID NO:16. In embodiments, the one or more nucleic acids is SEQ ID NO:16.

In embodiments, the one or more nucleic acids includes a sequence having at least 80% identity to SEQ ID NO:17. In embodiments, the one or more nucleic acids includes a sequence having at least 85% identity to SEQ ID NO:17. In embodiments, the one or more nucleic acids includes a sequence having at least 90% identity to SEQ ID NO:17. In embodiments, the one or more nucleic acids includes a sequence having at least 95% identity to SEQ ID NO:17. In embodiments, the one or more nucleic acids includes a sequence having at least 98% identity to SEQ ID NO:17. In embodiments, the one or more nucleic acids includes the sequence of SEQ ID NO:17. In embodiments, the one or more nucleic acids includes SEQ ID NO:17. In embodiments, the one or more nucleic acids is SEQ ID NO:17.

In embodiments, the one or more nucleic acids includes a sequence having at least 80% identity to SEQ ID NO:18. In embodiments, the one or more nucleic acids includes a sequence having at least 85% identity to SEQ ID NO:18. In embodiments, the one or more nucleic acids includes a sequence having at least 90% identity to SEQ ID NO:18. In embodiments, the one or more nucleic acids includes a sequence having at least 95% identity to SEQ ID NO:18. In embodiments, the one or more nucleic acids includes a sequence having at least 98% identity to SEQ ID NO:18. In embodiments, the one or more nucleic acids includes the sequence of SEQ ID NO:18. In embodiments, the one or more nucleic acids includes SEQ ID NO:18. In embodiments, the one or more nucleic acids is SEQ ID NO:18.

In embodiments, the one or more nucleic acids includes a sequence having at least 80% identity to SEQ ID NO:19. In embodiments, the one or more nucleic acids includes a sequence having at least 85% identity to SEQ ID NO:19. In embodiments, the one or more nucleic acids includes a sequence having at least 90% identity to SEQ ID NO:19. In embodiments, the one or more nucleic acids includes a sequence having at least 95% identity to SEQ ID NO:19. In embodiments, the one or more nucleic acids includes a sequence having at least 98% identity to SEQ ID NO:19. In embodiments, the one or more nucleic acids includes the sequence of SEQ ID NO:19. In embodiments, the one or more nucleic acids includes SEQ ID NO:19. In embodiments, the one or more nucleic acids is SEQ ID NO:19.

In embodiments, the one or more nucleic acids includes a sequence having at least 80% identity to SEQ ID NO:20. In embodiments, the one or more nucleic acids includes a sequence having at least 85% identity to SEQ ID NO:20. In embodiments, the one or more nucleic acids includes a sequence having at least 90% identity to SEQ ID NO:20. In embodiments, the one or more nucleic acids includes a sequence having at least 95% identity to SEQ ID NO:20. In embodiments, the one or more nucleic acids includes a sequence having at least 98% identity to SEQ ID NO:20. In embodiments, the one or more nucleic acids includes the sequence of SEQ ID NO:20. In embodiments, the one or more nucleic acids includes SEQ ID NO:20. In embodiments, the one or more nucleic acids is SEQ ID NO:20.

In embodiments, the one or more nucleic acids includes a sequence having at least 80% identity to SEQ ID NO:21. In embodiments, the one or more nucleic acids includes a sequence having at least 85% identity to SEQ ID NO:21. In embodiments, the one or more nucleic acids includes a sequence having at least 90% identity to SEQ ID NO:21. In embodiments, the one or more nucleic acids includes a sequence having at least 95% identity to SEQ ID NO:21. In embodiments, the one or more nucleic acids includes a sequence having at least 98% identity to SEQ ID NO:21. In embodiments, the one or more nucleic acids includes the sequence of SEQ ID NO:21. In embodiments, the one or more nucleic acids includes SEQ ID NO:21. In embodiments, the one or more nucleic acids is SEQ ID NO:21.

In embodiments, the one or more nucleic acids includes a sequence having at least 80% identity to SEQ ID NO:22. In embodiments, the one or more nucleic acids includes a sequence having at least 85% identity to SEQ ID NO:22. In embodiments, the one or more nucleic acids includes a sequence having at least 90% identity to SEQ ID NO:22. In embodiments, the one or more nucleic acids includes a sequence having at least 95% identity to SEQ ID NO:22. In embodiments, the one or more nucleic acids includes a sequence having at least 98% identity to SEQ ID NO:22. In embodiments, the one or more nucleic acids includes the sequence of SEQ ID NO:22. In embodiments, the one or more nucleic acids includes SEQ ID NO:22. In embodiments, the one or more nucleic acids is SEQ ID NO:22.

In embodiments, the one or more nucleic acids includes a sequence having at least 80% identity to SEQ ID NO:87. In embodiments, the one or more nucleic acids includes a sequence having at least 85% identity to SEQ ID NO:87. In embodiments, the one or more nucleic acids includes a sequence having at least 90% identity to SEQ ID NO:87. In embodiments, the one or more nucleic acids includes a sequence having at least 95% identity to SEQ ID NO:87. In embodiments, the one or more nucleic acids includes a sequence having at least 98% identity to SEQ ID NO:87. In embodiments, the one or more nucleic acids includes the sequence of SEQ ID NO:87. In embodiments, the one or more nucleic acids includes SEQ ID NO:87. In embodiments, the one or more nucleic acids is SEQ ID NO:87.

In embodiments, the one or more nucleic acids includes a sequence having at least 80% identity to SEQ ID NO:88. In embodiments, the one or more nucleic acids includes a sequence having at least 85% identity to SEQ ID NO:88. In embodiments, the one or more nucleic acids includes a sequence having at least 90% identity to SEQ ID NO:88. In embodiments, the one or more nucleic acids includes a sequence having at least 95% identity to SEQ ID NO:88. In embodiments, the one or more nucleic acids includes a sequence having at least 98% identity to SEQ ID NO:88. In embodiments, the one or more nucleic acids includes the sequence of SEQ ID NO:88. In embodiments, the one or more nucleic acids includes SEQ ID NO:88. In embodiments, the one or more nucleic acids is SEQ ID NO:88.

In embodiments, the one or more nucleic acids includes a sequence having at least 80% identity to SEQ ID NO:89. In embodiments, the one or more nucleic acids includes a sequence having at least 85% identity to SEQ ID NO:89. In embodiments, the one or more nucleic acids includes a sequence having at least 90% identity to SEQ ID NO:89. In embodiments, the one or more nucleic acids includes a sequence having at least 95% identity to SEQ ID NO:89. In embodiments, the one or more nucleic acids includes a sequence having at least 98% identity to SEQ ID NO:89. In embodiments, the one or more nucleic acids includes the sequence of SEQ ID NO:89. In embodiments, the one or more nucleic acids includes SEQ ID NO:89. In embodiments, the one or more nucleic acids is SEQ ID NO:89.

In embodiments, the one or more nucleic acids includes a sequence having at least 80% identity to SEQ ID NO:90. In embodiments, the one or more nucleic acids includes a sequence having at least 85% identity to SEQ ID NO:90. In embodiments, the one or more nucleic acids includes a sequence having at least 90% identity to SEQ ID NO:90. In embodiments, the one or more nucleic acids includes a sequence having at least 95% identity to SEQ ID NO:90. In embodiments, the one or more nucleic acids includes a sequence having at least 98% identity to SEQ ID NO:90. In embodiments, the one or more nucleic acids includes the sequence of SEQ ID NO:90. In embodiments, the one or more nucleic acids includes SEQ ID NO:90. In embodiments, the one or more nucleic acids is SEQ ID NO:90.

In embodiments, the one or more nucleic acids include a first nucleic acid including a sequence having at least 80% identity to SEQ ID NO:1 and a second nucleic acid including a sequence having at least 80% identity to SEQ ID NO:2. In embodiments, the one or more nucleic acids include a first nucleic acid including a sequence having at least 80% identity to SEQ ID NO:3 and a second nucleic acid including a sequence having at least 80% identity to SEQ ID NO:4. In embodiments, the one or more nucleic acids include a first nucleic acid including a sequence having at least 80% identity to SEQ ID NO:5 and a second nucleic acid including a sequence having at least 80% identity to SEQ ID NO:6. In embodiments, the one or more nucleic acids include a first nucleic acid including a sequence having at least 80% identity to SEQ ID NO:7 and a second nucleic acid including a sequence having at least 80% identity to SEQ ID NO:8. In embodiments, the one or more nucleic acids include a first nucleic acid including a sequence having at least 80% identity to SEQ ID NO:9 and a second nucleic acid including a sequence having at least 80% identity to SEQ ID NO:10. In embodiments, the one or more nucleic acids include a first nucleic acid including a sequence having at least 80% identity to SEQ ID NO:11 and a second nucleic acid including a sequence having at least 80% identity to SEQ ID NO:12. In embodiments, the one or more nucleic acids include a first nucleic acid including a sequence having at least 80% identity to SEQ ID NO:13 and a second nucleic acid including a sequence having at least 80% identity to SEQ ID NO:14. In embodiments, the one or more nucleic acids include a first nucleic acid including a sequence having at least 80% identity to SEQ ID NO:15 and a second nucleic acid including a sequence having at least 80% identity to SEQ ID NO:16. In embodiments, the one or more nucleic acids include a first nucleic acid including a sequence having at least 80% identity to SEQ ID NO:17 and a second nucleic acid including a sequence having at least 80% identity to SEQ ID NO:18. In embodiments, the one or more nucleic acids include a first nucleic acid including a sequence having at least 80% identity to SEQ ID NO:19 and a second nucleic acid including a sequence having at least 80% identity to SEQ ID NO:20. In embodiments, the one or more nucleic acids include a first nucleic acid including a sequence having at least 80% identity to SEQ ID NO:21 and a second nucleic acid including a sequence having at least 80% identity to SEQ ID NO:22. In embodiments, the one or more nucleic acids include a first nucleic acid including a sequence having at least 80% identity to SEQ ID NO:87 and a second nucleic acid including a sequence having at least 80% identity to SEQ ID NO:88. In embodiments, the one or more nucleic acids include a first nucleic acid including a sequence having at least 80% identity to SEQ ID NO:89 and a second nucleic acid including a sequence having at least 80% identity to SEQ ID NO:90.

For the methods provided herein, in embodiments, the guanitoxin-producing bacteria are cyanobacteria. In embodiments, the cyanobacteria are Sphaerospermopsis torques-reginae, Chrysosporum ovalisporum, Cuspidothrix, Cylindrospermopsis, Cylindrospermum, Dolichospermum, Microcystis, Oscillatoria, Planktothrix, Phormidium, Anabaenaflos-aquae, A. lemmermannii Raphidiopsis mediterranea, Tychonema, or Woronichinia. In embodiments, the cyanobacteria are Sphaerospermopsis torques-reginae. In embodiments, the cyanobacteria are Chrysosporum ovalisporum. In embodiments, the cyanobacteria are Cuspidothrix. In embodiments, the cyanobacteria are Cylindrospermopsis. In embodiments, the cyanobacteria are Cylindrospermum. In embodiments, the cyanobacteria are Dolichospermum. In embodiments, the cyanobacteria are Microcystis. In embodiments, the cyanobacteria are Oscillatoria. In embodiments, the cyanobacteria are Planktothrix. In embodiments, the cyanobacteria are Phormidium. In embodiments, the cyanobacteria are Anabaenaflos-aquae. In embodiments, the cyanobacteria are A. lemmermannii Raphidiopsis mediterranea. In embodiments, the cyanobacteria are Tychonema. In embodiments, the cyanobacteria are Woronichinia.

For the method provided herein, including embodiments thereof, the aqueous liquid may be derived from a body of water which may be ingested by, inhaled by, or in contact with a subject. In embodiments, the aqueous liquid is derived from a potable water source. In embodiments, the aqueous liquid is derived from a lake, river, or pond. In embodiments, the aqueous liquid is derived from a lake. In embodiments, the aqueous liquid is derived from a river. In embodiments, the aqueous liquid is derived from a pond.

In embodiments, the aqueous liquid is derived from a public water system or a private water system. The public water system or private water system may be a potable water source. In embodiments, the aqueous liquid is derived from a public water system. “Public water system” is used according to its commonly known meaning in the art, and refers to water provided to humans for consumption through a constructed conveyance (e.g. pipes, etc.) that has 15 or more service connections, or serves at least 25 people for at least 60 days out of the year. In embodiments, the public water system has a constructed conveyance (e.g. pipes, etc.) that has 15 or more service connections. In embodiments, the public water system serves at least 25 people for at least 60 days out of the year. “Service connection” is used in accordance to its commonly known meaning in the art and refers to the point of connection between the constructed conveyance of the water system and the constructed conveyance used by a subject. In embodiments, the aqueous liquid is a community water system, a non-transient non-community water system, or a transient non-community water system.

In embodiments, the aqueous liquid is derived from a private water system. The term “private water system” is used according to its commonly known meaning in the art and refers to water systems that serve no more than 25 people at least 60 days out of the year or have no more than 15 service connections. In embodiments, the private water system serves no more than 25 people for at least 60 days out of the year. In embodiments, the private water system has no more than 15 service connections. In embodiments, the private water system is water from a spring, stream, pond, or shallow well. In embodiments, the private water system is private ground water, a residential well, or a cistern. In embodiments, the private water system is bottled water (commercial or filled individually). In embodiments, the private water system supplies water to an individual residence.

For the methods provided herein, in embodiments, the aqueous liquid is ingested by, inhaled by, or contacted with a subject. In embodiments, the aqueous liquid is ingested by the subject. In embodiments, the aqueous liquid is ingested by the subject as drinking water (e.g. drinking water from a bottle, a public or private water system, a spring, etc.). In embodiments, the aqueous liquid is unintentionally ingested (e.g. while a subject is swimming). In embodiments, the aqueous liquid is inhaled by the subject. In embodiments, the aqueous liquid is inhaled as aerosolized liquid. In embodiments, the aqueous liquid is contacted with the subject (e.g. the skin or mucous membrane of the subject).

For the method provided herein, in embodiments, the subject is treated for guanitoxin-induced toxicity when the one or more guanitoxin biosynthetic genes are detected.

In embodiments, the treatment includes ameliorating a symptom of guanitoxin-induced toxicity. In embodiments, the symptom of guanitoxin-induced toxicity is tremors and/or seizure. In embodiments, the treatment includes administering an effective amount of a muscle relaxant, benzodiazepine, or barbiturate to the subject. In embodiments, the treatment includes administering an effective amount of atropine to the subject. In embodiments, the treatment includes administering an effective amount of glycopyrrolate to the subject. In embodiments, the treatment includes administering an effective amount of physostigmine to the subject. In embodiments, the treatment includes administering an effective amount of 2-PAM to the subject.

### Kits

Provided herein are, inter alia, kits including components, such as such as reagents and reaction mixtures, for detecting guanitoxin biosynthetic genes (e.g. GntA, GntB, GntC, GntD, GntE, GntF, GntG, GntH, GntI, GntJ, GntT, or a combination thereof) as described herein including embodiments thereof. In embodiments, the kit includes materials and instructions (e.g., for storage and use of kit components). In embodiments, the kit includes reagents capable of detecting the presence of one or more of GntA, GntB, GntC, GntD, GntE, GntF, GntG, GntH, GntI, GntJ, GntT, or any combination thereof in an aqueous liquid. In embodiments, the kit includes one or more nucleic acids at least partially complementary to a portion to one or more guanitoxin biosynthetic genes (e.g. GntA, GntB, GntC, GntD, GntE, GntF, GntG, GntH, GntI, GntJ, GntT, or a combination thereof) as described herein including embodiments thereof. In embodiments, the one or more nucleic acids is a probe that can hybridize to one or more guanitoxin biosynthetic genes. In embodiments, the one or more nucleic acids is a primer or pairs of primers for amplifying one or more guanitoxin biosynthetic genes. In embodiments, the kits include reagents and reaction mixtures for a PCR method or an isothermal amplification method. In embodiments, the kit includes instructions. In embodiments, the kit includes a label or insert indicating regulatory approval for diagnostic use. Thus, in an aspect is provided a kit for detecting guanitoxin-producing bacteria in an aqueous liquid, the kit including one or more nucleic acids each at least partially complementary to a portion of one or more guanitoxin biosynthetic genes, wherein the one or more guanitoxin biosynthetic genes are GntA, GntB, GntC, GntD, GntE, GntF, GntG, GntH, GntI, GntJ, GntT, or a combination thereof.

In embodiments, the one or more guanitoxin biosynthetic genes are GntA, GntJ, GntC, or a combination thereof. In embodiments, the one or more guanitoxin biosynthetic genes includes GntA. In embodiments, the one or more guanitoxin biosynthetic genes includes GntJ. In embodiments, the one or more guanitoxin biosynthetic genes includes GntC.

In embodiments, the portion of the one or more guanitoxin biosynthetic genes includes a coding sequence, a promoter region sequence, a terminator region sequence, or an intergene region sequence. In embodiments, the portion of the one or more guanitoxin biosynthetic genes includes a coding sequence.

In embodiments, the one or more nucleic acids each independently includes a sequence having at least 80% identity to any one of SEQ ID NO:1 to SEQ ID NO:22, wherein each nucleic acid of the one or more nucleic acids is different. In embodiments, the one or more nucleic acids each independently includes a sequence having at least 80% identity to any one of SEQ ID NO:1 to SEQ ID NO:4. In embodiments, the one or more nucleic acids includes a first nucleic acid including a sequence having at least 80% identity to SEQ ID NO:1 and a second nucleic acid including a sequence having at least 80% identity to SEQ ID NO:2. In embodiments, the one or more nucleic acids includes a first nucleic acid including a sequence having at least 80% identity to SEQ ID NO:3 and a second nucleic acid including a sequence having at least 80% identity to SEQ ID NO:4.

In embodiments, the guanitoxin-producing bacteria are cyanobacteria. In embodiments, the cyanobacteria are Sphaerospermopsis torques-reginae, Chrysosporum ovalisporum, Cuspidothrix, Cylindrospermopsis, Cylindrospermum, Dolichospermum, Microcystis, Oscillatoria, Planktothrix, Phormidium, Anabaenaflos-aquae, A. lemmermannii Raphidiopsis mediterranea, Tychonema, or Woronichinia. In embodiments, the cyanobacteria are Sphaerospermopsis torques-reginae.

In embodiments, the aqueous liquid is derived from a lake, river, or pond. In embodiments, the aqueous liquid is derived from a public water system or a private water system. In embodiments, the aqueous liquid is ingested, inhaled, or contacted by a subject.

In embodiments, the kit includes an enzyme, deoxynucleoside triphosphates (dNTPs), a control DNA, a detectable compound, or a combination thereof. In embodiments, the kit includes an enzyme. In embodiments, the enzyme is a reverse transcriptase. In embodiments, the enzyme is a polymerase. In embodiments, the polymerase is Taq polymerase. In embodiments, the kit includes dNTPs. In embodiments, the kit includes a control DNA. In embodiments, the control DNA includes a guanitoxin biosynthetic gene or a fragment thereof as provided herein including embodiments thereof. In embodiments, the control DNA includes one or more of SEQ ID NO:23-75 or a portion or fragment thereof, as provided herein including embodiments thereof. In embodiments, the kit includes a detectable label. In embodiments, the detectable label is a fluorescent compound. In embodiments, the fluorescent compound binds non-specifically to double-stranded DNA. The detectable label may include any number of DNA detecting probes or compounds useful for detecting DNA (e.g. SYBR Green, TAQMAN™).

In embodiments, the kit further includes a therapeutic effective for treating guanitoxin-induced toxicity.

### Nucleic Acid Compositions

The compositions provided herein include nucleic acids at least partially complementary to a portion of a guanitoxin biosynthetic gene (e.g. GntA, GntB, GntC, GntD, GntE, GntF, GntG, GntH, GntI, GntJ, GntT) as provided herein including embodiments thereof. The nucleic acid at least partially complementary to a portion of a guanitoxin biosynthetic gene is described in detail throughout this application (including in the description above and in the examples section). Thus, in an aspect is provided a composition including one or more nucleic acids each independently comprising a sequence having at least 80% identity to any one of SEQ ID NO:1 to SEQ ID NO:22, wherein each nucleic acid of the one or more nucleic acids is different.

### EMBODIMENTS

Embodiment 1. A method of detecting guanitoxin-producing bacteria in an aqueous liquid, the method comprising detecting one or more guanitoxin biosynthetic genes in the aqueous liquid, wherein the one or more guanitoxin biosynthetic genes are GntA, GntB, GntC, GntD, GntE, GntF, GntG, GntH, GntI, GntJ, GntT, or a combination thereof.

Embodiment 2. The method of embodiment 1, wherein the one or more guanitoxin biosynthetic genes are GntA, GntJ, GntC, or a combination thereof.

Embodiment 3. The method of embodiment 1 or 2, the method comprising contacting the aqueous liquid with one or more nucleic acids, wherein each of the one or more nucleic acids are at least partially complementary to a portion of the one or more guanitoxin biosynthetic genes.

Embodiment 4. The method of any one of embodiments 1 to 3, wherein the detecting comprises performing a PCR method, an isothermal amplification method, a sequencing method, or a combination thereof.

Embodiment 5. The method of embodiment 3 or 4, wherein the portion of the one or more guanitoxin biosynthetic genes comprises a coding sequence, a promoter region sequence, a terminator region sequence, or an intergene region sequence.

Embodiment 6. The method of embodiment 5, wherein the portion of the one or more guanitoxin biosynthetic genes comprises a coding sequence.

Embodiment 7. The method of any one of embodiments 3 to 6, wherein the one or more nucleic acids each independently comprises a sequence having at least 80% identity to any one of SEQ ID NO:1 to SEQ ID NO:22, wherein each nucleic acid of the one or more nucleic acids is different.

Embodiment 8. The method of embodiment 7, wherein the one or more nucleic acids each independently comprises a sequence having at least 80% identity to any one of SEQ ID NO:1 to SEQ ID NO:4.

Embodiment 9. The method of embodiment 8, wherein the one or more nucleic acids comprises a first nucleic acid comprising a sequence having at least 80% identity to SEQ ID NO:1 and a second nucleic acid comprising a sequence having at least 80% identity to SEQ ID NO:2.

Embodiment 10. The method of embodiment 8, wherein the one or more nucleic acids comprises a first nucleic acid comprising a sequence having at least 80% identity to SEQ ID NO:3 and a second nucleic acid comprising a sequence having at least 80% identity to SEQ ID NO:4.

Embodiment 11. The method of any one of embodiments 1 to 10, wherein the guanitoxin-producing bacteria are cyanobacteria.

Embodiment 12. The method of embodiment 11, wherein the cyanobacteria are Sphaerospermopsis torques-reginae, Chrysosporum ovalisporum, Cuspidothrix, Cylindrospermopsis, Cylindrospermum, Dolichospermum, Microcystis, Oscillatoria, Planktothrix, Phormidium, Anabaenaflos-aquae, A. lemmermannii Raphidiopsis mediterranea, Tychonema, or Woronichinia.

Embodiment 13. The method of any one of embodiments 1 to 12, wherein the aqueous liquid is derived from a lake, river, or pond.

Embodiment 14. The method of any one of embodiments 1 to 12, wherein the aqueous liquid is derived from a public water system or a private water system.

Embodiment 15. The method of any one of embodiments 1 to 14, wherein the aqueous liquid is ingested by, inhaled by, or contacted with a subject.

Embodiment 16. The method of embodiment 15, wherein the subject is treated for guanitoxin-induced toxicity when the one or more guanitoxin biosynthetic genes are detected.

Embodiment 17. A kit for detecting guanitoxin-producing bacteria in an aqueous liquid, the kit comprising one or more nucleic acids each at least partially complementary to a portion of one or more guanitoxin biosynthetic genes, wherein the one or more guanitoxin biosynthetic genes are GntA, GntB, GntC, GntD, GntE, GntF, GntG, GntH, GntI, GntJ, GntT, or a combination thereof.

Embodiment 18. The kit of embodiment 17, wherein the one or more guanitoxin biosynthetic genes are GntA, GntJ, GntC, or a combination thereof.

Embodiment 19. The kit of embodiment 17 or 18, wherein the portion of the one or more guanitoxin biosynthetic genes comprises a coding sequence, a promoter region sequence, a terminator region sequence, or an intergene region sequence.

Embodiment 20. The kit of embodiment 19, wherein the portion of the one or more guanitoxin biosynthetic genes comprises a coding sequence.

Embodiment 21. The kit of any one of embodiments 17 to 20, wherein the one or more nucleic acids each independently comprises a sequence having at least 80% identity to any one of SEQ ID NO:1 to SEQ ID NO:22, wherein each nucleic acid of the one or more nucleic acids is different.

Embodiment 22. The kit of embodiment 21, wherein the one or more nucleic acids each independently comprises a sequence having at least 80% identity to any one of SEQ ID NO:1 to SEQ ID NO:4.

Embodiment 23. The kit of embodiment 22, wherein the one or more nucleic acids comprises a first nucleic acid comprising a sequence having at least 80% identity to SEQ ID NO:1 and a second nucleic acid comprising a sequence having at least 80% identity to SEQ ID NO:2.

Embodiment 24. The kit of embodiment 22, wherein the one or more nucleic acids comprises a first nucleic acid comprising a sequence having at least 80% identity to SEQ ID NO:3 and a second nucleic acid comprising a sequence having at least 80% identity to SEQ ID NO:4.

Embodiment 25. The kit of any one of embodiments 17 to 24, wherein the guanitoxin-producing bacteria are cyanobacteria.

Embodiment 26. The kit of embodiment 25, wherein the cyanobacteria are Sphaerospermopsis torques-reginae, Chrysosporum ovalisporum, Cuspidothrix, Cylindrospermopsis, Cylindrospermum, Dolichospermum, Microcystis, Oscillatoria, Planktothrix, Phormidium, Anabaenaflos-aquae, A. lemmermannii Raphidiopsis mediterranea, Tychonema, or Woronichinia.

Embodiment 27. The kit of any one of embodiments 17 to 26, wherein the aqueous liquid is derived from a lake, river, or pond.

Embodiment 28. The kit of any one of embodiments 17 to 26, wherein the aqueous liquid is derived from a public water system or a private water system.

Embodiment 29. The kit of any one of embodiments 17 to 27, wherein the aqueous liquid is ingested, inhaled, or contacted by a subject.

Embodiment 30. The kit of any one of embodiments 17 to 29, further comprising an enzyme, deoxynucleoside triphosphates (dNTPs), a control DNA, a detectable label, or a combination thereof.

Embodiment 31. The kit of any one of embodiments 17 to 30, further comprising a therapeutic effective for treating guanitoxin-induced toxicity.

### EXAMPLES

### Example 1: Introduction to Exemplary Studies

The complete guanitoxin BGC and pathway and diagnostic intermediates are described herein. Inventors have validated intermediates with synthetic and chemoenzymatic standards, and surveyed environmental metatranscriptomic and metagenomic datasets to reveal the global prevalence of guanitoxin biosynthetic capability in both rural and populated watersheds. These discoveries are contemplated to accelerate understanding of the distribution and impact of lethal, yet essentially unmonitored, guanitoxin.

### Example 2: Results and Discussion

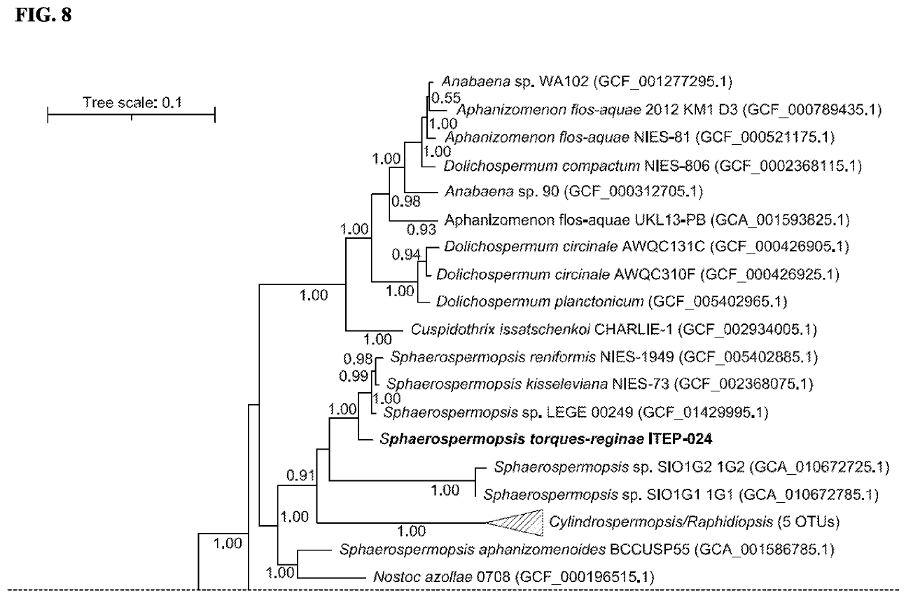

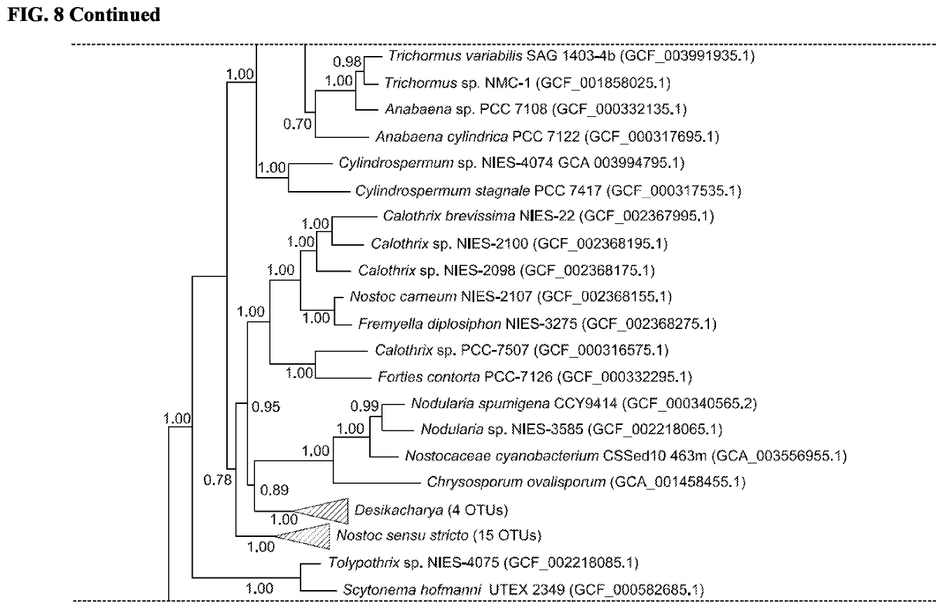

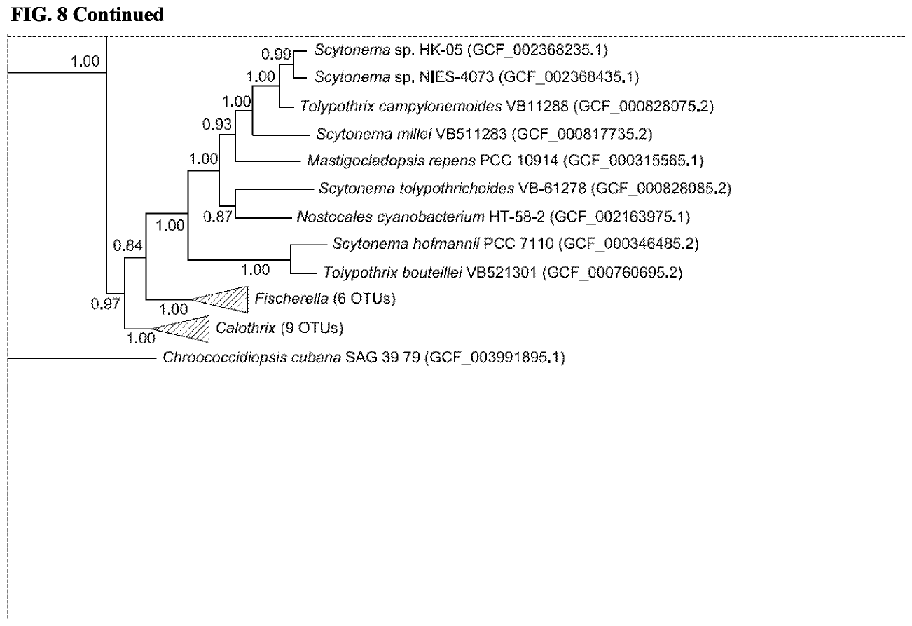

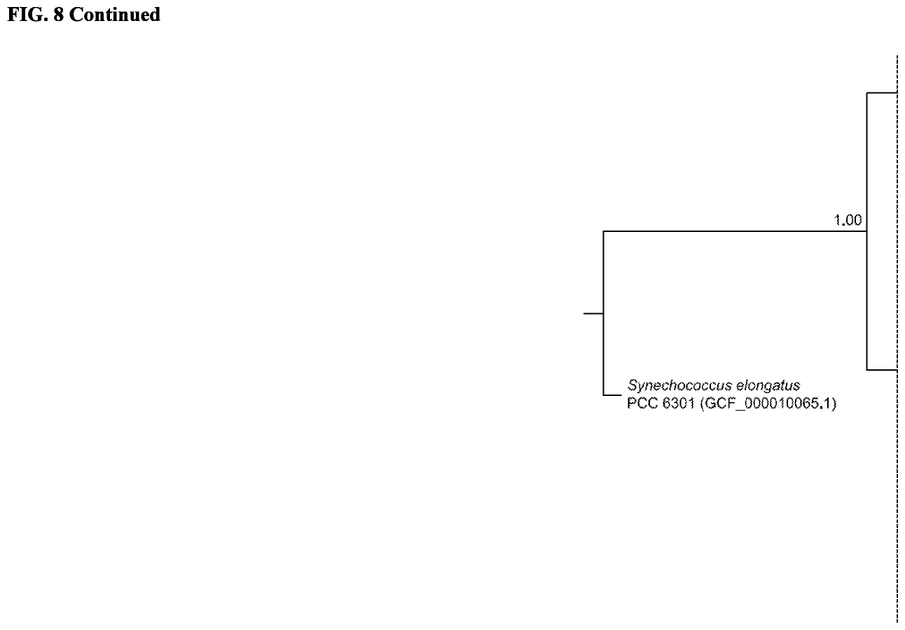

Sequencing of the genome of the guanitoxin-producing cyanobacterial strain Sphaerospermopsis torques-reginae ITEP-024 (GenBank accession no. CP080598)²⁶ acquired from a toxic cyanoHAB in the Tapacura Reservoir, Pernambuco, Brazil (FIGS. 7-8)²⁷ is described herein. Bioinformatic analysis of the resulting 5.24 Mbp genome with prediction tools such as antiSMASH¹⁸ identified 11 natural productBGCs (FIG. 2A, Table 3). None, however, were consistent with guanitoxin biogenesis involving predicted amino acid and organophosphate biochemistry.

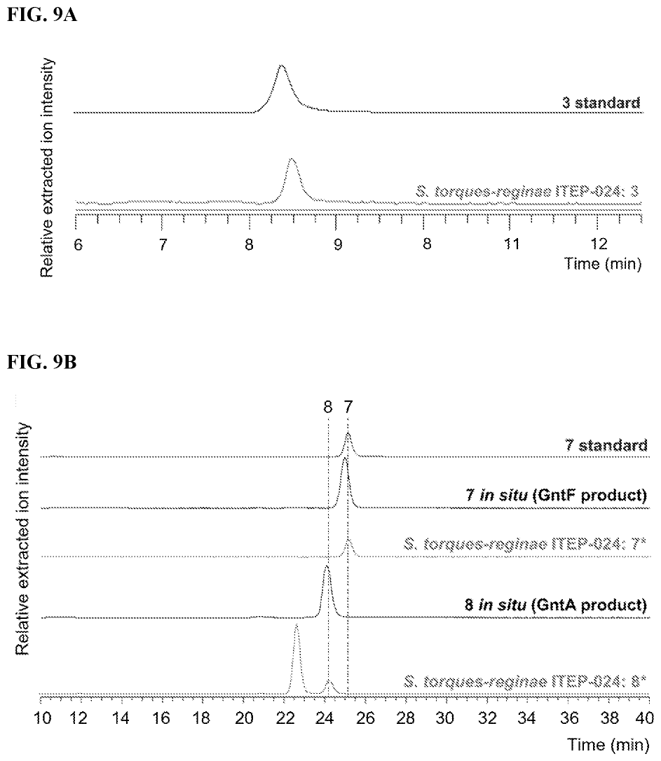

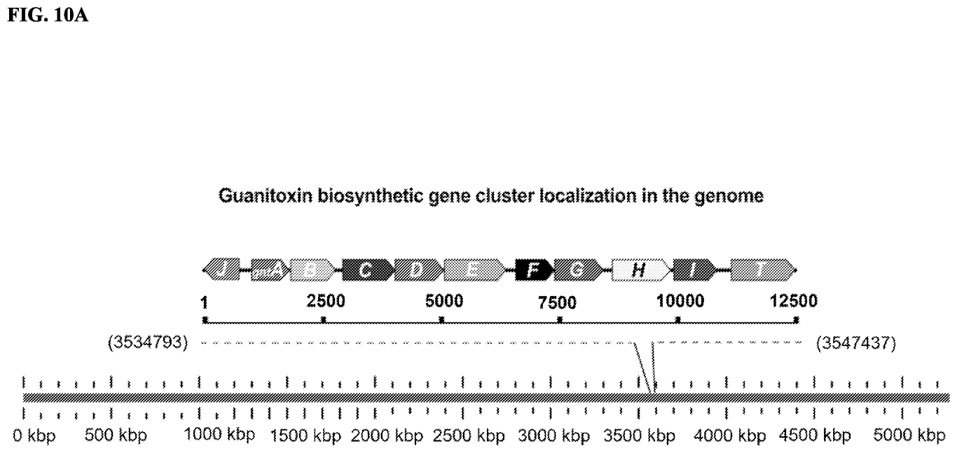

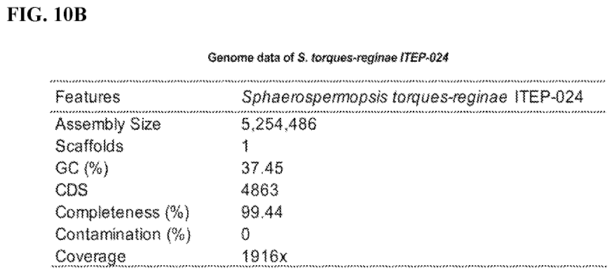

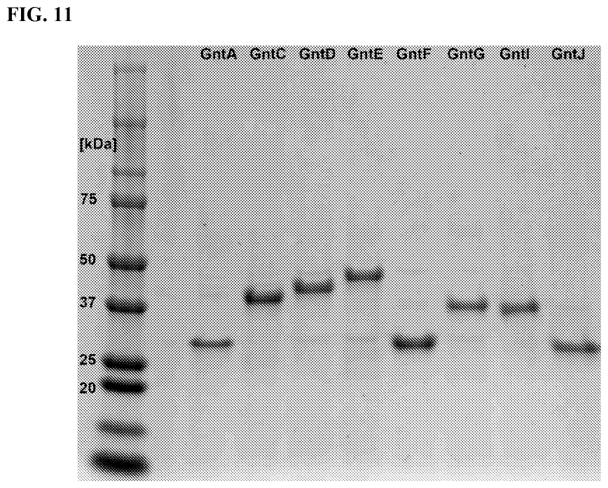

To identify candidate guanitoxin BGCs, genes associated with the arginine metabolites (S)-ψ-hydroxy-L-arginine (2) and L-enduracididine (3) were under scrutiny. Both 2 and 3 were previously isolated from guanitoxin-producing Anabaenaflos-aquae^{24,25} and converted in vivo to guanitoxin via stable isotope labeling experiments (FIG. 1B).²⁵ Subsequently identification of 3 in Sphaerospermopsis torques-reginae ITEP-024 extracts were done, further supporting its intermediacy in guanitoxin biosynthesis (FIGS. 9A-9B). As amino acid 3 and its epimers are incorporated in actinobacterial non-ribosomal peptide antibiotics such as a mannopeptimycin,²⁸ a teixobactin,²⁹ and an enduracidin,³⁰ and constructed via a pyridoxal-5′-phosphate (PLP)-based enzymatic transformations, Sphaerospermopsis torques-reginae ITEP-024 genome was queried for homologous genes to known 3-producing enzymes mppP/mppQ/mppR from the mannopeptimycin biosynthetic pathway.^{31 33} While the mannopeptimycin biosynthetic pathway did not locate close homologs, it did identify a candidate BGC encoding three PLP-dependent enzymes embedded within a unique 12.5 kilobytes (kb) gene cluster consisting of 10 metabolic enzymes (gntA-J) and a putative transporter (gntT) (FIG. 2B). The identified candidate gnt BGC is unique and not found in any reference cyanobacterial genome currently available in the National Center for Biotechnology Information (NCBI) database, nor in the metagenome-assembled genomes (MAGs) of the JGI Earth Microbiome Project.³⁴ While most of the encoded enzymes did not have high sequence similarity to characterized homologs (FIGS. 10A-10B, Table 9), initial sequence and structural homology analyses identified multiple biosynthetic functions consistent with the guanitoxin assembly; these included oxidation (gntA, gntB, gntD), PLP-dependent reactions (gntC, gntE, gntG), S-adenosylmethionine (SAM)-dependent methylations (gntF and gntJ), and phosphate biochemistry (gntI and gntH) (FIG. 2B, FIGS. 10A-OB, Table 9). Therefore, a putative guanitoxin biosynthetic pathway was constructed based on previously isolated chemical intermediates and gnt bioinformatic annotations (FIG. 2C). To experimentally link candidate Gnt enzymes to toxin biosynthesis, the gnt genes were synthesized and then expressed and purified the majority to homogeneity as Escherichia coli codon optimized N-terminal His₆ fusion proteins (FIG. 11).

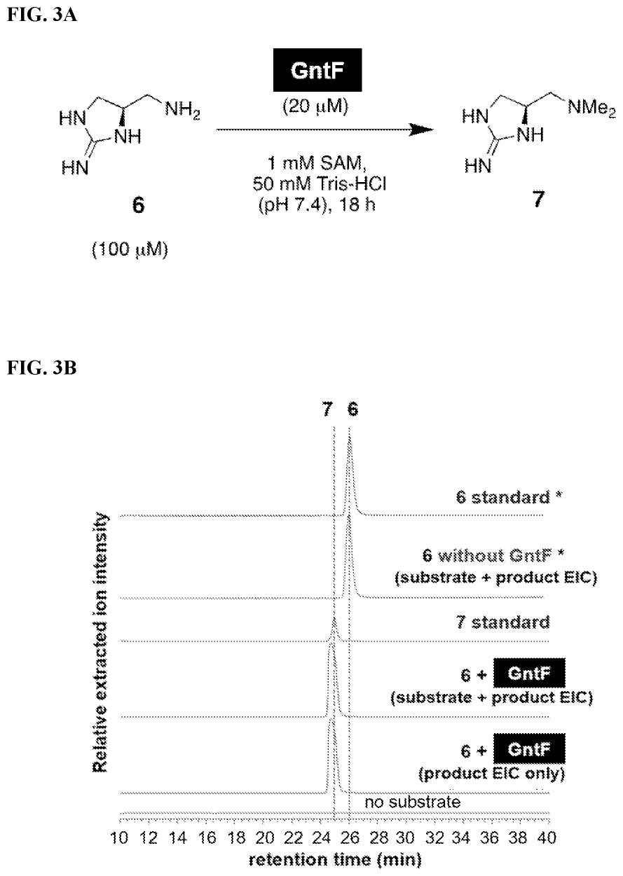

Provided herein is a guanitoxin biosynthetic reaction focused on the presumed N,N-dimethylation of 6 to 7 by predicted N-methyltransferase GntF. The chemical structures of putative intermediates 6 and 7 are novel molecules yet to be observed in other characterized biosynthetic pathways, so their interconversion would strongly implicate the gnt BGC. Intermediates 6 and 7 were chemically synthesized from (S)-Garner's aldehyde in 6 and 5 steps respectively; upon incubation of synthetic 6 with GntF in the presence of excess SAM, its efficient conversion to dimethylated 7 following hydrophilic interaction liquid chromatography mass spectrometry (HILIC-MS) analyses (FIGS. 3A-3B) were observed. It was also observed 7 in Sphaerospermopsis torques-reginae ITEP-024 extracts, lending further physiological relevance to its role in guanitoxin biogenesis (FIGS. 9A-9B). With this encouraging result in hand, it was next sought to connect these key biosynthetic intermediates to their primary metabolic precursors and the mature toxin itself.

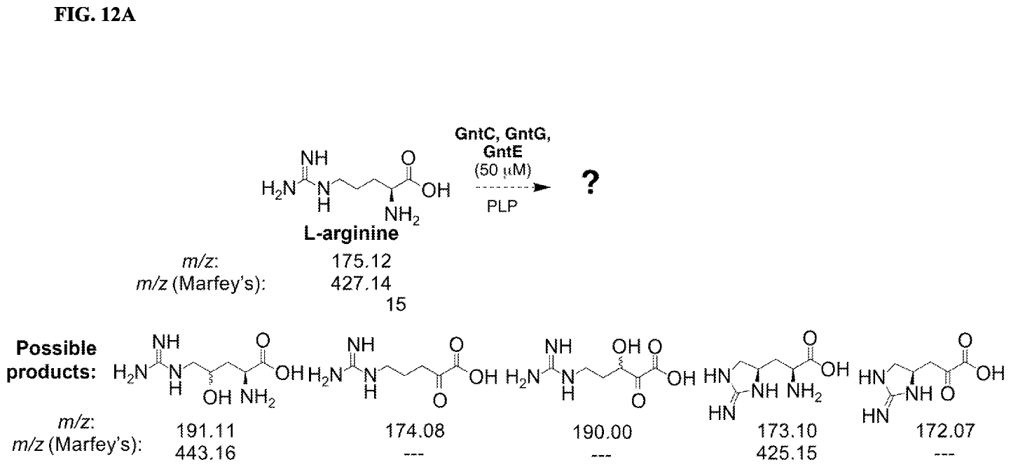

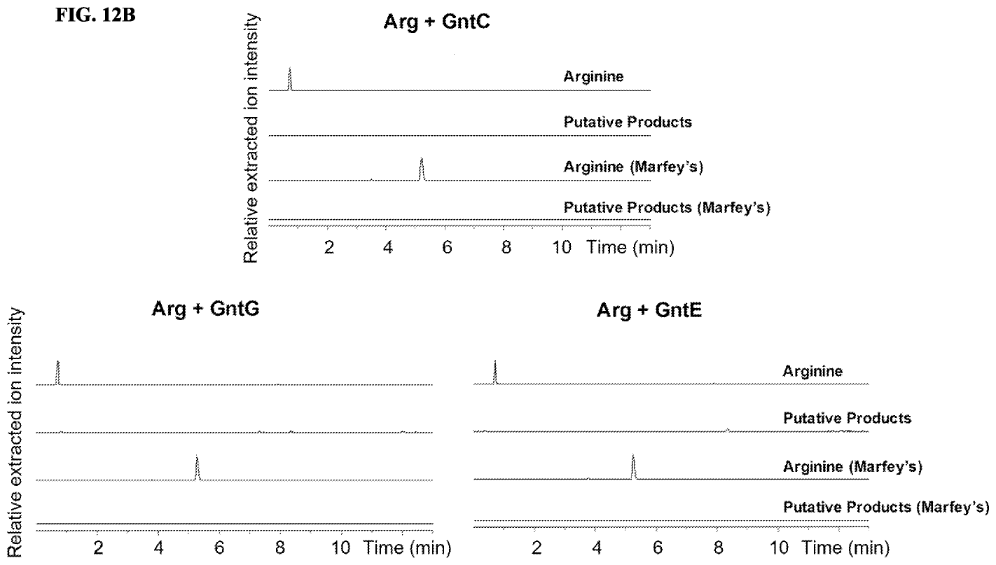

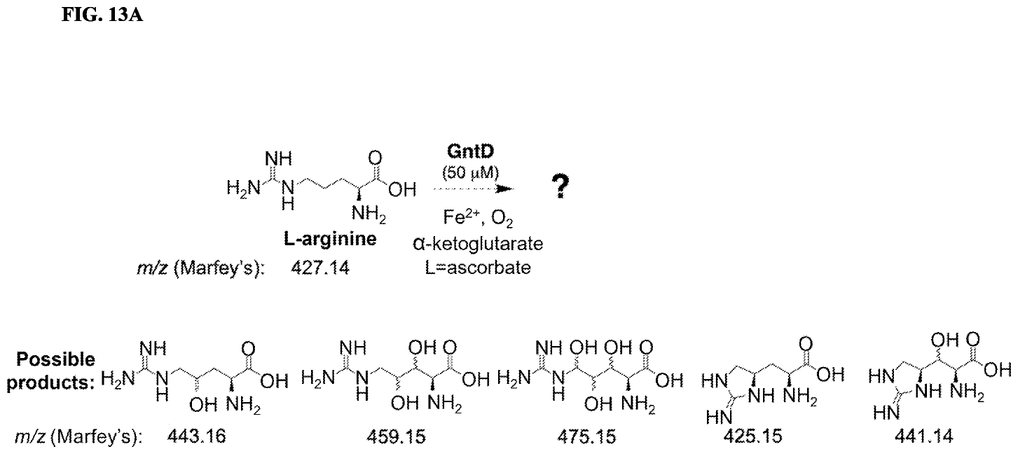

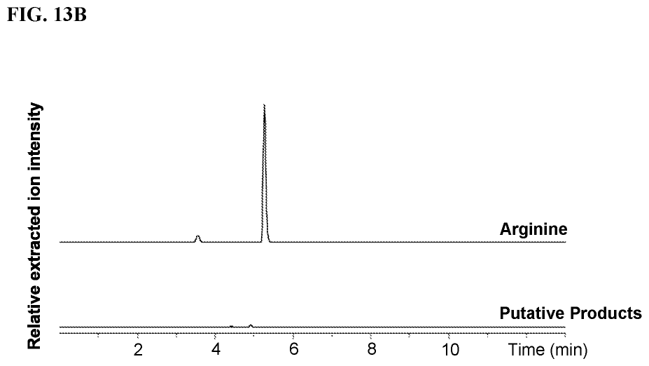

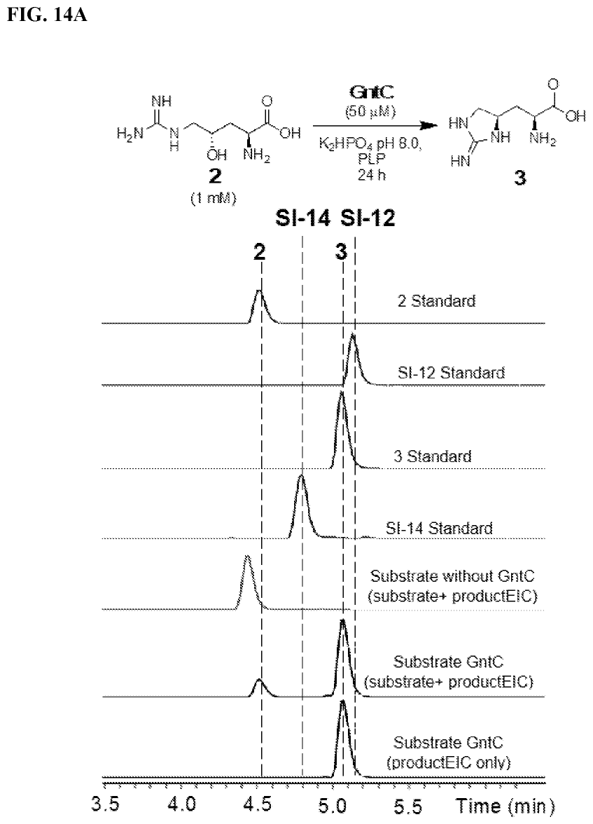

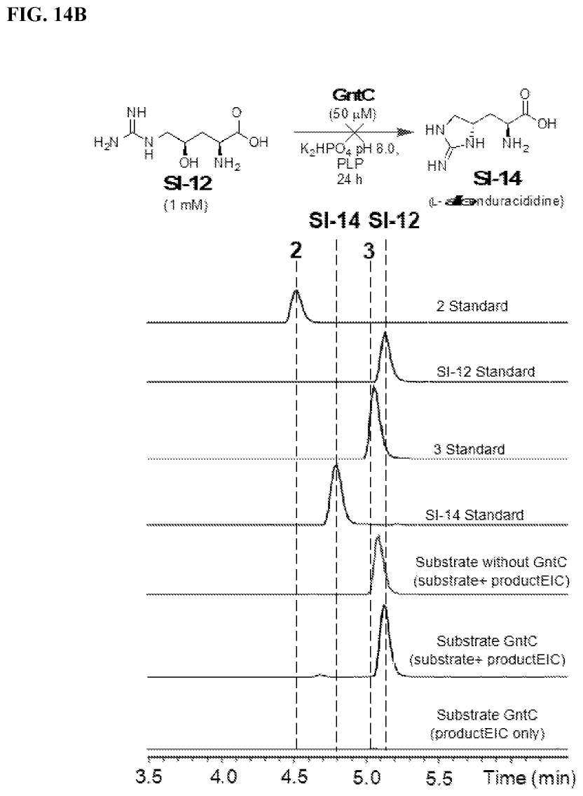

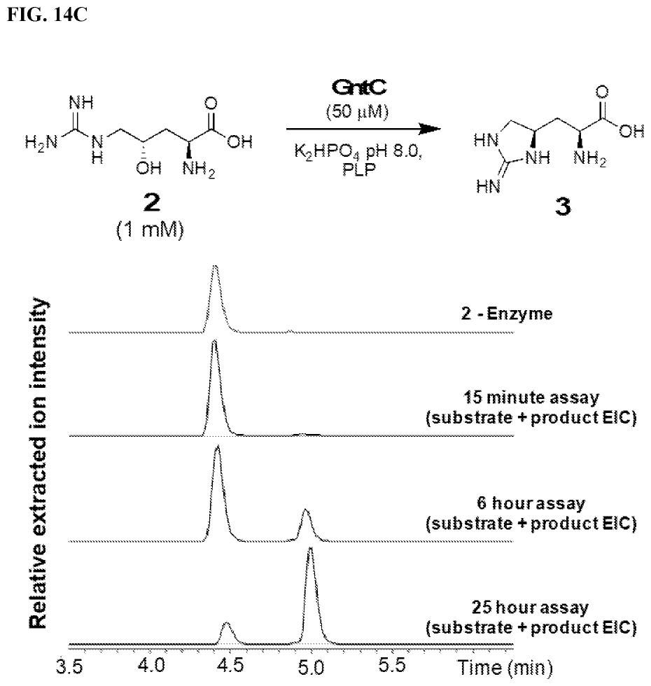

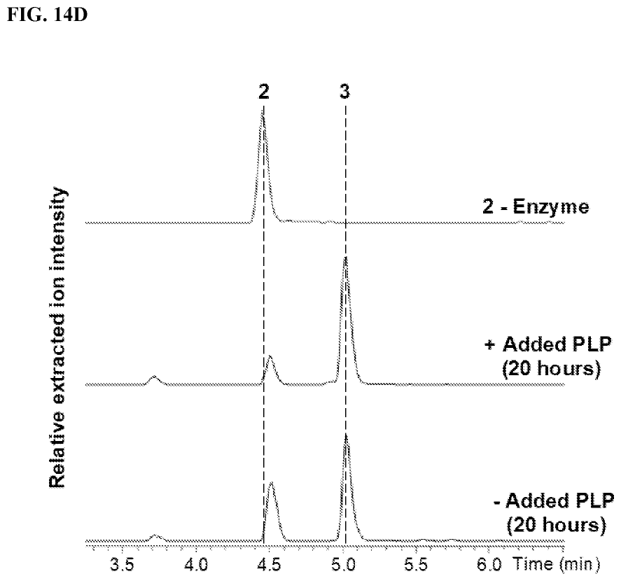

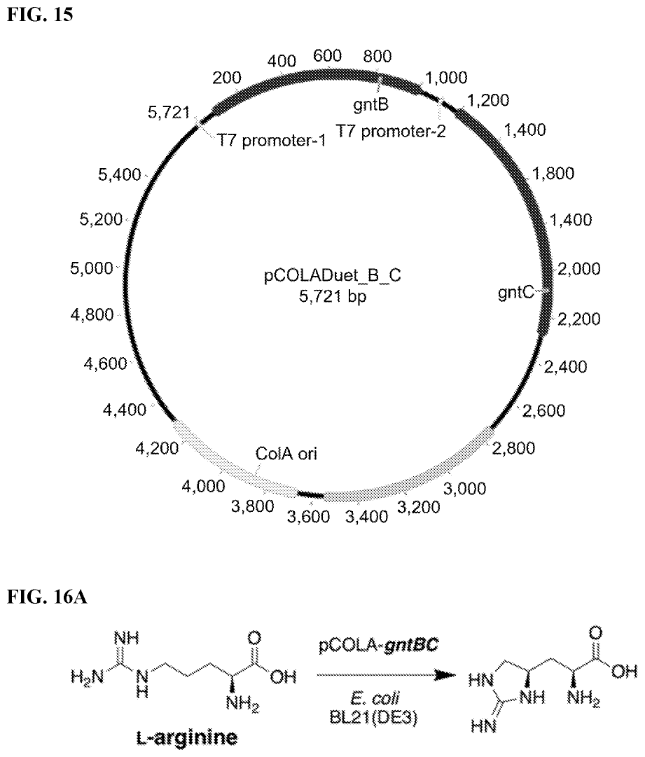

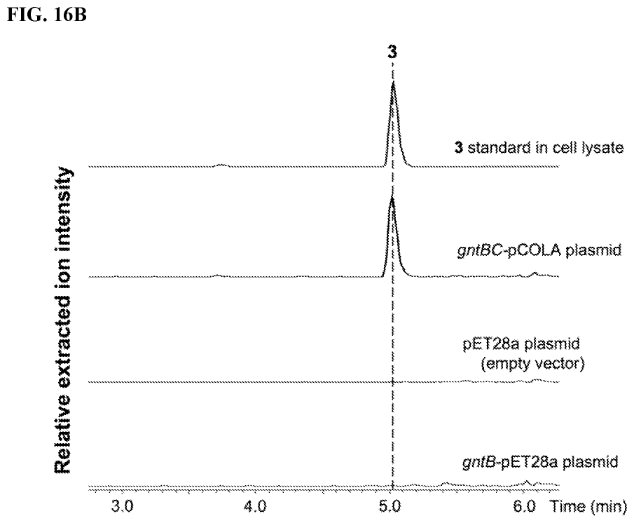

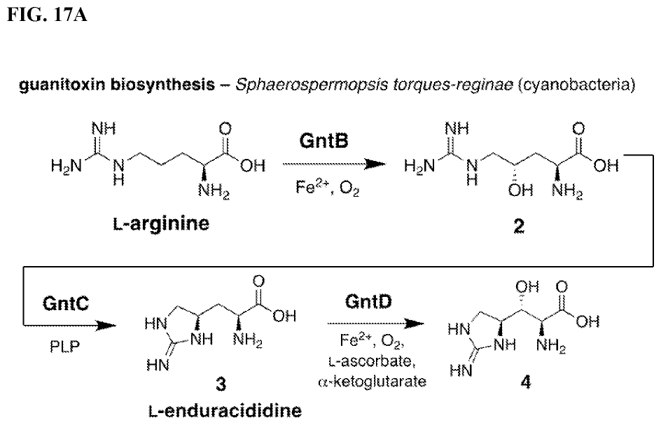

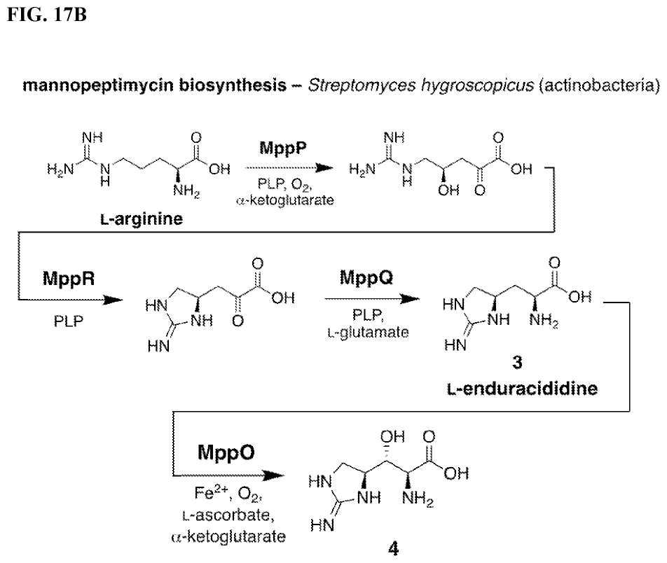

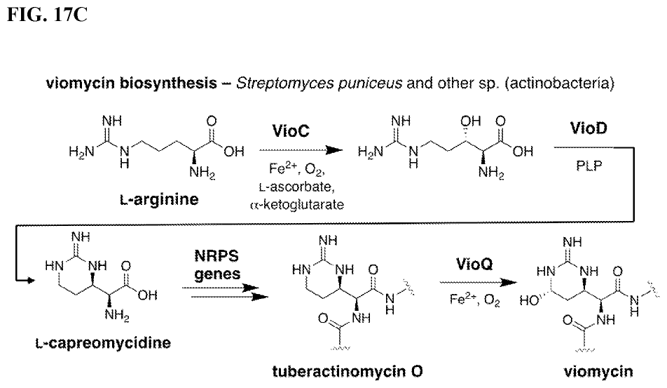

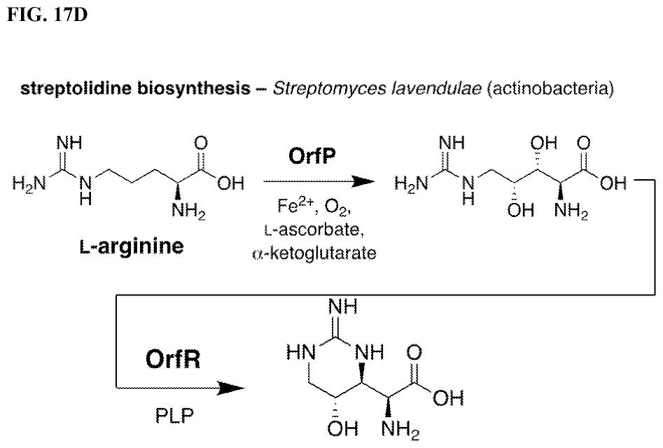

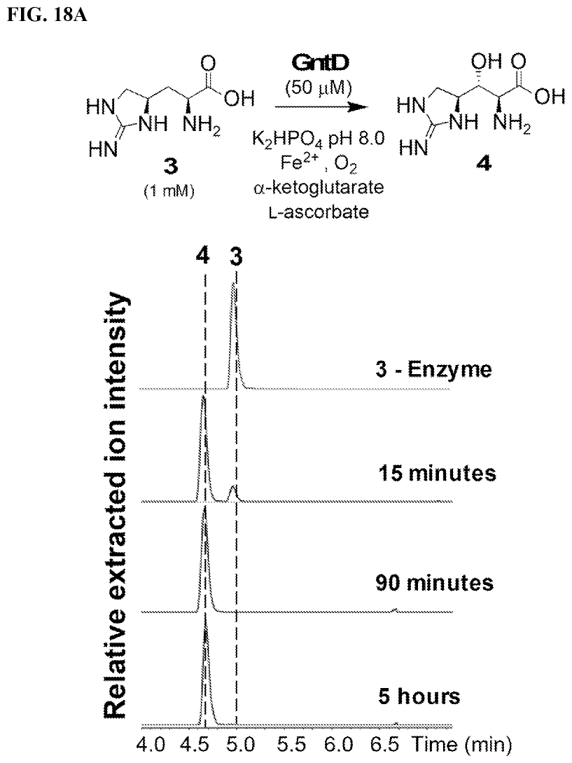

Multiple characterized biosynthetic pathways employ PLP-dependent enzymes to explore their dehydrogenation, oxidation, and transamination chemistries on L-arginine.³⁵ However, none of the three PLP-dependent enzymes (GntC, GntE, GntG) nor the soluble a-ketoglutarate ferrous (Fe²⁺)-dioxygenase GntD showed any clear activity towards this amino acid (FIGS. 12A-13B). Therefore, initialization of in vitro enzymology efforts with known biosynthetic intermediates 2 and 3 were done, which were chemically synthesized following adaptation of previously established synthetic procedures.^{36,37} Surprisingly, GntC catalyzed the highly diastereoselective PLP-dependent cyclodehydration of 2 into 3, without any additional enzymes or co-substrates (FIGS. 14A-14D). The stereochemical differences between chemically synthesized substrate and product diastereomers were magnified via 1-fluoro-2,4-dinitrophenyl-5-L-alanine amide (L-FDAA, Marfey's reagent) derivatization and ultra-performance liquid chromatography mass spectrometry (UPLC-MS) analysis, enabling to determine that GntC was highly diastereoselective for the (S)-hydroxy stereoisomer of ψ-hydroxy-L-arginine while exclusively generating 3 over its L-allo-enduracididine epimer (FIGS. 14A-14D). While transmembrane protein GntB proved intractable to soluble protein expression, the in vivo expression of a gntB-gntC-containing plasmid in E. coli successfully produced 3 (FIGS. 15-16B), thus corroborating GntB's initiating role in the construction of 2. Cyanobacterial 3 biosynthesis presents a divergent reaction strategy to known actinobacterial cyclic arginine biosyntheses that either rely on multiple PLP-dependent enzymes,^{31 33} or form 6-membered capreomycidine rings from linear hydroxylated arginine precursors (FIGS. 17A-17D).^{38 41}

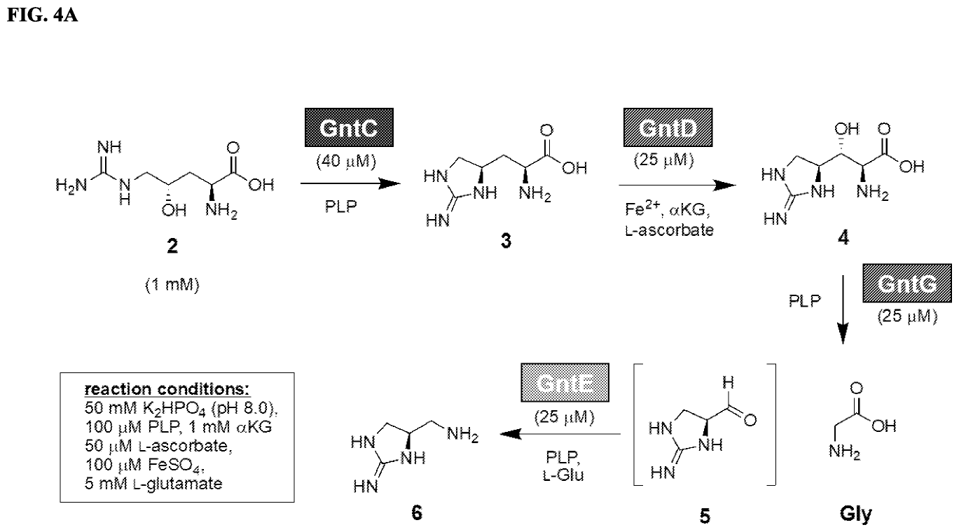

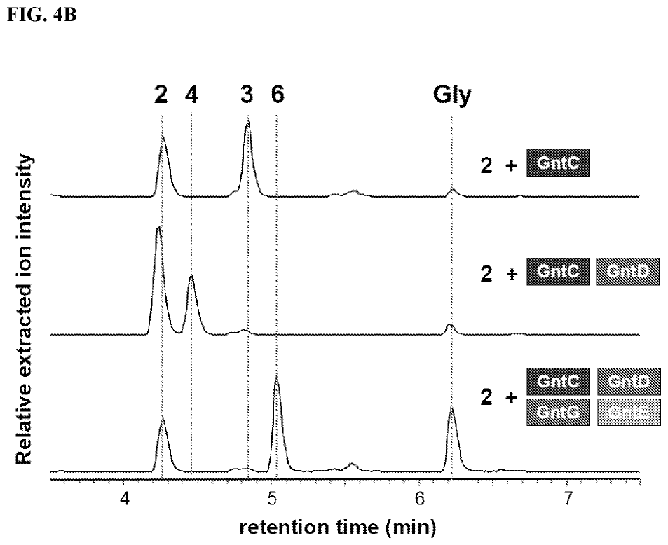

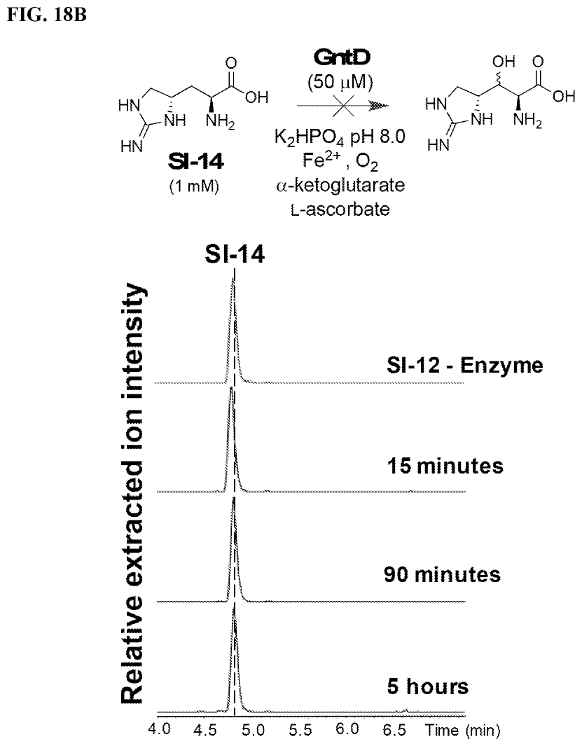

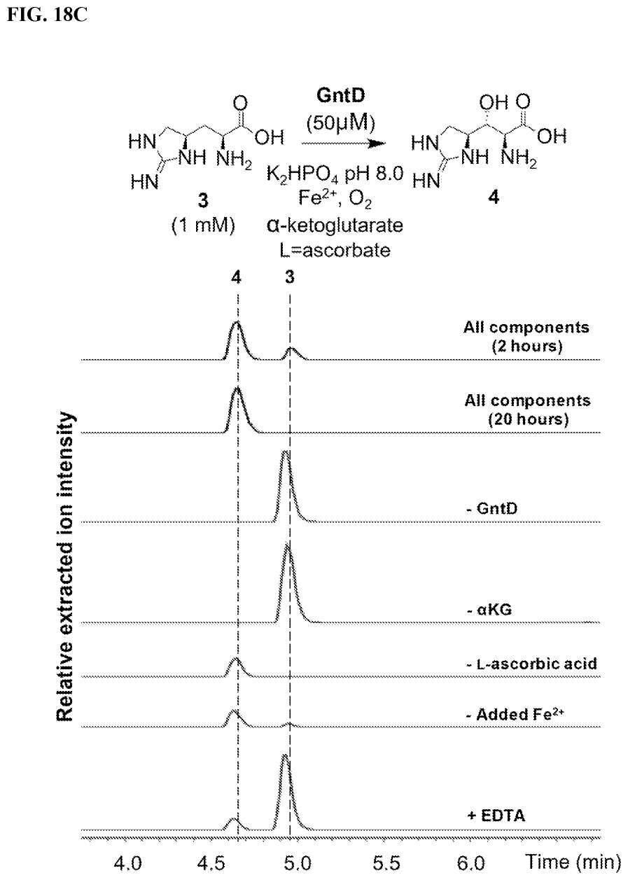

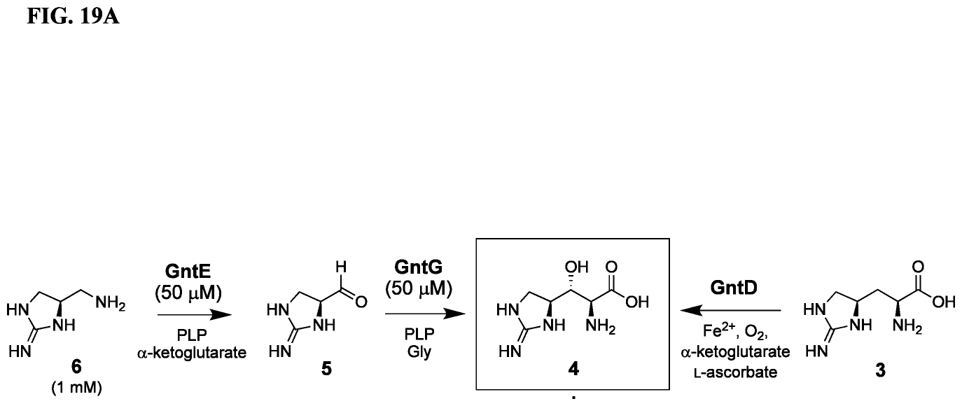

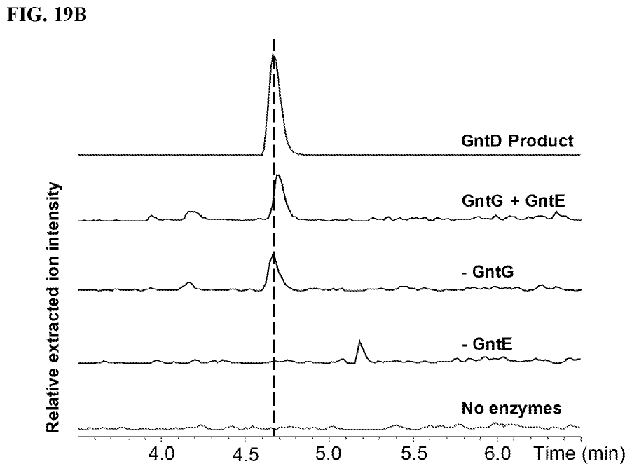

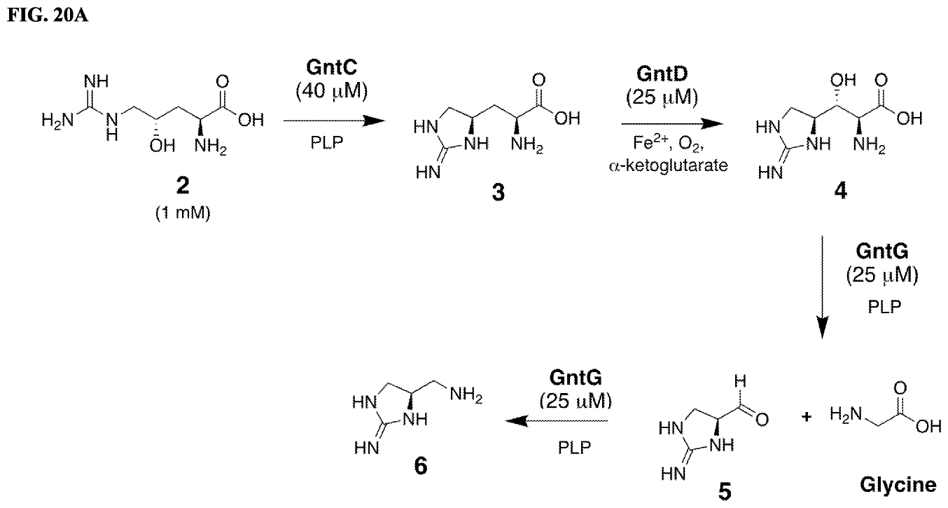

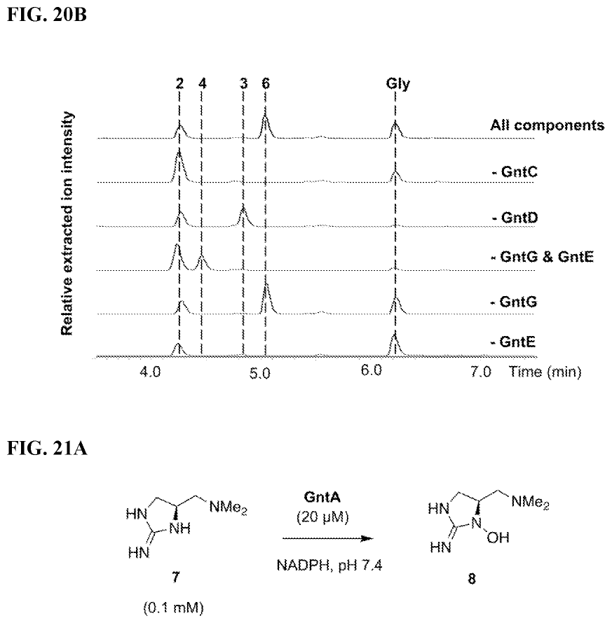

Next GntD was established as an enduracididine B-hydroxylase in converting 3 to 4 in the presence of ferrous Fe²⁺, α-ketoglutarate, and L-ascorbate as previously reported for the Streptomyces enzyme Mannopeptimycin biosynthesis protein O (MppO) (FIGS. 18A-18C).⁴² To connect 4 with diagnostic intermediate 6, a two-enzyme cascade with aldolase GntG and transaminase GntE excising glycine and converting the resultant aldehyde to a primary amine, respectively was proposed. The GntE/GntG cascade was validated by performing the reaction in reverse to construct 4 from 6, glycine, α-ketoglutarate, PLP, and the two PLP-dependent enzymes (FIGS. 19A-19B). Applying all four biosynthetic enzymes and their requisite cofactors and co-substrates simultaneously in one pot converted 2 to 6 and the glycine byproduct, with intermediate aldehyde 5 not observed due to its presumed instability and absence of a primary amine for L-FDAA derivatization (FIGS. 4A-4B). Exclusion of individual enzymes halted progression along this four-step pathway in a manner consistent with our biosynthetic proposal (FIGS. 20A-20B).

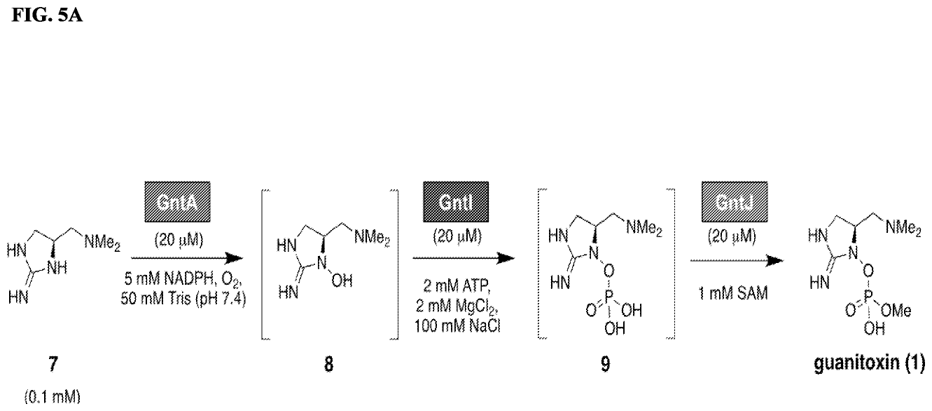

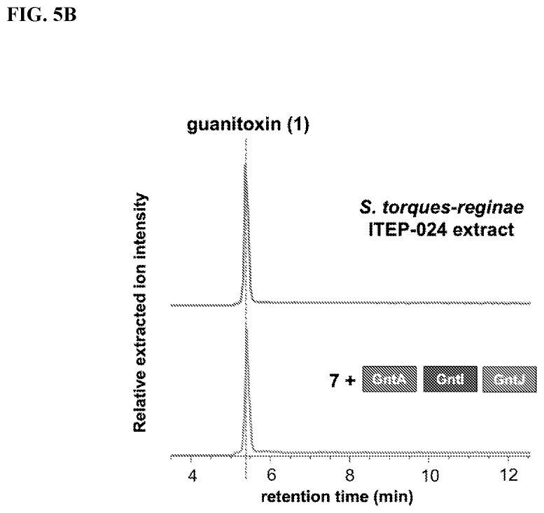

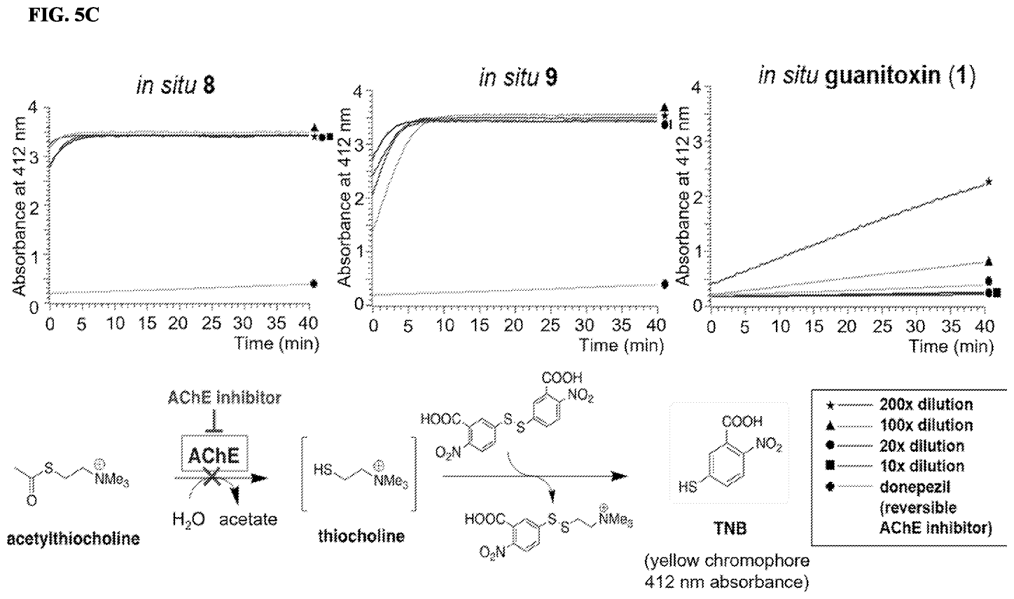

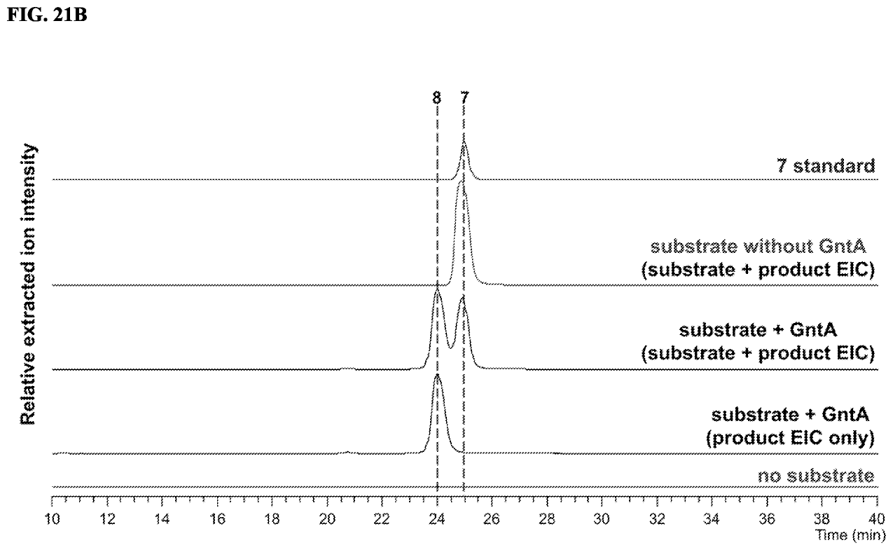

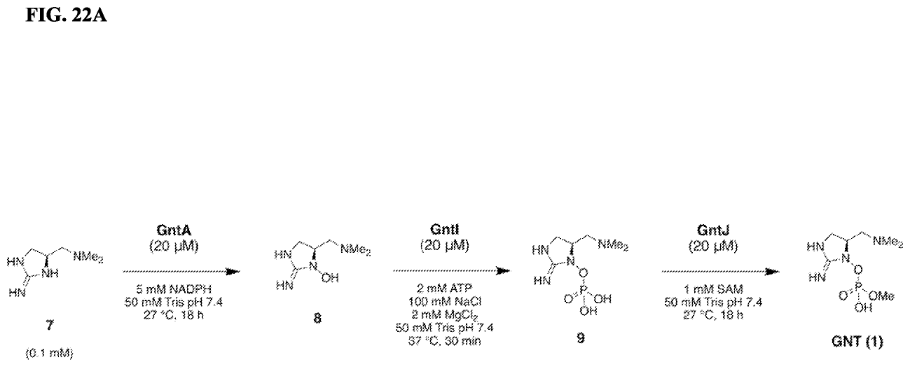

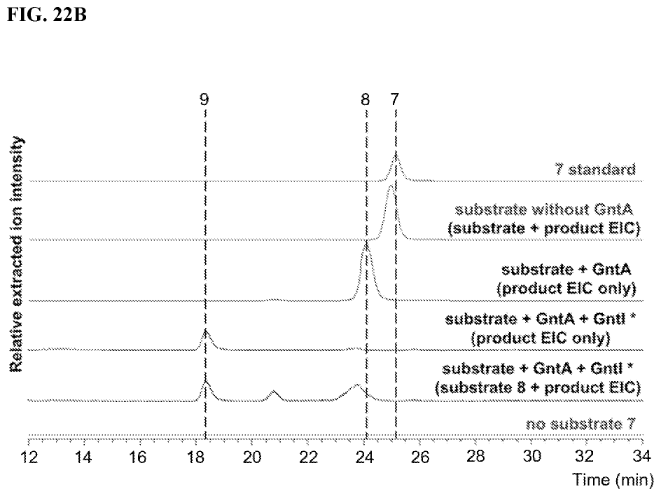

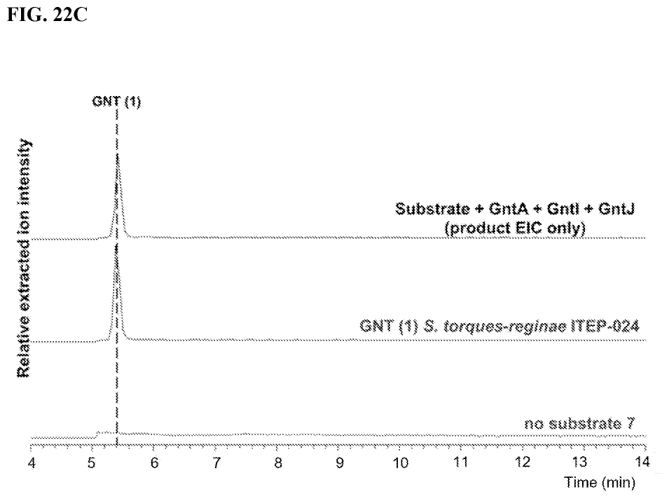

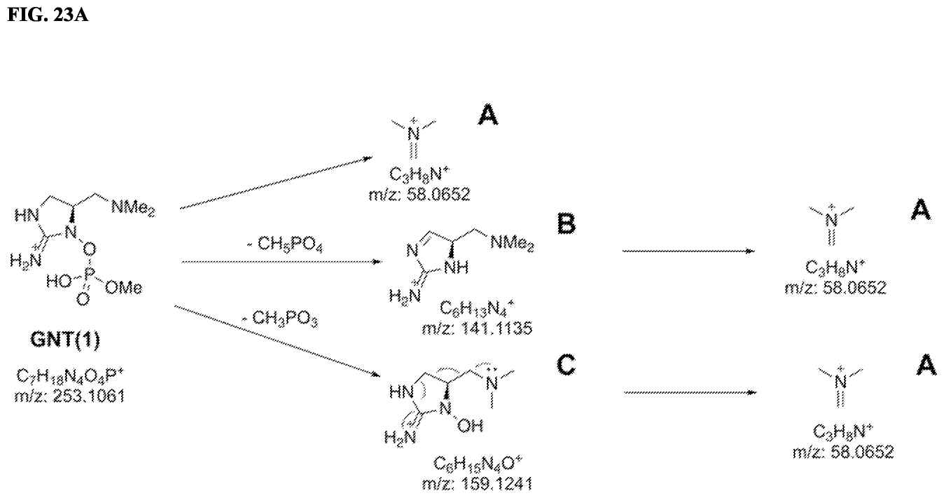

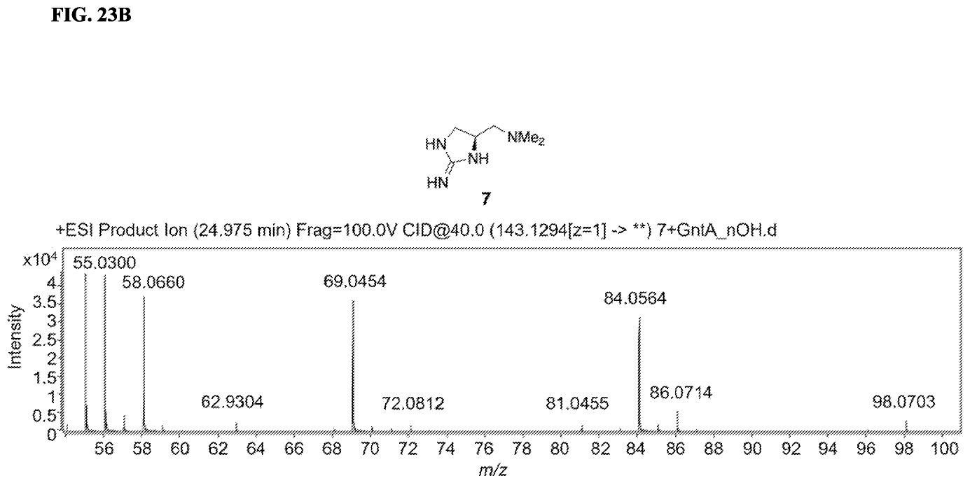

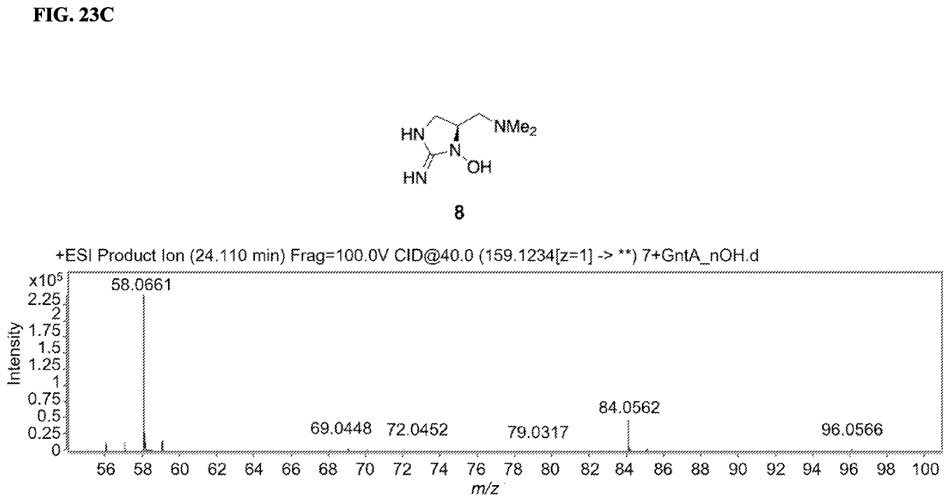

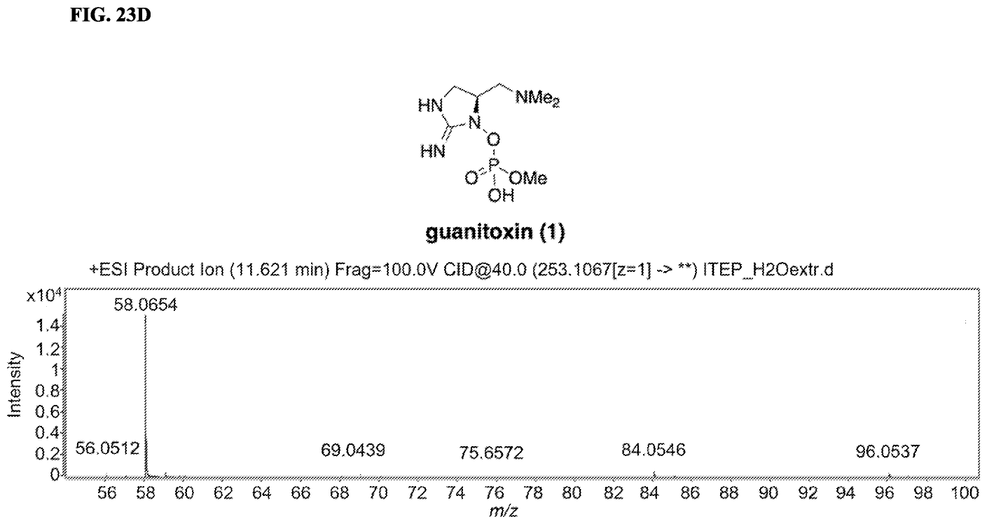

After assembling the guanitoxin carbon backbone via six biosynthetic transformations, focus was on the enzymatic construction of the N-hydroxylated methyl phosphate that functions as the anticholinesterase pharmacophore (FIG. 5A). In the presence of the unusual heme-containing GntA and excess nicotinamide adenine dinucleotide phosphate (NADPH) under ambient aerobic conditions, the selective mono-oxygenation of tertiary amine 7 to product 8 (FIGS. 21A-21B) was observed. This reaction is reminiscent of the homologous heme-oxygenase dcsA gene encoded N^{ω}-hydroxylation of L-arginine that initiates a specialized pathway to D-cycloserine in Streptomyces bacteria.⁴³ Ultrafiltration of the GntA reaction mixture, followed by the addition of kinase GntI, O-methyltransferase GntJ, and their cofactors and co-substrates generated phosphorylated intermediate 9 and guanitoxin, with identical HILIC-MS properties to those observed in Sphaerospermopsis torques-reginae ITEP-024 extracts (FIGS. 5B, 9A-9B, 22A-22C, 23A-23D). Subsequently investigation was done on recombinant human acetylcholinesterase inhibition with in situ generated 8, 9, and guanitoxin (FIG. 5C). Only the mature neurotoxin possessed inhibitory activity, establishing that the GntJ-catalyzed O-methylation is crucial for toxicity. Overall, the chemical, biochemical, and metabolomic analyses strongly connect guanitoxin biosynthesis to the gnt BGC and provide a genetic handle to investigate this cyanotoxin environmentally.

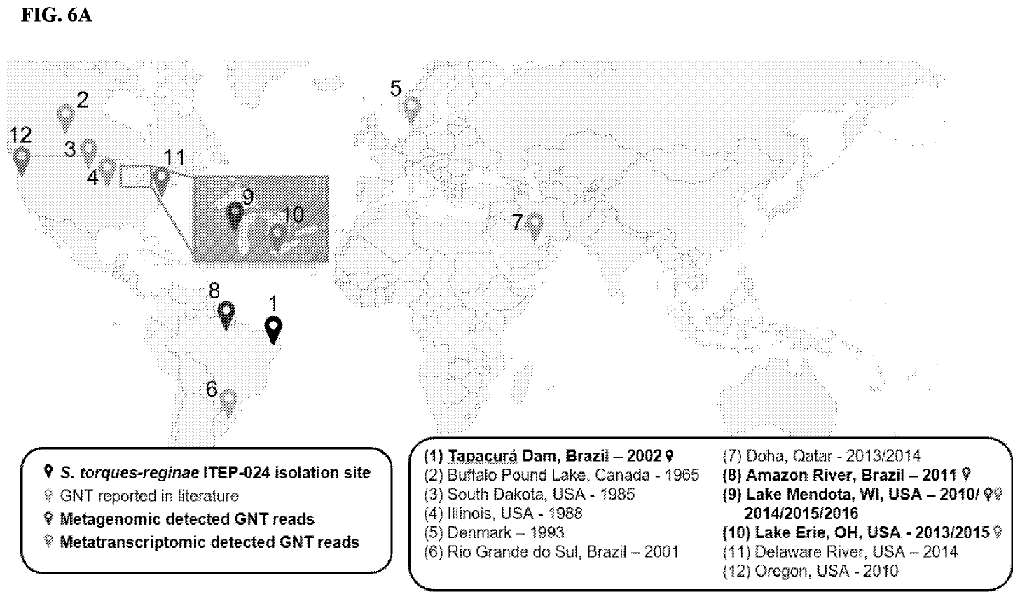

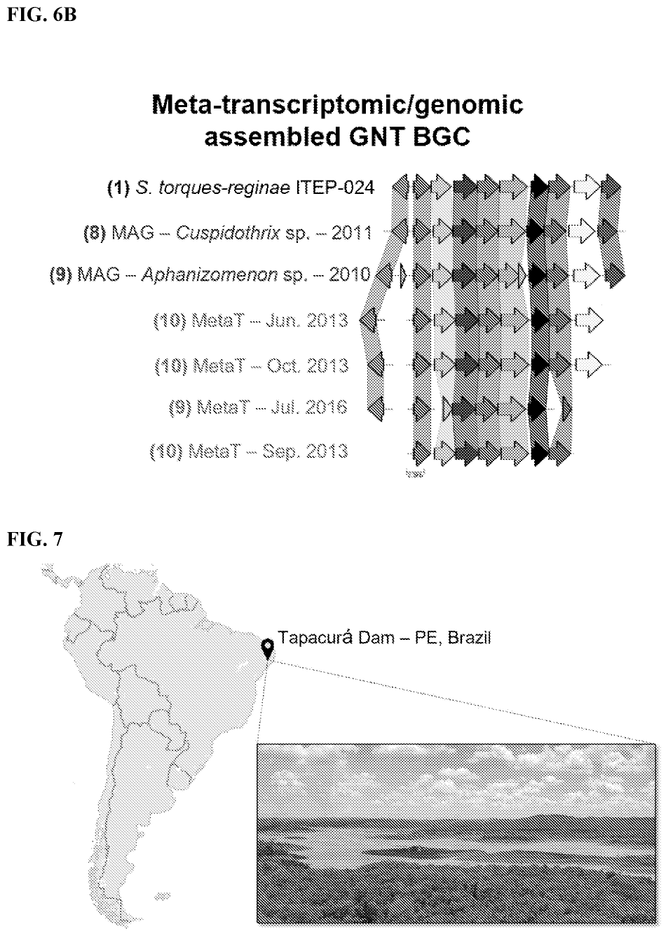

After validating the guanitoxin biosynthetic genes in Sphaerospermopsis torques-reginae ITEP-024, next their environmental distribution was explored. As observed previously that no gnt BGC homologs were found in publicly available assembled genomes and metagenomes, instead queries were done for raw, unassembled environmental sequencing data. Specifically, all available (108,465) NCBI Sequence Read Archive (SRA) metagenomic (metaG) and metatranscriptomic (metaT) datasets were searched using the SearchSRT tool. The search identified 13 datasets with at least one read aligning to the gnt BGC with translated-nucleotide to protein sequence identity, despite the fact that the computationally efficient SearchSRA tool is sensitivity-limited to detect only highly abundant (metaG) or highly expressed (metaT) genes. Through independent de novo assembly it was confirmed regarding the complete or nearly complete gnt BGC from six of these SRA datasets (FIGS. 6A-6B), including two metaT samples. In the metaT samples, the gnt BGC was discovered as a polycistronic transcript including all genes except gntJ. The abovementioned polycistronic structure suggested that even limited transcriptomic detection of the gnt genes might be diagnostic for active guanitoxin biosynthesis. Therefore, to explore the feasibility of metaT data for environmental gnt BGC surveys, all 2,610 freshwater metatranscriptomic SRA datasets were comprehensively queried using a custom full-sensitivity SRA search pipeline, and ultimately identified 78 gnt BGC hits from Lake Erie, Ohio, United States of America (USA); Lake Mendota, Wisconsin, USA; the Amazon River, Brazil; the Columbia River, Oregon, USA; and the Delaware River, Delaware, USA (FIGS. 6A-6B, Table 4). These detections occur in independent sequencing datasets within the same watershed over multiple years, such as in Lake Mendota from 2010-2016, indicating the long-term undetected presence of guanitoxin biosynthetic capacity in publicly accessible freshwater sources.

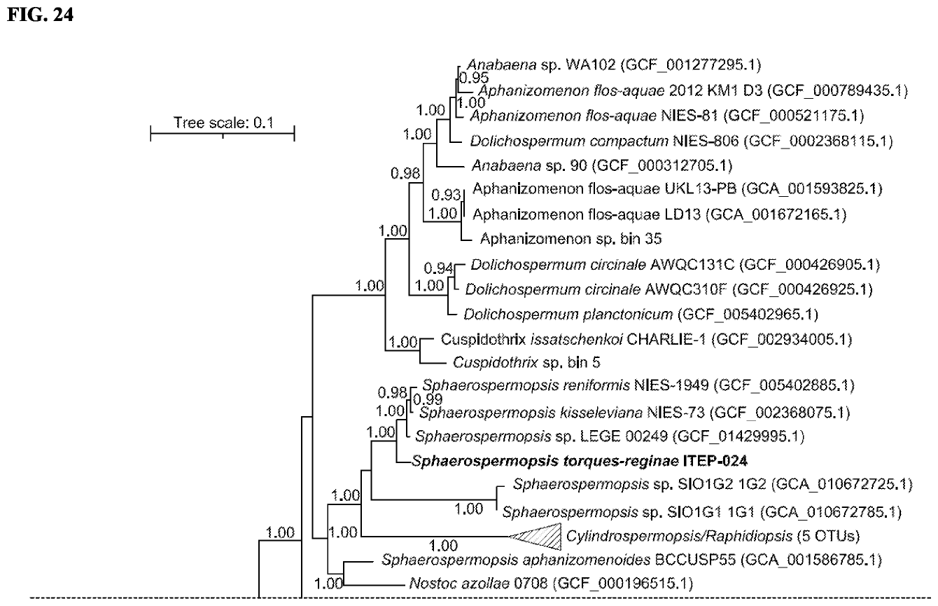

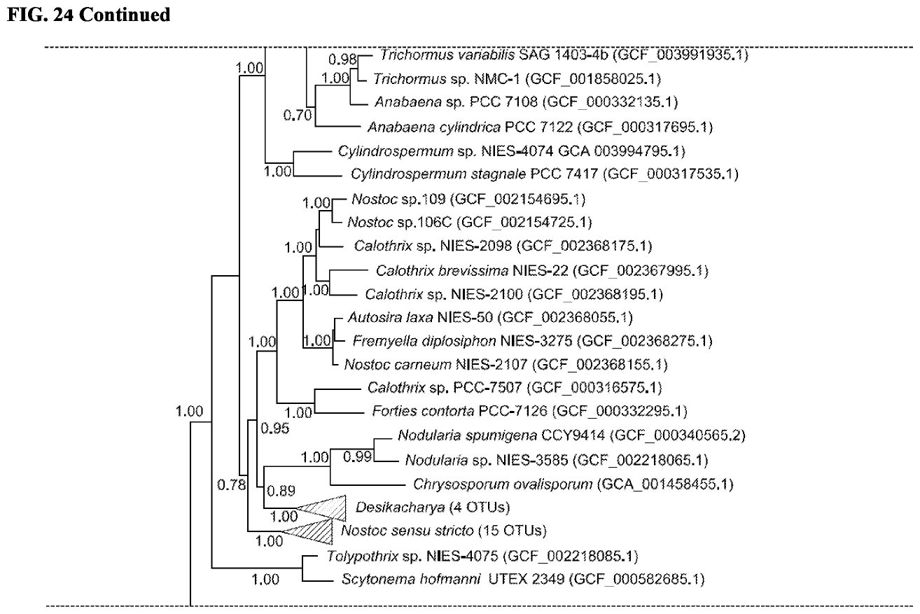

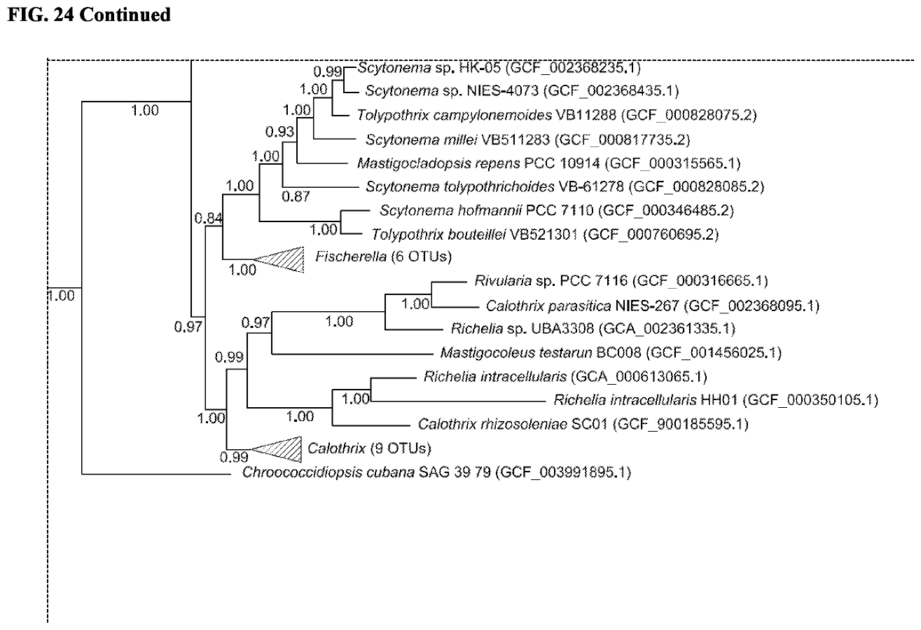

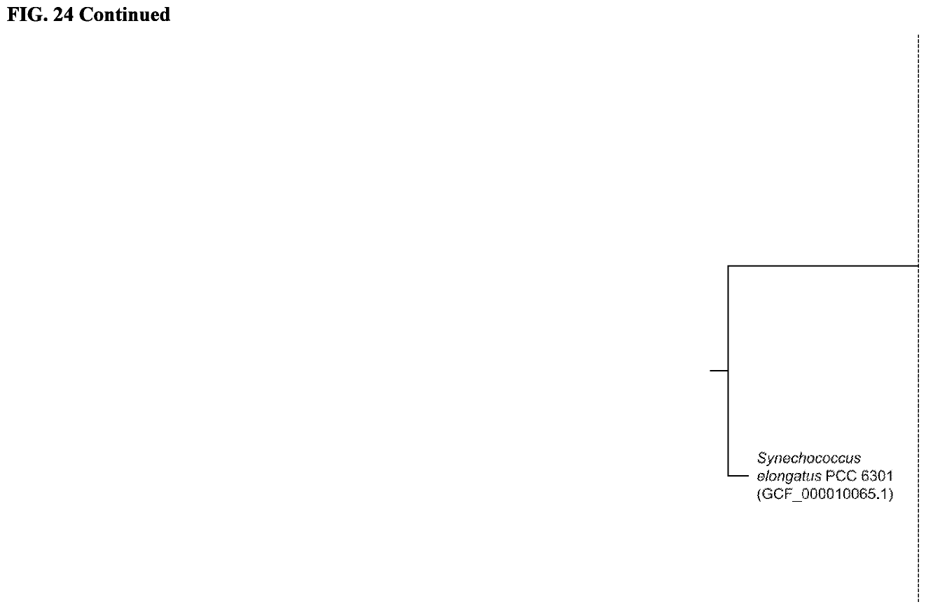

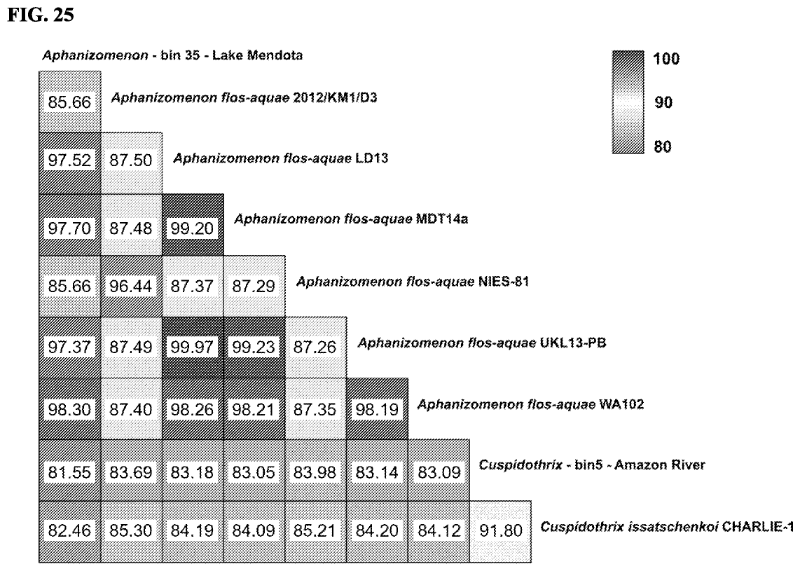

Surprisingly, inventors were unable to link the known guanitoxin producer genera as the source taxa of the two environmental metaG-derived gnt BGCs. Guanitoxin producer strains have been rarely isolated in the literature, and the taxonomy of guanitoxin producer genera has been unstable, suggesting the full taxonomic extent of guanitoxin production is not yet known. To explore if other cyanobacterial genera could harbor gnt biosynthetic capacity, two environmental metagenome-derived gnt BGCs were linked to their best-supported taxonomic origins. Via full MAG assembly, the gnt BGCs from the Amazon River and Lake Mendota datasets were connected to the Cuspidothrix and Aphanizomenon cyanobacterial genera, respectively (FIGS. 24-25). While these genera are known to produce other cyanotoxins such as anatoxin-a, microcystin, and cylindrospermopsin,⁴⁴ they have not yet been reported to produce guanitoxin. However, their genomic potential warrants future environmental surveying for guanitoxin biosynthesis.

The characterization of how guanitoxin is biochemically assembled from the standard amino acid L-arginine represents the first biosynthetic report of a natural organophosphate neurotoxin. Significantly, the discovery of the architecturally unique gnt BGC enabled identification of environmental hotspots in unaware rural and populated areas for the potent yet unmonitored neurotoxin guanitoxin, suggesting that revised cyanoHAB monitoring protocols are warranted in public watersheds vulnerable to toxic harmful algal/cyanobacterial blooms.

### Example 3: Materials and Methods

Sphaerospermopsis torques-reginae ITEP-024 Culture

A non-axenic culture of Sphaerospermopsis torques-reginae strain ITEP-024, a known producer of guanitoxin ((5S)-5-[(dimethylamino)methyl]-1-[hydroxy(methoxy)phosphoryl]oxy)-4,5-dihydro-1H-imidazol-2-amine) was a gift from V. R. Werner (Museum of Natural Sciences, Porto Alegre, Brazil), and was maintained in conditions similar to previous descriptions⁴⁵.

In brief, ITEP-024 cultures were grown in 50 mL autoclaved ASM-1 medium⁴⁶, excepting the final ASM-1 ZnCl and CuCl₂ concentrations were 2.5 μM and 0.01 μM, respectively, and the pH was 7.0-7.4. Cultures were grown in 125 mL borosilicate glass Erlenmeyer flasks, sealed with gas-permeable waxed paper. Cultures were either maintained under ambient laboratory light cycle, light intensity, and temperature on the benchtop, or in lighted incubators under previously described conditions⁴⁵. Cultures were harvested either by centrifugation, or by GF/F (Whatman, Cytiva) glass fiber filtration of trichomes. All culture manipulations were performed in biosafety or chemical fume hoods as appropriate. Cultures were biologically and chemically inactivated by 10% bleach treatment before disposal.

Transformation into E. coli

The plasmids were transformed into E. coli DH10B chemically competent cells for storage and BL21(DE3) for expression. The transformation by heat shock proceeded according to the following protocol: 0.5 μL of plasmid was added to the chemical competent cells and maintained into ice for 30 minutes. After this, the cells were heated to 42° C. for 45 seconds and placed in the ice again for 3 minutes; 900 μL of LB medium was added in the tube and the cells were incubated for 50 minutes at 37° C. and 200 rpm of agitation. After this step, the cells were plated on LB agar plates supplemented with the corresponding antibiotics. The plates were incubated at 37° C., overnight. An inoculum with colonies was grown overnight in the same condition of LB agar plates at 37° C. and 200 rpm of agitation and purified following the protocol of Plasmid DNA Purification QIAprep Spin Miniprep Kit (QIAGEN). After plasmid purification, concentrations were measured by NanoDrop UV-vis spectrophotometry and stored at −20° C.

### Extraction and LC-MS Analyses

Lyophilized culture was extracted with 5 mL of ethanol/acetic acid 0.1M (20:80 v/v), sonicated for 1 minute on ice and centrifuged at 5,000×g for 15 minutes. The supernatant was lyophilized and resuspended in methanol and filtered with a syringe into an autosampler vial. The Hydrophilic Interaction Liquid Chromatography (HILIC) separation was carried out on a SeQuant® ZIC-HILIC, 150×2.1 mm, 5 μm, 200 Å (Merck) column similar to the method described in previous studies⁴⁷.

HILIC Method: Separation was achieved under gradient elution at 0.2 m/min where elution A was 5 mM ammonium formate containing 0.01% (v/v) formic acid, and elution B was acetonitrile/water (90:10 v/v) with 0.01% (v/v) formic acid. Elution started with a linear gradient of 90% B to 20% until 35 min, second isocratic gradient of 20% B until 37.50 min and a third isocratic gradient of 90% B until 45 min.

RP Method: For this analysis a Synergi Polar-RP 4p 250×4.6 mm column (Phenomenex) used at 0.75 m/min with the following method: 0% B (12 min), 0 to 100% B (5 min), 100% B (3 min), 100 to 0% B (2.5 min), 0% B (2.5 min), wherein A=0.1% aqueous formic acid, and B=0.1% formic acid in acetonitrile.

General LC-MS measurements were measured on a Bruker Elute UHPLC system coupled with a Bruker amaZon SL ESI-Ion Trap mass spectrometer in positive mode. Compounds were separated via reversed-phase chromatography on a Bruker Intensity Solo C18⁴⁶, 2 μm-2×100 mm column with the eluents water+0.1% formic acid (Solvent A) and acetonitrile+0.1% formic acid (Solvent B). The LC method uses a flow rate of 0.300 mL/min and the following gradient: 10% to 20% B over 3 minutes, 20% to 45% B over 3 minutes, 45%-100% B over 2 minutes, hold at 100% B for 2 minutes, 100%-10% B over 1 minute, hold at 10% B for 2 minutes.

Semi-preparative HPLC purification was performed using a Shimadzu Prominence preparative HPLC system with a SPD-20A model UV/Vis detector. Analytical HPLC purification was performed using an Agilent 1260 Infinity system with a G1314B model VWD. The monitored wavelength was 210 nm for all runs.

Sphaerospermopsis torques-reginae ITEP-024 Genome Sequencing and Assembly

For Illumina genome sequencing: Illumina data for ITEP-024 that had been previously quality and adaptor trimmed, was prepared in a previously described project⁴⁵. In brief, a Nextera XT (Illumina) sequencing library was prepared and sequenced on a MiSeq instrument to a depth of ˜26M reads with 300×300 bp paired-end (PE) sequencing. In total, 13.4 Gbp (˜2400× coverage) of quality and adaptor trimmed data was used for downstream analyses. The library insert size of was ˜125-400 bp with a tail up to 800 bp, as determined by Qualimap v2.2.1⁴⁸ analysis of reads aligned to the ITEP-024 genome assembly with bowtie2 v2.4.2⁴⁹. These quality and adaptor trimmed reads are available on NCBI SRA as accession (SRR15608978).

For nanopore genome sequencing: 50 mL of Sphaerospermopsis torques-reginae ITEP-024 was harvested via centrifugation, and the pellet was resuspended in 500 μL of 10 mM Tris pH 8.0. 7 mg of lysozyme was added, and the suspension incubated at 37° C. for 30 minutes. 1.5 mL of cetrimonium bromide (CTAB) buffer (3% CTAB, 1.4 M NaCl, 20 mM EDTA, 100 mM Tris pH 8.0, 3% polyvinylpolypyrrolidone (PVPP), 0.2% β-mercaptoethanol) was added, followed by 10 μL of proteinase K at 20 mg/mL and a 2-hour 50° C. incubation with mixing every 15 minutes. The sample was centrifuged at 15,000×g for 15 minutes at room temperature, and the supernatant transferred to a new tube and added 400 μL of 5M potassium acetate pH 8.0, the tube was then kept on ice for 30 minutes to precipitate polysaccharides. The tube was centrifuged at 15,000×g for 15 minutes at 4° C. Half the volume of the supernatant was transferred to a new tube and extracted with 1 equivalent of phenol:chloroform:isoamyl alcohol (25:24:1). The tube was centrifuged at 12,000×g for 5 minutes at 4° C., and supernatant and precipitate DNA were transferred to a new tube with 1 equivalent in volume of ice-cold isopropanol. The sample was harvested at 12,000×g for 15 minutes at 4° C. After discarding the supernatant, the DNA pellet was washed 2 times with 0.5 mL of 75% ethanol. The DNA was air dried for 30 minutes at 37° C. and resuspended in 40 μL of 10 mM Tris pH 8.5. Sample was maintained at −20° C. until sequencing. A Nanopore ligation sequencing library (P/N SQK-LSK109) was prepared from this DNA following the manufacturer's instructions, and the resulting library sequenced via a Flongle flowcell on an Oxford Nanopore MinION sequencer. The resulting dataset was checked via NanoPlot v1.27.0⁵⁰, and had an N50 of ˜5.5 Kbp and a yield of ˜0.2 Gbp. A systematic error was noted with an unexpectedly high (>25%) proportion of palindromic reads. Speculation was made that these errors may be due to the Flongle system being a new product from Oxford Nanopore at the time of the experiment.

Sphaerospermopsis torques-reginae ITEP-024 Genome Assembly & Annotation

For the Illumina MiSeq dataset, read quality was checked using FastQC v0.10.1 (https://www.bioinformatics.babraham.ac.uk/projects/fastqc/). Reads were additionally quality filtered to trim bases with a Phred score below 25 using Prinseq⁵¹. To help reduce the effect of the aforementioned Nanopore Flongle palindromic errors, palindromic sequences from the Nanopore dataset were consensus-corrected and trimmed with Canu⁵². These quality and palindrome trimmed reads are available on NCBI SRA as accession (SRR15608977). A second correction was applied on these Canu trimmed sequences by mapping the Illumina reads with CoLoRMap⁵³. Two de novo hybrid assemblies were produced with Unicycler⁵⁴ and MaSuRCA⁵⁵ using both the Illumina and Nanopore datasets as inputs. The generated contigs from both assemblers were improved using SSPACE⁵⁶ to merge scaffolds, Pilon⁵⁷ for polishing and GapFiller⁵⁸ for filling of gaps. 26 total iterations of scaffolding, gap-filling, and polishing were performed. Subsequently, an overall assembly was generated by merging both assemblies with Flye using the subassembly feature⁵⁹. The quality assessment of the assemblies was done with Quast 5.0.2⁶⁰ and lineage-expected gene completeness statistics and lineage-incongruent gene contamination statistics were measured with CheckM⁶¹ (FIG. 9B). Two ˜185 Kbp plasmids and some limited sequences from presumed contaminating heterotrophic bacteria were detected in some of the assemblies. They however did not have clear specialized metabolism related genes and could not be clearly linked as ITEP-024 sequences, so were not analyzed further. The post-assembly circularization tool Circulator version⁶² was used to check for circularization of the Sphaerospermopsis torques-reginae ITEP-024 chromosomal genome. However, the software could not provide clear conclusions about the linearity or circularization of the genome. Since linear contigs produced by genome assemblers can contain the absence of short sequences that would join the contig ends, it is difficult to ensure whether the Sphaerospermopsis torques-reginae ITEP-024 genome is linear or circular, or if it was not circularized due to the absence of sequences in both ends. The determination of a linear or circular chromosomal genome for ITEP-024 was unresolved, but as circular chromosomes are most common in the cyanobacterial phylum, speculation that the genome would be circular was done. The intermediate working gene annotation was performed with Prokka⁶³, while the final gene annotation on the NCBI submitted chromosomal assembly (NCBI accession CP080598.1) was produced via the NCBI Prokaryotic Gene Annotation Pipeline (PGAP).

### Molecular Biology/Biochemical Methods

### gnt Cloning

The enzyme coding sequences were optimized for expression in E. coli and synthesized by GenScript Inc. Synthetic guanitoxin biosynthetic genes were sub cloned into the pET28a(+) kanamycin resistant expression vector containing an N-terminal hexahistidine (His₆) tag. The pET28a(+) plasmids containing synthetic gnt genes were resuspended in 100 μL of sterilized ultrapure water and transformed into E. coli DH10B chemically competent cells for plasmid storage and BL21(DE3) for protein expression as previously described.

pCOLADuet-gntB-gntC Vector Assembly and Transformation

Using their individual pET28a(+) plasmids as templates, gntB and gntC were amplified by PCR using the primers listed in Table 5 and following amplification conditions: For primer set gntB-F/R, the following program was used: an initial denaturation at 98° C. (30 s); 30 cycles of 98° C. (10 s), 70° C. (30 s), and 72° C. (30 s); and a final extension at 72° C. (2 min). For primer set gntC-F/R, the following program was used: an initial denaturation at 98° C. (30 s); 30 cycles of 98° C. (10 s), 62° C. (30 s), and 72° C. (30 s); and a final extension at 72° C. (2 min). For primer set pCOLADuet-F/R, the following program was used: an initial denaturation at 98° C. (30 s); 30 cycles of 98° C. (10 s), 61° C. (30 s), and 72° C. (2 min); and a final extension at 72° C. (2 min). PCR-amplified gntB (957 bp) and gntC (1113 bp) were individually and sequentially added into multiple cloning sites 1 and 2 of pCOLADuet-1 respectively following Gibson Assembly Master Mix protocols (New England Biolabs).

### Gnt Protein Expression

A general method was followed for each of the guanitoxin pathway enzymes: GntA*, GntC, GntD, GntE, GntF, GntG, GntI, and GntJ. A 20 mL starter culture of LB media containing 50 μg/mL kanamycin was inoculated with E. coli BL21(DE3) containing the appropriate Gnt-containing expression plasmid from glycerol stocks and shaken overnight at 37° C. and 200 rpm. The next day, 10 mL of starter culture was used to inoculate 1 L of TB media containing 50 μg/mL kanamycin in a 2.8 L flask and was shaken at 37° C. and 200 rpm until the OD₆₀₀ reached 0.9. The incubation temperature was decreased to 18° C. for one hour, followed by the addition of 100 μM isopropyl-β-D-thiogalactopyranoside (IPTG) to induce protein expression. The cultures were incubated at 18° C. and shook at 200 RPM for 20 hours. Cultures were harvested by centrifuging at 2500×g and 4° C. for 30 minutes. Pellets were resuspended in 30 mL lysis buffer (20 mM Tris-HCl pH 8.0, 1 M NaCl, 20 mM imidazole, and 10% glycerol) and stored at −70° C. until purification.

*In the case of GntA (heme-dependent N-hydroxylase), a slightly modified growth protocol was used to supply heme required for proper folding of GntA. Once the OD₆₀₀ had reached 0.4, 7 μM porcine hemin (Chem-Impex) was added to the culture and was then allowed to continue growing until an OD₆₀₀ of 0.9 was reached.

### Gnt Protein Purification

E. coli cell pellets containing gnt genes were thawed and sonicated on ice (FisherBrand Model 505 Sonic Dismembrator, 3.2 mm microtip, 40% amplitude, 15 s pulse on/45 s pulse off for a total of 7 minutes). The lysate was centrifuged at 16,000×g for 30 minutes at 4° C. or until the supernatant had clarified. Each protein was purified using an AKTAGo FPLC system at 4° C. and buffers that had been filtered through a 0.22 μM nitrocellulose membrane. Clarified lysate was loaded onto a 5 mL HisTrap FF Column (GE Healthcare Life Sciences) that had been equilibrated with at least 25 mL of Buffer A (20 mM Tris-HCl pH 8.0, 1 M NaCl, and 20 mM imidazole) at a maximum flow rate of 2 mL/min. After loading, the column was rinsed with Buffer A until UV absorbance had returned to baseline. The column was then washed with 10% Buffer B (20 mM Tris-HCl pH 8.0, 1 M NaCl, and 250 mM imidazole) to remove weakly bound protein with at least 25 mL buffer or until UV absorbance returned to baseline. His₆-tagged protein was eluted with a linear gradient from 100% Buffer A to 100% Buffer B over 60 mL, while collecting 5 mL fractions. Fractions were assessed for purity through SDS-PAGE (10% or 12% acrylamide, depending on protein size) and were combined if they were at least 90% pure. Protein was concentrated to a volume of 2.5 mL or less using a 10 kDa or 30 kDa cutoff (based on each protein's size) Amicon Ultra-15 concentrator. Protein was buffer exchanged into GF Buffer (50 mM HEPES pH 8.0 and 300 mM KCl) using a pre-equilibrated PD-10 gravity flow column, or further purified by size exclusion chromatography using a HiLoad 16/60 Superdex 75 column or HiLoad 16/60 Superdex 200 column, based on protein sizes and possibility of dimers (GE Healthcare Life Sciences) using a 20 mM HEPES (pH 7.5) and 300 mM KCl buffer. Protein concentration was estimated using the Bradford assay based on a Bovine Serum Albumin standard; if necessary, protein was further concentrated after this exchange. Each protein was aliquoted and stored at −70° C. for future use. The following yields of pure protein were obtained: GntA (60 mg/L), GntC (18 mg/L), GntD (35 mg/L), GntE^{∧} (13 mg/L), GntF (225 mg/L), GntG (29 mg/L), GntI (70 mg/L), and GntJ^{#} (180 mg/L).

^{#}For GntJ (O-methyltransferase) the hexahistidine (His₆) tag was cut off using 10 units of thrombin per mg of recombinant protein incubated overnight, retaining some uncleaved protein as a control. The extent of His₆ tag cleavage was assessed by SDS-PAGE (12% acrylamide) and size exclusion chromatography using a HiLoad 16/60 Superdex 75 column was used to purify GntJ as described previously.

For GntE (PLP-dependent aminotransferase), the protein eluted in the absence of PLP and upon fractionation began to aggregate in a concentration-dependent manner. 500 μM PLP and 20% glycerol was added to the combined fractions to stabilize GntE for concentration and buffer exchange. Prior to buffer exchange, any aggregate was removed by centrifuging at 20,000×g for 10 minutes at 4° C. After desalting into GF buffer, GntE retained PLP and was not concentrated further. No further aggregation was observed before aliquots were made.

### General Procedure for Gnt Enzyme Assays: Marfey's Analysis and UPLC-MS Conditions

A 50 μL aliquot of the Gnt reaction mixture (typically 100 μL) was removed and added to 20 μL of a saturated sodium bicarbonate solution. The addition of 100 μL of freshly-prepared 1% w/v of 1-fluoro-2,4-dinitrophenyl-5-L-alanine amide (L-FDAA, Marfey's reagent) in acetone began the derivatization reaction, which was incubated at 37° C. for 90 minutes. After incubation, reactions were quenched by the addition of 25 μL of 1 N HCl, before centrifuging (15000 rpm for 5 minutes). The clarified supernatant was extracted and subjected to RP-UPLC-MS (Bruker Intensity Solo C18(2), 2.1×100 mm) at a flow rate of 0.300 mL/min using the following method: 10-20% B (3 min), 20-45% B (3 min), 45-100% B (2 min), 100% B (2 min), 100-10% B (1 min), 10% B (2 min), where A=0.1% aqueous formic acid, and B=0.1% formic acid in acetonitrile.

### In Vivo GntB/GtnC Enzyme Assay

The pCOLA-Duet-1 pET28a plasmids containing no insert (empty pET28a vector), only gntB (pET28a vector), and gntB and gntC in tandem, were transformed into BL21(DE3) E. coli. 5 mL LB starter cultures containing 50 mg/mL kanamycin of BL21(DE3) colonies grew overnight. Cells were centrifuged (3400 rpm, 5 min, 4° C.), decanted, and resuspended in 5 mL M9 minimal media. Resuspended BL21(DE3) cells (1 mL) were inoculated into 50 mL aliquots of M9 minimal media containing 30 mg/mL kanamycin. Cultures were incubated (200 rpm, 37° C.) until reaching an OD₆₀₀ of 0.7, then were cooled and incubated for one additional hour (200 rpm, 18° C.). Cultures were induced with 1 mM IPTG to induce GntB and GntB/C protein expression. After 5 days of incubation (200 rpm, 18° C.), 1 mL aliquots of each culture were extracted. Aliquots were sonicated (40% amplitude, 10 seconds on, 10 seconds off, for 1.5 minutes) to lyse the cells, and then cell lysates were centrifuged for 10 minutes (15000 rpm, 4° C.). From the centrifuged cell lysate, 50 μL of the supernatant was aliquoted for Marfey's derivatization and subsequent UPLC-MS analysis.

### In Vitro GNT Enzyme Assays

### GntC Functional Assays

GntC activity assays were conducted in 50 mM K₂HPO₄ buffer (pH 8.0) using 1 mM substrate, 100 μM PLP, and 50 μM of purified GntC enzyme. Total reaction volumes were brought to 100 μL of total volume with MilliQ water. Assays were incubated at room temperature overnight, then a 50 μL aliquot was extracted for Marfey's derivatization before subsequent LC-MS analysis.

### GntD Functional Assay

GntD activity assays were conducted in 50 mM K₂HPO₄ buffer (pH 8.0) using 1 mM substrate, 100 μM FeSO₄, 2.5 mM α-ketoglutarate, 50 μM L-ascorbic acid and 50 μM of purified GntD enzyme. Total reaction volumes were brought to a total volume of 100 μL using MilliQ water. Assays were incubated at room temperature for 6 hours and 50 μL aliquots were extracted after 90 minutes and 6 hours, derivatized via Marfey's reagent and analyzed by LC-MS.

### GntD Scale Up Reactions

Scaled up reactions for NMR characterization were run using the conditions previously described in 1 mL aliquots and 43 total reactions were set up. Scaled reactions ran for 14 hours at room temperature. The reactions were pooled, quenched with an equivalent amount of HPLC-grade methanol, then centrifuged at 11000 rpm for 15 min. The supernatant was concentrated in vacuo to remove methanol and the remaining aqueous reaction was frozen and lyophilized overnight.

### HPLC Purification of GntD Product β-Hydroxy-L-Enduracididine (4)

The lyophilized aqueous layer was resuspended in MilliQ water and purified by semi-preparative RP-HPLC (Phenomenex Synergi 4 μm Polar RP 80 Å, 10×250 mm) at a flow rate of 3.5 mL/min using the following method: 0.5% B (5 min), 0.5-95% B (1 min), 95% B (4 min), 95%-0.5% B (1 min), 0.5% B (4 min), where A=0.1% aqueous formic acid, and B=0.1% formic acid in acetonitrile. The peak containing 4 was manually collected (retention time ˜3.90 min), concentrated in vacuo and lyophilized. The sample was then further purified by analytical RP-HPLC (Phenomenex Synergi 4 μm Polar RP 80 Å, 4.6×250 mm) at a flow rate of 1.0 mL/min. Compound 4 eluted in 0.5% B (retention time ˜2.85 min) and was manually collected, concentrated in vacuo and lyophilized to afford the product as a white solid. ¹H NMR (500 MHz, D₂O+0.1% MeOH): S 4.34 (ddd, J=9.7, 6.8, 5.5 Hz, 1H), 4.06 (dd, J=6.7, 3.1 Hz, 1H), 3.90 (d, J=3.1 Hz, 1H), 3.83 (dd, J=10.0, 10.0 Hz, 1H), 3.65 (dd, J=10.3, 5.5 Hz, 1H); ¹³C NMR (500 MHz, D₂O+0.1% MeOH): δ 171.4, 159.4, 71.5, 56.5, 55.9, 44.9; HRMS (TOF) Calculated for C₆H₁₃N₄O₃ 189.0982. found 189.0982 (M+H)⁺

### GntCDGE Dependence Assay

The one pot assay to assess if 2 could be converted to 6 was conducted in 50 mM K₂HPO₄ buffer (pH 8.0) using 1 mM 2. Cofactors used in this assay included 100 μM PLP, 1 mM αKG, 50 μM L-ascorbic acid, 100 μM FeSO₄, and 5 mM L-glutamate, with 40 μM of purified GntC, and 25 μM of purified GntD, GntG, and GntE. Assays were incubated at room temperature overnight and then 50 μL aliquots were extracted for Marfey's derivatization prior to LC-MS analysis.

### GntE/G Functional Assay

GntE/G activity assays were performed in 50 mM K₂HPO₄ buffer (pH 8.0) using 1 mM substrate 6, 100 μM PLP, 1 mM α-ketoglutarate, 1 mM glycine, and 50 μM purified GntG enzyme and 50 μM purified GntE enzyme. Total reaction volumes were brought to 100 μL using MilliQ water. Assays were incubated at room temperature overnight and then a 50 μL aliquot was extracted for Marfey's derivatization before subsequent LC-MS analysis.

### GntF: N-Methyltransferase

GntF activity assays were performed in 50 mM Tris buffer (pH 7.4) using 0.1 mM substrate 6, 1 mM S-adenosylmethionine (SAM), and 20 μM purified GntF enzyme. Total reaction volumes were brought to 500 μL with MilliQ water and incubated at 27° C. for 18 hours. The reaction was quenched with one volume of acetonitrile on ice and filtered at 14000×g, 4° C. for 10 minutes using both 3 kDa cutoff filters and 0.2 μm filters. The supernatant was then removed and subjected to LC-MS analysis.

### GntA: N-Hydroxylase

GntA activity assays were performed in 50 mM Tris buffer (pH 7.4) using 0.1 mM substrate 7, 5 mM NADPH, and 20 μM purified GntA enzyme. Total reaction volumes were brought to 500 μL with MilliQ water and incubated at 27° C. for 18 hours. The reaction was quenched with one volume of acetonitrile on ice and filtered at 14000×g, 4° C. for 10 minutes using 0.2 μm filters. The supernatant was then removed and subjected to LC-MS analysis.

### GntI: Kinase

GntI activity assays were performed using the filtrate of the GntA enzymatic assay, sequentially adding 2 mM ATP, 100 mM NaCl, 2 mM MgCl₂ and 20 μM purified GntI enzyme. The reaction volume was brought to 200 μL with 50 mM Tris buffer pH 7.4 and incubated at 37° C. for 30 minutes. The reaction mixture was quenched with one volume of acetonitrile on ice and filtered at 14000×g, 4° C. for 10 minutes using 0.2 μm filters. Supernatant was then removed and subjected to LC-MS analysis.

### GntJ: O-Methyltransferase

GntJ activity assays were performed using the filtrate of the GntI enzymatic assay, sequentially adding 1 mM of SAM and 20 μM of GntJ enzyme without the His₆ tag. The reaction volume was brought to 100 μL with 50 mM Tris buffer pH 7.4 and incubated at 27° C. for 18 hours. The reaction mixture was quenched with one volume of acetonitrile on ice and filtered at 14000×g, 4° C. for 10 minutes using 0.2 μm filters. Supernatant was then removed and subjected to LC-MS analysis.

### Example 4: Guanitoxin Biosynthetic Gene Cluster (BGC) in Environmental Sequencing

### Initial Searches for Guanitoxin BGC

Using the SearchSRA tool^{49, 64-68} 108,465 SRA metagenomic and metatranscriptomic datasets were searched for the guanitoxin biosynthetic gene cluster using a translated nucleotide (diamond) search with the GNT A-J peptide sequences. Hits within the ‘results.zip’ file provided by SearchSRA were filtered to those reads with a >=90% protein sequence identity using a custom script (https://github.com/photocyte/gnt_paper/searchSRA_workflow).

### Full Sensitivity Search of Metatranscriptomic Data for the Guanitoxin BGC

All freshwater Illumina sequencing metatranscriptomic samples on SRA were selected via the following search on the NCBI web interface: “(lake[All Fields] OR pond[All Fields] OR reservoir[All Fields] OR river[All Fields] OR stream[All Fields] OR bay[All Fields] or bog[All Fields] or cyano[All Fields]) AND “ecological metagenomes”[Organism] NOT “marine metagenome”[Organism] NOT “seawater metagenome”[Organism] NOT “marine sediment metagenome”[Organism] AND “biomol rna”[Properties] AND “platform illumina”[Properties]”. These search terms were empirically derived, as there was no single metadata field which applied to all freshwater metatranscriptomic samples. Metadata for these datasets was exported using the NCBI SRA Run Selector tool in CSV format. A brief script was used to filter this CSV file to a list of SRA target accession ids. A custom Nextflow⁶⁹ workflow (https://github.com/photocyte/gnt_paper/metaT_search_workflow) was used to download and search these datasets. The workflow was run with parameters “--sra_ids_file target_SRAs.txt --fasta ./target_genes/gnt_cds.fna-resume-with-trace-with-report”. In brief, SRA reads were downloaded using fasterq-dump. Datasets resulting in .fastq files with a filesize of <160 KB were discarded and not analyzed further. Next, reads were aligned to the gnt BGC genes (gntA-J) coding nucleotide (CDS) regions, using bowtie2⁴⁹ with parameters “--very-sensitive-local --no-unal”. Mapped reads were summarized and processed from the resulting .bam files using a custom Jupyter⁷⁰ notebook (https://github.com/photocyte/gnt_paper/metaT_search_workflow/jupyter). In brief, reads were parsed into and manipulated using pandas⁷¹⁻⁷² dataframes, while extensive metadata from the SRA database was re-added using pysradb⁷³. The results of this analysis are shown in Table 3 and FIG. 9B.

### Metatranscriptomic De Novo Assembly

De novo assembling of gnt BGCs from the identified metatranscriptomic SRA data was done, using a custom Nextflow⁶⁹ workflow. The workflow was run with parameters “--targets SRR5249014;SRR5249015;SRR1601415;SRR1601417;SRR1601416;SRR5834679;SRR583 4616;SRR5194978 --peps gntpeps.faa --nucl gnt_cds.fna -with-trace -resume”. In brief, the workflow downloads SRA reads in .fastq format from each of the SRA datasets which had reads mapping to all 8 gnt CDS sequences (see Full sensitivity search of metatranscriptomic data for the guanitoxin BGC methods; Table 3) using fasterq-dump (NCBI sra-tools v2.10.8) via a bioconda environment⁷⁴. De novo transcriptome assembly was performed on the downloaded .fastq files using rnaSPAdes v3.14.1⁷⁵ via a bioconda/quay.io Singularity container⁷⁶. The resulting de novo transcriptome assemblies were filtered to just the gnt BGC contigs using similarity searches with the gnt CDSs and GNT peptides (externally provided to the workflow) via tblastn/BLAST+⁷⁷ via a Singularity container. Lastly, Prokka⁶³ via a Singularity container was used to annotate the selected gnt BGCs. Externally provided GNT peptides were provided to Prokka to propagate our standardized gnt BGC naming scheme.

### Metagenome Assembled Genomes & Taxonomic Identification of GNT BGC Source

For metagenomic datasets, the read adaptors were trimmed, and quality filtered with Cutadapt⁷⁸. The metagenome sequences were assembled with metaSPAdes⁷⁹ and the resulting scaffolds were clustered into bins using the automated binning by MetaBAT⁸⁰. The Metagenome Assembled Genomes (MAGs) were filtered based on its sequence completeness above 70% and contamination below 10% measured by CheckM⁶¹. The quality assessment of the assemblies was done with Quast 5.0.2⁶⁰. GNT gene clusters were screened with BLAST+⁷⁷ using an in-house script.

Taxonomic classification and phylogenomic analyses were performed with GTDB-Tk⁸¹, using the Genome Taxonomy Database (GTDB)⁸². The pipeline generated the tree through the identification and alignment of 120 bacterial single-copy conserved marker genes, then inferred the phylogeny of the concatenated sequences using FastTree⁸³ with the WAG+GAMMA models and maximum likelihood algorithm. Drawing of trees and annotations were done with iTOL v4⁸⁴ and Inkscape (https://inkscape.org/). The MAGs and its closest related genomes extracted from the NCBI Genbank Database⁸⁵ had their average nucleotide identities calculated with OrthoANI v1.4⁸⁶.

### Example 5: Chemical Synthesis

### Synthesis of Primary Amine Intermediate 6

A solution of (S)-Garner's aldehyde (0.50 mL, 2.31 mmol) and dibenzylamine (0.47 mL, 2.43 mmol) in methanol (10 mL) had acetic acid (0.1 mL) added and was stirred for 15 minutes at room temperature. To this solution, sodium cyanoborohydride (0.145 g, 2.31 mmol) was added over a few minutes and stirred at room temperature under an argon atmosphere for 20 h. The reaction mixture was cooled to 0° C. and an aqueous solution of saturated sodium bicarbonate (25 mL) was added, followed by ethyl acetate (50 mL). The layers were separated and the aqueous layer was further extracted with ethyl acetate (2×50 mL). Pooled organic layers were washed with brine (50 mL), dried over magnesium sulfate, filtered and concentrated in vacuo. The crude reaction mixture was purified by silica flash chromatography and eluted over a gradient of 9:1 to 4:1 hexanes:ethyl acetate+0.1% triethylamine. Pooled fractions were concentrated in vacuo, yielding the desired product as a clear light yellow oil (0.514 g, 54%). Rf 0.9 (2:1 hexane:ethyl acetate); IR (CH₂Cl₂ cast) 3062, 2977, 2935, 2877, 2802, 1695, 1454, 1386, 1253, 1170, 1087, 1027 cm⁻¹; ¹H-NMR (CD₃OD; 500 MHz, mixture of 2 rotamers) δ 7.36-7.28 (m, 8H), 7.26-7.20 (m, 2H), 4.04 15-3.99 (m, 0.4H), 3.89-3.84 (m, 0.6H), 3.84-3.69 (m, 4H), 3.40 (d, J=13.5 Hz, 1H), 3.32 (d, J=11.2 Hz, 1H), 2.56 (d, J=9.8 Hz, 1H), 2.49 (d, J=12.0 Hz, 1H), 1.50-1.46 (m, 10H), 1.40-1.36 (m, 5H), 1.31 (s, 1H); ¹³C-NMR (CD₃OD; 125 MHz, mixture of 2 rotamers) δ 153.4, 140.8, 130.2, 130.0, 129.3, 129.3, 128.2, 128.1, 94.8, 81.6, 81.2, 79.5, 67.4, 67.1, 60.3, 59.9, 57.4, 57.1, 56.6, 28.8, 28.7, 28.0, 27.1, 24.6, 23.2; HRMS (ESI) Calculated for C₂₅H₃₅N₂O₃ 411.2642. found 411.2637 (M+H)⁺.

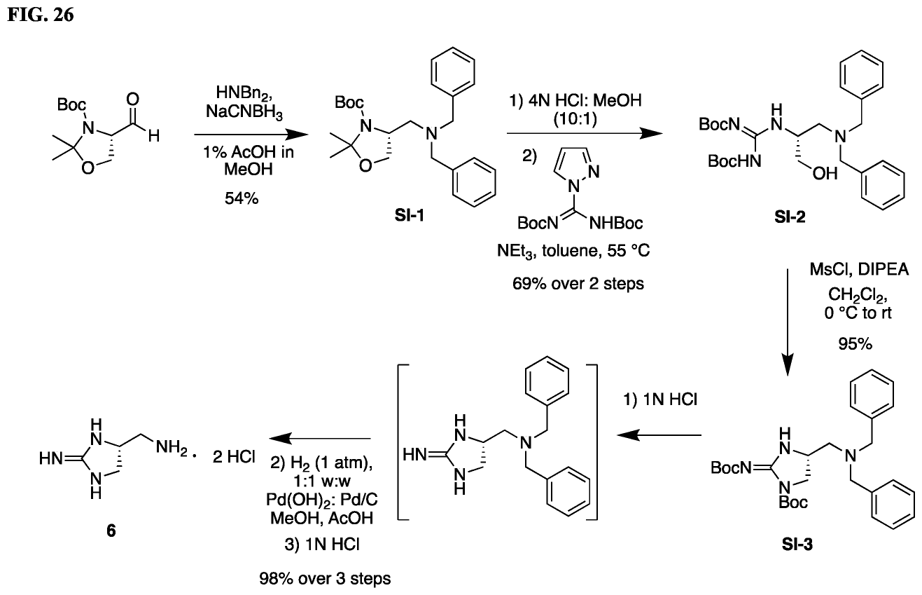

FIG. 26 shows the intermediates involved in synthesis of primary amine intermediate 6. A solution of SI-1 (FIG. 26) (0.328 g, 0.80 mmol) in 4 N aqueous HCl (10 mL) and methanol (1 mL) was stirred at room temperature for 2 h. The reaction mixture was concentrated in vacuo, using water and toluene co-evaporations to remove additional HCl and water respectively. The crude material was resuspended in toluene (15 mL) and had triethylamine (0.56 mL, 3.99 mmol) and N,N′-di-Boc-1H-pyrazole-1-carboxamidine (0.273 g, 0.88 mmol) sequentially added. The reaction mixture was heated to 55° C. and stirred for 12 h, then was cooled to room temperature, diluted with ethyl acetate (50 mL) and washed with water (2×25 mL). The organic layer was dried over magnesium sulfate, filtered, and concentrated in vacuo. The crude reaction mixture was purified by silica flash chromatography and eluted over a gradient of 9:1 to 4:1 hexanes:ethyl acetate+0.1% triethylamine. Pooled fractions were concentrated in vacuo, yielding the desired product as a sticky white foam (0.283 g, 69/6). Rf 0.65 (2:1 hexane:ethyl acetate); IR (CH₂Cl₂ cast) 3416, 2974, 1725, 1638, 1416, 1357, 1147 cm⁻¹; ¹H-NMR (CD₃OD; 500 MHz) δ 7.90 (s, 1H), 7.38 (d, J=7.1 Hz, 4H), 7.27 (t, J=7.5 Hz, 4H), 7.20 (t, J=7.3 Hz, 2H), 5.49 (s, 1H), 4.14 (dq, J=8.7, 4.3 Hz, 1H), 3.77 (d, J=13.6 Hz, 2H), 3.58 (dd, J=11.5, 3.6 Hz, 1H), 3.47 (dd, J=11.5, 4.5 Hz, 1H), 3.42 (d, J=13.5 Hz, 2H), 2.73 (dd, J=13.0, 10.3 Hz, 1H), 2.51 (dd, J=13.1, 4.8 Hz, 1H), 1.61 (s, 9H), 1.42 (s, 9H); ¹³C-NMR (CD₃OD; 125 MHz) δ 164.2, 157.6, 154.0, 140.5, 130.1, 129.3, 128.1, 84.5, 80.3, 79.5, 64.2, 59.8, 55.7, 52.5, 28.5, 28.3; HRMS (ESI) Calculated for C₂₈H₄₁N₄O₅ 513.3071. found 513.3068 (M+H)⁺.

To a 0° C. solution of SI-2 (FIG. 26) (0.091 g, 0.18 mmol) in dry CH₂Cl₂ (10 mL) was sequentially added DIPEA (62 μL, 0.36 mmol) and methanesulfonyl chloride (15 μL, 0.20 mmol). The reaction mixture was slowly warmed to room temperature over 21 h, then quenched by the addition of saturated NH₄Cl (20 mL) and diluted with CH₂Cl₂ (10 mL). The layers were separated and the organic layer was further washed with water (20 mL), and brine (20 mL), dried over magnesium sulfate, filtered and concentrated in vacuo. The crude reaction mixture was purified by silica flash chromatography and eluted over a stepwise gradient of 99:1 to 49:1 chloroform:methanol+0.1% triethylamine. Pooled fractions were concentrated in vacuo, yielding the desired product as a white solid (0.083 g, 95%). Rf 0.35 (49:1 chloroform:methanol+1 drop triethylamine); IR (CH₂Cl₂ cast) 3412, 2977, 2809, 1754, 1702, 1649, 1604, 1537, 1446, 1373, 1311, 1144 cm⁻¹; ¹H-NMR (CD₃OD; 500 MHz) δ 7.30-7.23 (m, 8H), 7.20-7.15 (m, 2H), 3.88 (dd, J=9.8, 5.5 Hz, 1H), 3.59-3.48 (m, 5H), 3.26-3.21 (m, 1H), 2.52 (dd, J=13.0, 5.7 Hz, 1H), 2.41 (dd, J=12.7, 7.1 Hz, 1H), 1.45 (s, 9H), 1.41 (s, 9H); ¹³C-NMR (CD₃OD; 125 MHz) δ 165.3, 152.1, 151.8, 140.6, 130.1, 130.0, 129.4, 128.2, 84.1, 81.2, 60.5, 60.2, 58.5, 53.2, 46.3, 28.7, 28.4, 28.3, 28.2; HRMS (ESI) Calculated for C₂₈H₃₉N₄O₄ 495.2966. found 495.2958 (M+H)⁺.

A solution of SI-3 (FIG. 26) (0.045 g, 0.091 mmol) in 1N aqueous HCl (10 mL) was stirred at room temperature for 16 h, then concentrated in vacuo, using water and methanol co-evaporations to remove residual solvents. The crude reaction mixture was resuspended in methanol (10 mL) and had 20% Pd(OH)₂ (15 mg, wet) and 10% Pd/C (30 mg) sequentially added. The reaction mixture was bubbled with hydrogen gas using a balloon (1 atm) and monitored by LCMS (C18 RP-HPLC) for consumption of the di-benzylated starting material (consumed after 30 minutes) and the mono-benzylated intermediate (consumed after 22 h overnight incubation). The reaction mixture was filtered through a pad of Celite, rinsed with methanol (30 mL), then 0.1% aqueous acetic acid (30 mL) and concentrated in vacuo. 1 N aqueous HCl (5 mL) was added to the filtrate to obtain the HCl salt, then concentrated in vacuo and lyophilized. The desired product was obtained as a light yellow solid (0.017 g, 99%). ¹H-NMR (D₂O+0.1% CH₃OH; 500 MHz): δ 4.42 (dddd, J=10.9, 5.7, 5.7, 5.7 Hz, 1H), 3.94 (t, J=10.2 Hz, 1H), 3.54 (dd, J=10.5, 6.0 Hz, 1H), 3.31-3.19 (m, 2H); ¹³C-NMR (D₂O+0.1% CH₃OH 500 MHz): δ 159.8, 52.3, 46.0, 42.1; HRMS (ESI) Calculated for C₄H₁₁N₄ 115.0978. found 115.0976 (M+H)⁺.

### Synthesis of Dimethylamine Intermediate 7

A solution of (S)-Garner's aldehyde (0.560 g, 2.44 mmol) and dimethylamine (1.28 mL, [2.0 M solution in THF], 2.56 mmol) in methanol (10 mL) had acetic acid (0.1 mL) added and was stirred for 5 minutes at room temperature. To this solution, sodium cyanoborohydride (0.169 g, 2.69 mmol) was added over a few minutes and stirred at room temperature under an argon atmosphere for 24 h. The reaction mixture was cooled to 0° C. and an aqueous solution of saturated sodium bicarbonate (25 mL) was added, followed by ethyl acetate (50 mL). The layers were separated and the aqueous layer was further extracted with ethyl acetate (2×50 mL). Pooled organic layers were washed with brine (50 mL), dried over magnesium sulfate, filtered and concentrated in vacuo. The crude reaction mixture was purified by silica flash chromatography and eluted over a gradient of 50:1 to 10:1 ethyl acetate:methanol+0.1% triethylamine. Pooled fractions were concentrated in vacuo, yielding the desired product as a clear colorless oil (0.313 g, 50%). Rf 0.18 (10:1 ethyl acetate:methanol+0.1% triethylamine); IR (CH₂Cl₂ cast) 3433, 2977, 2825, 2773, 1697, 1641, 1461, 1386, 1254, 1173, 1074 cm⁻¹; ¹H-NMR (CD₃OD; 500 MHz, mixture of 2 rotamers) δ 4.01-3.89 (m, 3H), 2.53-2.44 (m, 1H), 2.42-2.27 (m, 7H), 1.52 (s, 3H), 1.50-1.44 (m, 12H); ¹³C-NMR (CD₃OD; 125 MHz, mixture of 2 rotamers) δ 153.2, 94.8, 94.6, 81.8, 81.3, 79.5, 67.7, 67.3, 62.8, 62.0, 56.8, 56.6, 46.3, 46.1, 28.8, 28.7, 28.1, 27.3, 24.5, 23.2; HRMS (ESI) Calculated for C₁₃H₂₇N₂O₃ 259.2016. found 259.2014 (M+H)⁺.

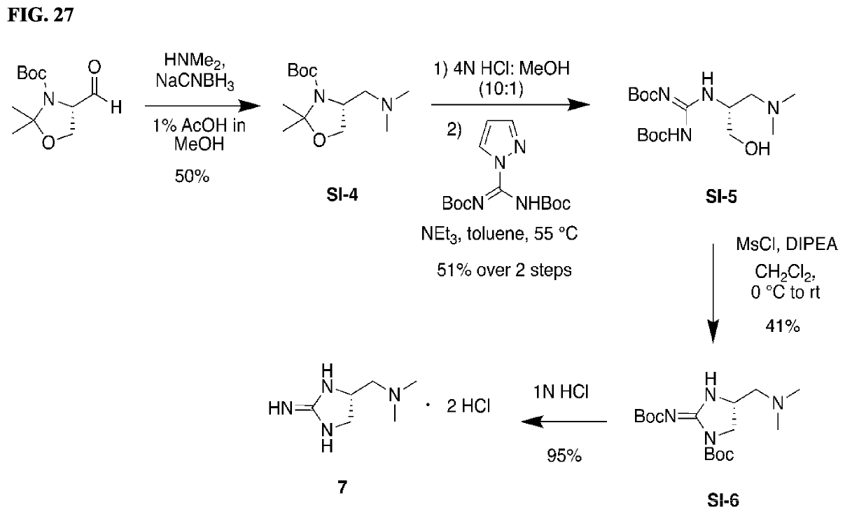

FIG. 27 shows the intermediates involved in Synthesis of dimethylamine intermediate 7. A solution of SI-4 (FIG. 27) (0.245 g, 0.95 mmol) in 4 N aqueous HCl (10 mL) and methanol (1 mL) was stirred at room temperature for 2.5 h. The reaction mixture was concentrated in vacuo, using water and toluene co-evaporations to remove additional HCl and water respectively. The crude material was resuspended in toluene (15 mL) and had triethylamine (0.66 mL, 4.74 mmol) and N,N′-di-Boc-1H-pyrazole-1-carboxamidine (0.324 g, 1.04 mmol) sequentially added. The reaction mixture was heated to 55° C. and stirred for 17 h, then was cooled to room temperature, diluted with ethyl acetate (50 mL) and washed with water (2×25 mL). The organic layer was dried over magnesium sulfate, filtered, and concentrated in vacuo. The crude reaction mixture was purified by silica flash chromatography and eluted over a stepwise gradient of 50:1 to 33:1 to 20:1 to 9:1 ethyl acetate:methanol+0.1% triethylamine. Pooled fractions were concentrated in vacuo, yielding the desired product as a light yellow oil (0.175 g, 51%). Rf 0.15 (9:1 ethyl acetate:methanol+0.1% triethylamine); IR (CH₂Cl₂ cast) 3324, 3139, 2978, 1726, 1639, 1565, 1462, 1415, 1320, 1257, 1161, 1053 cm⁻¹; ¹H-NMR (CD₃OD; 500 MHz) δ 7.90 (s, 1H), 4.26-4.20 (m, 1H), 3.62 (d, J=4.1 Hz, 2H), 2.58 (dd, J=12.8, 9.0 Hz, 1H), 2.46 (dd, J=12.8, 5.3 Hz, 1H), 2.31 (s, 6H), 1.53 (s, 9H), 1.46 (s, 9H); ¹³C-NMR (CD₃OD; 125 MHz) δ 164.5, 157.7, 154.1, 84.4, 80.3, 63.5, 61.3, 51.6, 46.0, 28.5, 28.2; HRMS (ESI) Calculated for C₁₆H₃₃N₄O₅ 361.2445. found 361.2440 (M+H)⁺.

To a 0° C. solution of SI-5 (FIG. 27) (0.122 g, 0.34 mmol) in dry CH₂Cl₂ (10 mL) was sequentially added DIPEA (118 μL, 0.68 mmol) and methanesulfonyl chloride (29 μL, 0.37 mmol). The reaction mixture was slowly warmed to room temperature over 18 h, then quenched by the addition of saturated NH₄Cl (20 mL) and diluted with CH₂Cl₂ (10 mL). The layers were separated and the organic layer was further washed with water (20 mL), and brine (20 mL), dried over magnesium sulfate, filtered and concentrated in vacuo. The crude reaction mixture was purified by silica flash chromatography and eluted over a gradient of 20:1 to 9:1 chloroform:methanol+0.1% triethylamine. Pooled fractions were concentrated in vacuo, yielding the desired product as a white solid (0.047 g, 41%). Rf 0.4 (9:1 chloroform:methanol+0.1% triethylamine); IR (CH₂Cl₂ cast) 3411, 2980, 1643, 1600, 1512, 1474, 1340, 1257, 1164, 1106 cm⁻¹; ¹H-NMR (CD₃OD; 500 MHz) δ 4.10 (dddd, J=9.4, 6.7, 6.7, 6.7 Hz, 1H), 3.92 (dd, J=10.7, 9.4 Hz, 1H), 3.55 (dd, J=10.6, 6.4 Hz, 1H), 2.60 (dd, J=12.4, 6.4 Hz, 1H), 2.49 (dd, J=12.4, 7.1 Hz, 1H), 2.37 (s, 6H), 1.53 (s, 9H), 1.49 (s, 9H); ¹³C-NMR (CD₃OD; 125 MHz) δ 154.7, 152.8, 152.5, 84.5, 81.8, 64.7, 50.0, 49.8, 45.9, 28.4, 28.3; HRMS (ESI) Calculated for C₂₂H₄₅N₈O₄ 485.3558. found 485.3557 (2M-2Boc+H)⁺.

A solution of SI-6 (FIG. 27) (0.032 g, 0.093 mmol) in 1 N aqueous HCl (5 mL) was stirred at room temperature for 14 h, then concentrated in vacuo and lyophilized. The desired product was obtained as a light yellow solid (0.019 g, 95%). ¹H-NMR (D₂O+0.1% CH₃OH; 500 MHz): δ 4.53 (dddd, J=9.6, 9.6, 5.3, 5.3 Hz, 1H), 3.96 (dd, J=10.2, 10.2 Hz, 1H), 3.52-3.42 (m, 2H), 3.36 (dd, J=13.5, 4.8 Hz, 1H), 2.95 (s, 6H); ¹³C-NMR (D₂O+0.1% CH₃OH 500 MHz): δ 159.9, 60.4, 50.3, 46.9, 44.5, 42.8; HRMS (ESI) Calculated for C₆H₁₅N₄ 143.1291. found 143.1291 (M+H)⁺.

### Synthesis of γ-Hydroxy-L-Arginine Diastereomers SI-7 and SI-8

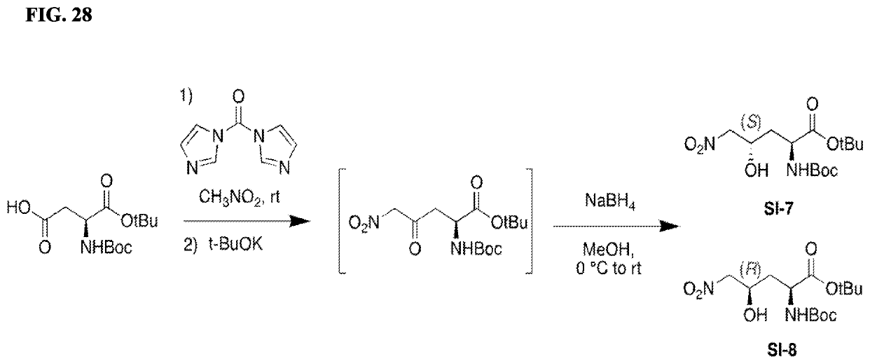

FIG. 28 shows the synthesis of γ-hydroxy-L-arginine diastereomers SI-8. This procedure was adapted from a literature reference⁸⁸. Briefly, a solution of Boc-L-Asp-OtBu (2.008 g, 6.94 mmol) and 1,1′-carbonyldiimidazole (1.490 g, 6.95 mmol) in nitromethane (40 mL) was stirred at room temperature under Ar gas for 45 minutes. Potassium tert-butoxide (1.558 g, 13.88 mmol) was added at once and the resulting solution was stirred for an additional 4.5 hours at room temperature. The reaction mixture was quenched by the addition of 50% aqueous glacial acetic acid (50 mL), then extracted with ethyl acetate (3×60 mL). Pooled organic layers were washed with water (50 mL), saturated aqueous sodium bicarbonate (50 mL), water (50 mL), then brine (50 mL). The organic layer was dried over magnesium sulfate, filtered, and concentrated in vacuo. The crude material was resuspended in methanol (20 mL) then stirred and cooled to 0° C. Sodium borohydride (0.262 g, 6.94 mmol) was added portion-wise to this solution over 2 minutes at 0° C., stirred at this temperature for 45 minutes, then slowly warmed to room temperature over 16 hours. The reaction mixture was quenched by the addition of 1 N aqueous HCl until a pH of 3, concentrated in vacuo, then resuspended in water (25 mL) and extracted with EtOAc (3×25 mL). Pooled organic layers were washed with brine (25 mL), dried over magnesium sulfate, filtered, and concentrated in vacuo. The crude reaction mixture was purified by silica flash chromatography and the two desired diastereomers were eluted over a slow gradient of 50:1 to 50:3 dichloromethane:diethyl ether. If additional silica flash chromatography was needed to further purify the two diastereomers, an isocratic 18:1:1 toluene:tetrahydrofuran:ethyl acetate eluant system was used. Pooled fractions were concentrated in vacuo, yielding both desired products as flaky white solids.

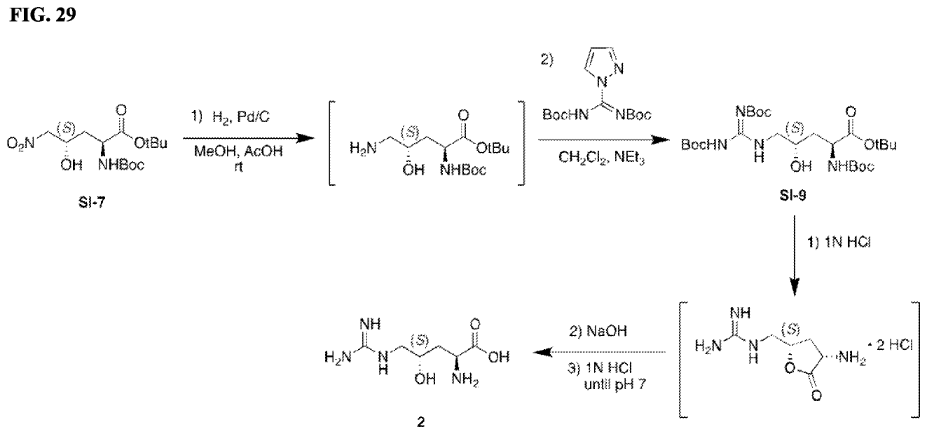

SI-7 (FIG. 29) (S)-diastereomer (0.628 g, 27%). Rf 0.65 (9:1 CH₂Cl₂:Et₂O); IR (CH₂Cl₂ cast) 3412, 2980, 2932, 1695, 1558, 1506, 1369, 1253, 1155 cm⁻¹; ¹H NMR (500 MHz, CDCl₃) δ 5.42 (d, J=7.5 Hz, 1H), 4.72 (d, J=4.3 Hz, 1H), 4.50 (dd, J=12.0, 8.3 Hz, 1H), 4.45-4.30 (m, 3H), 1.95 (ddd, J=13.9, 11.0, 3.1 Hz, 1H), 1.57-1.50 (m, 1H), 1.47 (s, 9H), 1.46 (s, 9H); ¹³C NMR (126 MHz, CDCl₃) δ 171.1, 157.3, 83.2, 81.4, 80.0, 65.2, 50.7, 38.7, 28.4, 28.1; HRMS (ESI) Calculated for C₁₄H₂₇N₂O₇ 335.1813. found 335.1831 (M+H)⁺.

SI-8 (FIG. 28) (R)-diastereomer (0.579 g, 25%). Rf 0.5 (9:1 CH₂Cl₂:Et₂O); IR (CH₂Cl₂ cast) 3419, 2979, 2918, 1695, 1557, 1384, 1369, 1252, 1155 cm⁻¹; ¹H NMR (500 MHz, CDCl₃) δ 5.42 (s, 1H), 4.54 (dt, J=9.5, 4.8 Hz, 1H), 4.45 (d, J=5.9 Hz, 2H), 4.27 (d, J=6.3 Hz, 1H), 3.36 (s, 1H), 2.08 (d, J=14.2 Hz, 1H), 1.90 (ddd, J=14.6, 9.0, 6.0 Hz, 1H), 1.48 (s, 9H), 1.45 (s, 9H); ¹³C NMR (126 MHz, CDCl₃) δ 171.0, 155.9, 83.1, 80.8, 80.3, 66.3, 51.5, 37.1, 28.4, 28.1; HRMS (ESI) Calculated for C₁₄H₂₇N₂O₇ 335.1813. found 335.1828 (M+H)⁺.

### Synthesis of (S)-g-Hydroxy-L-Arginine 2

FIG. 29 shows the intermediates for Synthesis of (S)-γ-hydroxy-L-arginine 2. This procedure was adapted from a literature reference⁸⁸. A solution of SI-7 (FIG. 29) (0.454 g, 1.36 mmol) in MeOH (15 mL) and acetic acid (0.078 mL, 1.36 mmol) had 10% Pd/C (0.145 g, 0.136 mmol) added to it and was sparged with Ar gas, then left stirring under a H₂ atmosphere for 15 hours. The reaction mixture was filtered through a pad of Celite and concentrated in vacuo. The crude reaction mixture was resuspended in toluene (20 mL) and had N,N′-di-Boc-1H-pyrazole-1-carboxamidine (0.464 g, 1.49 mmol) and triethylamine (0.95 mL, 6.79 mmol) added sequentially. The reaction mixture was heated to 55° C. and stirred for 17 hours, then quenched by the addition of a saturated aqueous NH₄Cl solution (25 mL). Organic components were extracted with EtOAc (3×25 mL), dried over magnesium sulfate, filtered and concentrated in vacuo. The crude reaction mixture was purified by silica flash chromatography using an eluent of 4:1 hexanes:ethyl acetate+0.1% triethylamine. Pooled fractions were concentrated in vacuo, yielding the desired product as a clear light yellow oil (0.374 g, 50%). Rf 0.6 (2:1 hexane:ethyl acetate); IR (CH₂Cl₂ cast) 3337, 2980, 2932, 1728, 1645, 1621, 1368, 1155, 1055, 1027 cm⁻¹; ¹H NMR (500 MHz, CDCl₃) δ 11.44 (s, 1H), 8.70 (t, J=5.3 Hz, 1H), 5.46 (d, J=8.0 Hz, 1H), 4.99-4.77 (m, 1H), 4.46-4.26 (m, 1H), 3.84-3.73 (m, 1H), 3.73-3.64 (m, 1H), 3.28 (ddd, J=13.0, 7.7, 4.1 Hz, 1H), 1.88 (ddd, J=13.9, 10.7, 3.4 Hz, 1H), 1.59-1.53 (m, 1H), 1.49 (s, 9H), 1.48 (s, 9H), 1.46 (s, 9H), 1.44 (s, 9H); ¹³C NMR (126 MHz, CDCl₃) δ 171.8, 163.4, 156.9, 156.8, 153.1, 83.3, 82.5, 80.5, 79.5, 77.4, 67.1, 51.3, 46.5, 39.0, 28.4, 28.4, 28.2, 28.1; HRMS (ESI) Calculated for C₂₅H₄₇N₄O₉ 547.3338. found 547.3329 (M+H)⁺.

A solution of SI-9 (FIG. 29) (0.037 g, 0.068 mmol) in 1 N aqueous HCl (5 mL) was stirred for 3 hours then concentrated in vacuo, using additional water washes to remove excess HCl. The crude reaction mixture was resuspended in water, and an aqueous solution of sodium hydroxide (0.014 g, 0.34 mmol) was added and stirred for 2 hours. The reaction mixture was neutralized by the addition of 1 N HCl until pH 7.0, then lyophilized. The desired product was obtained as a white solid alongside sodium chloride and was quantified using ¹H NMR with a MeOH internal standard (0.012 g, 93%). ¹H NMR (500 MHz, D₂O+0.1% MeOH) δ 4.19 (dd, J=5.9, 5.9 Hz, 1H), 3.99 (dddd, J=8.2, 8.2, 4.1, 4.0 Hz, 1H), 3.34 (dd, J=14.7, 3.8 Hz, 1H), 3.23 (dd, J=14.6, 7.3 Hz, 1H), 2.15-2.04 (m, 2H); ¹³C-NMR (126 MHz, D₂O+0.1% MeOH) δ 172.2, 157.4, 66.6, 51.2, 46.9, 33.0; HRMS (ESI) Calculated for C₆H₁₅N₄O₃ 191.1139. found 191.1136 (M+H).

### Synthesis of L-Enduracididine (3)

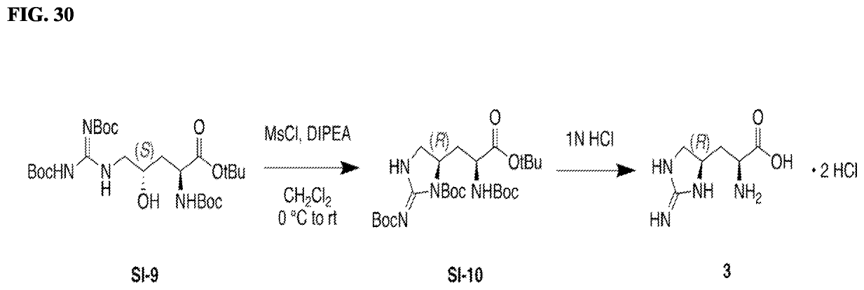

FIG. 30 shows the intermediates involved in the Synthesis of L-enduracididine (3). To a 0° C. solution of SI-9 (FIG. 29) (0.246 g, 0.45 mmol) in dry CH₂Cl₂ (10 mL) was sequentially added DIPEA (0.235 mL, 1.35 mmol), then methanesulfonyl chloride (0.038 mL, 0.50 mmol). The resulting solution was stirred at 0° C. then slowly warmed to room temperature over 17 hours. The reaction mixture was diluted with additional CH₂Cl₂ (10 mL), quenched by the addition of saturated aqueous NH₄Cl (20 mL), then the layers were separated. Organic materials were extracted using subsequent washes with CH₂Cl₂ (2×25 mL), then pooled organic layers were washed with brine (25 mL), dried with magnesium sulfate, filtered, and concentrated in vacuo. The crude reaction mixture was purified using silica flash chromatography over a gradient of 2:1 to 1:1 to 1:2 hexanes:EtOAc+0.1% triethylamine. Pooled fractions were concentrated in vacuo, yielding the desired product as a sticky clear colorless oil (0.083 g, 35%). Rf 0.2 (1:1 hexane:ethyl acetate+1 drop triethylamine); IR (CH₂Cl₂ cast) 3349, 2979, 2933, 1756, 1713, 1653, 1606, 1532, 1384, 1370, 1142 cm⁻¹; ¹H NMR (500 MHz, CD₃OD) δ 4.29 (t, J=9.4 Hz, 1H), 4.05 (d, J=8.5 Hz, 1H), 3.81 (dd, J=12.6, 9.1 Hz, 1H), 3.54 (dd, J=12.7, 3.1 Hz, 1H), 2.12 (ddd, J=11.3, 9.4, 3.6 Hz, 1H), 1.95 (dd, J=12.3, 12.0 Hz, 1H), 1.56 (s, 9H), 1.49 (s, 9H), 1.47 (s, 9H), 1.44 (s, 9H); ¹³C-NMR (126 MHz, CD₃OD) δ 172.6, 157.8, 152.3, 152.2, 152.2, 84.9, 83.1, 81.6, 80.6, 55.6, 52.3, 49.9, 37.0, 28.8, 28.4, 28.4, 28.2; HRMS (ESI) Calculated for C₂₅H₄₅N₄O₈ 529.3232. found 529.3261 (M+H)⁺.

A solution of SI-10 (FIG. 30) (0.067 g, 0.13 mmol) in 1N HCl (10 mL) was stirred for 3 hours and then concentrated in vacuo, using additional water washes to remove excess HCl, then lyophilized. The desired product was obtained as a white solid (0.032 g, quantitative). ¹H NMR (500 MHz, D₂O+0.1% MeOH) δ 4.34 (dddd, J=6.8, 6.8, 6.1, 6.1 Hz, 1H), 4.08 (dd, J=9.2, 5.6 Hz, 1H), 3.88 (dd, J=9.7, 9.7 Hz, 1H), 3.42 (dd, J=10.1, 6.0 Hz, 1H), 2.26 (ddd, J=14.8, 8.8, 5.8 Hz, 1H), 2.18 (ddd, J=13.9, 6.7, 6.7 Hz, 1H); ¹³C-NMR (126 MHz, D₂O+0.1% MeOH) δ 171.4, 159.6, 52.0, 50.0, 48.3, 35.5; HRMS (ESI) Calculated for C₆H₁₃N₄O₂ 173.1033. found 173.1030 (M+H)⁺.

### Synthesis of (R)-g-Hydroxy-L-Arginine (SI-12)

This procedure was adapted from a literature reference⁸⁸. A solution of SI-8 (0.200 g, 0.60 mmol) in MeOH (10 mL) and acetic acid (0.034 mL, 0.60 mmol) had 10% Pd/C (0.064 g, 0.060 mmol) added to it and was sparged with Ar gas, then left stirring under a H₂ atmosphere for 15 hours. The reaction mixture was filtered through a pad of Celite and concentrated in vacuo. The crude reaction mixture was resuspended in toluene (20 mL) and had N,N′-di-Boc-1H-pyrazole-1-carboxamidine (0.204 g, 0.66 mmol) and triethylamine (0.42 mL, 2.99 mmol) added sequentially. The reaction mixture was heated to 55° C. and stirred for 17 hours, then quenched by the addition of a saturated aqueous NH₄Cl solution (25 mL). Organic components were extracted with EtOAc (3×25 mL), dried over magnesium sulfate, filtered and concentrated in vacuo. The crude reaction mixture was purified by silica flash chromatography using an eluent of 4:1 hexanes:ethyl acetate+0.1% triethylamine. Pooled fractions were concentrated in vacuo, yielding the desired product as a clear light yellow oil (0.142 g, 43%). Rf 0.6 (2:1 hexane:ethyl acetate); IR (CH₂Cl₂ cast) 3339, 2980, 2920, 1726, 1645, 1621, 1368, 1157, 1054, 1027 cm¹; ¹H NMR (500 MHz, CDCl₃) δ 11.42 (s, 1H), 8.69 (s, 1H), 5.48-5.38 (m, 1H), 5.22 (s, 1H), 4.21 (d, J=7.9 Hz, 1H), 3.92 (d, J=9.0 Hz, 1H), 3.59 (dd, J=15.5, 5.9 Hz, 1H), 3.43 (dd, J=13.5, 6.7 Hz, 1H), 1.99-1.92 (m, 1H), 1.86 (ddd, J=14.4, 8.7, 6.4 Hz, 1H), 1.49 (s, 9H), 1.46 (s, 18H), 1.44 (s, 9H); ¹³C NMR (126 MHz, CDCl₃) δ 171.7, 162.9, 157.7, 155.8, 153.1, 83.7, 82.2, 80.0, 79.7, 69.2, 52.1, 47.5, 37.6, 28.5, 28.3, 28.2, 28.1; HRMS (ESI) Calculated for C₂₅H₄₇N₄O₉ 547.3338. found 547.3328 (M+H)⁺.

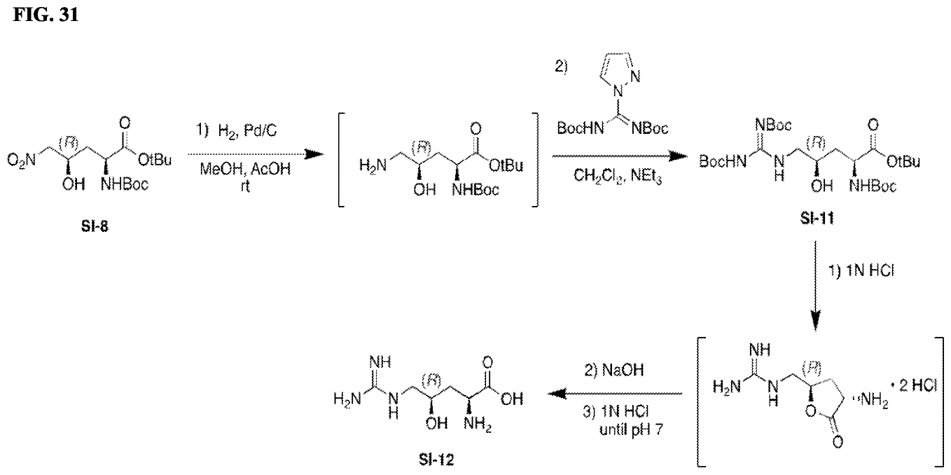

FIG. 31 shows the intermediates involved in the Synthesis of (R)-γ-hydroxy-L-arginine (SI-12). A solution of SI-11 (FIG. 31) (0.034 g, 0.062 mmol) in 1 N aqueous HCl (5 mL) was stirred for 3 hours then concentrated in vacuo, using additional water washes to remove excess HCl. The crude reaction mixture was resuspended in water, and an aqueous solution of sodium hydroxide (0.012 g, 0.31 mmol) was added and stirred for 2 hours. The reaction mixture was neutralized by the addition of 1 N HCl until pH 7.0, then lyophilized. The desired product was obtained as a white solid alongside sodium chloride and was quantified using ¹H NMR with a MeOH internal standard (0.010 g, 88%). ¹H NMR (500 MHz, D₂O+0.1% MeOH) δ 4.08 (ddd, J=7.4, 7.2, 3.6 Hz, 1H), 3.83 (dd, J=8.1, 5.3 Hz, 1H), 3.33 (dd, J=14.9, 3.1 Hz, 1H), 3.21 (dd, J=14.6, 7.4 Hz, 1H), 2.10 (ddd, J=14.9, 5.3, 2.9 Hz, 1H), 1.83 (dt, J=14.8, 9.3 Hz, 1H); ¹³C-NMR (126 MHz, D₂O+0.1% MeOH) δ 174.3, 157.3, 68.4, 53.5, 47.0, 34.1; HRMS (ESI) Calculated for C₆H₁₅N₄O₃ 191.1139. found 191.1135 (M+H)⁺.

### Synthesis of L-Allo-Enduracididine (SI-14)

To a 0° C. solution of SI-11 (FIG. 31) (0.124 g, 0.23 mmol) in dry CH₂Cl₂ (10 mL) was sequentially added DIPEA (0.12 mL, 0.68 mmol), then methanesulfonyl chloride (0.019 mL, 0.25 mmol). The resulting solution was stirred at 0° C. for 2 hours, then slowly warmed to room temperature over 17 hours. The reaction mixture was diluted with additional CH₂Cl₂ (10 mL), quenched by the addition of saturated aqueous NH₄Cl (20 mL), then the layers were separated. Organic materials were extracted using subsequent washes with CH₂Cl₂ (2×25 mL), then pooled organic layers were washed with brine (25 mL), dried with magnesium sulfate, filtered, and concentrated in vacuo. The crude reaction mixture was purified using silica flash chromatography over a gradient of 4:1 to 1:1 hexanes:EtOAc+0.1% triethylamine. Pooled fractions were concentrated in vacuo, yielding the desired product as a sticky clear colorless oil (0.094 g, 78%). Rf 0.2 (1:1 hexane:ethyl acetate+1 drop triethylamine); IR (CH₂Cl₂ cast) 3366, 2979, 2929, 1747, 1714, 1606, 1539, 1384, 1369, 1311, 1252, 1149 cm⁻¹; ¹H NMR (500 MHz, CD₃OD) δ 4.30 (dddd, J=12.2, 8.3, 3.3, 3.1 Hz, 1H), 4.09 (dd, J=8.9, 5.4 Hz, 1H), 3.84 (dd, J=12.8, 9.2 Hz, 1H), 3.55 (dd, J=12.8, 3.5 Hz, 1H), 2.23 (ddd, J=13.9, 5.4, 3.0 Hz, 1H), 1.90 (ddd, J=13.9, 8.8, 8.8 Hz, 1H), 1.56 (s, 9H), 1.49 (s, 9H), 1.48 (s, 9H), 1.45 (s, 9H); ¹³C-NMR (126 MHz, CD₃OD) δ 172.7, 157.8, 152.4, 152.4, 152.4, 85.0, 83.1, 81.8, 80.6, 56.4, 53.3, 37.2, 28.8, 28.4, 28.4, 28.3; HRMS (ESI) Calculated for C₂₅H₄₅N₄O₈ 529.3232. found 529.3266 (M+H)⁺.

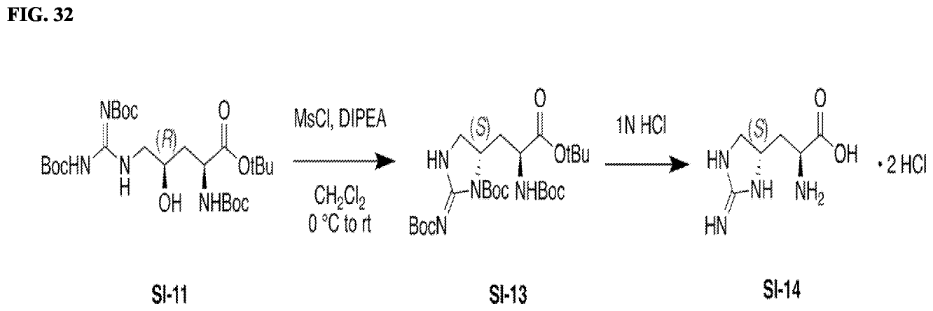

FIG. 32 shows the intermediates involved in Synthesis of L-allo-enduracididine (SI-14). A solution of SI-13 (FIG. 32) (0.069 g, 0.13 mmol) in 1 N HCl (10 mL) was stirred for 3 hours and then concentrated in vacuo, using additional water washes to remove excess HCl, then lyophilized. The desired product was obtained as a white solid (0.032 g, quantitative). ¹H NMR (500 MHz, D₂O+0.1% MeOH) δ 4.31 (ddd, J=13.4, 6.6 Hz, 1H), 4.09 (dd, J=7.0 Hz, 1H), 3.89 (dd, J=9.7 Hz, 1H), 3.44 (dd, J=10.0, 6.4 Hz, 1H), 2.35 (ddd, J=14.5, 7.2 Hz, 1H), 2.14 (ddd, J=14.0, 6.5 Hz, 1H); ¹³C-NMR (126 MHz, D₂O+0.1% MeOH) δ 171.5, 159.5, 52.2, 50.2, 48.0, 35.2; HRMS (ESI) Calculated for C₆H₁₃N₄O₂ 173.1033. found 173.1038 (M+H)⁺.

### Example 5: Primer Design

Primers which targeted conserved regions of guanitoxin biosynthetic genes were designed as follows:

Allelic CDS sequences (e.g. coding sequences) for guanitoxin biosynthetic genes (3-5 alleles per gene) were harvested from assemblies of the Gnt biosynthetic gene cluster (BGC) from public environmental freshwater metagenomic and metatranscriptomic sequencing datasets. These datasets were selected, as they had initial hits for the Gnt BGC via the SearchSRA tool (https://www.searchsra.org/). The datasets included (alongside the original Gnt CDS sequences from ITEP-024) are shown in Table 10.

These assemblies were created from the datasets with SPAdes or rnaSPAdes, respectively (https://github.com/ablab/spades). Genes were annotated within these assemblies with Prokka (https://github.com/tseemann/prokka), and the Gnt genes found within this gene annotation using blastp or tblastn via SequenceServer (https://sequenceserver.com). The multiple CDSs per gene were input into Primer-BLAST (https://www.ncbi.nlm.nih.gov/tools/primer-blast/), under the “Primers common for a group of sequences” mode, with default parameters excepting the primer melting temperatures (Tₘ) were: Min=55° C., Max=65° C., and Max Tₘ difference=5°. If Primer-BLAST was unable to find a suitable primer pair, the allele with the least sequence identity to the canonical Gnt gene from Sphaerospermopsis torques-reginae ITEP-024 was removed, and Primer-BLAST was rerun with success.

TABLE 1
¹H NMR table for compounds 1-4 and 6-8

| | 1* | 8* | 7* | 7 | 6 | 4 | 3 | 2 |
|---|---|---|---|---|---|---|---|---|
| ¹H | (isolated) | (isolated) | (synthetic) | (synthetic) | (synthetic) | (enzymatic) | (synthetic) | (synthetic) |
| 4a | 4.01, dd | 3.93, dd | 3.98, dd | 3.96, dd | 3.94, dd | 3.83, dd | 3.88, dd | 3.34, dd |
| | (10.1, 9.4) | (10.0, 8.5) | (10.1, 9.7) | (10.2, 10.2) | (10.2, 10.2) | (10.0, 10.0) | (9.7, 9.7) | (14.7, 3.8) |
| 4b | 3.51, dd | 3.46, dd | 3.50, dd | 3.48, m | 3.54, dd | 3.65, dd | 3.42, dd | 3.23, dd |
| | (10.1, 9.7) | (10.0, 8.0) | (10.1, 5.4) | | (10.5, 6.0) | (10.3, 5.5) | (10.1, 6.0) | (14.6, 7.3) |
| 5 | 4.71, m | 4.48, | 4.55, dddd | 4.53, dddd | 4.42, dddd | 4.34, ddd | 4.34, m | 3.99, dddd |
| | | dddd | | (9.6, 9.6, | (10.9, 5.7, | (9.7, 6.8, | | (8.2, 8.2, |
| | | | | 5.3, 5.3) | 5.7, 5.7) | 5.5) | | 4.1, 4.0) |
| 6a | 3.75, dd | 3.75, dd | 3.48, dd | 3.45, m | 3.25, m | 4.06, dd | 2.26, ddd | 2.09, m |
| | (13.9, 9.3) | (13.9, 6.8) | (13.4, 8.1) | | | (6.7, 3.1) | (14.8, 8.8, | |
| | | | | | | | 5.8) | |
| 6b | 3.47, dd | 3.47, dd | 3.38, dd | 3.36, dd | 3.25, m | — | 2.18, ddd | 2.09, m |
| | (13.9, 2.9) | (13.9, 4.5) | (13.4, 4.8) | (13.5, 4.8) | | | (13.9, 6.7, | |
| | | | | | | | 6.7) | |
| 7 | — | — | — | — | — | 3.90, d | 4.08, dd | 4.19, dd |
| | | | | | | (3.1) | (9.2, 5.6) | (5.9, 5.9) |
| NMe₂ | 3.00, s | 3.00, s | 2.96, s | 2.95, s | — | — | — | — |
| OMe | 3.79, d | — | — | — | — | — | — | — |
| | (11.0) | | | | | | | |

*from (45); spectra ran in D₂O + 1% acetic acid-d₄
all other spectra were collected in D₂O + 0.1% CH₃OH and referenced to methanol (δ 3.34)

TABLE 2
¹³C NMR table for compounds 1-4 and 6-8

| | 1* | 8* | 7* | 7 | 6 | 4 | 3 | 2 |
|---|---|---|---|---|---|---|---|---|
| ¹³C | (isolated) | (isolated) | (synthetic) | (synthetic) | (synthetic) | (enzymatic) | (synthetic) | (synthetic) |
| 2 | 163.7 | 162.5 | 160.6 | 159.9 | 159.8 | 159.4 | 159.5 | 157.4 |
| 4 | 56.1 | 44.9 | 47.6 | 46.9 | 46.0 | 44.9 | 48.3 | 46.9 |
| 5 | 60.3 | 58.8 | 51.0 | 50.3 | 52.3 | 56.5 | 52.1 | 66.6 |
| 6 | 58.7 | 58.7 | 61.1 | 60.4 | 42.1 | 71.5 | 35.5 | 33.0 |
| 7 | — | — | — | — | — | 55.9 | 50.0 | 51.2 |
| 8 | — | — | — | — | — | 171.4 | 171.3 | 172.2 |
| NMe₂ | 43.9 | 44.7 | 44.3 | 42.8 | — | — | — | — |
| OMe | 56.1 | — | — | — | — | — | — | — |

*from (45); spectra ran in D₂O + 1% acetic acid-d₄
all other spectra were collected in D₂O + 0.1% CH₃OH and referenced to methanol (δ 49.0)

TABLE 3
antiSMASH annotation of the Sphaerospermopsis torques-reginae
ITEP-024 genome. In total, 11 candidate BGC clusters were detected
by antiSMASH. Notably, one location overlapped with the previously
characterized anabaenopeptin and spumigin BGCs⁴⁵.

| Genomic | Region | | |
|---|---|---|---|
| coordinates | size | BGC type | Most similar to |
| CP080598.1: | 91,043 bp | NRPS, | CP080598.1: |
| 29917-120960 | | betalactone, | 43683-105015, |
| | | microviridin | 100% alignment to |
| | | | prev. characterized |
| | | | KX788858.1: |
| | | | anabaenopeptin and |
| | | | spumigin BGCs⁴⁵. |
| CP080598.1: | 49,430 bp | T1-PKS, | |
| 286410-335840 | | hglE-KS | |
| CP080598.1: | 20,861 bp | terpene | |
| 875865-896726 | | | |
| CP080598.1: | 25,031 bp | cyanobactin | anacyclamide D8P |
| 1152826-1177857 | | | |
| CP080598.1: | 52,115 bp | hglE-KS, | heterocyte glycolipids |
| 1195859-1247974 | | T1-PKS | |
| CP080598.1: | 42,243 bp | T1-PKS | |
| 1518085-1560328 | | | |
| CP080598.1: | 28,416 bp | terpene, | |
| 1983890-2012306 | | oligosaccharide | |
| CP080598.1: | 41,612 bp | lanthipeptide- | |
| 2335145-2376757 | | class-V | |
| CP080598.1: | 20,932 bp | terpene | |
| 2990394-3011326 | | | |
| CP080598.1: | 42,405 bp | lanthipeptide- | |
| 4204921-4247326 | | class-V | |
| CP080598.1: | 73,652 bp | NRPS | |
| 4412989-4486641 | | | |

T1-PKS = type-1 PKS, hglE-KS = heterocyte glycolipid synthase-like PKS.

TABLE 4
Sequence Read Archive (SRA) metagenomic and metatranscriptomic datasets with reads that match the gnt BGC.

| SRA | # reads | Matched | | | | | | |
|---|---|---|---|---|---|---|---|---|
| dataset | (AS >=150) | genes | geo_loc_name | env_biome | env_feature | lat_lon | | collection_date |
| SRR5249014 | 1912 | gntA, gntB, | USA: | N/A | N/A | 41.474889 | N | Jun. 11, 2013 |
| | | gntC, gntD, | Sandusky Bay, | | | 82.854137 | W | |
| | | gntE, gntF, | Ohio | | | | | |
| | | gntG, gntH, | | | | | | |
| | | gntI, gntJ | | | | | | |
| SRR5249015 | 1887 | gntA, gntB, | USA: | N/A | N/A | 41.474889 | N | Jun. 11, 2013 |
| | | gntC, gntD, | Sandusky Bay, | | | 82.854137 | W | |
| | | gntE, gntF, | Ohio | | | | | |
| | | gntG, gntH, | | | | | | |
| | | gntI, gntJ | | | | | | |
| SRR1601415 | 1274 | gntA, gntB, | USA | Lake | Western | 41.7394 | N | 8 Oct. 13 |
| | | gntC, gntD, | | Erie | Basin | 83.3750 | W | |
| | | gntE, gntF, | | | | | | |
| | | gntG, gntH, | | | | | | |
| | | gntI, gntJ | | | | | | |
| SRR1601417 | 1188 | gntA, gntB, | USA | Lake | Western | 41.6989 | N | 8 Oct. 13 |
| | | gntC, gntD, | | Erie | Basin | 83.4589 | W | |
| | | gntE, gntF, | | | | | | |
| | | gntG, gntH, | | | | | | |
| | | gntI, gntJ | | | | | | |
| SRR1601416 | 938 | gntA, gntB, | USA | Lake | Western | 41.6989 | N | 8 Oct. 13 |
| | | gntC, gntD, | | Erie | Basin | 83.4589 | W | |
| | | gntE, gntF, | | | | | | |
| | | gntG, gntH | | | | | | |
| | | gntI, gntJ, | | | | | | |
| SRR5834679 | 160 | gntA, gntB, | USA: | N/A | N/A | 43.099 | N | Jul. 14, 2016 |
| | | gntC, gntD, | Wisconsin | | | 89.405 | W | |
| | | gntE, gntF, | | | | | | |
| | | gntG, gntH | | | | | | |
| | | gntI, gntJ | | | | | | |
| SRR6435700 | 148 | gntA, gntC, | USA: | N/A | N/A | 43.099 | N | Jul. 15, 2016 |
| | | gntD, gntE, | Wisconsin | | | 89.405 | W | |
| | | gntF, gntG, | | | | | | |
| | | gntH, gntI, | | | | | | |
| | | gntJ | | | | | | |
| SRR5194976 | 111 | gntC, gntD, | USA: Wisconsin, | freshwater | freshwater | 43.082834 | N | Aug. 21, 2015 |
| | | gntE, gntF, | Dane, Madison, | biome | lake | 89.409982 | W | |
| | | gntG, gntH, | Lake | | | | | |
| | | gntI, gntJ | Mendota | | | | | |
| SRR6435855 | 100 | gntA, gntB, | USA: | N/A | N/A | 43.099 | N | Jul. 15, 2016 |
| | | gntC, gntD, | Wisconsin | | | 89.405 | W | |
| | | gntE, gntF, | | | | | | |
| | | gntG, gntH, | | | | | | |
| | | gntJ | | | | | | |
| SRR6435865 | 94 | gntA, gntC, | USA: | N/A | N/A | 43.099 | N | Jul. 16, 2016 |
| | | gntD, gntE, | Wisconsin | | | 89.405 | W | |
| | | gntF, gntG, | | | | | | |
| | | gntH, gntI, | | | | | | |
| | | gntJ | | | | | | |
| SRR5834683 | 78 | gntC, gntD, | USA: | N/A | N/A | 43.099 | N | Jul. 15, 2016 |
| | | gntE, gntF, | Wisconsin | | | 89.405 | W | |
| | | gntG, gntH | | | | | | |
| | | gntI, gntJ, | | | | | | |
| SRR5834616 | 73 | gntA, gntB, | USA: | N/A | N/A | 43.099 | N | Jul. 14, 2016 |
| | | gntC, gntD, | Wisconsin | | | 89.405 | W | |
| | | gntE, gntF, | | | | | | |
| | | gntG, gntH | | | | | | |
| | | gntI, gntJ, | | | | | | |
| SRR5194978 | 72 | gntA, gntB, | USA: Wisconsin, | freshwater | freshwater | 43.082834 | N | Aug. 20, 2015 |
| | | gntC, gntD, | Dane, Madison, | biome | lake | 89.409982 | W | |
| | | gntE, gntF, | Lake | | | | | |
| | | gntG, gntH, | Mendota | | | | | |
| | | gntI, gntJ | | | | | | |
| SRR5834684 | 48 | gntA, gntC, | USA: | N/A | N/A | 43.099 | N | Jul. 14, 2016 |
| | | gntD, gntE, | Wisconsin | | | 89.405 | W | |
| | | gntF, gntG, | | | | | | |
| | | gntH, gntI, | | | | | | |
| | | gntJ | | | | | | |
| SRR5194977 | 47 | gntA, gntC, | USA: Wisconsin, | freshwater | freshwater | 43.082834 | N | Aug. 20, 2015 |
| | | gntD, gntE, | Dane, Madison, | biome | lake | 89.409982 | W | |
| | | gntF, gntG, | Lake | | | | | |
| | | gntH, gntI, | Mendota | | | | | |
| | | gntJ | | | | | | |
| SRR6435857 | 46 | gntA, gntC, | USA: | N/A | N/A | 43.099 | N | Jul. 15, 2016 |
| | | gntD, gntE, | Wisconsin | | | 89.405 | W | |
| | | gntF, gntG, | | | | | | |
| | | gntJ | | | | | | |
| SRR6435866 | 44 | gntA, gntC, | USA: | N/A | N/A | 43.099 | N | Jul. 16, 2016 |
| | | gntD, gntE, | Wisconsin | | | 89.405 | W | |
| | | gntF, gntG, | | | | | | |
| | | gntH, gntJ | | | | | | |
| SRR5194979 | 40 | gntB, gntC, | USA: Wisconsin, | freshwater | freshwater | 43.082834 | N | Aug. 20, 2015 |
| | | gntE, gntF, | Dane, Madison, | biome | lake | 89.409982 | W | |
| | | gntG, gntI, | Lake | | | | | |
| | | gntJ | Mendota | | | | | |
| SRR5834613 | 34 | gntA, gntB, | USA: | N/A | N/A | 43.099 | N | Jul. 14, 2016 |
| | | gntC, gntD, | Wisconsin | | | 89.405 | W | |
| | | gntE, gntG, | | | | | | |
| | | gntH, gntI, | | | | | | |
| | | gntJ | | | | | | |
| SRR5834391 | 34 | gntC, gntD, | USA: | N/A | N/A | 43.099 | N | Jul. 14, 2016 |
| | | gntE, gntF, | Wisconsin | | | 89.405 | W | |
| | | gntG, gntH, | | | | | | |
| | | gntJ | | | | | | |
| SRR5834678 | 34 | gntC, gntD, | USA: | N/A | N/A | 43.099 | N | Jul. 14, 2016 |
| | | gntE, gntF, | Wisconsin | | | 89.405 | W | |
| | | gntG, gntI, | | | | | | |
| | | gntJ | | | | | | |
| SRR6435860 | 27 | gntE, gntF, | USA: | N/A | N/A | 43.099 | N | Jul. 15, 2016 |
| | | gntG, gntH, | Wisconsin | | | 89.405 | W | |
| | | gntJ | | | | | | |
| SRR5834691 | 26 | gntA, gntC, | USA: | N/A | N/A | 43.099 | N | Jul. 15, 2016 |
| | | gntD, gntE, | Wisconsin | | | 89.405 | W | |
| | | gntF, gntG, | | | | | | |
| | | gntH, gntI | | | | | | |
| SRR5834392 | 26 | gntC, gntE, | USA: | N/A | N/A | 43.099 | N | Jul. 14, 2016 |
| | | gntF, gntG, | Wisconsin | | | 89.405 | W | |
| | | gntI | | | | | | |
| SRR5834609 | 25 | gntC, gntE, | USA: | N/A | N/A | 43.099 | N | Jul. 14, 2016 |
| | | gntG, gntH, | Wisconsin | | | 89.405 | W | |
| | | gntJ | | | | | | |
| SRR6435858 | 25 | gntC, gntE, | USA: | N/A | N/A | 43.099 | N | Jul. 15, 2016 |
| | | gntF, gntI,g | Wisconsin | | | 89.405 | W | |
| | | ntJ | | | | | | |
| SRR6435861 | 24 | gntA, gntB, | USA: | N/A | N/A | 43.099 | N | Jul. 16, 2016 |
| | | gntC, gntD, | Wisconsin | | | 89.405 | W | |
| | | gntE, gntF, | | | | | | |
| | | gntG, gntJ | | | | | | |
| SRR5834493 | 24 | gntC, gntE, | USA: | N/A | N/A | 43.099 | N | Jul. 14, 2016 |
| | | gntF, gntG, | Wisconsin | | | 89.405 | W | |
| | | gntJ | | | | | | |
| SRR5834393 | 22 | gntC, gntD, | USA: | N/A | N/A | 43.099 | N | Jul. 14, 2016 |
| | | gntE, gntF, | Wisconsin | | | 89.405 | W | |
| | | gntG, gntI, | | | | | | |
| | | gntJ | | | | | | |
| SRR5834688 | 19 | gntC, gntD, | USA: | N/A | N/A | 43.099 | N | Jul. 15, 2016 |
| | | gntE, gntF, | Wisconsin | | | 89.405 | W | |
| | | gntG, gntI | | | | | | |
| SRR6823695 | 19 | gntC, gntD, | USA: Madison | N/A | N/A | 43.0990 | N | Jul. 16, 2016 |
| | | gntE, gntF, | | | | 89.4050 | W | |
| | | gntG, gntJ | | | | | | |
| SRR5834395 | 17 | gntE, gntG, | USA: | N/A | N/A | 43.099 | N | Jul. 14, 2016 |
| | | gntH, gntI, | Wisconsin | | | 89.405 | W | |
| | | gntJ | | | | | | |
| SRR6823701 | 16 | gntA, gntC, | USA: | N/A | N/A | 43.0990 | N | Jul. 16, 2016 |
| | | gntD, gntE, | Madison | | | 89.4050 | W | |
| | | gntF | | | | | | |
| SRR5834682 | 16 | gntA, gntC, | USA: | N/A | N/A | 43.099 | N | Jul. 14, 2016 |
| | | gntD, gntE, | Wisconsin | | | 89.405 | W | |
| | | gntF, gntG | | | | | | |
| SRR5834614 | 12 | gntC, gntE, | USA: | N/A | N/A | 43.099 | N | Jul. 14, 2016 |
| | | gntG, gntH | Wisconsin | | | 89.405 | W | |
| SRR1781811 | 12 | gntE, gntF, | Brazil: | river | Amazon | 1.519367 | S | 13 May 11 |
| | | gntI, gntJ | Amazon River | | River | 48.917950 | W | |
| SRR7990772 | 12 | gntJ | USA: | N/A | N/A | 46.1840 | N | Aug. 4, 2010 |
| | | | Washington | | | | | |
| SRR5834394 | 12 | gntC, gntD, | USA: | N/A | N/A | 43.099 | N | Jul. 14, 2016 |
| | | gntE, gntF, | Wisconsin | | | 89.405 | W | |
| | | gntI | | | | | | |
| SRR5834492 | 12 | gntC, gntE, | USA: | N/A | N/A | 43.099 | N | Jul. 14, 2016 |
| | | gntG, gntH, | Wisconsin | | | 89.405 | W | |
| | | gntI, gntJ | | | | | | |
| SRR5834686 | 12 | gntC, gntE, | USA: | N/A | N/A | 43.099 | N | Jul. 15, 2016 |
| | | gntF, gntG, | Wisconsin | | | 89.405 | W | |
| | | gntI, gntJ | | | | | | |
| SRR5834612 | 10 | gntC, gntE, | USA: | N/A | N/A | 43.099 | N | Jul. 14, 2016 |
| | | gntF, gntG, | Wisconsin | | | 89.405 | W | |
| | | gntJ | | | | | | |
| SRR1781915 | 10 | gntC, gntI | Brazil: | river | Amazon | 1.519367 | S | 13 May 2011 |
| | | | Amazon River | | River | 48.917950 | W | |
| SRR12458608 | 8 | gntD, gntG | USA: Harsha | N/A | N/A | 39.0 | N | Aug. 12, 2015 |
| | | | Lake | | | 84.1 | W | |
| SRR5834687 | 8 | gntD, gntG, | USA: | N/A | N/A | 43.099 | N | Jul. 15, 2016 |
| | | gntJ | Wisconsin | | | 89.405 | W | |
| SRR6823694 | 8 | gntE, gntF, | USA: | N/A | N/A | 46.0410 | N | Jul. 8, 2016 |
| | | | | | | 89.6860 | W | |
| SRR12458606 | 8 | gntA, gntD, | USA: Harsha | N/A | N/A | 39.0 | N | Aug. 19, 2015 |
| | | gntJ | Lake | | | 84.1 | W | |
| SRR5834396 | 7 | gntD, gntF, | USA: | N/A | N/A | 43.099 | N | Jul. 14, 2016 |
| | | gntG, gntJ | Wisconsin | | | 89.405 | W | |
| SRR8269088 | 6 | gntJ | USA: Oregon | N/A | N/A | 46.234 | N | Aug. 1, 2010 |
| | | | | | | 123.909 | W | |
| SRR6823687 | 6 | gntC, gntJ | USA: Madison | N/A | N/A | 43.0990 | N | Jul. 16, 2016 |
| | | | | | | 89.4050 | W | |
| SRR5860223 | 6 | gntB, gntC, | USA: Delaware | N/A | N/A | 39.283 | N | Mar. 19, 2014 |
| | | gntJ | River, USA | | | 75.3633 | W | |
| SRR6823698 | 5 | gntE, gntH | USA: Madison | N/A | N/A | 43.0990 | N | Jul. 16, 2016 |
| | | | | | | 89.4050 | W | |
| SRR12458605 | 4 | gntF, gntH | USA: Harsha | N/A | N/A | 39.0 | N | Sep. 2, 2015 |
| | | | Lake | | | 84.1 | W | |
| SRR6823501 | 4 | gntC, gntD, | USA: Madison | N/A | N/A | 43.0990 | N | Jul. 14, 2016 |
| | | gntI | | | | 89.4050 | W | |
| SRR6466476 | 4 | gntJ | USA: Ohio, | N/A | N/A | 41.6995 7 | N | missing |
| | | | Lake Erie | | | 83.29410 | W | |
| SRR6048585 | 2 | gntJ | USA: Ohio, | N/A | N/A | missing | | missing |
| | | | Lake Erie | | | | | |
| SRR12458604 | 2 | gntJ | USA: Harsha | N/A | N/A | 39.0 | N | Sep. 2, 2015 |
| | | | Lake | | | 84.1 | W | |
| SRR6823494 | 2 | gntD | USA: Madison | N/A | N/A | 43.0990 | N | Jul. 15, 2016 |
| | | | | | | 89.4050 | W | |
| SRR9599516 | 2 | gntE | USA | N/A | N/A | 39.0367 | N | Jun. 25, 2016 |
| | | | | | | 84.1381 | W | |
| SRR6987382 | 2 | gntD, gntE | USA: Ohio | N/A | N/A | 39.0367 | N | May 2015 |
| | | | | | | 84.1381 | W | |
| SRR1601412 | 2 | gntA, gntJ | USA | Lake | Western | 41.7667 | N | 8 Oct. 2013 |
| | | | | Erie | Basin | 83.3086 | W | |
| SRR9599519 | 2 | gntE | USA | N/A | N/A | 39.0367 | N | Jun. 25, 2016 |
| | | | | | | 84.1381 | W | |
| SRR6466487 | 2 | gntC | USA: Ohio, | N/A | N/A | 41.69957 | N | missing |
| | | | Lake Erie | | | 83.29410 | W | |
| SRR5834685 | 2 | gntJ | USA: | N/A | N/A | 43.099 | N | Jul. 15, 2016 |
| | | | Wisconsin | | | 89.405 | W | |
| SRR7084784 | 2 | gntJ | USA: Oregon | N/A | N/A | 46.2320 | N | Aug. 21, 2007 |
| | | | | | | 123.8890 | W | |
| SRR7085878 | 2 | gntJ | USA: Oregon | N/A | N/A | 46.2340 | N | Aug. 1, 2010 |
| | | | | | | 123.9090 | W | |
| SRR12458595 | 2 | gntE | USA: Harsha | N/A | N/A | 39.0 | N | Jun. 25, 2015 |
| | | | Lake | | | 84.1 | W | |
| SRR12458617 | 2 | gntA | USA: Harsha | N/A | N/A | 39.0 | N | Jul. 13, 2015 |
| | | | Lake | | | 84.1 | W | |
| SRR6987381 | 2 | gntD, gntE | USA: Ohio | N/A | N/A | 39.0367 | N | May 2015 |
| | | | | | | 84.1381 | W | |
| SRR5830085 | 2 | gntC | USA: | N/A | N/A | 39.2637 | N | Aug. 17, 2015 |
| | | | Chesapeake | | | | | |
| | | | Bay | | | 76.0017 | W | |
| SRR12458607 | 2 | gntJ | USA: Harsha | N/A | N/A | 39.0 | N | Aug. 19, 2015 |
| | | | Lake | | | 84.1 | W | |
| SRR12458618 | 2 | gntE | USA: Harsha | N/A | N/A | 39.0 | N | Jun. 30, 2015 |
| | | | Lake | | | 84.1 | W | |
| SRR9599514 | 2 | gntE | USA | N/A | N/A | 39.0367 | N | Jun. 25, 2016 |
| | | | | | | 84.1381 | W | |
| SRR8438318 | 1 | gntG | USA: | N/A | N/A | 29.8571 | N | Jul. 12, 2016 |
| | | | Louisiana | | | 89.9778 | W | |
| SRR5209160 | 1 | gntH | USA: Delaware | N/A | N/A | 39.283 | N | missing |
| | | | River, USA | | | 75.3633 | W | |
| SRR6987346 | 1 | gntH | USA: Ohio | N/A | N/A | 39.0367 | N | May 2015 |
| | | | | | | 84.1381 | W | |
| SRR5834496 | 1 | gntG | USA: | N/A | N/A | 43.099 | N | Jul. 14, 2016 |
| | | | Wisconsin | | | 89.405 | W | |
| SRR6987351 | 1 | gntH | USA: Ohio | N/A | N/A | 39.0367 | N | May 2015 |
| | | | | | | 84.1381 | W | |

AS = alignment score. geo_loc_name, env_biome, env_feature, lat_lon, collection_date, represents data shown in the NCBI SRA metadata field of identical name.

TABLE 5
Primer sequences

| | |
|---|---|
| gntB-F | TTGTTTAACTTTAATAAGGAGATATACCATGAAGAAA |
| | AACATCAAGAAATACCGTTTC (SEQ ID NO: 87) |
| gntB-R | GCATTATGCGGCCGCAAGCTTGTTAGCTGCTAACTTG |
| | GGTCAGA (SEQ ID NO: 88) |
| gntC-F | GTTAAGTATAAGAAGGAGATATACATATGAAGATCCA |
| | GCCGGCGCTG (SEQ ID NO: 89) |
| gntC-R | CCGATATCCAATTGAGATCTGCCATATGTTAGCTGGT |
| | TTCAATAACGATCGCCAG (SEQ ID NO: 90) |
| pCOLA-F | CAAGCTTGCGGCCGCATAATGC |
| | (SEQ ID NO: 91) |
| pCOLA-R | CATGGTATATCTCCTTATTAAAGTTAAACAA |
| | (SEQ ID NO: 92) |

TABLE 6
Primer Sequences

| | | SEQ | |
|---|---|---|---|
| Primer | | ID | |
| name | Primer sequence | NO | Notes |
| GntA-Fv2 | TTGCTGTGTTTGAAA | 1 | 379 bp product |
| | AACCAATACC | | |
| GntA-Rv2 | GGACGACTATGGTCA | 2 | (Primer Pair 5) |
| | TATTTTGCT | | |
| GntJ-Fv2 | ATATCCAAACGGCAC | 3 | 233 bp product |
| | TGGAG | | |
| GntJ-Rv2 | TTGAGTTCCATGACC | 4 | (Primer Pair 9) |
| | AGACC | | |
| GntB-Fv1 | ACAAGTTTGCAATTT | 5 | Primer pair 4 |
| | CTCAAGG | | |
| GntB-Rv1 | TTAAAGGGTAATGCT | 6 | 312 bp product |
| | CGGCT | | |
| GntC-Fv1 | GCCTCATGAATAACG | 7 | Primer pair 2 |
| | ACACCC | | |
| GntC-Rv1 | TTGGTACTGGAACAA | 8 | 174 bp product |
| | CGACA | | |
| GntD-Fv1 | TGTTTGTTTTGTGCA | 9 | Primer pair 2 |
| | GTGCTT | | |
| GntD-Rv1 | AGTAGTCACCACGAG | 10 | 183 bp product |
| | CAGGA | | |
| GntE-Fv1 | ACCAGGAGGAGCAGG | 11 | Primer pair 6 |
| | TGAAT | | |
| GntE-Rv1 | TCAACAACATCACGC | 12 | 375 bp product |
| | CAATCT | | |
| GntF-Fv1 | AGAGCAGGTTCAGCT | 13 | Primer pair 3 |
| | TTGGC | | |
| GntF-Rv1 | ACAGAAAGAAGAAAC | 14 | 265 bp product |
| | CACCGC | | |
| GntG-Fv1 | CTGTGATGGAGCGCG | 15 | Primer pair 1 |
| | TTTG | | |
| GntG-Rv1 | GGGTAACTGCGCGAA | 16 | 461 bp product |
| | TTGTG | | |
| GntH-Fv1 | GGGTGTACCCTATTC | 17 | Primer pair 4 |
| | GCGG | | |
| GntH-Rv1 | CCGTGTGCCAGAGAC | 18 | 206 bp product |
| | ACATA | | |
| GntI-Fv1 | ATCGATTGGGAGTAC | 19 | Primer pair 1 |
| | GCAGG | | |
| GntI-Rv1 | TACTGACGCACTGTT | 20 | 269 bp product |
| | TGCCA | | |
| GntT-Fv1 | TACAGGTCAAGGTGC | 21 | Primer pair 2 |
| | GAAGG | | |
| GntT-Rv1 | TCCTTCCAGTGCAAG | 22 | 223 bp product |
| | TGCTA | | |

TABLE 7
Nucleic acid sequences of guanitoxin biosynthetic genes

| SEQ ID NO | Sequence |
|---|---|
| 23 | ATGCGAAAAAATCCTTTTGAAAGTAATCTAGCACTGGAACACAGTTGTTATGCCAAGTTTGATGG |
| | AACAGAATTTAAAATACTTGAGAGTGGTACAAAAATATCAGAGAAGGTAACTGAAGTACACGCTG |
| | AATTTCGGGAGAAAATATTGCATCGCACTCCTCCTTTTACTTGTATGGGTGCAACATCCGTATTT |
| | AACCAGAATAACTATCGTTTCAGTATGTACAGTGATATGTGTACAAAAGAAACTACCTTAGCATT |
| | GGCTCGAAATTTATTCACTTTTGTGAATGAACAAGATAGCATGAATAGTCACTTCACAGCATTTA |
| | TTGCTGTGTTTGAAAAACCAATACCAGAAGATGAACACCACTTTGAAAAACTGGTGTGGAGTCAA |
| | CTACAAATGTTACATGATGAAGATAGCCGTTACCATGATTGGGACACCCAATTCAGCAACGATAT |
| | CAATAATCATAAATTTTCATACAGTTTTGCTGAAAGAAGCTTTTTCATTATTGGAATGCACCCTG |
| | GAAGTTCTCGAATTGCTAGGCAATTTTCCTACCCACTTCTAGTATTTAATGCCAGTCGTCAATTT |
| | GATCATTTAGTTGAAACTAACCAATTCCATAACTTCGTCAGAATTATTCGGGAGCGTGACGTAGC |
| | TCTGCAAGGTAGTATTAATCCTAATCTGCCAGCAAAATATGACCATAGTCGTCCTGAAGAACCTG |
| | AAGTCAGACAATATTCTGGACGTGCAGTAGAAAAAGATTGGCAATGTCCTTTAGTTGTACACCAG |
| | AAGAAACATAAGCATAATCAATAA |
| 24 | ATGCCAGGCAATCCTTTTGACAGTAATCTAGCAATGGAACACAGTTGTTATGCCAAATTTGATGG |
| | AACAGAATTTAAAATCATTGAGAGTGGTACAAAAATATCAAAGAAAGTAACTGAAGTACACGCTC |
| | AATTTCGGGAGAAAATACTGCATCGCACTCCGCCTTTTACTTGTATGGGTGCAACATCCGTATTT |
| | AACCAAAATAACTATCGTTTCAGTATGTACCGTGAGATGTGTACAAAAGAAACTACCTTAGCATT |
| | GGCTAGAAATTTATTCACTTTTGTGGATGAACAAGATATCATGAATAGTCACTTTACAGCATTTA |
| | TTGCTGTGTTTGAAAAACCAATACCACAAGATGAACACGAATTTGAAAAACTGGTGTGGACTCAA |
| | CTGCAAATGTTACATGATGAAGATATCCGTTACCATGATTGGGACATAAAATTCAGCAACGATAT |
| | CAATAATCATAAGTTTTCATACAGCTTTGCTGAAAGAAGCTTTTTCATTATTGGAATGCACCCCG |
| | GAAGTTCTCGCATTGCCAGACAATTTTCCTACCCACTTCTAGTATTTAATGCCAGTCGCCAATTT |
| | GACCATTTAGTTGAAACTAACCAATTCCATAACTTCGTCAGAATTATTCGGGAGCGTGACATGGC |
| | TTTGCAAGGTAGTATTAATCCCAATCTGCCAGCAAAATATGACCATAGTCGTCCAGAAGAACCTG |
| | AAGTCAGACAATATTCTGGACGTGCAGTAGAAAAAGATTGGCAGTGTCCTTTAGTTGTACACCAG |
| | AAGAAACATGAGCATAATCAATAA |
| 25 | ATGCCAGGCAATCCTTTTGACAGTAATCTAGCAATGGAACACAGTTGTTATGCCAAATTTGATGG |
| | AACAGAATTTAAAATCATTGAGAGTGGTACAAAAATATCAAAGAAAGTAACTGAAGTACACGCTC |
| | AATTTCGGGAGAAAATACTGCATCGCACTCCGCCTTTTACTTGTATGGGTGCAACATCCGTATTT |
| | AACCAAAATAACTATCGTTTCAGTATGTACCGTGAGATGTGTACAAAAGAAACAACCTTAGCTTT |
| | GGCTCGAAATTTATTCACTTTTGTGGATGAACAAGATAGCATGAATAGTCACTTCACAGCATTTA |
| | TTGCTGTGTTTGAAAAACCAATACCACAAGATGAACACGAATTTGAAAAACTGGTGTGGACTCAA |
| | CTGCAAATGTTACATGATGAAGATATCCGTTACCATGATTGGGACATAAAATTCAGCAACGATAT |
| | CAATAATCATCAGTTTTCATACAGCTTTGCTGAAAGAAGCTTTTTCATTATTGGAATGCACCCCG |
| | GAAGTTCTCGCATTGCCAGACAATTTTCCTACCCACTTCTAGTATTTAATGCCAGTCGCCAATTT |
| | GACCATTTAGTTGAAACTAACCAATTCCATAACTTCGTCAGAATTATTCGGGAGCGTGACATGGC |
| | TTTGCAAGGTAGTATTAATCCCAATTTGCCAGCAAAATATGACCATAGTCGTCCAGAAGAACCTG |
| | AAGTCAGACAATATTCTGGACGTGCAGTAGAAAAAGATTGGCAGTGTCCTTTAGTTGTACACCAG |
| | AAGAAACATGAGCATAATCAATAA |
| 26 | ATGCGAAAAAATCCTTTTGAAAGTAATCTAGCACTGGAACACAGTTGTTATGCCAAGTTTGATGG |
| | AACAGAATTTAAAATACTTGAGAGTGGTGGAAAAATATCAGATAAGGTAACTGAAGTACACGCTG |
| | AATTTCGGAAGAAAATACTGCATCGCACTCCGCCTTTTACTTGTATGGGTGCAACGTCGGTATTT |
| | AACCAGAATAACTATCGTTTCAGTATGTACAGTGATATGTGTACTAAAGAAACTACCTTAGCATT |
| | GGCGAGAAATTTATTCACTTTTGTGGATGAACAAGATAGCATGAATAGTCACTTCACAGCATTTA |
| | TTGCTGTGTTTGAAAAACCAATACCAGAAGATGAACACCACTTTGAAAAACTGGTGTGGAGTCAA |
| | CTACAAATGTTGCATGACGAAGATAGCCGTTACCATGATTGGGACACCCAATTCAGCAACGATAT |
| | CAATAATCATAAATTTTCATACAGTTTTGCTCAAAGAAGCTTTTTCATTATTGGAATGCACCCTG |
| | GAAGTTCTCGAATTGCTAGGCAATTTTCCTACCCACTTCTAGTATTTAATGCCAGTCGTCAATTT |
| | GATCATTTAGTTGAAACTAACCAATTCCATAACTTCGTCAGAATTATTCGGGAACGTGATATGGC |
| | CTTGCAAGGTAGTATTAATCCCAATCTGCCAGCAAAATATGACCATAGTCGTCCCGAAGAACCTG |
| | AAGTTAGACAATATTCTGGACGTGCAGTAGAAAAAGATTGGCAATGTCCCCTAATTGTACACCAG |
| | AAGAAAGATAGGCATAATGAATAG |
| 27 | ATGCCAGGCAATCCTTTTGACAGTAATCTAGCAATGGAACACAGTTGTTATGCCAAATTTGATGG |
| | AACAGAATTTAAAATCATTGAGAGTGGTACAAAAATATCAAAGAAAGTAACTGAAGTACACGCTC |
| | AATTTCGGGAGAAAATACTGCATCGCACTCCGCCTTTTACTTGTATGGGTGCAACATCCGTATTT |
| | AACCAAAATAACTATCGTTTCAGTATGTACCGTGAGATGTGTACAAAAGAAACTACCTTAGCATT |
| | GGCTAGAAATTTATTCACTTTTGTGGATGAACAAGATATCATGAATAGTCACTTTACAGCATTTA |
| | TTGCTGTGTTTGAAAAACCAATACCACAAGATGAACACGAATTTGAAAAACTGGTGTGGACTCAA |
| | CTGCAAATGTTACATGATGAAGATATCCGTTACCATGATTGGGACATAAAATTCAGCAACGATAT |
| | CAATAATCATAAGTTTTCATACAGCTTTGCTGAAAGAAGCTTTTTCATTATTGGAATGCACCCCG |
| | GAAGTTCTCGCATTGCCAGACAATTTTCCTACCCACTTCTAGTATTTAATGCCAGTCGCCAATTT |
| | GACCATTTAGTTGAAACTAACCAATTCCATAACTTCGTCAGAATTATTCGGGAGCGTGACATGGC |
| | TTTGCAAGGTAGTATTAATCCCAATCTGCCAGCAAAATATGACCATAGTCGTCCAGAAGAACCTG |
| | AAGTCAGACAATATTCTGGACGTGCAGTAGAAAAAGATTGGCAGTGTCCTTTAGTTGTACACCAG |
| | AAGAAACATGAGCATAATCAATAA |
| 28 | ATGAAAAAAAATATTAAAAAATATCGCTTCAGCAATTCTATTTATCATCAAATAAAACCGCTTTT |
| | AAAACTTGATCAGTGGCATTGTTGGTTGGCTTTAGCAGAAGACTGGATAGTTATTTGTTTTGCTA |
| | TTTCCATTAGCTATTATTTGTCTTGGTACTTTTATCCTGTTACCATACTACTCATCGGTTCACGT |
| | CAGAGAGCATTAGCCACATTGCTCCATGAAGCAGCCCATCAAGTAATTGCCAAAAATCGCACCTT |
| | AAACTTTATGATGGGTACTTTTTTCTCAGGATATTTCATTCTGCAAACAATGTCATCTTATCGGA |
| | AATCTCATGTTGAAAAACATCATAGATATTTTGGCAATCCGGACGAAGATCCTGACTACAAGTTT |
| | GCAATTTCTCAAGGATTATATCAACAGAGTTTGGATGATAAAACCTTTCAGATGCTTTATATTTT |
| | TTCTCCTTTGCTACTTGCTCAAGTACCTAAATATATAAAATCTTTATTAATTCACCGTTTCCTAG |
| | AAGATCAAAATTATCTAGAACTGGTTGCTATTGGATTGTGGTGGCTAACAATTATTAGCCTCAGC |
| | ATTTTATTTAACCTATGGCAATATTTAATTCTCTTCTGGTTAATTCCTTATTTGACGGTTTTTCA |
| | AATCATTGGCTGGTTTATTGAATTAGCCGAGCATTACCCTTTAATGAATAATGATATCAATTTGT |
| | ATATGAGTCGTAACCGCAACAGCCATTTTATTGAGAAATTTCTAACAGGTATGCATAATGAAAAC |
| | TACCATTTAGTACATCACCTATTTCCCAATATTCCTTTCTGGAATGTGCCTAAAGCACACGAAAT |
| | ATTGATGCAAGATCCAAATTATGCCCAACATCACAGTCAAAGTGGAGGAATAGTCTGGTCAAATA |
| | ATAATGCACCTAGTATTTTATCAGGACTTACACAAGTGTCATCATAA |
| 29 | ATGAAATACAATTTTCAGTTGAATCCTACCATGAAAAAAATTTTTAAAAAATATCGCTTCAGTAA |
| | TTCTGTTTATCATCAAATTAAGCCGCTTTTTCAACTTGATAACTGGCATTGCTTTTTAGCTTTAG |
| | TAGAAAACTGGATAATTATTTGTTTTGTGATTTCTATTAGTTATTTTTTATCTTGGTACTTTTAT |
| | CCTTTAACCATACTAATTATTGGTTCACGTCAGAGGGCATTAGCCACACTGCTCCACGAAGCTGC |
| | CCATCAAATAATTGCCAAAAATCCGACCTTAAACTTTATGATGGGTACTTGTTTTTCAGGATATT |
| | TTATTTTCCAAACAATGTCATCTTATCGAAAATCTCATGTGGAAAACCATCATGGGAATTTCGGC |
| | AATCCAGATAAAGATCCTGACTACAAGTTTGCAATTTCTCAAGGATTATATCAACAAGATATAAA |
| | TGATAAAATCTTTATTAAACGTTCTATTTTTTTAACTTTACTATTTATTGGAGTACCTAAATATA |
| | TAAAATCTCTATTAGTTCACCGTTTCCTACAAAAGCAAAATTACCTAGAAGTGTTTGCTATTAGC |
| | TTATGGTGGCTAATAATTATTAGCCTCAGTATTCTATGTAACCTATGGCAATATTTAATTCTATT |
| | TTGGGTAATTCCTTATTTGACGGTTTTTCAAATCATTGGTTGGTTTATTGAATTAGCCGAGCATT |
| | ACCCTTTAATGAATAATCATATCAATTTGTATATGAGTCGTAACCGTAACAGTCATTATATTGAA |
| | CGATTTCTAACGGGTATGCATAATGAAAACTACCATTTAGTACATCACCTATTTCCAAATATTCC |
| | TTTCTGGAATGTACCTAAAGCACACGAAATATTGATGCAAGACCAAAATTATGCCCAACATCATA |
| | GTCAAAGTGGCGGAATATTCTGGTCAAATAATGATGCACCCAGTATTTTATATCTAAAGTCTGAA |
| | CATAGCTGCACAATTCCAATGGGGGAAAATAAATGA |
| 30 | ATGAAAAAAAATATTAAAAAATATCACTTCAGTAATTCTGTTTATCATCAAATTAAGCCGCTTTT |
| | TCAACTTGATAACTGGCATTGCTTCTTAGCTTTAGCAGAAGACTGGATAATTATTTGTTTTGCGA |
| | TTTCTATTAGTTATTTTTTATCTTGGTACTTTTATCCTTTAACCATACTAATTATTGGTTCACGT |
| | CAGAGGGCATTAGCCACACTGCTCCACGAAACTGCCCATCAAATAATTGCCAAAAATCCGACCTT |
| | AAACTTTATGATGGGTACTTGTTTTTCAGGATATTTTATTTTCCAAACAATGTCATCTTATCGAA |
| | AATCTCATGTGGAAAACCATCATGGTAATTTCGGCAATCCAGATAAAGATCCTGACTACAAGTTT |
| | GCAATTTCTCAAGGATTATATCAACAAGATATAAATGATAAAATCTTTATTAAACGTTCTATTCT |
| | TTTAACTTTACTATTTATTGGAGTACCTAAATATATAAAATCTCTATTAGTTCACCGTTTCCTAC |
| | AAAAGCAAAATTACCTAGAAGTGTTTGCTATTAGCTTATGGTGGCTAATAATTATTAGCCTCAGT |
| | ATTCTATGTAACCTATGGCAATATTTAATTCTATTTTGGGTAATTCCTTATTTGACGGTTTTTCA |
| | AATCATTGGTTGGTTTATTGAATTAGCCGAGCATTACCCTTTAATGAATAATCATATCAATTTGT |
| | ATATGAGTCGTAACCGCAACAGCCATTTTATTGAGCGATTTTTAACGGGTATGCATAATGAAAAC |
| | TACCATCTAATACATCACCTATTCCCAAGTATTCCTTTCTGGAATATACCTAAAGCACACGAAAT |
| | CTTGATGCAAGACCCAAATTATGCCCAACATCATCGTCAAAGTGGCGGAATATTCTGGTCTAATA |
| | ATGATGCACCCAGTATTTTTCTAAAGTCTAAACATAGCTGCACAATTCCAATGGAGGAAAATAAA |
| | TGA |
| 31 | ATGGAAAAAAAGATTCAAAATTATCGCTTCAATAATTCTGTCTATCGTCAAATTAAGCCACTTTT |
| | TGAACTTGATAACTGGCATTGTTTTGGAGCTTTAGCAGAAGACTGGATAATTATTTGTTTTGCGA |
| | TTTATATTAGTTGTTTTTTATCTTGGTACTTTTATCCTTTAAGTATACTAATTATTGGTTCACGT |
| | CAGAGGGCATTAGCCACAATGCTCCACGAATCCGCCCATAGAATACTGGCAAAAAATCCCAACTT |
| | AAACTTGATAATGGGTACTTTTTTTTCAGGATATTTTATTTTGCAAATAATGTCGTCTTATCGGA |
| | AATCTCATGTGGAAAACCATCATGGAAATTTTGGACATCCAGATAAAGATCCCGACTACAAATTT |
| | GCCATTTCTCAAGGATTATATCCAACAAATATAGATGATTTAAGATTTGCTTCACTACTGAGGCG |
| | ATTACCTAAATATATTAAATCTTTATTAGTTCACCGTTTCCTAGAAGATCAAAAATCCTTAGAAA |
| | TTCTATCTATTAGCTTATGGTGGCTAGTAATTATTAGCGTGAGTATTTTATGTAACCTATGGCAA |
| | TATTTAATTCTATTTTGGTTAATTCCTTATTTAACCTTTTTTCAAATCATAGGTTGGTTTATTGA |
| | ATTAGCCGAACATTACCCTTTAATGAACCACCAGATAAATTTGTATATGAGTCGTAACCGTAACA |
| | GTCATGACATTGAGCTATTTTTAACGGGTATGCATAATGAAAACTACCATTTGGTACATCACCTA |
| | TTTCCAAATATTCCTTTCTGGAATGTACCTAAAGCACACGCAATATTGATGCAAGATCCAAATTA |
| | TGCCCAACATCACAATCAAAGTGGCGGAATAGTGTGGTCAAATAATCATGCAGCCAGTATTTTAT |
| | CAGGACTGTACAATTGCAATGAGGGAAAATAA |
| 32 | ATGAAATACAATTTTCAGTTGAATCCTACCATGAAAAAAATTTTTAAAAAATATCGCTTCAGTAA |
| | TTCTGTTTATCATCAAATTAAGCCGCTTTTTCAACTTGATAACTGGCATTGCTTTTTAGCTTTAG |
| | TAGAAAACTGGATAATTATTTGTTTTGTGATTTCTATTAGTTATTTTTTATCTTGGTACTTTTAT |
| | CCTTTAACCATACTAATTATTGGTTCACGTCAGAGGGCATTAGCCACACTGCTCCACGAAGCTGC |
| | CCATCAAATAATTGCCAAAAATCCGACCTTAAACTTTATGATGGGTACTTGTTTTTCAGGATATT |
| | TTATTTTCCAAACAATGTCATCTTATCGAAAATCTCATGTGGAAAACCATCATGGGAATTTCGGC |
| | AATCCAGATAAAGATCCTGACTACAAGTTTGCAATTTCTCAAGGATTATATCAACAAGATATAAA |
| | TGATAAAATCTTTATTAAACGTTCTATTTTTTTAACTTTACTATTTATTGGAGTACCTAAATATA |
| | TAAAATCTCTATTAGTTCACCGTTTCCTACAAAAGCAAAATTACCTAGAAGTGTTTGCTATTAGC |
| | TTATGGTGGCTAATAATTATTAGCCTCAGTATTCTATGTAACCTATGGCAATATTTAATTCTATT |
| | TTGGGTAATTCCTTATTTGACGGTTTTTCAAATCATTGGTTGGTTTATTGAATTAGCCGAGCATT |
| | ACCCTTTAATGAATAATCATATCAATTTGTATATGAGTCGTAACCGTAACAGTCATTATATTGAA |
| | CGATTTCTAACGGGTATGCATAATGAAAACTACCATTTAGTACATCACCTATTTCCAAATATTCC |
| | TTTCTGGAATGTACCTAAAGCACACGAAATATTGATGCAAGACCAAAATTATGCCCAACATCATA |
| | GTCAAAGTGGCGGAATATTCTGGTCAAATAATGATGCACCCAGTATTTTATATCTAAAGTCTGAA |
| | CATAGCTGCACAATTCCAATGGGGGAAAATAAATGA |
| 33 | ATGAAAATTCAACCTGCTTTATTAGAAATGTGGCTGTCTGAATATGAACGAGTACCTTATAACTT |
| | AGGAGAAAGCAGTGTTGATAATTTTACCTTGGGAGAATTACTAAATCTTACAGGAGATCGTGATG |
| | CTCTTGATCAACTCAGCCTCATGAATAACGACACCCACGGCTCCCTGAGATTACGTGAAGCGATC |
| | GCCTCATTAGATAAATCAGTATCCCCAGATGATATTTTAGTGACTGCTGGGACAACAGAAGCCAT |
| | ATTAATTTATTTTAAAGTACGTTACCGTTCCGGTGCTAATGTCGTTGTTCCAGTACCAACCTTCC |
| | ATGTACTTTATGAAACTCCAGCTTTTTTAGGTTACGAGGTACGCTATCTGCAATTACGAGCAGAA |
| | AATGGTTTTCGCATTGATCCTCAAGAATTAGCCAAGTTAGTTGATGATAATACTGAAGTAATAGT |
| | TTTGAATACTCCACAAAATCCTAGTGGTGTTGTTTGCTCAGAAACAGAAATTCAGTCCATAATTG |
| | AGATTGCAGAAAAGCATAATGCAGAAATTTTAGCTGATGAGCATTACCGTTTTTTACCCCATGAT |
| | CAAGACACAGAAATTCTGCCTTCGTTGTATGGACTTTCACCCAAAATCATCAGTTTAGGTTCTAC |
| | AGGTAAATGCTTTGGTTGTATTGGGTTACGCATTGGTTGGCTGATTGGCAACCCAGAAATAATTA |
| | AAGCTTGTCACTTTTTCAAAGACTACACAACCCACACTGTCTGCGTCCTTAATGATTACATCGCT |
| | GCTGGAGTCTTATTACACAAAGGCAAAATTTTACCCCGTTATCGTCAGATGATTCAGCACAATAT |
| | TCAGCAGTTTGAAACTTTTATCAAACAACAACGGGGATTAATTGACTGGGTTAAACCTGAAGCGG |
| | GTACTATCGCTTTTCCGTTTTTTACCGATCCAAATATCAACAGCAAAATAGTAGCCAAACGTCTG |
| | GTAGAAGATCATGGAGTGTTGTTGCTACCTGGTGAAGCATTTGATCGCCCCAGTCATTTCCGCAT |
| | AGCTTTGGGTGTTGAACCAAGCCTTTTTCAGTACGCATTGGAAAAACTTGCTATTGTGATAGAAA |
| | CTTCATGA |
| 34 | ATGAAAATTCAACCTGCTTTATTAGAAATGTGGCTGTCTGAATATGAACGAGTACCTTATAACTT |
| | AGGAGAAAGCAGTGTTAATAATTTTACCCTTGGAGAATTACTAAGCCTTACAGGAGATCGTGACG |
| | CTCTTGATAAACTCAGCCTCATGAATAACGACACCCACGGCTCTCGGAGATTGCGTGAAGCGATC |
| | GCCTCATTAGATGAATCAGTATCCCCAGATGATATTCTAGTTACGGCTGGGACAACAGAAGCTAT |
| | TTTAATTTATTTTAAAATACGTTACCGTTCTGGTGCTAATGTCGTTGTTCCAGTACCAACATTCC |
| | ATGTACTTTATGAAACTCCAGCCTTCTTTGGTTACGAGGTACGCTATCTGCAATTGCGAGCAGAA |
| | AATGGTTTTCGCATTGATCCTGAAGAATTAGCCAAGTTAGTTGATGATAATACTGAAATAATTGT |
| | TTTAAATACTCCACAAAATCCTAGTGGTGTTGTTTGCTCAGAAACAGAAATTCATTCCATAATTG |
| | AGATTGCAGAAAAGCATAACGCAGAAATTTTAGCTGATGAGCATTATCGTTTTTTACCCCATGAT |
| | CAAGACACAGAAATTCTGCCTTCGTTGTATGGACTTTCACCCAAAATTATCAGTTTAAGTTCTAC |
| | AGGTAAATGCTTTGGTTGTATTGGTTTACGCATTGGTTGGCTGATTGGCAACCCAGAAGTAATTA |
| | AAGCTTGTCACTTTTTCAAAGACTACACAACCCACACTGTTTGCGTTCTCAACGATTACATTGCA |
| | TCTGGAGTCTTATTACACAGAGACAAAATTTTACCCCGTTATCGTCAGATGATTCAGCATAATAT |
| | TCAGCAGTTTGAAACTTTTATCAAACAACAACGGGGATTAATTGACTGGGTTAAACCTGAAGCGG |
| | GTACTATTGCTTTTCCGTTTTTTACAAATCCAAATATCAACAGCAAAACACTAGCCAAACGTCTG |
| | GTGGAAGATCATGGAGTGTTGTTGCTTCCTGGTGAAGCGTTTGATCGACCCAGTCATTTCCGCAT |
| | AGCTTTGGGTGTTGAACCAAGCCTTTTTCAGTACGCATTGGAAAAACTTGCTATTGTGATAAAAA |
| | CTTCATTATGA |
| 35 | ATGAAAATTCAACCTGCTTTATTAGAAATCTGGCTATCTGAATATGAACGAGTACCTTATAACTT |
| | AGGAGAAAGCAGTGTTAATAATTTTACCCTGGGAGAATTACTAAGCCTTACAGGAGATCGTGATG |
| | CTCTTGATAAACTCAGCCTCATGAATAACGACACCCACGGCTCTCTGAGATTGCGTGAAGCGATC |
| | GCCTCATTAGATAAATCAGTATCCCCAGATAATATTTTAGTGACTGCTGGGACAACAGAAGCTAT |
| | ATTAATTTATTTTAAAGTACGTTACCGTTCTGGAGCTAATGTCGTTGTTCCAGTACCAACCTTTC |
| | ATGTACTTTATGAAACTCCGGCCTTCTTTGGTTACGAGGTACGCTATCTGCAATTGCGAGCAGAA |
| | AATGGTTTTCGCATTGATCCTGAAGAATTAGCCAAGTTAGTTGATGATAATACTGAAGTAATAGT |
| | TTTGAATACTCCACAAAATCCTAGTGGTGTTGTCTGCTCAGAAACAGAAATTCAGTCCATAATTG |
| | AGATTGCAGAAAAGCATAACGCAGAAATTTTAGCTGATGAGCATTATCGTTTTTTACCCCATGAT |
| | CTAGACACAGAAATTCTGCCTTCGTTGTATGGACTTTCACCCAAAATCATCAGTTTAGGTTCTAC |
| | AGGTAAATGCTTTGGTTGTATTGGGTTACGCATTGGTTGGTTGATTGGCAACCCAGAAATAATTA |
| | AAGCTTGTCACTTTTTCAAAGACTACACAACCCACACTGTCTGCGTCCTTAATGATTACATCGCT |
| | GCTGGAGTTTTATTACACAAAGACAAAATTTTACCACGTTATCGTCAGATGATTCAGTATAATAT |
| | TCAGCAGTTTGAAACTTTTATCAAACAACAACGGGGATTAATTGACTGGGTAAAACCTGAAGCGG |
| | GTACTATCGCTTTTCCGTTTTTTACAGATCCAAATATCAACAGCAAAACAGTAGCCAAACTTCTG |
| | GTAGAAGATCATGGAGTATTGTTGCTACCTGGTGAAGCATTTGATCGACCCAGTCATTTCCGCAT |
| | AGCTTTGGGTGTTGAACCAAGCCTTTTTCAGTACGCATTGGAAAAACTTGCTATTGTGATAAAAA |
| | CTTCATTATGA |
| 36 | ATGAAAATTCAACCTGCTTTATTAGAAATGTGGCTGTCTGAATATGAACGAGTACCTTATAACTT |
| | AGGAGAAAGCAGTGTTAATAATTTTACGTTTGGAGAATTACTAAGCCTTACCGGAGATCGTGATG |
| | CTCTTGATAAACTTAGCCTCATGAATAACGACACCCACGGATCTTTGAGATTGCGTGAAGCGATC |
| | GCCTCATTAGATGAATCAGTATCCCCAGATGATATTCTAGTTACGGCTGGGACAACAGAAGCTAT |
| | TTTAATTTATTTTAAAATACGTTACCGTCCCGGAGCTAATGTCGTTGTTCCAGTACCAACCTTTC |
| | ATGTACTTTATGAAACTCCAGCTTTCTGTGGTTATGAGATACGCTATCTGCAATTGCGAGCAGAA |
| | AATGGATTTCACATTGATCCTGAAGAATTAGCCAAGTTAGTTGATGATAATACTGAAATAATTGT |
| | TTTAAATACTCCACAAAATCCTAGTGGTGTTGTTTGCTCAGAAACAGAAATTCACTCCATAATTG |
| | AAATTGCACAAAAGCATAATGCAGAAATTTTAGCTGATGAGCATTATCGTTTTTTACCCCATGAT |
| | CAAAACACAGAAATTCTCCCTTCTTTGTATGGACTTTCACCCCAAATAACCAGTTTAGGTTCTAC |
| | AGGTAAATGCTTTGGTTGTATTGGTTTACGCATTGGTTGGCTGATTGGCAACCCAGAAATAATAA |
| | AAGCTTGTCACTTTTTCAAAGACTACACAACCCACACGGTTTGTGTTCTCAACGATTACATTGCA |
| | TCTGGAGTCTTATTACACAGAGACAAAATTTTACCCCGTTATCGTCAGATGATTCAGCATAATAT |
| | TCAGCAGTTTGAAACTTTTATCAAACAACAACGGGGATTGATTGACTGGGTTAAACCTGAAGCGG |
| | GTACGGTCGCTTTTCCGTTTTTTACAGATCCGAGTATCAACAGCAAAACACTAGCCAAACGGCTG |
| | GTGGAAGATCACGGAGTGTTGTTGCTTCCTGGTGAAGCGTTTGATCGTCCCAGTCATTTCCGGAT |
| | AGCTTTAGGTGTTGAATCAAACTTTTTTCAATATGCATTGGAAAAACTTACTATTGTGATGGCAG |
| | ATTGTTTATGA |
| 37 | ATGAAAATTCAACCTGCTTTATTAGAAATGTGGCTGTCTGAATATGAACGAGTACCTTATAACTT |
| | AGGAGAAAGCAGTGTTAATAATTTTACCCTTGGAGAATTACTAAGCCTTACAGGAGATCGTGACG |
| | CTCTTGATAAACTCAGCCTCATGAATAACGACACCCACGGCTCTCGGAGATTGCGTGAAGCGATC |
| | GCCTCATTAGATGAATCAGTATCCCCAGATGATATTCTAGTTACGGCTGGGACAACAGAAGCTAT |
| | TTTAATTTATTTTAAAATACGTTACCGTTCTGGTGCTAATGTCGTTGTTCCAGTACCAACATTCC |
| | ATGTACTTTATGAAACTCCAGCCTTCTTTGGTTACGAGGTACGCTATCTGCAATTGCGAGCAGAA |
| | AATGGTTTTCGCATTGATCCTGAAGAATTAGCCAAGTTAGTTGATGATAATACTGAAATAATTGT |
| | TTTAAATACTCCACAAAATCCTAGTGGTGTTGTTTGCTCAGAAACAGAAATTCATTCCATAATTG |
| | AGATTGCAGAAAAGCATAACGCAGAAATTTTAGCTGATGAGCATTATCGTTTTTTACCCCATGAT |
| | CAAGACACAGAAATTCTGCCTTCGTTGTATGGACTTTCACCCAAAATTATCAGTTTAAGTTCTAC |
| | AGGTAAATGCTTTGGTTGTATTGGTTTACGCATTGGTTGGCTGATTGGCAACCCAGAAGTAATTA |
| | AAGCTTGTCACTTTTTCAAAGACTACACAACCCACACTGTTTGCGTTCTCAACGATTACATTGCA |
| | TCTGGAGTCTTATTACACAGAGACAAAATTTTACCCCGTTATCGTCAGATGATTCAGCATAATAT |
| | TCAGCAGTTTGAAACTTTTATCAAACAACAACGGGGATTAATTGACTGGGTTAAACCTGAAGCGG |
| | GTACTATTGCTTTTCCGTTTTTTACAAATCCAAATATCAACAGCAAAACACTAGCCAAACGTCTG |
| | GTGGAAGATCATGGAGTGTTGTTGCTTCCTGGTGAAGCGTTTGATCGACCCAGTCATTTCCGCAT |
| | AGCTTTGGGTGTTGAACCAAGCCTTTTTCAGTACGCATTGGAAAAACTTGCTATTGTGATAAAAA |
| | CTTCATTATGA |
| 38 | ATGATGAGTATTCAAACGTTAAATTTTACTCAATCTGAACTTGAAGAAATAGACATTCTTTTAGA |
| | TCAAGTTACTGCTAACTATAGCCATGCAGAACAACCGGAATTGATTGACACTGCTGCGGTATTAG |
| | CCCAAAGTTTGCCCCTACGCCTGCGTCAATTTTTACATGAGTTCAAATTGCGAGAAACCGCTCCT |
| | GCTTGTCTCATCAAGGGTTATCCAGTTAATGATAATACTATTGGCGCTACTCCTGAACATTGGGC |
| | AGGTCATGAAGATGCCAAGCGCACTAAACGAGAGCAAATTGTGTTTGTTTTGTGCAGTGCTTTAC |
| | TGGGAGAAATATTTGGTTGGCTAACACAGCAAAATGCCTATCTGATCCACGATGTTTTTCCAATT |
| | AAAGGCTATGAACAGGATCAAATTGGTTCTGGTAGTGAAGCACTACTGCTTTGGCATACTGAAGA |
| | TGCTTATCATCCTGCTCGTGGTGACTACTTGGGGCTAATGTGTTTGCGTAATCCTTGCCAGAGTA |
| | TAACCACTATTGGTTCAGTGGATTCATTAAAAAAACTGACTCAGCAGCAATTAGATATCTTGTTT |
| | CAACCACGTTTTATTATTCGCCCAGATAATGCCCATCATGCCGATAATAATTATCAAACAGAAAC |
| | TGATTTAGATACTCATCTTCAGAATGCTTACAATCAAATAAAGCATAAAATTATCTCCCAAGATA |
| | ATGCTATGGCTTTGCTATATGGAGAACCTAGCGCACCCTATTTGTGTGTTGATCCAGCTTATATG |
| | GAAGCAACAGATGAAGAAGCTAAACAGACTTTGCAAGCCTTGATTGATGCTATTGACATTGAAAT |
| | CATGGATGTAATTTTACAACCCGGTGATATTTGTTTCGTAGATAATTTCCGTGCTGTGCATGGGA |
| | GAAAACCATTTAAAGCCAATTATGATGGTAATGATCGGTGGTTAAAGCGAATTAATATCACACGC |
| | GATCTTCGCAAATCTAAAACATATCGTTCTCATCCTACATCTCGAATTATTGATAAAGTACCTTC |
| | ATAA |
| 39 | ATGAGTATTCAAACGTTAAATTTGACTGAAGCCGAACTTGAAGAAATAGACATTCTTTTAGATCA |
| | AGTTACTGCTAACTATAGCCATGCAGAACAACCAGAATTGATTGACACTGCTGCGGTATTAGCCC |
| | AAAGTTTGCCCCTACGCCTACGTCAATTTTTACATGAGTTCAAATTGCGAGAAACCGCTCCTTCT |
| | TGTCTCATCAAGGGTTATCCAGTTAATGATAATACTATTGGCACTACTCCTAAACATTGGGCAGG |
| | TCATGAAGATGCCAACCGCACCAAACGAGAGCAAATTGTGTTTGTTTTGTGCAGTGCTTTACTGG |
| | GAGAAATATTTGGTTGGCTAACACAGCAAAATGCCTATCTGATCCACGATGTTTTTCCAATTAAA |
| | GGCTATGAACAGGATCAAATAGGTTCTGGTAGTGAAGCACTACTGCTTTGGCATACTGAAGATGC |
| | TTATCATCCTGCTCGTGGTGACTACTTCGGGCTAATGTGTTTACGTAATCCTGCTCAGGCTGTAA |
| | CCACTATTGGTTCAGTTGATTCTTTAAAAAAGTTGACTCAGCAGCAATTAGATATCTTGTTTCAA |
| | CCACGTTTTATTATTCACCCAGATAATGCCCATCACGCAGATAATAATTATCAAACAGAAACTCA |
| | TTTAGATGCCCATATTAAGAATGCTTACAATCAAATCAAGCAGAAAATTATCTCCCAAGATAACG |
| | CTGTATCTTTGTTATATGGAGAGCCTAGCGCACCCTATTTATGCGTCGATCCTGCTTATATGGAA |
| | GCAACAGATGAAGAAGCTAAACAGACGTTACAAGCATTGATTAATGCTATTGACATTGAAATCAT |
| | GGATGTAATTTTACAACCCGGTGATATTTATTTCATAGATAACTTCCGTGCTGTCCATGGGAGAA |
| | AACCATTTAAAGCCAATTATGATGGTAATGATCGGTGGTTAAAGCGGATTAATATCACCCGTGAT |
| | CTTCGCAAATCTAGAACATATCGTTCTAATCCTACATCTCGAATTATTGATAAAGTACCTTCATA |
| | A |
| 40 | ATGAGTATTCAAACGTTAAATTTGACTGAAGCCGAACTTGAAGAAATAGACATTCTTTTAGATCA |
| | AGTTACTGCTAACTATAGCCATGCAGAACAACCAGAATTGATTGACACTGCTGCGGTATTAGCCC |
| | AAAGTTTGCCCCTACGCCTACGTCAATTTTTACATGAGTTCAAATTGCGAGAAACCGCTCCTTCT |
| | TGTCTCATCAAGGGTTATCCAGTTAATGATAATACTATTGGCACTACTCCTAAACATTGGGCAGG |
| | TCATGAAGATGCCAACCGCACCAAACGAGAGCAAATTGTGTTTGTTTTGTGCAGTGCTTTACTGG |
| | GAGAAATATTTGGTTGGCTAACACAGCAAAATGCCTATCTGATCCACGATGTTTTTCCAATTAAA |
| | GGCTATGAACAGGATCAAATAGGTTCTGGTAGTGAAGCACTACTGCTTTGGCATACTGAAGATGC |
| | TTATCATCCTGCTCGTGGTGACTACTTCGGGCTAATGTGTTTACGTAATCCTGCTCAGGCTGTAA |
| | CCACTATTGGTTCAGTTGATTCTTTAAAAAAGTTGACTCAGCAGCAATTAGATATCTTGTTTCAA |
| | CCACGTTTTATTATTCACCCAGATAATGCCCATCACGCAGATAATAATTATCAAACAGAAACTCA |
| | TTTAGATGCCCATATTAAGAATGCTTACAATCAAATCAAGCAGAAAATTATCTCCCAAGATAACG |
| | CTGTATCTTTGTTATATGGAGAGCCTAGCGCACCCTATTTATGCGTCGATCCTGCTTATATGGAA |
| | GCAACAGATGAAGAAGCTAAACAGACGTTACAAGCATTGATTAATGCTATTGACATTGAAATCAT |
| | GGATGTAATTTTACAACCCGGTGATATTTATTTCATAGATAACTTCCGTGCTGTCCATGGGAGAA |
| | AACCATTTAAAGCCAATTATGATGGTAATGATCGGTGGTTAAAGCGGATTAATATCACCCGTGAT |
| | CTTCGCAAATCTAGAACATATCGTTCTAATCCTACATCTCGAATTATTGATAAAGTACCTTCATA |
| | A |
| 41 | ATGACTATTCAAATCTTAAACCTTACGCAAACTGAACTTCAACAAATAGACATTCTTTTGGATCA |
| | AGTTACAGCTAACTATAACCATGCGGAACAGCCAGAACTGATTGATACTGCTGCGGTATTAGCTC |
| | AAAGTCTTCCCCTGCGCTTGTGTCAATTTTTATATGAGTTCAAATTAAGGGAAACCGCCCCCGCT |
| | TGTCTCATTAAGGGTTATCCAGTTAGTGATAATACTATTGGTGTTACTCCTAAACATTGGGCAGG |
| | ACATGAAGATGCCAACCGCACCAAACGAGAGCAAATTCTGTTTGTTTTGTGCAGTGCTTTACTCG |
| | GAGAAATATTTGGTTGGGTAACACAGCAAAATGCTTATTTGATCCATGATGTTTTTCCTATTCCC |
| | GGCTATGAACAGGATCAAATTGGTTCTGGTAGTGAAGCATTACTACTTTGGCATACTGAAGATGC |
| | TTATCATCCTGCTCGTGGTGACTACTTGGGACTAATGTGCTTACGTAATTATGCTCAGGCTGTAA |
| | CCACTATTGGTTCAGTTGATTCTTTAAAAAAGTTAACTCAGCAGCAATTAGATATTTTGTTTCAA |
| | CCACGTTTTATTATTCGCCCAGATAATGCACACCATGCAAATAATAATTATCAAACAGAAACTCA |
| | TTTGGATGCTAATCTAGAGAATGCTTACAATCAAATCAAGCAGAAAATTATTTCACAGGATAACG |
| | CTGTACCTTTGTTATATGGAGAGCCTAGCGCACCCTATTTATGTGTCGATCCTGCTTATATGGTA |
| | GCGACTGATCAAGAAGCTAAAGAAACTTTACAAGCATTGATTGATGCCATTGACATTGAAATCAT |
| | GGATGTAATTTTAGAACCTGGTGATATTTGCTTTGTGGATAATTTCCGTGCTGTCCATGGCAGAA |
| | AACCATTTAAAGCCAACTATGATGGCTCCGACCGTTGGTTAAAGCGAATTAATATCACCCGTGAT |
| | CTTCGCAAATCTAGAATATATCGCTCTAATCCCACATCTCGAATTATTGATAAAGTACCTTCATA |
| | A |
| 42 | ATGAGTATTCAAACGTTAAATTTGACTGAAGCCGAACTTGAAGAAATAGACATTCTTTTAGATCA |
| | AGTTACTGCTAACTATAGCCATGCAGAACAACCAGAATTGATTGACACTGCTGCGGTATTAGCCC |
| | AAAGTTTGCCCCTACGCCTACGTCAATTTTTACATGAGTTCAAATTGCGAGAAACCGCTCCTTCT |
| | TGTCTCATCAAGGGTTATCCAGTTAATGATAATACTATTGGCACTACTCCTAAACATTGGGCAGG |
| | TCATGAAGATGCCAACCGCACCAAACGAGAGCAAATTGTGTTTGTTTTGTGCAGTGCTTTACTGG |
| | GAGAAATATTTGGTTGGCTAACACAGCAAAATGCCTATCTGATCCACGATGTTTTTCCAATTAAA |
| | GGCTATGAACAGGATCAAATAGGTTCTGGTAGTGAAGCACTACTGCTTTGGCATACTGAAGATGC |
| | TTATCATCCTGCTCGTGGTGACTACTTCGGGCTAATGTGTTTACGTAATCCTGCTCAGGCTGTAA |
| | CCACTATTGGTTCAGTTGATTCTTTAAAAAAGTTGACTCAGCAGCAATTAGATATCTTGTTTCAA |
| | CCACGTTTTATTATTCACCCAGATAATGCCCATCACGCAGATAATAATTATCAAACAGAAACTCA |
| | TTTAGATGCCCATATTAAGAATGCTTACAATCAAATCAAGCAGAAAATTATCTCCCAAGATAACG |
| | CTGTATCTTTGTTATATGGAGAGCCTAGCGCACCCTATTTATGCGTCGATCCTGCTTATATGGAA |
| | GCAACAGATGAAGAAGCTAAACAGACGTTACAAGCATTGATTAATGCTATTGACATTGAAATCAT |
| | GGATGTAATTTTACAACCCGGTGATATTTATTTCATAGATAACTTCCGTGCTGTCCATGGGAGAA |
| | AACCATTTAAAGCCAATTATGATGGTAATGATCGGTGGTTAAAGCGGATTAATATCACCCGTGAT |
| | CTTCGCAAATCTAGAACATATCGTTCTAATCCTACATCTCGAATTATTGATAAAGTACCTTCATA |
| | A |
| 43 | ATGTTAAAAGATTCCCCAAAATCCCATGCTTTCTTTGAACGCGCACAAAAAGTTATCCCCTTTGG |
| | TGTTAATTCCAGCCACCGCTACTGGGGCGAAAATACACCTGTAATTCAGCGTGGTGAAGGAGCGT |
| | ATGTTTATGACTTTGACGGAAAAAGCTATATTGATTACCGTCTAGGTTTTGGTCCAGTTATTCTT |
| | GGTCATGCTCATCCTTTTGTTAATCAGCGTGTCATTGCAGCAATACAAAATGGTGTAAGTTTTTC |
| | TGCAACTCAAGAATATGAAGTCCGGGTAGCTGAACATATCATCAAGATGTGTCCTTCTGTTGACA |
| | TGGTACGTCTGGATAATAGTGGTTCTGATGCTACCCGCCATGCAATTCGTCTGGCCAGAGGTTAC |
| | ACTGGACGAGATCTAGTGTTGAAATTTGAAGGTGCTTATCACGGTGATTACGATTATATGCTTTG |
| | GACAACACCAGATAGTCTTAAAAGCCGTGTGGGAAAACGAGGCGAGCCAATTGGACATAAAACCA |
| | GTCAAGGTGTACCAGACTTAGTATCTCAATTGTTATTAATAGCAGTTTGGAATGATCGTGAGGGA |
| | ATAGAAAAACTGTTAAAGGAAAAAGGAGATAGTATTGCTGCCATTATTGTCGAGCCGATTCTTGC |
| | TAATGCAGGTGGCTTGATGCCGGAACCTGGATTTTTGCAATTCCTCCGTAATCAATGTGATATTT |
| | ATGGTATTGTTTTAATTTTCGATGAAGTTAAAACTGGTTTTCGCATCGCACCAGGAGGAGCAGGT |
| | GAATATTTTGGCGTACAGGCTGACTTGAGTGCCTATGCTAAAGCTATGGGTAATGGCTATCCGAT |
| | TTCTGCTATTGGTGGCAAGCGTGACATTATGATGACCATAGCACCAGGTAAGGTGGTACATGCAG |
| | GAACTTATACAGGAAATGTAGTAGTTACTGCTGCGGCCGATGCTACCCTTGAATATATGCAACAG |
| | CAGGATGTATTTGGGCATCTCAACCACGTTGGACAACAACTGATGGACGGTTTAGATGAAATTCT |
| | GACTCGCCATCAAATTAATCATCATATTCATGGTTTACCCTCATTGTTTGGTTTAACTTTGTCAG |
| | AATCTAATCCCAAAGATTGGCGTGATGTTGTTGATACCAATTTGGAATTATCTGAACAACTACTG |
| | ATTGAATTGGTCAATGCTGGTATTATGACTGATTCTGATCCACAACAAACTTGGTATGTGTGTGA |
| | GTCTCACACGTCTGAAGATATTGCAGAAACACTAAATAGGTTTGAAACTGCCCTGCGTAAAGCTT |
| | TACAGAAATAA |
| 44 | ATGTTAAAAGATTCCCCAAAATCCCATGCTTTCTTTGAACGCGCACAAAAAGTTATCCCCTTTGG |
| | TGTTAATTCCAGCCACCGCTACTGGGGTGAAAATACACCTGTAATTCAGCGTGGTGAAGGAGCTT |
| | ATGTTTATGACTTTGACGGGAAACGCTATATTGATTACCGTCTAGGTTTTGGTCCAGTTATTCTT |
| | GGTCATTCTCATCCTTTTGTTAATCAGCGTGTCATTGCAGCGATACAAAATGGTGTATGTTTTTC |
| | TGCAACTCAAGAATATGAAGTCCGGGTAGCTGAACATATCATCAAGATGTGTCCTTCTGTTGACA |
| | TGGTACGTCTGGACAATAGTGGTTCTGATGCTACCCGCCATGCAATTCGTCTGGCCAGAGGTTAC |
| | ACTGGACGAGATATAGTGTTGAAATTTGAAGGTGCTTATCACGGTGATTACGATTATATGCTTTG |
| | GACAACACCAGATAGTCTGAAAAGCCGTGTGGGAAAACGAGGCGAGCCAATTGGACATAAAACCA |
| | GCCAAGGTGTACCAGACTTAGTATCTCAATTGTTATTAATAGCAGTTTGGAATGATCGTGAGGGA |
| | ATAGAAAAACTGTTAAAGGAAAAAGGAGATAGTATTGCTGCCATTATTGTCGAGCCAATTCTTGC |
| | TAATGCAGGTGGCTTGATGCCGGAACCAGGATTTTTGCAATTTCTCCGTAATCAATGTGATATTT |
| | ATGGTATTGTTTTAATTTTCGATGAAGTTAAAACTGGTTTTCGCATCGCACCAGGAGGAGCAGGT |
| | GAATACTTTGGCGTACAGGCTGATTTGAATGCCTATGCTAAAGCTATGGGGAATGGTTATCCAAT |
| | TTCTGCTATTGGTGGCAAGCGTGAAATTATGATGACTATAGCACCAGGTAAGGTAGTACACGCAG |
| | GAACTTATACAGGAAATGTAGTAGTTACTGCCGCAGCCGATGCGACCCTTGAATATATGCAACAG |
| | CATGATGTATTTGGGCATCTCAACTGTGTTGGACAACAACTGATGGACGGTTTAGATGAAATTCT |
| | GACTCGCCATCAAATTGATCATCATATTCATGGTTTACCCTCATTGTTTGGTTTAACTTTGTCAA |
| | CAACTAGTCCCAAAGATTGGCGTGATGTTGTTGATACCAATTTGGAATTATCTGAACAATTACTA |
| | ATTGAATTAGTCAATGCTGGCATTATGACTGATTCTGACCCACAACAAACTTGGTATGTATCTGA |
| | TTCTCACACCTCTGAAGATATTGCACAAACATTAGATAAGTTTGAAACTGCCTTGCATAAAGCTT |
| | TACGTGCATAA |
| 45 | ATGTTAAAAGATTCCCCAAAATCCCATGCTTTCTTTGAACGCGCACAAAAAGTTATCCCCTTTGG |
| | TGTTAATTCCAGCCACCGCTACTGGGGTGAAAATACACCTGTAATTCAGCGTGGTGAAGGAGCTT |
| | ATGTTTATGACTTTGACGGGAAACGCTATATTGATTACCGTCTAGGTTTTGGTCCAGTTATTCTT |
| | GGTCATGCTCATCCTTTTGTTAATCAGCGTGTCATTGCAGCGATACAAAATGGTGTATGTTTTTC |
| | TGCAACTCAAGAATATGAAGTCCGGGTAGCTGAACATATCATCAAGATGTGTCCTTCTGTTGACA |
| | TGGTACGTCTGGACAATAGTGGTTCTGATGCTACCCGCCATGCAATTCGTCTGGCCAGAGGTTAC |
| | ACTGGACGAGATATAGTGTTGAAATTTGAAGGTGCTTATCACGGTGATTACGATTATATGCTTTG |
| | GACAACACCAGATAGTCTGAAAAGCCGTGTGGGAAAACGAGGCGAGCCAATTAGACATAAAACCA |
| | GCCAAGGTGTACCAGACTTAGTATCTCAATTGTTATTAATGGCAGTTTGGAATGATCGTGAGGGA |
| | ATAGAAAAACTGTTAAAGGAAAAAGGAGATAGTATTGCTGCCATTATTGTCGAGCCAATTCTTGC |
| | TAATGCAGGTGGCTTGATGCCGGAACCAGGATTTTTGCAATTTCTCCGTAATCAATGTGATATTT |
| | ATGGTATTGTTTTAATTTTCGATGAAGTTAAAACTGGTTTTCGCATCGCACCAGGAGGAGCAGGT |
| | GAATACTTTGGCGTACAGGCTGATTTGAGTGCCTATGCTAAAGCTATGGGGAATGGTTATCCAAT |
| | TTCTGCTATTGGTGGCAAGCGTGAAATTATGATGACTATAGCACCAGGTAAGGTAGTACACGCAG |
| | GAACTTATACAGGAAATGTAGTAGTTACTGCCGCAGCCGATGCGACCCTTGAATATATGCAACAG |
| | CATGATGTATTTGGGCATCTCAACTGTGTTGGACAACAACTGATGGACGGTTTAGATGAAATTCT |
| | GACTCGCCATCAAATTGATCATCATATTCATGGTTTACCCTCATTGTTTGGTTTAACTTTGTCAA |
| | CAACTAGTCCCAAAGATTGGCGTGATGTTGTTGATACCAATTTGGAATTATCTGAACAATTACTA |
| | ATTGAATTAGTCAATGCTGGCATTATGACTGATTCTGACCCACAACAAACTTGGTATGTATCTGA |
| | TTCTCACACCTCTGAAGATATTGCACAAACATTAGATAAGTTTGAAACTGCCTTGCATAAAGCTT |
| | TACGTGCATAA |
| 46 | ATGTTAAAAGATTCCCCAAAATCCCGAACTTTTTTTGAACGCGCACAAAAAGTTATCCCCTTTGG |
| | TGTTAATTCCAGTCATCGTTACTGGGGAAAAAATACACCTGTTATTGAGCGTGGCAAAGGAGCTT |
| | ATGTTTATGACTTTGATGGTAAACGCTATATTGATTACCGTCTAGGTTTTGGTCCAGTCATCCTG |
| | GGTCATGCTCATCCTTTTGTCAATCAGCGTGTAATTGCAGCAATCGAAAATGGTGTGACTTTTTC |
| | TGCAACTCAAGAATATGAAGTCCGAGTAGCTGAACACATCATCAAAATGTGTCCTTCTGTTGACA |
| | TGGTACGTCTAGATAATAGTGGTTCTGATGCTACGCGCCATGCAATACGCCTGGCAAGAGGTTAC |
| | ACTGGGCGAGATTTAGTGTTGAAATTTGAAGGTGCTTATCACGGTGATTATGATTATATGCTGTG |
| | GACAACACCAGATAGTCTGAAAAACTGTGTGGGAAAACGAGGTAAGCCAATTGGACATAAAACCA |
| | GTCACGGTGTACCAGACTTAGTATCTCAACTTTTATTAATAGCAATTTGGAATGATCGTGAGGGA |
| | ATAGAAAAGCTATTAAAGGAAAAAGGAGATAGCATTGCTGCGATTATTGTTGAGCCGATTCTTGC |
| | TAATGCTGGTGGCTTGATGCCAGAACCAGGATTTTTACAATTTCTCCGTAGTCAATGTGATATTT |
| | ATGGTATTGTTTTGATTTTTGATGAAGTTAAAACTGGTTTTCGCATTGCACCAGGAGGAGCAGGT |
| | GAATACTTTGGCGTGCAGGCTGATTTGAGTGCTTATGCTAAAGCTATGGGAAATGGTTATCCAAT |
| | TTCTGCCATTGGTGGCAAGCGTGAAATTATGATGAATATAGCACCAGGTAAGGTAGTACAAGCAG |
| | GAACTTATACAGGAAATGTAGTAGTTACCGCCGCCGCCGATGCGACCCTGGAATATATGCAAAAG |
| | CAGGATGTATTTGGGCATCTCAACTGTGTTGGAACACAACTGATGGAGGGTTTAGATGAAATTCT |
| | AACTCGCCATCAAATTGATCATCATATTCATGGTTTACCCTCATTGTTTGGTTTAACTTTGTCAA |
| | AAACTAATCCCACAGATTGGCGTGATGTTGTTGATACAAATTTGGAATTATCTGAAAAATTACTC |
| | ATTGAATTGGTCAATGCTGGTGTTATGACTGATTCTGATCCACAACAAACTTGGTATGTATCTGA |
| | TTCTCACACCTCTGAAGATATTGCACAAACATTAGATAAGTTTGAAACTGCTTTACATAAAGCTG |
| | TACGTATATAA |
| 47 | ATGTTAAAAGATTCCCCAAAATCCCATGCTTTCTTTGAACGCGCACAAAAAGTTATCCCCTTTGG |
| | TGTTAATTCCAGCCACCGCTACTGGGGTGAAAATACACCTGTAATTCAGCGTGGTGAAGGAGCTT |
| | ATGTTTATGACTTTGACGGGAAACGCTATATTGATTACCGTCTAGGTTTTGGTCCAGTTATTCTT |
| | GGTCATTCTCATCCTTTTGTTAATCAGCGTGTCATTGCAGCGATACAAAATGGTGTATGTTTTTC |
| | TGCAACTCAAGAATATGAAGTCCGGGTAGCTGAACATATCATCAAGATGTGTCCTTCTGTTGACA |
| | TGGTACGTCTGGACAATAGTGGTTCTGATGCTACCCGCCATGCAATTCGTCTGGCCAGAGGTTAC |
| | ACTGGACGAGATATAGTGTTGAAATTTGAAGGTGCTTATCACGGTGATTACGATTATATGCTTTG |
| | GACAACACCAGATAGTCTGAAAAGCCGTGTGGGAAAACGAGGCGAGCCAATTGGACATAAAACCA |
| | GCCAAGGTGTACCAGACTTAGTATCTCAATTGTTATTAATAGCAGTTTGGAATGATCGTGAGGGA |
| | ATAGAAAAACTGTTAAAGGAAAAAGGAGATAGTATTGCTGCCATTATTGTCGAGCCAATTCTTGC |
| | TAATGCAGGTGGCTTGATGCCGGAACCAGGATTTTTGCAATTTCTCCGTAATCAATGTGATATTT |
| | ATGGTATTGTTTTAATTTTCGATGAAGTTAAAACTGGTTTTCGCATCGCACCAGGAGGAGCAGGT |
| | GAATACTTTGGCGTACAGGCTGATTTGAATGCCTATGCTAAAGCTATGGGGAATGGTTATCCAAT |
| | TTCTGCTATTGGTGGCAAGCGTGAAATTATGATGACTATAGCACCAGGTAAGGTAGTACACGCAG |
| | GAACTTATACAGGAAATGTAGTAGTTACTGCCGCAGCCGATGCGACCCTTGAATATATGCAACAG |
| | CATGATGTATTTGGGCATCTCAACTGTGTTGGACAACAACTGATGGACGGTTTAGATGAAATTCT |
| | GACTCGCCATCAAATTGATCATCATATTCATGGTTTACCCTCATTGTTTGGTTTAACTTTGTCAA |
| | CAACTAGTCCCAAAGATTGGCGTGATGTTGTTGATACCAATTTGGAATTATCTGAACAATTACTA |
| | ATTGAATTAGTCAATGCTGGCATTATGACTGATTCTGACCCACAACAAACTTGGTATGTATCTGA |
| | TTCTCACACCTCTGAAGATATTGCACAAACATTAGATAAGTTTGAAACTGCCTTGCATAAAGCTT |
| | TACGTGCATAA |
| 48 | ATGGAACTCAAGTTTCAAGAAAATGATTTTCAATCTTTTGAACCAATACCTTATCTGAATGAATA |
| | CTTTAATTATCCTTCAGATTATTATGGGGGTGTAGGATTTGAGAATGAACAATTCCTACAATTCT |
| | TTGCTGAAGTTGCCCATGAATATCATCTCAATAACTATTTGCTATTAGATTTCGGTTGTGGTCCT |
| | ACTATTTATTCTATCCTCAGCTTAGGACGGAATTGCCGTGAAATACATATGAGTGATTATTTACA |
| | ACAAAATTTAGAGCAGGTTCAGCTTTGGCAACAAGGAAAGCCCCAAGCTTTCGATTGGTATCCTT |
| | ATCTGCGTCGCGCCTTGCAAATGGAAACTGCGTTAAATCAGAATCAGCCTTTAGATAGTTTTTTA |
| | GATGTTACAGATGAAGAAATAGAAGAACGGGCAAGATTGCTACGGGAAAAAATAACTTCTATCAG |
| | AAAAGGCAATGCTCGTGCTACTAATCCTGTGGGAGAAGAAGGAAAGGCATTATATGAAGCGGTGG |
| | TTTCTTCTTTCTGTTTAGAAGGTGTAGCACAAGATTTAGCTGAATGGAAAAATTTGATAACTAAT |
| | TTGTCTTCCACAATTAAACCAGGGGGTTTATTGATATTTGCTACTCAAATTGAAGCTGATAGTTA |
| | TCGAATTGGAGAGGGTTACGGCACAGTGGTTAATCTCACTGAACAAGATATTGTGCAGACATTAT |
| | TGAATTGTAATTTCGAGTCAGGTTCAATCAAAACAAAGGAACTTAAAGGCGGTCCGAATCACACT |
| | GAGTACGATAAATTCCTGATGTTGACAGCCAGACTTGCTGTGTGA |
| 49 | AACAATTCCTACAATTCTTTGCTGAAGTTGCCCATGAACATGATCTCAAAAATTGTTTGCTACTA |
| | GATTTTGGTTGTGGTCCTACTATTTATTCTATCCTCAGCTTAGGACTGAACTGCTGTGAAATACA |
| | CATGAGTGATTATTTACAACAAAATTTAGAGCAGGTTCAGCTTTGGCAACAAGGAAAGCCCCAAG |
| | CTTTTGATTGGCATCCTTATCTGCGTCGCGCCCTGCAAATGGAAACTGCGTTAAATCAGCATCAG |
| | CCTCTAGATAGTTTTTTAGATGTTACAGATGAAGAAATAGAACAACGGGCGAGATTGCTACGGGG |
| | AAAAATAACTTCTATCAGACAAGGCAATGCTCGTGCTATTAATCCAGTGGGAGAAGAAGGAAAGG |
| | CATTATATGAAGCGGTGGTTTCTTCTTTCTGTTTAGAAGGTGTAGCACAAGATTTAGCTGAATGG |
| | AAAAAGTTGATGACTAATTTGTCTTCCACCATTAAACCAGGGGGTTTATTGATATTTGCTACTCA |
| | AATTGAAGCTGATAGTTATCGAATTGGCGATGGTTACGGCACAGTGGTTAATCTCAGTGAACAAG |
| | ATATTGTGGAAACATTATTGGATTGTAATTTCGAGTCAAGCTCAATCAAAACGAAGGAACTTGAA |
| | GGCGGTCCGAATCACACAGAGTACGAGAAATTCCTTATGCTGACAGCAAGACTTGCTTTGTGA |
| 50 | ATGCAACTGAAATTTAAAGAAAATGATTTTCAATCTTTTGAGCCAATACCCTATCTGAATGAATA |
| | CTTTAATTATCCTTCAGATTATTACGGAGGTGTAGGATTTGAGAACGAACAATTCCTACAATTCT |
| | TTGCTGAAGTTGCCCATGAACATGATCTCAAAAATTGTTTGCTACTAGATTTTGGTTGTGGTCCT |
| | ACTATTTATTCTATCCTCAGCTTAGGACTGAACTGCTGTGAAATACACATGAGTGATTATTTACA |
| | ACAAAATTTAGAGCAGGTTCAGCTTTGGCAACAAGGAAAGCCCCAAGCTTTTGATTGGCATCCTT |
| | ATCTGCGTCGCGCCCTGCAAATGGAAACTGCGTTAAATCAGCATCAGCCTCTAGATAGTTTTTTA |
| | GATGTTACAGATGAAGAAATAGAACAACGGGCGAGATTGCTACGGGGAAAAATAACTTCTATCAG |
| | ACAAGGCAATGCTCGTGCTATTAATCCAGTGGGAGAAGAAGGAAAGGCATTATATGAAGCGGTGG |
| | TTTCTTCTTTCTGTTTAGAAGGTGTAGCACAAGATTTAGCTGAATGGAAAAAGTTGATGACTAAT |
| | TTGTCTTCCACCATTAAACCAGGGGGTTTATTGATATTTGCTACTCAAATTGAAGCTGATAGTTA |
| | TCGAATTGGCGATGGTTACGGCACAGTGGTTAATCTCAGTGAACAAGATATTGTGGAAACATTAT |
| | TGGATTGTAATTTCGAGTCAAGCTCAATCAAAACGAAGGAACTTGAAGGCGGTCCGAATCACACA |
| | GAGTACGAGAAATTCCTTATGCTGACAGCAAGACTTGCTTTGTGA |
| 51 | ATGGAACTGAAATTTAAAGAAAATGATTTTCAATCTTTTGAGCCAATACCCTATCTGAATGAATA |
| | CTTTAATTATCCTTCAGATTATTACGGAGGTGTAGGATTTGAGAATGAACAATTCCTACAATTCT |
| | TTGCCGAAGTTGCCCATGAACATGATCTCAAAAATTGTTTGCTACTAGATTTTGGTTGTGGACCT |
| | ACTATTTACTCTATCCTTAGCTTAGGACGGAACTGCCGTGAAATACACATGAGTGATTATTTACA |
| | GCAAAATTTAGAGCAGGTTCAGCTTTGGCAACAGGGAAAACCTCAAGCTTTTGATTGGCATCCGT |
| | ATCTGCGTCGCGCCCTGCAAATGGAAACCTTGTTAAATCAGAATCAGCCTCTAGAGAGTTTTATG |
| | GATCAACAGATAGAACAACGGGCGAGATTGCTACGGGGAAAAATAACTTCTATCAGACAAGGGAA |
| | TGCTCGTGCTATTAATCCAGTGGGAGAAGAAGGCAAGGGATTATATGAAGCGGTGGTTTCTTCTT |
| | TCTGTTTAGAAGGCGTAGCACAAGATTTAGCTGAGTGGAAAAGCTTAATAACTAATTTGTCTTCG |
| | ACAATTAAACCAGGGGGTTTATTGATATTTGCTACTCAAATTGAAGCTGATAGTTATCGAATTGG |
| | CGATGGTTACGGCACAGTGGTTAATCTGAGTGAACAAGATATTGTGCAAACATTATTAGATTGTA |
| | ATTTCCAGTCAAACTCAATCAAAACCAGGGAACTTCAAGGCGGTCCGAATCACACTGAGTACGAG |
| | AAATTCCTAATGTTGACAGCAAGACTCGCTTTGTAA |
| 52 | ATGGAACTGAAATTTAAAGAAAATGATTTTCAATCTTTTGAGCCAATACCCTATCTGAATGAATA |
| | CTTTAATTATCCTTCAGATTATTACGGAGGTGTAGGATTTGAGAACGAACAATTCCTACAATTCT |
| | TTGCTGAAGTTGCCCATGAACATGATCTCAAAAATTGTTTGCTACTAGATTTTGGTTGTGGTCCT |
| | ACTATTTATTCTATCCTCAGCTTAGGACTGAACTGCTGTGAAATACACATGAGTGATTATTTACA |
| | ACAAAATTTAGAGCAGGTTCAGCTTTGGCAACAAGGAAAGCCCCAAGCTTTTGATTGGCATCCTT |
| | ATCTGCGTCGCGCCCTGCAAATGGAAACTGCGTTAAATCAGCATCAGCCTCTAGATAGTTTTTTA |
| | GATGTTACAGATGAAGAAATAGAACAACGGGCGAGATTGCTACGGGGAAAAATAACTTCTATCAG |
| | ACAAGGCAATGCTCGTGCTATTAATCCAGTGGGAGAAGAAGGAAAGGCATTATATGAAGCGGTGG |
| | TTTCTTCTTTCTGTTTAGAAGGTGTAGCACAAGATTTAGCTGAATGGAAAAAGTTGATGACTAAT |
| | TTGTCTTCCACCATTAAACCAGGGGGTTTATTGATATTTGCTACTCAAATTGAAGCTGATAGTTA |
| | TCGAATTGGCGATGGTTACGGCACAGTGGTTAATCTCAGTGAACAAGATATTGTGGAAACATTAT |
| | TGGATTGTAATTTCGAGTCAAGCTCAATCAAAACGAAGGAACTTGAAGGCGGTCCGAATCACACA |
| | GAGTACGAGAAATTCCTTATGCTGACAGCAAGACTTGCTTTGTGA |
| 53 | ATGAGTGAGCATTTTCCCATTGATTTACGCAGTGATACAGTTACCCAACCGACACCATCTATGCG |
| | TCAAGCTATGGCTGAAGCGATAGTAGGTGACGATGTTTATGGAGAAGATCCAACGGTTAACCGAC |
| | TGGAACGAGTAGCAGCAGAACTTTTAGGTAAAGAGTCTGCTATGTTTGTACCTAGTGGCACGATG |
| | GGTAATCTCATCTGTATGTTAACTCATTGTGGACGTGGAGAAGAAGTGATTATTGGTGATCAAGC |
| | TCATCTGTTTTTGTATGAACGTGGTGGCAGTGCTGTATTGGGGGGTATTCATCCTTACACTGTCA |
| | GCACAGCAACAGATGGTACTCTTCCTTTATCAGAAATCCGCGCTGCTGTAAGACCTGATGATATT |
| | CATTGTCCGAGAACAAAGCTATTATGTGTGGAAAATACACAAGCACAACGAGGCGGTTTGCCTTT |
| | ATCATTAACTTATTTACACAGCTTGCGTGATTTGGCGAATGAGTTAGGATTAAGATTGCACTGTG |
| | ATGGAGCGCGTTTGTGGAACGCATCTGTGGCAATTGGGGTTGAACCTAGTGTTTTATCTTCACCT |
| | TTTGATTCTTTATCTGTCTGTTTATCAAAGGGTCTGGCTGCACCTATTGGTTCTCTTGTGATCGG |
| | AACACAGTCATTTATTGATGAAGCACGGCGGTTACGTAAACTATTGGGTGGTGGTATGCGTCAAG |
| | TTGGAGCGATCGCCTCTGCTGGTTTAATTGCCCTAGAACAAATGAGGGAAAGACTGGCTATTGAT |
| | CATGAAAACGCCCAATTTTTAGCTGAAGGTTTACGTGAATGTGGTTATGAAATCACTCATCCTGT |
| | GCGGACTAATATCATATTTTTTCGCCAGCATGAGCAAGATACCTTATCTCTCCAGGAAAGATTAG |
| | CTGTTTGGAAAAACTCTGGCTTGTTAGTCTCAGATTTTGGCGATCGCACAATTCGCGCAGTTACC |
| | CATTATGGTATTGAACGCAAAGATATTAATCAAGCAATTTCTATTTTACAACAAGTCTAG |
| 54 | ATGACTCAGCATTTTACCATTGATTTACGCAGTGATACAGTCACCCAACCCACACCCTCTATGCG |
| | TAAAGCTATGGCTGAAGCGATAGTAGGTGACGATGTTTATGGAGAAGATCCAACGGTTAACCGCC |
| | TGGAACGAGTAACAGCAGAACTTTTAGGTAAGGAGTCTGCAATGTTTGTACCTAGTGGCACGATG |
| | GGTAATCTCATCTGTGTGTTAACTCATTGTGGACGTGGGGAAGAAGTGATTATTGGTGATCAAGC |
| | TCATCTGTTTTTGTATGAACGTGGTGGCAGTGCTGTATTGGGGGGTATTCATCCTTACACTGTCA |
| | GCACAGCAACAGATGGTTCTCTTCCTTTATCAGAAATCCGCGCTGCTGTACGACCTGATGATATT |
| | CATTGTCCGAGAACAAAGCTATTATGTGTGGAAAATACACAAGCACAACGAGGCGGTTTGCCTTT |
| | ATCATTAACTTATTTACAGAGCTTGCGTGATTTGGCAAATGAGTTAGGATTTGCATTGCACTGTG |
| | ATGGAGCGCGTTTGTGGAATGCATCTGTAGCAACTGGGATTGAACCAAGTGTTTTATCTTCACCT |
| | TTTGATTCTTTATCTGTCTGTCTATCAAAAGGGCTGGCTGCACCTATTGGTTCCCTTGTGATCGG |
| | AACACAGTCATTTATTGATGAAGCACGGCGGTTACGTAAACTATTGGGTGGCGGTATGCGTCAAG |
| | TTGGAGCGATCGCCTCTGCTGGTTTAATTGCCATACAAGAAATGAGGGAAAGACTGGCTATTGAT |
| | CATGAAAACGCCCAATTTTTAGCTGAAGGTCTACGTGAATGTGGTTATGAAATCACCCATCCGGT |
| | GCGGACTAATATCATATTTTTTCGCCAGCATGAGCAAGATACCTTATCTCTCCAGGAAAGATTAG |
| | CTGTTTGGAAAAACTCTGGATTGTTAGTCTCAGATTTTGGCGATTGCACAATTCGCGCAGTTACC |
| | CATTACGGTATTGAACGCAAAGATATTGATCAAGTAATTTCTATTTTACAACAAGTCTAG |
| 55 | ATGACTCAGCATTTTACCATTGATTTACGCAGTGATACAGTCACCCAACCCACACCCTCTATGCG |
| | TAAAGCTATGGCTGAAGCGATAGTAGGTGACGATGTTTATGGAGAAGATCCAACGGTTAACCGCC |
| | TGGAACGAGTAACAGCAGAACTTTTAGGTAAGGAGTCTGCAATGTTTGTACCTAGTGGCACGATG |
| | GGTAATCTCATCTGTGTGTTAACTCATTGTGGACGTGGGGAAGAAGTGATTATTGGTGATCAAGC |
| | TCATCTGTTTTTGTATGAACGTGGTGGCAGTGCTGTATTGGGGGGTATTCATCCTTACACTGTCA |
| | GCACAGCAACAGATGGTTCTCTTCCTTTATCAGAAATCCGCGCTGCTGTACGACCTGATGATATT |
| | CATTGTCCGAGAACAAAGCTATTATGTGTGGAAAATACACAAGCACAACGAGGCGGTTTGCCTTT |
| | ATCATTAACTTATTTACAGAGCTTGCGTGATTTGGCAAATGAGTTAGGATTTGCATTGCACTGTG |
| | ATGGAGCGCGTTTGTGGAATGCATCTGTAGCAACTGGGATTGAACCAAGTGTTTTATCTTCACCT |
| | TTTGATTCTTTATCTGTCTGTCTATCAAAAGGGCTGGCTGCACCTATTGGTTCCCTTGTGATCGG |
| | AACACAGTCATTTATTGATGAAGCACGGCGGTTACGTAAACTATTGGGTGGCGGTATGCGTCAAG |
| | TTGGAGCGATCGCCTCTGCTGGTTTAATTGCCATACAAGAAATGAGGGAAAGACTGGCTATTGAT |
| | CATGAAAACGCCCAATTTTTAGCTGAAGGTCTACGTGAATGTGGTTATGAAATCACCCATCCGGT |
| | GCGGACTAATATCATATTTTTTCGCCAGCATGAGCAAGATACCTTATCTCTCCAGGAAAGATTAG |
| | CTGTTTGGAAAAACTCTGGATTGTTAGTCTCAGATTTTGGCGATTGCACAATTCGCGCAGTTACC |
| | CATTACGGTATTGAACGCAAAGATATTGATCAAGTAATTTCTATTTTACAACAAGTCTAG |
| 56 | ATGACCGAGTATTTTACCATTGATTTACGCAGTGATACAGTGACCCTACCCACACCTTCTATGCG |
| | TAAAGCCATGGCTGAAGCGATAGTAGGTGACGATGTTTATGGAGAAGATCCAACGGTTAACCGCC |
| | TAGAACGAGTAGCAGCAGAACTTTTAGGTAAAGAGTCTGCAATGTTTGTACCTAGTGGTACGATG |
| | GGTAATCTCATCTGTGTTTTAACTCATTGTGGACGTGGGGAAGAAGTGATTATTGGCGATCAAGC |
| | TCATCTGTTTTTGTACGAACGTGGGGGCAGTGCAGTGTTGGGGGGTATTCATCCGTACACTGTTA |
| | AGACAGCAACAGATGGCTCTCTCCCTTTATCAGCAATCCGTGAAGCTGTACGACCGGATGATATT |
| | CATTGCCCGAGAACAAAACTATTATGTCTAGAAAATACACAAGCACAACGAGGAGGTGCGCCTTT |
| | ATCATTAACTTATTTACAAAGTCTGCGTGATTTGGCAAATGAGTTAGGACTGGCATTACACTGTG |
| | ATGGAGCGCGTTTGTGGAATGCATCCGTCGCAACTGGAGTTGAGCCAAGTGTTTTATCTTCACCT |
| | TTTGATTCTTTATCTGTCTGTTTATCAAAGGGGTTGGGTGCGCCTATTGGTTCCCTTGTAATCGG |
| | AACACAGTCATTTATTGATGAAGCACGGCGATTACGCAAACTACTGGGTGGGGGTATGCGTCAAG |
| | CTGGAGCGATCGCGGCTGCTGGTTTAATTGCCATACAAGAAATGAGGGAAAGACTGGCTACCGAT |
| | CATGAAAATGCTCAATTTTTAGCCGCAGGTTTACGTGAATGTGGTTATGAAATCACCCATCCGGT |
| | GCGGACTAATATCATATTTTTTCGCCAGCATAAGGAAGATACCTTATCTGTCCGGGAAAGATTAG |
| | CTGTTTGGAAAAACTCTGGCTTGTTAGTCTCAGATTTTGGTGATTGCACAATTCGCGCAGTTACC |
| | CATTACGGTATTGAACGCAAAGATATTGATCAAGTAATTTCTATTGTACAAAAAGTCTAG |
| 57 | ATGACTCAGCATTTTACCATTGATTTACGCAGTGATACAGTCACCCAACCCACACCCTCTATGCG |
| | TAAAGCTATGGCTGAAGCGATAGTAGGTGACGATGTTTATGGAGAAGATCCAACGGTTAACCGCC |
| | TGGAACGAGTAACAGCAGAACTTTTAGGTAAGGAGTCTGCAATGTTTGTACCTAGTGGCACGATG |
| | GGTAATCTCATCTGTGTGTTAACTCATTGTGGACGTGGGGAAGAAGTGATTATTGGTGATCAAGC |
| | TCATCTGTTTTTGTATGAACGTGGTGGCAGTGCTGTATTGGGGGGTATTCATCCTTACACTGTCA |
| | GCACAGCAACAGATGGTTCTCTTCCTTTATCAGAAATCCGCGCTGCTGTACGACCTGATGATATT |
| | CATTGTCCGAGAACAAAGCTATTATGTGTGGAAAATACACAAGCACAACGAGGCGGTTTGCCTTT |
| | ATCATTAACTTATTTACAGAGCTTGCGTGATTTGGCAAATGAGTTAGGATTTGCATTGCACTGTG |
| | ATGGAGCGCGTTTGTGGAATGCATCTGTAGCAACTGGGATTGAACCAAGTGTTTTATCTTCACCT |
| | TTTGATTCTTTATCTGTCTGTCTATCAAAAGGGCTGGCTGCACCTATTGGTTCCCTTGTGATCGG |
| | AACACAGTCATTTATTGATGAAGCACGGCGGTTACGTAAACTATTGGGTGGCGGTATGCGTCAAG |
| | TTGGAGCGATCGCCTCTGCTGGTTTAATTGCCATACAAGAAATGAGGGAAAGACTGGCTATTGAT |
| | CATGAAAACGCCCAATTTTTAGCTGAAGGTCTACGTGAATGTGGTTATGAAATCACCCATCCGGT |
| | GCGGACTAATATCATATTTTTTCGCCAGCATGAGCAAGATACCTTATCTCTCCAGGAAAGATTAG |
| | CTGTTTGGAAAAACTCTGGATTGTTAGTCTCAGACTTTGGCGATTGCACAATTCGCGCAGTTACC |
| | CATTACGGTATTGAACGCAAAGATATTGATCAAGTAATTTCTATTTTACAACAAGTCTAG |
| 58 | ATGAGTAACAACAAACAAGACATCAATCACACAGAATTAAATCCCATGGCTCATAGCCGCAGGGG |
| | ATTTTTTACTGTGTTTATTGCAGGAATTTCTACATCTGTATCTAGTATTTTCAGGAAAGATGTTG |
| | CTGTAGCTGAGAACCATAAAGCCTTCACCGCCGAAAACAAATCAATGCTCTCTACCAATAACCAG |
| | TCATTTTTTTTTCCAGGTGAATTGTTAGCATCTGACGAAATGCGGATCACTATTGTAGGGTCTTC |
| | TTCCGTCGCACGCAAAACCCAATCTGGAACAAGCATCTTTCTCGAACTGGGAAACGGGGACAGTT |
| | TCATCTTTGATGCCGGACCTGGTGTAGCTGCCAACTATACAGCGTTGGGTGTACCCTATTCGCGG |
| | ATGGATAGAATTTTCCTAAGTCACCTTCATTGCGATCACATAACGGATCTTGCCTACATATATGG |
| | TCAAGGATCAATGTATGATCGCAAAACACCCCTACGTATCTGGGGACCGAGTGGCAAGGAGTCGA |
| | AGTTTGGCACAAATGCTTTCGTCGAGTCAATGAAGGCTATGTGTCTCTGGCACACGGAGAGTCTT |
| | TCCTACCACCAGAACGGTAATGGTTATGAAACAGAGGTACATGAATTTGACTATGCTCAAAATCC |
| | AGGTGTTGCATACAACGAGAAAGGAGTTGTGATTAAGCACTTTCCCGCAGTTCACCTGAAAGACG |
| | GTGCGGTAAGCTACCGTGTGGATTGGAATGGTCTCAGTTTTGTCTTCTCTGGCGACTCGCAACCG |
| | AATACATTTATGGTTGAGAACGCACAAAATGTTGATATCTTGATTCACCAAACAATTCCTCCAGC |
| | ACAGGTTTGGGCAAAAGAATACAATATGCCCCTCGAACAAGTTATAGAACTGCAAAACAGATTTG |
| | ATTCTCCTCCCCATGCTGTTGGTGCGATTTTCGACCAAACTAGACCACGCTTGGGTGTACTAACT |
| | CATTTATATCTAAATGAAAATATAAAAGTTGCCATTCTTGACTCGTGCCAAGAGGTTTACGACGG |
| | ACCTATTGAAATTGCTCAGGATTTGATGGTTTTCAATATCTCCAAGAATGAAATCAAGCAGCGCA |
| | TAGCGATCGTCCCTGAATTAGCGTTGCAAAACACCGTCAAGAAGTACGAAAACCTTCCACCACCT |
| | AAGTATAAACCAAGGGAGCAGTTGTCCCAGTGGCTTCTGGACGCGGTAATTCCGCAAGAACAATG |
| | GAAATAG |
| 59 | ATGAGTAACAACAAGCAAGACATTAATAACAGAAAATTAAATCCAATGGCTCATAGCCGCAGGGG |
| | ATTTTTAACTGTGTCTATTGCAGGAATTTCTACGACTGTATCTAGTATTCTCAGGAAAGATGTTG |
| | CTGCGGCTGAGAACAATAAAGCTTTCACTGCCGAAAACAAATCAATGCTCTCCACAAATAAGCAG |
| | TTTTTTTTTCCAGGTGAATTGCTAGCATCTGACGAAATGCGGATCACTATTGTAGGGTCTTCCTC |
| | AGTCGCACGCAAAACGCAATCTGGCACAAGCATCTTTCTCGAACTGGGAAACGGGGACAACTTCA |
| | TCTTTGATGCCGGACCTGGTGCCGCTGCCAACTATACGGCGTTGGGTGTACCCTATTCGCGGATG |
| | GATAGAATTTTTCTGAGCCACCTCCATTGCGATCACATAACAGATATTGCCTACATATATGGTCA |
| | AGGTCCAATGTATGATCGCAAAACGCCCCTACGTATCTGGGGACCGAGTGGCAAGGAGTCGAAGT |
| | TCGGCACAAATGCTTTCATCGAGTCCATGAAGGCTATGTGTCTCTGGCACACGGAGAGTCTTTCC |
| | TACCACCAGAACGGGAATGGTTACGAAACAGAGGTACATGAATTTGACTATACTCAAAATCCAGG |
| | TGTTGCATACAACGAGAAAGGAGTTGTGATCAAGCACTTCCCCGCAGTTCACCTGAAAGACGGTG |
| | CCGTAAGCTACCGTGTGGATTGGAATGGTCTCAGTTTTGTCTTTTCTGGTGACTCGCAACCGAAT |
| | ACATTTATGGTTGAGAACGCACAAAATGTTGACATCTTGATTCACCAGACAATTCCCCCAGCACA |
| | GGTTTGGGCGAAAGAATACAATATGCCGCTAAAACAAGTTATAGAACTGCAAAACAGATTTGATT |
| | CTCCTCCCGATGCTGTTGGTGCGATTTTCGCCCGAACTAGACCACGCTTAGGTGTACTAACTCAT |
| | ATACATCTGAATGAGAATATAAAAGTTTCTATTCTTGACTCCTGCCAAGAGGTTTACGACGGACC |
| | TATTGAAATTGCTCAGGATTTGATGGTGTTCAATATCTCCAAGAATGAAATCAAGCAGCGCATAG |
| | CGATCGTCCCTGAATTAGCGTTGCAAAACACCGTCAAGAAGTACGAAAACCTTCCGCCACCGAAG |
| | TATAAACCAAGAGAGCAGTTGTCCCAGTG |
| 60 | ATGAGTAACAACAAGCAAGACATTAATAACAGAAAATTAAATCCAATGGCTCATAGCCGCAGGGG |
| | ATTTTTAACTGTGTCTATTGCAGGAATTTCTACGACTGTATCTAGTATTCTCAGGAAAGATGTTG |
| | CTGCGGCTGAGAACAATAAAGCTTTCACTGCCGAAAACAAATCAATGCTCTCCACAAATAAGCAG |
| | TTTTTTTTTCCAGGTGAATTGCTAGCATCTGACGAAATGCGGATCACTATTGTAGGGTCTTCCTC |
| | AGTCGCACGCAAAACGCAATCTGGCACAAGCATCTTTCTCGAACTGGGAAACGGGGACAACTTCA |
| | TCTTTGATGCCGGACCTGGTGCCGCTGCCAACTATACGGCGTTGGGTGTACCCTATTCGCGGATG |
| | GATAGAATTTTTCTGAGCCACCTCCATTGCGATCACATAACAGATATTGCCTACATATATGGTCA |
| | AGGTCCAATGTATGATCGCAAAACGCCCCTACGTATCTGGGGACCGAGTGGCAAGGAGTCGAAGT |
| | TCGGCACAAATGCTTTCATCGAGTCCATGAAGGCTATGTGTCTCTGGCACACGGAGAGTCTTTCC |
| | TACCACCAGAACGGGAATGGTTACGAAACAGAGGTACATGAATTTGACTATACTCAAAATCCAGG |
| | TGTTGCATACAACGAGAAAGGAGTTGTGATCAAGCACTTCCCCGCAGTTCACCTGAAAGACGGTG |
| | CCGTAAGCTACCGTGTGGATTGGAATGGTCTCAGTTTTGTCTTTTCTGGTGACTCGCAACCGAAT |
| | ACATTTATGGTTGAGAACGCACAAAATGTTGACATCTTGATTCACCAGACAATTCCCCCAGCACA |
| | GGTTTGGGCGAAAGAATACAATATGCCGCTAAAACAAGTTATAGAACTGCAAAACAGATTTGATT |
| | CTCCTCCCGATGCTGTTGGTGCGATTTTCGCCCGAACTAGACCACGCTTAGGTGTACTAACTCAT |
| | ATACATCTGAATGAGAATATAAAAGTTTCTATTCTTGACTCCTGCCAAGAGGTTTACGACGGACC |
| | TATTGAAATTGCTCAGGATTTGATGGTGTTCAATATCTCCAAGAATGAAATCAAGCAGCGCATAG |
| | CGATCGTCCCTGAATTAGCGTTGCAAAACACCGTCAAGAAGTACGAAAACCTTCCGCCACCGAAG |
| | TATAAACCAAGAGAGCAGTTGTCCCAGTGGCTTTTGGACGCGGTAATTCCGCAAGAAGAATGGAA |
| | GAAAGTCTAA |
| 61 | ATGAGTAACGACATCAATAAAACAGAATTAAATCCAATGGCTTCTAGCCGCAGGGGATTTTTTAG |
| | TCTGTCGGTTGCAGGAGTTGTAACGGCTGTATCTAGTATTCTCAGAAAAGATGTTACTGCGGCTC |
| | AGAACCATAAAGCTTTCACTGTTGAAAATAAATCAATGCTCTCAAAAAATAACCAGTTATTTTTT |
| | TTTCCCGGTGAATTGTTAGCACCCGACGAAATGCGGATCACTATTATCGGGTCTTCTCCAGTCGC |
| | ACGCAAAACCCAATCTGGTACAAGCATCTTTCTGGAACTGGGAAACGGTGACAACTTCATCTTTG |
| | ATGCTGGACCTGGTACTGCCGCCAACTATACTGCCTTGGGTGTACCCTATTCGCGGATGGATAGA |
| | ATTTTTCTGACTCACCTCCATTGTGATCACATCACCGATCTTCCCTACATCTATGGTCAAGGTGC |
| | GATGTACGATCGCAAAACCCCCCTACATATCTGGGGTCCGAGTGGTAAGGAGTCGAAGTTCGGCA |
| | CAAATGCTTTTGTCGAGTCAATGAAGGCTATGTGTCTCTGGCACACGGAAAGTCTTTCCTACCAC |
| | CAGAACGGTAATGGTTACGAAACGGAAGTACATGAATTTGACTATACTCAAAATCCAGGTGTTGC |
| | ATATAACAAGAAAGGAGTTGTGATCAAGCACTTCCCCGCAGTCCACCTGAAAGACGGTGCCGTAA |
| | GCTACCGTGTAGATTGGAATGGTCTCAGTTTTGTCTTCTCTGGTGACTCACAACCGAATACATTT |
| | ATGGTTGAGAATGCACAAAATGTTGATATCTTGATTCACCAGACAATTCCTCCAGCAACGGTTTG |
| | GGCGAAAGAATACAATATGCCGCTCGAAAAAGTTATAGAACTGCAAAACAGATTTGATTCTCCTC |
| | CCAATGCTGTTGGTCAGATTTTCGCCCGCACTAAACCACGTTTGGGTATACTAACCCATATATAT |
| | ATTAATAAAAATATTAAACTTTCTCTTCTCAAATCCTGCCAAGAGGTTTACGACGGATATATTGA |
| | AATTGCTCAGGATTTGATGGTATTCAATATCTCCAAAAATCAAATCAAGCGGCGCATAGCGATCG |
| | TCCCTGAATTAGTCCTACAAAATATTGTCAAGAAGTACGAAAACCTGCCGCCACCGAAGTATAAA |
| | CCAAGGGAGCAGTTGTCCCAGTGGCTTCTCGACGCGGTAATTCCACCCGAAGAATGGAAGATGTA |
| | A |
| 62 | ATGAGTAACAACAAGCAAGACATTAATAACAGAAAATTAAATCCAATGGCTCATAGCCGCAGGGG |
| | ATTTTTAACTGTGTCTATTGCAGGAATTTCTACGACTGTATCTAGTATTCTCAGGAAAGATGTTG |
| | CTGCGGCTGAGAACAATAAAGCTTTCACTGCCGAAAACAAATCAATGCTCTCCACAAATAAGCAG |
| | TTTTTTTTTCCAGGTGAATTGCTAGCATCTGACGAAATGCGGATCACTATTGTAGGGTCTTCCTC |
| | AGTCGCACGCAAAACGCAATCTGGCACAAGCATCTTTCTCGAACTGGGAAACGGGGACAACTTCA |
| | TCTTTGATGCCGGACCTGGTGCCGCTGCCAACTATACGGCGTTGGGTGTACCCTATTCGCGGATG |
| | GATAGAATTTTTCTGAGCCACCTCCATTGCGATCACATAACAGATATTGCCTACATATATGGTCA |
| | AGGTCCAATGTATGATCGCAAAACGCCCCTACGTATCTGGGGACCGAGTGGCAAGGAGTCGAAGT |
| | TCGGCACAAATGCTTTCATCGAGTCCATGAAGGCTATGTGTCTCTGGCACACGGAGAGTCTTTCC |
| | TACCACCAGAACGGGAATGGTTACGAAACAGAGGTACATGAATTTGACTATACTCAAAATCCAGG |
| | TGTTGCATACAACGAGAAAGGAGTTGTGATCAAGCACTTCCCCGCAGTTCACCTGAAAGACGGTG |
| | CCGTAAGCTACCGTGTGGATTGGAATGGTCTCAGTTTTGTCTTTTCTGGTGACTCGCAACCGAAT |
| | ACATTTATGGTTGAGAACGCACAAAATGTTGACATCTTGATTCACCAGACAATTCCCCCAGCACA |
| | GGTTTGGGCGAAAGAATACAATATGCCGCTAAAACAAGTTATAGAACTGCAAAACAGATTTGATT |
| | CTCCTCCCGATGCTGTTGGTGCGATTTTCGCCCGAACTAGACCACGCTTAGGTGTACTAACTCAT |
| | ATACATCTGAATGAGAATATAAAAGTTTCTATTCTTGACTCCTGCCAAGAGGTTTACGACGGACC |
| | TATTGAAATTGCTCAGGATTTGATGGTGTTCAATATCTCCAAGAATGAAATCAAGCAGCGCATAG |
| | CGATCGTCCCTGAATTAGCGTTGCAAAACACCGTCAAGAAGTACGAAAACCTTCCGCCACCGAAG |
| | TATAAACCAAGAGAGCAGTTGTCCCAGTGGCTTTTGGACGCGGTAATTCCGCAAGAAGAATGGAA |
| | GAAAGTCTAA |
| 63 | TTGAAAAAATCCATCGACAAGATCATTACCAACTTACCGCTATGGGATACACAAGGTGCTCTGAA |
| | AATTGTGCCAATCGGTGACCAAAACTTCACCAATCAGAATTACCGAGTTGAGAGCAACGGACAGG |
| | TATTCAAGGTTCGCATCAGTGAAGGAAATCAAAACCTCATAGGGATTGCCAAAGAAGAAGAACTG |
| | ATTGTGCTTAAGGCAGTTGCTAAAATGGGTATAGGACCAGAGGTCATTGCATACATACCACCAGA |
| | AGGTCATCTGGTAACGCGATTTATAGAGGGTAAGCATTTTTCTCTACAGGAAATCACGCAACCGG |
| | AAAACATACATCGAATTGCCCAGATCCTAAAGCAAGTTCATAGTATTGAGGGGATTGACGCTGCA |
| | CCGCCACCATTTGAGCGAATTGAGGGGCTGATGAGGAACGCTGAACGCAAAAATGGCATATTTCC |
| | TGATGATTTTGAACAGTTACTCGCTGACCTACAAGCTATAAAGGCTGTACTGACTAAAGTCCACA |
| | GAAAACCATGTCTGTGTCATAACGACTTGGCTGGCTCGAATATCATTGAGGCCGATGGTTCAATT |
| | TTCCTGATCGATTGGGAGTACGCAGGTGTAGCAAATCCGATGTTTGACCTAGCTAACTTTTCTAT |
| | GAATCAGAACCTGGATAGAGAAGGTGATAAAATTCTCATTGAGAGCTACTTTGGGAAAGTTAGTC |
| | AGGTGGAGTTAGCAGAAATCAATCTTTGGAAGATAGTTGCTATTTTTATTGAAGGTGTTTGGGGT |
| | GTTTTGCAGGCAAAAATTTCTCAATTTGATCGGGACTATCAAGGTTTTGCTAACGAAAATTGGCA |
| | AACAGTGCGTCAGTACACGCAAAAGGAAAGCTTTTCAGAATGGCTGTCCATGATATCGTTATCAA |
| | ACCACTGA |
| 64 | TTGAAAAAATCCATAGACAAGATAATTACCAACTTACCGCTATGGGATACACAAGATGCTCTGAA |
| | AATTATGCCAATTGGTGATCAAAACTTCACCAATCAGAATTACCGAGTTGAGGGCAACGGACAGG |
| | TATTCAAGATTCGCATCAGCGAAGGAAATCAAAACCTAATAGGGATTGCTAGGGAAGAAGAACTA |
| | AATGTTCTTAAGGCAGTTGCTAAAATGGGTATAGGATCAGAAGTGATTGCATACATACCACCAGA |
| | AGGGCATCTGGTGACGCGATTTATAGAGGGTAGGCATTTTTCTCTAGAGGAAATTACACAACCGG |
| | AAAACATACATCGAATTGCCCAGATCCTAAAGCAAGTTCATAGTATTGAGGGGATTGACGCTGCA |
| | CCGCCACCATTTGAGCGAATTGAGGGGCTGATGAGGAACGCTGAACGCAAAAATGGCATATTTCC |
| | TGACGATTTTGAACAGCTACTCGCTAATCTACAAGCTATAAAGGCTGTACTGACTAAAGTCCACA |
| | GAAAACCATGTCTGTGTCATAACGACTTGGCTGGATCGAATATCATCGAGTCCGATGGTTCAATT |
| | TTCCTAATCGATTGGGAGTACGCAGGTCTAGCAAATCCGATGTTTGATCTAGCTAACTTTTCTAT |
| | GAATCAGAACCTGGATAGAGAAGGTGATCAAATTCTCATTGAGAGCTACTTTGGGAAAGTTAGTC |
| | AGATGGAGTTAGCAGAAATCAATCTTTGGAAGATAGTTGCTATCTTCATTGAAGGTGTTTGGGGT |
| | ATTTTACAGGCGAAAATTTCTCAATTTGATCGGGACTATCAAGGCTTTGCTAACGAAAATTGGCA |
| | AACAGTGCGTCAGTACACGCAAAAGGAAAGCTTTTCACAATGGCTCTCTATGATATCGTCATCAA |
| | ACCACTGA |
| 65 | TTGAAAAAATCCATAGACAAGATAATTACCAACTTACCGCTATGGGATACACAAGATGCTCTGAA |
| | AATTATGCCAATTGGTGATCAAAACTTCACCAATCAGAATTACCGAGTTGAGGGCAACGGACAGG |
| | TATTCAAGATTCGCATCAGCGAAGGAAATCAAAACCTAATAGGGATTGCTAGGGAAGAAGAACTA |
| | AATGTTCTTAAGGCAGTTGCTCAAATGGGACTGGGATCAGAAGTGATTACATACATACCACCAGA |
| | AGGGCATCTGGTGACGCGATTTATAGAGGGTAGGCATTTTTCTCTAGAGGAAATTACACAACCGG |
| | AAAACATACATCGAATTGCCCAGATCCTAAAGCAAGTTCATAGTATTGAGGGGATTGACGCTGCA |
| | CCGCCACCATTTGAGCGAATTGAGGGGCTGATGAGGAACGCTGAACGCAAAAATGGCATATTTCC |
| | TGACGATTTTGAACAGCTACTCGCTAATCTACAAGCTATAAAGGCTGTACTGACTAAAGTCCACA |
| | GAAAACCATGTCTGTGTCATAACGACTTGGCTGGATCGAATATCATCGAGTCCGATGGTTCAATT |
| | TTCCTAATCGATTGGGAGTACGCAGGTCTAGCAAATCCGATGTTTGATCTAGCTAACTTTTCTAT |
| | GAATCAGAACCTGGATAGAGAAGGTGATCAAATTCTCATTGAGAGCTACTTTGGGAAAGTTAGTC |
| | AGATGGAGTTAGCAGAAATCAATCTTTGGAAGATAGTTGCTATCTTTATTGAAGGTGTTTGGGGT |
| | ATTTTACAGGCGAAAATTTCTCAATTTGATCGGGACTATCAAGGCTTTGCTAACGAAAATTGGCA |
| | AACAGTGCGTCAGTACACGCAAAAGGAAAGCTTTTCACAATGGCTCTCTATGATATCGTCATCAA |
| | ACCACTGA |
| 66 | TTGAAAAAATCCATTGAGCAGATAATTGCCAACTTACGGCTATGGGATACACAGGGTGATCTGAA |
| | AATTGCGCCCATCGGTGACCATAACTTCACTAATCAGAATTACCGAGTTGAGAGCAATGGACAGG |
| | TATTCAAAGTTCGCATCAGCGAAGGAAATCAAAACCTAATAGGGAGTGCTAGGGAAGAAGAACTA |
| | ATTGTTCTTCAAACAGTTGCTAAAATGGGCATAGGACCAGAGGTGATTGCGTACATACCACCAGA |
| | GGGACATCTGGTGACGCGATTTATAGAGGGTAAGCATTTTTCTCTACGAGAAATCACGCAATCGG |
| | AAAACATATATCGAATTGTCCAGATTCTCAAGCAAGTTCATGCTATTGAGGGGATTGACGCAGTA |
| | CCGTCACCATTTGAGCGAATTGAAGGGCTAATAAGGAACGCGGAACGCAAAAATGCCATATTTCC |
| | TGATGATTTTAAACAGCTACTGGCTAATCTACAAGCTATAAAGGCTGTACTGACTAAAGTGTACA |
| | GAAAACCATGTCTGTGTCATAACGACTTGGCCAGATCAAATATCATCGAGTCCAATGGTTCAATT |
| | TTCCTCATCGATTGGGAGTACGCAGGTATGGGAAATCCGATGTTTGATCTAGCTAACTTTTCTAT |
| | GAATCAGAACCTGGACACAGAAGGTGATCAAATTCTCATTGAGAGCTACTTTGGGAAAGTCAGTC |
| | CAGTGGAGTTAGCAGAAATCAATCTTTGGAAGATAGTTTCCATCTTTATTGAAGCTGTTTGGGGA |
| | ATTTTGCAGGCGAAAATTTCTCAATTTGATCGGAACTATCAAGGTTTTTCTAACGAGAATTGGCA |
| | AACAGTGCGTCAGTACATACAAAAGGAAAGCTTTTCAGAATGGCTGTCCATAGTATCGTTCTCAT |
| | CAAACAACTGA |
| 67 | TTGAAAAAATCCATAGACAAGATAATTACCAACTTACCGCTATGGGATACACAAGATGCTCTGAA |
| | AATTATGCCAATTGGTGATCAAAACTTCACCAATCAGAATTACCGAGTTGAGGGCAACGGACAGG |
| | TATTCAAGATTCGCATCAGCGAAGGAAATCAAAACCTAATAGGGATTGCTAGGGAAGAAGAACTA |
| | AATGTTCTTAAGGCAGTTGCTAAAATGGGTATAGGATCAGAAGTGATTGCATACATACCACCAGA |
| | AGGGCATCTGGTGACGCGATTTATAGAGGGTAGGCATTTTTCTCTAGAGGAAATTACACAACCGG |
| | AAAACATACATCGAATTGCCCAGATCCTAAAGCAAGTTCATAGTATTGAGGGGATTGACGCTGCA |
| | CCGCCACCATTTGAGCGAATTGAGGGGCTGATGAGGAACGCTGAACGCAAAAATGGCATATTTCC |
| | TG |
| 68 | ATGGTAAAATCAAATATTTTAAGTCGTAATGTTGTAGGTTATAATTCCACTGATTTAAGTTCAGA |
| | TCACATTCAGGCAGAAGCACGTGATTTTTATGCAAGACAAGCAGGAGTATTTGCTAAGATTCTCA |
| | ACGAATCTTGGTCAGAATACACGAATATTTCTCATCCTGGCTTCAAAAATGATCTCGACCTCATT |
| | CGTCAAGTAAAAGAACTTGTTGCTCCTGGTGCAAAAGGAATTGATGCTGGTTGTGGTCCTCTTGC |
| | CCGGGAGGTATTCTGCTTTTATGAAGATGGTTATGATATGTATGGTATGGATGTAGTTCCAGAAA |
| | ATATCCAAACGGCACTGGAGTATCATCCTTCATTGGCTGATCGGCTTTTTTTGGCTGATTTGACG |
| | CAGCCACTTGATGTCCCTGATCATTCCTTTGATTTTATTATTTGCAATACAGTAATTCAACACAT |
| | TAATCTCAATTCTTTAACTAATGTAACCTTTCCAGAGTGGACACGAATACTTAAGCCTGGTGGAA |
| | TCATTCAGTTAATGTTTAGGTCTGGTCATGGAACTCAAATTATTCATGATGTAGAATTTAACGAT |
| | CAACGTAGTTTTTATCTGTATCAACCAGAGGAAATTCTAGAAATACTGGCGAGATATGGATTGTC |
| | TTTAATTCCAGAAGAGAATGGCAATTTAGGAGGAATTATTTATGTTACTGATGTCAGACCAATGG |
| | AAAATTGTATTTTCTGTGTGCGGAAGACAGATTAA |
| 69 | ATGGTACAACCAGAGATTTTAAATCGTAATGTTGTAGGTCATAAGTCTACTGATTTAAGTTTGGA |
| | TCAAATTCAGGCAGAAGCGCGTGATTTTTATGCAAGACAAGCAGGAGTATTTGCTAAGATTCTAA |
| | ACGAATCTTGGTCTGAATACACGAATATTTCTCATCCTGGATTCAAAAATGATCTTGACCTCATT |
| | CGTCAAGTAAAACAATTTATTGCTCCTGGTGCAAAAGGAATTGATGCTGGTTGTGGTCCTCTTGC |
| | CCGTGAGGTATTCTGCTTTTATGAAGATGGTTATGATATGTATGGCATGGATGTAGTGCCAGAAA |
| | ATATCCAAACGGCACTGGAGTATCATCCTTCATTGGCTGATCGGCTTTTTTTGGCTGATTTGACG |
| | CAACCACTTGATTTCCCTGATAATTCCTTTGATTTTATTATTTGCAATACAGTCATTCAACACAT |
| | TAATCTCAATTCTTTAACTAATGTAACCTTTCCAGAGTGGACACGAATACTTAAGCCTGGTGGAA |
| | TCATTCAGTTAATGTTTAGGTCTGGTCATGGAACTCAAACTATTCAGGATGTAGAATTTAACGAT |
| | CAACGTAGTTTTTATCTGTATCAACCACAGGAAATTCTAGAAATACTTGCGCGATATGGATTGTC |
| | TTTAATTCCAGAAGAGAATGGCAATTTAGGAGGAATTATTTATGTTACTGATGTCAGACCAATGG |
| | AAAATTGTATTTTCTGTGTGCGGAAGACACATGATTGA |
| 70 | ATGGTACAACCAGAGATTTTAAATCGTAATGTTGTAGGTCATAAGTCTACTGATTTAAGTTTGGA |
| | TCAAATTCAGGCAGAAGCGCGTGATTTTTATGCAAGACAAGCAGGAGTATTTGCTAAGATTCTAA |
| | ACGAATCTTGGTCTGAATACACGAATATTTCTCATCCTGGATTCAAAAATGATCTTGACCTCATT |
| | CGTCAAGTAAAACAATTTATTGCTCCTGGTGCAAAAGGAATTGATGCTGGTTGTGGTCCTCTTGC |
| | CCGTGAGGTATTCTGCTTTTATGAAGATGGTTATGATATGTATGGCATGGATGTAGTGCCAGAAA |
| | ATATCCAAACGGCACTGGAGTATCATCCTTCATTGGCTGATCGGCTTTTTTTGGCTGATTTGACG |
| | CAACCACTTGATTTCCCTGATAATTCCTTTGATTTTATTATTTGCAATACAGTCATTCAACACAT |
| | TAATCTCAATTCTTTAACTAATGTAACCTTTCCAGAGTGGACACGAATACTTAAGCCTGGTGGAA |
| | TCATTCAGTTAATGTTTAGGTCTGGTCATGGAACTCAAACTATTCAGGATGTAGAATTTAACGAT |
| | CAACGTAGTTTTTATCTGTATCAACCACAGGAAATTCTAGAAATACTTGCGCGATATGGATTGTC |
| | TTTAATTCCAGAAGAAAATGGCAATTTAGGAGGAATTATTTATGTTACTGATGTCAGACCAATGG |
| | AAAATTGTATTTTCTGTGTGCGGAAGACACATGATTGA |
| 71 | ATGGTCAAATCAAATATTTTAAGTCGTAATGTTGTAGGTTGTAATTCCACTGATTTAAGTTCGGA |
| | TCAAATTCAGGCAGAAGCGCGTGATTTTTATGCAAGACAAGCGGGAGTATTTGCTAAGATTCTCA |
| | AGGAATCTTGGTCAGAATACACGAATATTTCTCATCCTGGATTCAAAAATGATCTCGACCTGATT |
| | CGTCAAGTAAAAGAACTTGTTGCTCCTGGTGCAAAAGGAATTGATGCTGGTTGTGGTCCTCTTGC |
| | CCGTGAGGTATTCTGGTTTTATGAAGATGGTTATGATATGTATGGTATGGATGTAGTTCCAGAAA |
| | ATATCCAAACGGCACTGGAGTATCATCCTTCATTGACTGATCGGCTTTTTTTAGCTGATTTGACT |
| | CAGCCACTTGATTTCCCTGATCATTCCTTTGATTTTATTATTTGCAATACGGTAATTCAACACAT |
| | TAATCTCAATACTTTAACTAATGTAACCTTTCCAGAGTGGACACGAATACTTAAGCCTGGTGGAA |
| | TCATTCAGCTAATGTTTAGGTCTGGTCATGGAACTCAAATTATTCAGGATGTAGAATTTAATGAT |
| | CAACGTAGTTTCTATCTGTATCAACCAGAGGAAATTCTAGAAATACTGGCGAGATATGGATTGTC |
| | TTTAATTCCAGAAGAGAATGGCAATTTAGGAGGAATTATTTATGTTACTGATGTCAGACCAATGG |
| | AAAATTGTATTTTCTGTGTGCGGAAGATAGATGATTGA |
| 72 | ATGGTACAACCAGAGATTTTAAATCGTAATGTTGTAGGTCATAAGTCTACTGATTTAAGTTTGGA |
| | TCAAATTCAGGCAGAAGCGCGTGATTTTTATGCAAGACAAGCAGGAGTATTTGCTAAGATTCTAA |
| | ACGAATCTTGGTCTGAATACACGAATATTTCTCATCCTGGATTCAAAAATGATCTTGACCTCATT |
| | CGTCAAGTAAAACAATTTATTGCTCCTGGTGCAAAAGGAATTGATGCTGGTTGTGGTCCTCTTGC |
| | CCGTGAGGTATTCTGCTTTTATGAAGATGGTTATGATATGTATGGCATGGATGTAGTGCCAGAAA |
| | ATATCCAAACGGCACTGGAGTATCATCCTTCATTGGCTGATCGGCTTTTTTTGGCTGATTTGACG |
| | CAACCACTTGATTTCCCTGATAATTCCTTTGATTTTATTATTTGCAATACAGTCATTCAACACAT |
| | TAATCTCAATTCTTTAACTAATGTAACCTTTCCAGAGTGGACACGAATACTTAAGCCTGGTGGAA |
| | TCATTCAGTTAATGTTTAGGTCTGGTCATGGAACTCAAACTATTCAGGATGTAGAATTTAACGAT |
| | CAACGTAGTTTTTATCTGTATCAACCACAGGAAATTCTAGAAATACTTGCGCGATATGGATTGTC |
| | TTTAATTCCAGAAGAGAATGGCAATTTAGGAGGAATTATTTATGTTACTGATGTCAGACCAATGG |
| | AAAATTGTATTTTCTGTGTGCGGAAGACACATGATTGA |
| 73 | ATGTATTTGACACTCTTCAAACAGTATGACTTTCTGAACCGATTTATTCGGTTATCGGTTGCCAA |
| | TATAGTCTCCAATCTCATGATCCCTCTAGCTGGTTCCATCAGTCTAGTTTTTTTGGGTCATCTCT |
| | CAGGAGTCCATGATTTGGCCGGATCATCTTTTGCAGTAGTATTATTTGATTTTATCTACTACGGT |
| | TTTTACTTTTTACGACAGAGTACCACTGGCGTTACCGCTCAGGCAATAGGAAGAGAAGACCAGGA |
| | GGAGATGTTACTGATAGGACTGCGAAATATCTTAATTGCTTTAGTCTTAGGCATACTGCTTTTGA |
| | TTTTGCAGTACCCATTTAGGGAGATAGCATTTTCCATGTTAAATGATACTCCAGAGGTTAAAGCT |
| | TCGGCGGTTGCCTATTTCAATGCTCGGATTATTGGTGCGCCTGCGGTTTTCATCAATTACGTTCT |
| | GATTGGTTGTTTGCTAGGAAGGGAAGAGAGCGGTAAGGTGTTACTAATATCAGTAATGGGAAATG |
| | CTGTCAACATATTACTAGACTATTTGCTGATTATCCATTGGGGTTGGGGAGCTACAGGAGCTGGA |
| | CTCTCTATGGCTATTAGTCAGTATTTAATGTTATTGGTAGGATTAATATTTTTTTGCCTTCAAAT |
| | CCAGTGGCAAATGTTAAAAGCTATAACTGGAAAACTTTTGGATTGGTCAGCTTTGAAATCTGCCT |
| | TTGTCCTGAATGGCAATATATTTGTTAGTACCTTGATCCTGATCTCCACCACTGATATATTCAGT |
| | TTAGAGAGCAATACAATGGGGACAATCATCTTTACTCAAAATACCTTGATATTACAAATCATTTT |
| | CCTAGCTCTCTATTTTGTTGAAGGATTAGGATTAGCTACAGAGAGTTTAGTAGGCTACTTTACAG |
| | GTCAAGGTGCGAAGGAAAAGTTAGCTTCTTTGGTAGGGCTTTCTGCACAAGCTAGTCTTGTTTTG |
| | GGACTCGCCTTTGCTCTTGTACCTGCGCTTTTTCCTCAATGGGTATTTGGGTTATTCACCGACCA |
| | TATTGAAATAATTAGTCAAATTGATACATATATTTGGTGGTTACCACTTTTTTTGACATTTAGTT |
| | CAGTAGCACTTGCACTGGAAGGATACTTTCTAGGTTTAGCAGAAGGACGCACTCTTCGCAATATT |
| | AACTTAACTGCCATTATTTTAGGATTTATGCCTTTAAGTATAGCCGCTTGGCAGTTTCACAATAA |
| | CCATTTGTTGTGGCTTGCTTATTCTATGTTTATGTGCATCAGAATGGTGATGCTTAGTATACAAG |
| | TACCAAGAACTTTTGAAAGTGATTTTGGAGAGCGAAAAATTTTGGGGGAGATGGAGTAG |
| 74 | TTGATATTACAAATCATTTTCCTAGCTCTCTATGTTGTTGAAGGATTAGGATTAGCTACAGAGAG |
| | TTTAGTAGGCTACTTTACAGGTCAAGGTGCGAAGGAAAAGTTAGCTTCTTTGGTAAGGCTTTCTG |
| | GACAAGCTAGTCTTTTTTTGGGACTTGCCTTTGCTCTTGTACCTGCACTTTTTCCTCAAAGGATA |
| | TTTGGGTTGTTCACCGACCACGTTGAAATAATTAGTCAAATTGATACATATATTTGGTGGTTACC |
| | ACTTTTTTTGACGTTTAGTTCAGTAGCACTTGCACTGGAAGGATACTTTCTGGGTTTAGCAGAAG |
| | GACGTACTCTTCGCAACATTAACTTAACTGCCATTACTTTAGGATTTGCGCCTTTAAGTATAGTC |
| | GCTTGGCAGTTTCACAATAACCATTTGTTGTGGCTGGCTTATTCTATGTTCATGTGCATCAGAAT |
| | GGTGATGCTTGGTATACAAGTCCCAAAAACTTTTGAAAGTGATTTTGACACTCTCCAGGCTGAAG |
| | CAACGGAGATTCTTAAGTAG |
| 75 | ATGCATTTGACACTCTTCAAACAGTATGACTTTCTTAACCGATTTATTCGGTTATCGCTTGCCAA |
| | TATAATCTCCAATCTCATGATCCCGCTAGCTGGTTCAATCAGTCTAGTTTTTTTAGGTCATCTGT |
| | CAGAAGTCCATGATTTGGCAGGACCATCTTTTGCAGTAGTATTATTTGATTTCATTTACTACGGG |
| | TTTTACTTTCTACGACAGAGTACCACTGGAGTTATCGCTCAGGCAATAGGACGAGAAGACCAAAG |
| | GGAAGTGTTGCTGATAGGGCTACGAAATATCTTAATTGCTTTAGTTTTAGGCATACTGCTTCTGA |
| | TTTTGCAGTACCCATTAAGGGAGATAGGATTTTCCATGTTAAGTGACACTCCAGAGGTAAAAGCT |
| | TCGGCGGTTGCCCATTTCAATGCTCGGATTATTGGTGCGCCTGCGGTTTTCGCCAATTACGTTCT |
| | GATTGGTTGGTTACTAGG |

TABLE 8
Protein sequences of enzymes in the guanitoxin biosynthetic pathway

| Gene Name | SEQ ID NO | Amino Acid Sequence |
|---|---|---|
| GntA | 76 | mrknpfesnl alehscyakf dgtefkiles gtkisekvte |
| | | vhaefrekil hrtppftcmg atsvfnqnny rfsmysdmct |
| | | kettlalarn lftfvneqds mnshftafia vfekpipede |
| | | hhfeklvwsq lqmlhdedsr yhdwdtqfsn dinnhkfsys |
| | | faersffiig mhpgssriar qfsypllvfn asrqfdhlve |
| | | tnqfhnfvri irerdvalqg sinpnlpaky dhsrpeepev |
| | | rqysgravek dwqcplvvhq kkhkhnq |
| GntB | 77 | mkknikkyrf snsiyhqikp llkldqwhcw lalaedwivi |
| | | cfaisisyyl swyfypvtil ligsrqrala tllheaahqv |
| | | iaknrtlnfm mgtffsgyfi lqtmssyrks hvekhhryfg |
| | | npdedpdykf aisqglyqqs lddktfqmly ifsplllaqv |
| | | pkyiksllih rfledqnyle lvaiglwwlt iislsilfnl |
| | | wqylilfwli pyltvfqiig wfielaehyp lmnndinlym |
| | | srnrnshfie kfltgmhnen yhlvhhlfpn ipfwnvpkah |
| | | eilmqdpnya qhhsqsggiv wsnnnapsil sgltqvss |
| GntC | 78 | mkiqpallem wlseyervpy nlgessvdnf tlgellnltg |
| | | drdaldqlsl mnndthgslr lreaiasldk svspddilvt |
| | | agtteailiy fkvryrsgan vvvpvptfhv lyetpaflgy |
| | | evrylqlrae ngfridpqel aklvddntev ivlntpqnps |
| | | gvvcseteiq siieiaekhn aeiladehyr flphdqdtei |
| | | lpslyglspk iislgstgkc fgciglrigw lignpeiika |
| | | chffkdytth tvcvlndyia agvllhkgki lpryrqmiqh |
| | | niqqfetfik qqrglidwvk peagtiafpf ftdpninski |
| | | vakrlvedhg vlllpgeafd rpshfrialg vepslfqyal |
| | | eklaiviets |
| GntD | 79 | mmsiqtlnft qseleeidil ldqvtanysh aeqpelidta |
| | | avlaqslplr lrqflhefkl retapaclik gypvndntig |
| | | atpehwaghe dakrtkreqi vfvlcsallg eifgwltqqn |
| | | aylihdvfpi kgyeqdqigs gsealllwht edayhpargd |
| | | ylglmclrnp cqsittigsv dslkkltqqq ldilfqprfi |
| | | irpdnahhad nnyqtetdld thlqnaynqi khkiisqdna |
| | | mallygepsa pylcvdpaym eatdeeakqt lqalidaidi |
| | | eimdvilqpg dicfvdnfra vhgrkpfkan ydgndrwlkr |
| | | initrdlrks ktyrshptsr iidkvps |
| GntE | 80 | mlkdspksha fferaqkvip fgvnsshryw gentpviqrg |
| | | egayvydfdg ksyidyrlgf gpvilghahp fvnqrviaai |
| | | qngvsfsatq eyevrvaehi ikmcpsvdmv rldnsgsdat |
| | | rhairlargy tgrdlvlkfe gayhgdydym lwttpdslks |
| | | rvgkrgepig hktsqgvpdl vsqllliavw ndregiekll |
| | | kekgdsiaai ivepilanag glmpepgflq flrnqcdiyg |
| | | ivlifdevkt gfriapggag eyfgvqadls ayakamgngy |
| | | pisaiggkrd immtiapgkv vhagtytgnv vvtaaadatl |
| | | eymqqqdvfg hlnhvgqqlm dgldeiltrh qinhhihglp |
| | | slfgltlses npkdwrdvvd tnlelseqll ielvnagimt |
| | | dsdpqqtwyv ceshtsedia etlnrfetal rkalqk |
| GntF | 81 | melkfqendf qsfepipyln eyfnypsdyy ggvgfeneqf |
| | | lqffaevahe yhlnnyllld fgcgptiysi lslgrncrei |
| | | hmsdylqqnl eqvqlwqqgk pqafdwypyl rralqmetal |
| | | nqnqpldsfl dvtdeeieer arllrekits irkgnaratn |
| | | pvgeegkaly eavvssfcle gvaqdlaewk nlitnlssti |
| | | kpggllifat qieadsyrig egygtvvnlt eqdivqtlln |
| | | cnfesgsikt kelkggpnht eydkflmlta rlav |
| GntG | 82 | msehfpidlr sdtvtqptps mrqamaeaiv gddvygedpt |
| | | vnrlervaae llgkesamfv psgtmgnlic mlthcgrgee |
| | | viigdqahlf lyerggsavl ggihpytvst atdgtlplse |
| | | iraavrpddi hcprtkllcv entqaqrggl plsltylhsl |
| | | rdlanelglr lhcdgarlwn asvaigveps vlsspfdsls |
| | | vclskglaap igslvigtqs fidearrlrk llgggmrqvg |
| | | aiasaglial eqmrerlaid henaqflaeg lrecgyeith |
| | | pvrtniiffr qheqdtlslq erlavwknsg llvsdfgdrt |
| | | iravthygie rkdinqaisi lqqv |
| GntH | 83 | msnnkqdinh telnpmahsr rgfftvfiag istsvssifr |
| | | kdvavaenhk aftaenksml stnnqsfffp gellasdemr |
| | | itivgsssva rktqsgtsif lelgngdsfi fdagpgvaan |
| | | ytalgvpysr mdriflshlh cdhitdlayi ygqgsmydrk |
| | | tplriwgpsg keskfgtnaf vesmkamclw hteslsyhqn |
| | | gngyetevhe fdyaqnpgva ynekgvvikh fpavhlkdga |
| | | vsyrvdwngl sfvfsgdsqp ntfmvenaqn vdilihqtip |
| | | paqvwakeyn mpleqvielq nrfdspphav gaifdqtrpr |
| | | lgvlthlyln enikvailds cqevydgpie iaqdlmvfni |
| | | skneikqria ivpelalqnt vkkyenlppp kykpreqlsq |
| | | wlldavipqe qwk |
| GntI | 84 | mkksidkiit nlplwdtqga lkivpigdqn ftnqnyrves |
| | | ngqvfkvris egnqnligia keeelivlka vakmgigpev |
| | | iayippeghl vtrfiegkhf slqeitqpen ihriaqilkq |
| | | vhsiegidaa pppferiegl mrnaerkngi fpddfeqlla |
| | | dlqaikavlt kvhrkpclch ndlagsniie adgsiflidw |
| | | eyagvanpmf dlanfsmnqn ldregdkili esyfgkvsqv |
| | | elaeinlwki vaifiegvwg vlqakisqfd rdyqgfanen |
| | | wqtvrqytqk esfsewlsmi slsnh |
| GntJ | 85 | mvksnilsrn vvgynstdls sdhiqaeard fyarqagvfa |
| | | kilneswsey tnishpgfkn dldlirqvke lvapgakgid |
| | | agcgplarev fcfyedgydm ygmdvvpeni qtaleyhpsl |
| | | adrlfladlt qpldvpdhsf dfiicntviq hinlnsltnv |
| | | tfpewtrilk pggiiqlmfr sghgtqiihd vefndqrsfy |
| | | lyqpeeilei laryglslip eengnlggii yvtdvrpmen |
| | | cifcvrktd |
| GntT | 86 | myltlfkqyd flnrfirlsv anivsnlmip lagsislvfl |
| | | ghlsgvhdla gssfavvlfd fiyygfyflr qsttgvtaqa |
| | | igredqeeml liglrnilia lvlgilllil qypfreiafs |
| | | mlndtpevka savayfnari igapavfiny vligcllgre |
| | | esgkvllisv mgnavnilld ylliihwgwg atgaglsmai |
| | | sqylmllvgl iffclqiqwq mlkaitgkll dwsalksafv |
| | | lngnifvstl ilisttdifs lesntmgtii ftqntlilqi |
| | | iflalyfveg lglateslvg yftgqgakek laslvglsaq |
| | | aslvlglafa lvpalfpqwv fglftdhiei isqidtyiww |
| | | lplfltfssv alalegyflg laegrtlrni nltaiilgfm |
| | | plsiaawqfh nnhllwlays mfmcirmvml siqvprtfes |
| | | dfgerkilge me |

TABLE 9
Proposed functions and similarity for proteins in the Gnt biosynthetic
gene cluster (BGC). Gnt protein sequences were compared by Basic Local
Alignment Search Tool (BLAST) against publicly available data.

| | | | | AA | |
|---|---|---|---|---|---|
| | Amino | Proposed | Protein Homolog and | Similarity | Accession |
| Protein | Acids | Function | Origin | (%) | number |
| GntA | 267 | N-hydroxylase | Yqcl/YcgG family | 77% | WP_096685072.1 |
| | | | protein [Calothrix] | | |
| GntB | 318 | Arginine | Fatty acid desaturase | 52% | WP_096589832.1 |
| | | hydroxylase | [Calothrix sp. NIES- | | |
| | | | 2098] | | |
| GntC | 370 | Hydroxyarginine | Aminotransferase class | 62% | WP_096589833.1 |
| | | PLP-dependent | I/II-fold pyridoxal | | |
| | | Cyclase | phosphate-dependent | | |
| | | | enzyme [Calothrix sp. | | |
| | | | NIES-2098] | | |
| GntD | 347 | Enduracididine | arginine b-hydroxylase | 45% | WP_127087020.1 |
| | | b-hydroxylase | Fe(II)/a-ketoglutarate | | |
| | | | dependent enzyme | | |
| | | | [Calothrix desertica] | | |
| GntE | 436 | Aminotransferase | Aminotransferase class | 80% | WP_149684313.1 |
| | | PLP-dependent | III-fold pyridoxal | | |
| | | | phosphate-dependent | | |
| | | | enzyme | | |
| | | | [gammaproteobacterium | | |
| | | | BW-2] | | |
| GntF | 274 | N- | N-methyltransferase | 30% | WP_006513898.1 |
| | | methyltransferase | [Leptolyngbya sp. PCC | | |
| | | SAM-dependent | 7375] | | |
| GntG | 344 | Aldolase | Low-specificity L- | 63% | WP_060687126.1 |
| | | | threonine aldolase | | |
| | | | [Ardenticatena maritima] | | |
| GntH | 413 | Phosphatase | MBL fold metallo- | 45% | WP_052224966.1 |
| | | | hydrolase | | |
| | | | [Mesorhizobium sp. F7] | | |
| GntI | 249 | Kinase | Choline/Ethanolamine | 31% | KXK14598.1 |
| | | | kinase [Chloroflexi | | |
| | | | bacterium OLB14] | | |
| GntJ | 249 | O- | SAM-dependent | 44% | PZC47808.1 |
| | | methyltransferase | methyltransferase | | |
| | | SAM-dependent | [Chloroflexi bacterium] | | |
| GntT | 452 | Transporter | MATE family efflux | 57% | KYC3624.1 |
| | | | transporter [Scytonema | | |
| | | | hofmannii PCC 7110] | | |

TABLE 10
Metagenomic and metatranscriptomic sequencing datasets
for Gnt biosynthetic gene cluster (BGC)

| Description | Dataset identifier |
|---|---|
| Lake Erie, Ohio metatranscriptomics | SRR1601415, |
| | SRR1601416, |
| | SRR1601417 |
| Lake Mendota, Wisconsin | SRR3987090 |
| metagenomics | |
| Amazon river metagenomics | SRR5675727 |
| Sandusky Bay, Lake Erie, Ohio | SRR5249015 |
| metatranscriptomics | |

### REFERENCES

(1) Ho, J. C.; Michalak, A. M.; Pahlevan, N. Widespread global increase in intense lake phytoplankton blooms since the 1980s. Nature 2019, 574 (7780), 667 670.
(2) Huisman, J.; Codd, G. A.; Paerl, H. W.; Ibelings, B. W.; Verspagen, J. M. H.; Visser, P. M. Cyanobacterial blooms. Nat. Rev. Microbiol. 2018, 16 (8), 471 483.
(3) Plaas, H. E.; Paerl, H. W. Toxic cyanobacteria: A growing threat to water and air quality. Environ. Sci. Technol. 2021, 55 (1), 44 64.
(4) Jochimsen, E. M.; Cookson, S. T. Liver failure and death after exposure to microcystins at a hemodialysis center in Brazil. N. Engl. J. Med. 1998, 338, 873-878.
(5) Mishra, D. R.; Kumar, A.; Ramaswamy, L.; Boddula, V. K.; Das, M. C.; Page, B. P.; Weber, S. J. CyanoTRACKER: A cloud-based integrated multi-platform architecture for global observation of cyanobacterial harmful algal blooms. Harmful Algae 2020, 96, 101828.
(6) Rousso, B. Z.; Bertone, E.; Stewart, R.; Hamilton, D. P. A systematic literature review of forecasting and predictive models for cyanobacteria blooms in freshwater lakes. Water Res. 2020, 182, 115959.
(7) Seegers, B. N.; Werdell, P. J.; Vandermeulen, R. A.; Salls, W.; Stumpf, R. P.; Schaeffer, B. A.; Owens, T. J.; Bailey, S. W.; Scott, J. P.; Loftin, K. A. Satellites for long-term monitoring of inland U.S. lakes: The MERIS time series and application for chlorophyll-a. Remote Sens. Environ. 2021, 266, 112685.
(8) Baker, L.; Sendall, B. C.; Gasser, R. B.; Menjivar, T.; Neilan, B. A.; Jex, A. R. Rapid, multiplex-tandem PCR assay for automated detection and differentiation of toxigenic cyanobacterial blooms. Mol. Cell. Probes 2013, 27 (5 6), 208 214.
(9) Tillett, D.; Dittmann, E.; Erhard, M.; von Döhren, H.; Börner, T.; Neilan, B. A. Structural organization of microcystin biosynthesis in Microcystis aeruginosa PCC7806: An integrated peptide polyketide synthetase system. Chem. Biol. 2000, 7 (10), 753 764.
(10) Mihali, T. K.; Kellmann, R.; Muenchhoff, J.; Barrow, K. D.; Neilan, B. A. Characterization of the gene cluster responsible for cylindrospermopsin biosynthesis. Appl. Environ. Microbiol. 2008, 74 (3), 716 722.
(11) Kellmann, R.; Mihali, T. K.; Jeon, Y. J.; Pickford, R.; Pomati, F.; Neilan, B. A. Biosynthetic intermediate analysis and functional homology reveal a saxitoxin gene cluster in cyanobacteria. Appl. Environ. Microbiol. 2008, 74 (13), 4044 4053.
(12) Méjean, A.; Mann, S.; Maldiney, T.; Vassiliadis, G.; Lequin, O.; Ploux, O. Evidence that biosynthesis of the neurotoxic alkaloids anatoxin-a and homoanatoxin-a in the cyanobacterium Oscillatoria PCC 6506 occurs on a modular polyketide synthase initiated by L-proline. J. Am. Chem. Soc. 2009, 131 (22), 7512 7513.
(13) Fiore, M. F.; de Lima, S. T.; Carmichael, W. W.; McKinnie, S. M. K.; Chekan, J. R.; Moore, B. S. Guanitoxin, re-naming a cyanobacterial organophosphate toxin. Harmful Algae 2020, 92, 101737.
(14) Hyde, E. G.; Carmichael, W. W. Anatoxin-a(s), a naturally occurring organophosphate, is an irreversible active site-directed inhibitor of acetylcholinesterase (EC 3.1.1.7). J. Biochem. Toxicol. 1991, 6 (3), 195 201.
(15) Carmichael, W. W. Health effects of toxin-producing cyanobacteria: “The CyanoHABs.” Hum. Ecol. Risk Assess. Int. J. 2001, 7 (5), 1393 1407.
(16) Henriksen, P.; Carmichael, W. W.; An, J.; Moestrup, Ø. Detection of an anatoxin-a(s)-like anticholinesterase in natural blooms and cultures of cyanobacteria/blue-green algae from Danish lakes and in the stomach contents of poisoned birds. Toxicon 1997, 35 (6), 901 913.
(17) Chatziefthimiou, A. D.; Richer, R.; Rowles, H.; Powell, J. T.; Metcalf, J. S. Cyanotoxins as a potential cause of dog poisonings in desert environments. Vet. Rec. 2014, 174 (19), 484 485.
(18) Blin, K.; Shaw, S.; Kloosterman, A. M.; Charlop-Powers, Z.; van Wezel, G. P.; Medema, M. H.; Weber, T. antiSMASH 6.0: improving cluster detection and comparison capabilities. Nucleic Acids Res. 2021, 49 (W1), W29 W35.
(19) Krzywinski, M.; Schein, J.; Birol, t.; Connors, J.; Gascoyne, R.; Horsman, D.; Jones, S. J.; Marra, M. A. Circos: An information aesthetic for comparative genomics. Genome Res. 2009, 19 (9), 1639 1645.
(20) Gilchrist, C. L. M.; Chooi, Y.-H. Clinker& clustermap.js: Automatic generation of gene cluster comparison figures. Bioinformatics 2021, 37 (16), 2473 2475.
(21) Carmichael, W. W.; Gorham, P. R. Anatoxins from clones of Anabaena flos-aquae isolated from lakes of western Canada. Int. Ver. Für Theor. Angew. Limnol. 1978, 21 (1), 285 295.
(22) Matsunaga, S.; Moore, R. E.; Niemczura, W. P.; Carmichael, W. W. Anatoxin-a(s), a potent anticholinesterase from Anabaena flos-aquae. J. Am. Chem. Soc. 1989, 111 (20), 8021 8023.
(23) Fernandes, K.; Dorr, F. A.; Pinto, E. Stability analyses by HPLC-MS of guanitoxin isolated from Sphaerospermopsis torques-reginae. J. Braz. Chem. Soc. 2021, 32, 1559-1567.
(24) Moore, B. S.; Ohtani, I.; de Koning, C. B.; Moore, R. E.; Carmichael, W. W. Biosynthesis of anatoxin-a(s). Origin of the carbons. Tetrahedron Lett. 1992, 33 (44), 6595 6598.
(25) Hemscheidt, T.; Burgoyne, D. L.; Moore, R. E. Biosynthesis of anatoxin-a(s). (2S,4S)-4-Hydroxyarginine as an intermediate. J. Chem. Soc. Chem. Commun. 1995, 2, 205-206.
(26) Lima, S. T.; Alvarenga, D. O.; Etchegaray, A.; Fewer, D. P.; Jokela, J.; Varani, A. M.; Sanz, M.; Dorr, F. A.; Pinto, E.; Sivonen, K.; Fiore, M. F. Genetic organization of anabaenopeptin and spumigin biosynthetic gene clusters in the cyanobacterium Sphaerospermopsis torques-reginae ITEP-024. ACS Chem. Biol. 2017, 12 (3), 769 778.
(27) Molica, R. J. R.; Oliveira, E. J. A.; Carvalho, P. V. V. C.; Costa, A. N. S. F.; Cunha, M. C. C.; Melo, G. L.; Azevedo, S. M. F. O. Occurrence of saxitoxins and an anatoxin-a(s)-like anticholinesterase in a Brazilian drinking water supply. Harmful Algae 2005, 4 (4), 743 753.
(28) He, H.; Williamson, R. T.; Shen, B.; Graziani, E. I.; Yang, H. Y.; Sakya, S. M.; Petersen, P. J.; Carter, G. T. Mannopeptimycins, novel antibacterial glycopeptides from Streptomyces hygroscopicus, LL-AC98. J. Am. Chem. Soc. 2002, 124 (33), 9729 9736.
(29) Ling, L. L.; Schneider, T.; Peoples, A. J.; Spoering, A. L.; Engels, I.; Conlon, B. P.; Mueller, A.; Schäberle, T. F.; Hughes, D. E.; Epstein, S.; Jones, M.; Lazarides, L.; Steadman, V. A.; Cohen, D. R.; Felix, C. R.; Fetterman, K. A.; Millett, W. P.; Nitti, A. G.; Zullo, A. M.; Chen, C.; Lewis, K. A new antibiotic kills pathogens without detectable resistance. Nature 2015, 517 (7535), 455 459.
(30) Asai, M.; Muroi, M.; Sugita, N.; Kawashima, H.; Mizuno, K.; Miyake, A. Enduracidin, a new antibiotic. II Isolation and characterization. J. Antibiot. (Tokyo) 1968, 21 (2), 138 146.
(31) Han, L.; Schwabacher, A. W.; Moran, G. R.; Silvaggi, N. R. Streptomyces wadayamensis MppP is a pyridoxal 5′-phosphate-dependent L-arginine a-deaminase, y-hydroxylase in the enduracididine biosynthetic pathway. Biochemistry 2015, 54 (47), 7029 7040.
(32) Han, L.; Vuksanovic, N.; Oehm, S. A.; Fenske, T. G.; Schwabacher, A. W.; Silvaggi, N. R. Streptomyces wadayamensis MppP is a PLP-dependent oxidase, not an oxygenase. Biochemistry 2018, 57 (23), 3252 3264.
(33) Burroughs, A. M.; Hoppe, R. W.; Goebel, N. C.; Sayyed, B. H.; Voegtline, T. J.; Schwabacher, A. W.; Zabriskie, T. M.; Silvaggi, N. R. Structural and functional characterization of MppR, an enduracididine biosynthetic enzyme from Streptomyces hygroscopicus: Functional diversity in the acetoacetate decarboxylase-like superfamily. Biochemistry 2013, 52 (26), 4492 4506.
(34) Nayfach, S.; Roux, S.; Seshadri, R.; Udwary, D.; Varghese, N.; Schulz, F.; Wu, D.; Paez-Espino, D.; Chen, I.-M.; Huntemann, M.; Palaniappan, K.; Ladau, J.; Mukherjee, S.; Reddy, T. B. K.; Nielsen, T.; Kirton, E.; Faria, J. P.; Edirisinghe, J. N.; Henry, C. S.; Jungbluth, S. P.; Chivian, D.; Dehal, P.; Wood-Charlson, E. M.; Arkin, A. P.; Tringe, S. G.; Visel, A.; IMG/M Data Consortium; Woyke, T.; Mouncey, N. J.; Ivanova, N. N.; Kyrpides, N. C.; Eloe-Fadrosh, E. A. A genomic catalog of arth's microbiomes. Nat. Biotechnol. 2021, 39 (4), 499 509.
(35) Du, Y.-L.; Ryan, K. S. Pyridoxal phosphate-dependent reactions in the biosynthesis of natural products. Nat. Prod. Rep. 2019, 36 (3), 430 457.
(36) Rudolph, J.; Hannig, F.; Theis, H.; Wischnat, R. Highly efficient chiral-pool synthesis of (2S,4R)-4-hydroxyornithine. Org. Lett. 2001, 3 (20), 3153 3155.
(37) Giltrap, A. M.; Dowman, L. J.; Nagalingam, G.; Ochoa, J. L.; Linington, R. G.; Britton, W. J.; Payne, R. J. Total synthesis of teixobactin. Org. Lett. 2016, 18 (11), 2788 2791.
(38) Yin, X.; Zabriskie, T. M. VioC is a non-heme iron, a-ketoglutarate-dependent oxygenase that catalyzes the formation of 3S-hydroxy-L-arginine during viomycin biosynthesis. ChemBioChem 2004, 5 (9), 1274 1277.
(39) Ju, J.; Ozanick, S. G.; Shen, B.; Thomas, M. G. Conversion of (2S)-arginine to (2S,3R)-capreomycidine by VioC and VioD from the viomycin biosynthetic pathway of Streptomyces sp. strain ATCC1 1861. ChemBioChem 2004, 5 (9), 1281 1285.
(40) Yin, X.; McPhail, K. L.; Kim, K.; Zabriskie, T. M. Formation of the nonproteinogenic amino acid 2S,3R-capreomycidine by VioD from the viomycin biosynthesis pathway. ChemBioChem 2004, 5 (9), 1278 1281.
(41) Chang, C.-Y.; Lyu, S.-Y.; Liu, Y.-C.; Hsu, N.-S.; Wu, C.-C.; Tang, C.-F.; Lin, K.-H.; Ho, J.-Y.; Wu, C.-J.; Tsai, M.-D.; Li, T.-L. Biosynthesis of streptolidine involved two unexpected intermediates produced by a dihydroxylase and a cyclase through unusual mechanisms. Angew. Chem. Int. Ed. 2014, 53 (7), 1943 1948.
(42) Haltli, B.; Tan, Y.; Magarvey, N. A.; Wagenaar, M.; Yin, X.; Greenstein, M.; Hucul, J. A.; Zabriskie, T. M. Investigating B-hydroxyenduracididine formation in the biosynthesis of the mannopeptimycins. Chem. Biol. 2005, 12 (11), 1163 1168.
(43) Kumagai, T.; Takagi, K.; Koyama, Y.; Matoba, Y.; Oda, K.; Noda, M.; Sugiyama, M. Heme protein and hydroxyarginase necessary for biosynthesis of D-cycloserine. Antimicrob. Agents Chemother. 2012, 56 (7), 3682 3689.
(44) ITRC (Interstate Technology & Regulatory Council), Strategies for Preventing and Managing Harmful Cyanobacterial Blooms (Interstate Technology & Regulatory Council, HCB Team, Washington D.C., 2021; www.itrcweb.org).
(45) S. T. Lima, D. O. Alvarenga, A. Etchegaray, D. P. Fewer, J. Jokela, A. M. Varani, M. Sanz, F. A. Dorr, E. Pinto, K. Sivonen, M. F. Fiore, Genetic organization of anabaenopeptin and spumigin biosynthetic gene clusters in the cyanobacterium Sphaerospermopsis torques-reginae ITEP-024. ACS Chem. Biol. 12, 769-778 (2017).
(46) P. R. Gorham, J. McLachlan, U. T. Hammer, W. K. Kim, Isolation and culture of toxic strains of Anabaena flos-aquae (Lyngb.) de Bréb. Int. Ver. Fur Theor. Angew. Limnol. Verhandlungen. 15, 796-804 (1964).
(47) F. A. Dorr, V. Rodriguez, R. Molica, P. Henriksen, B. Krock, E. Pinto, Methods for detection of anatoxin-a(s) by liquid chromatography coupled to electrospray ionization-tandem mass spectrometry. Toxicon. 55, 92-99 (2010).
(48) K. Okonechnikov, A. Conesa, F. Garcia-Alcalde, Qualimap 2: advanced multi-sample quality control for high-throughput sequencing data. Bioinformatics. 32, 292-294 (2016).
(49) B. Langmead, S. L. Salzberg, Fast gapped-read alignment with Bowtie 2. Nat Methods. 9, 357-359 (2012).
(50) W. De Coster, S. D'Hert, D. T. Schultz, M. Cruts, C. Van Broeckhoven, NanoPack: visualizing and processing long-read sequencing data. Bioinformatics. 34, 2666-2669 (2018).
(51) R. Schmieder, R. Edwards, Quality control and preprocessing of metagenomic datasets. Bioinformatics. 27, 863-864 (2011).
(52) S. Koren, B. P. Walenz, K. Berlin, J. R. Miller, N. H. Bergman, A. M. Phillippy, Canu: scalable and accurate long-read assembly via adaptive k-mer weighting and repeat separation. Genome Res. 27, 722-736 (2017).
(53) E. Haghshenas, F. Hach, S. C. Sahinalp, C. Chauve, CoLoRMap: Correcting long reads by mapping short reads. Bioinforma. Oxf. Engl. 32, i545-i551 (2016).
(54) R. R. Wick, L. M. Judd, C. L. Gorrie, K. E. Holt, Unicycler: Resolving bacterial genome assemblies from short and long sequencing reads. PLOS Comput. Biol. 13, e1005595 (2017).
(55) A. V. Zimin, D. Puiu, M.-C. Luo, T. Zhu, S. Koren, G. Marcais, J. A. Yorke, J.
Dvorak, S. L. Salzberg, Hybrid assembly of the large and highly repetitive genome of Aegilops tauschii, a progenitor of bread wheat, with the MaSuRCA mega-reads algorithm. Genome Res., 27, 787-792 (2017).
(56) M. Boetzer, C. V. Henkel, H. J. Jansen, D. Butler, W. Pirovano, Scaffolding pre-assembled contigs using SSPACE. Bioinforma. Oxf. Engl. 27, 578-579 (2011).
(57) B. J. Walker, T. Abeel, T. Shea, M. Priest, A. Abouelliel, S. Sakthikumar, C. A.
Cuomo, Q. Zeng, J. Wortman, S. K. Young, A. M. Earl, Pilon: An integrated tool for comprehensive microbial variant detection and genome assembly improvement. PLOS ONE. 9, e112963 (2014).
(58) M. Boetzer, W. Pirovano, Toward almost closed genomes with GapFiller.
Genome Biol. 13, R56 (2012).
(59) M. Kolmogorov, J. Yuan, Y. Lin, P. A. Pevzner, Assembly of long, error-prone reads using repeat graphs. Nat. Biotechnol. 37, 540-546 (2019).
(60) A. Gurevich, V. Saveliev, N. Vyahhi, G. Tesler, QUAST: quality assessment tool for genome assemblies. Bioinformatics. 29, 1072-1075 (2013).
(61) D. H. Parks, M. Imelfort, C. T. Skennerton, P. Hugenholtz, G. W. Tyson, CheckM: assessing the quality of microbial genomes recovered from isolates, single cells, and metagenomes. Genome Res. 25, 1043-1055 (2015).
(62) M. Hunt, N. D. Silva, T. D. Otto, J. Parkhill, J. A. Keane, S. R. Harris, Circulator: automated circularization of genome assemblies using long sequencing reads. Genome Biol. 16, 294 (2015).
(63) T. Seemann, Prokka: rapid prokaryotic genome annotation. Bioinformatics. 30, 2068-2069 (2014).
(64) K. Levi, M. Rynge, E. Abeysinghe, R. A. Edwards, in Proceedings of the Practice and Experience on Advanced Research Computing (ACM, Pittsburgh PA USA, 2018; https://dl.acm.org/doi/10.1145/3219104.3229278), pp. 1-7.
(65) P. J. Torres, R. A. Edwards, K. A. McNair, PARTIE: a partition engine to separate metagenomic and amplicon projects in the Sequence Read Archive. Bioinformatics. 33, 2389-2391 (2017).
(66) J. Towns, T. Cockerill, M. Dahan, I. Foster, K. Gaither, A. Grimshaw, V. Hazlewood, S. Lathrop, D. Lifka, G. D. Peterson, R. Roskies, J. R. Scott, N. Wilkins-Diehr, XSEDE: Accelerating scientific discovery. Comput. Sci. Eng. 16, 62-74 (2014).
(67) C. A. Stewart, T. M. Cockerill, I. Foster, D. Hancock, N. Merchant, E. Skidmore, D. Stanzione, J. Taylor, S. Tuecke, G. Turner, M. Vaughn, N. I. Gaffney, in Proceedings of the 2015 XSEDE Conference: Scientific Advancements Enabled by Enhanced Cyberinfrastructure (ACM, 2015; https://dl.acm.org/citation.cfin?doid=2792745.2792774), p. 29.
(68) B. Buchfink, C. Xie, D. H. Huson, Fast and sensitive protein alignment using DIAMOND. Nat. Methods. 12, 59-60 (2015).
(69) P. Di Tommaso, M. Chatzou, E. W. Floden, P. P. Barja, E. Palumbo, C. Notredame, Nextflow enables reproducible computational workflows. Nat. Biotechnol. 35, 316-319 (2017).
(70) T. Kluyver, B. Ragan-Kelley, F. Perez, B. Granger, M. Bussonnier, J. Frederic, K. Kelley, J. Hamrick, J. Grout, S. Corlay, P. Ivanov, D. Avila, S. Abdalla, C. Willing, J. development team, in Positioning and Power in Academic Publishing: Players, Agents and Agendas, F. Loizides, B. Scmidt, Eds. (IOS Press, 2016; https://eprints.soton.ac.uk/403913/), pp. 87-90.
(71) The pandas development team, pandas-dev/pandas: Pandas 1.0.3 (Zenodo, 2020; https://zenodo.org/record/3715232).
(72) W. McKinney, in Proceedings of the 9th Python in Science Conference, S. van der Walt, J. Millman, Eds. (2010), pp. 56-61.
(73) S. Choudhary, pysradb: A Python package to query next-generation sequencing metadata and data from NCBI Sequence Read Archive (2019), doi:10.12688/f1000research.18676.1.
(74) B. Grüning, R. Dale, A. Sjödin, B. A. Chapman, J. Rowe, C. H. Tomkins-Tinch, R. Valieris, J. Koster, Bioconda: sustainable and comprehensive software distribution for the life sciences. Nat. Methods. 15, 475-476 (2018).
(75) E. Bushmanova, D. Antipov, A. Lapidus, A. D. Przhibelskiy, rnaSPAdes: a de novo transcriptome assembler and its application to RNA-Seq data. GigaScience. 8, giz100 (2019).
(76) G. M. Kurtzer, V. Sochat, M. W. Bauer, Singularity: Scientific containers for mobility of compute. PLOS ONE. 12, e0177459 (2017).
(77) C. Camacho, G. Coulouris, V. Avagyan, N. Ma, J. Papadopoulos, K. Bealer, T. L. Madden, BLAST+: architecture and applications. BMC Bioinformatics. 10, 421 (2009).
(78) M. Martin, Cutadapt removes adapter sequences from high-throughput sequencing reads. EMBnet.journal. 17, 10-12 (2011).
(79) S. Nurk, D. Meleshko, A. Korobeynikov, P. A. Pevzner, metaSPAdes: a new versatile metagenomic assembler. Genome Res. 27, 824-834 (2017).
(80) D. D. Kang, J. Froula, R. Egan, Z. Wang, MetaBAT, an efficient tool for accurately reconstructing single genomes from complex microbial communities. PeerJ. 3, e1165 (2015).
(81) P.-A. Chaumeil, A. J. Mussig, P. Hugenholtz, D. H. Parks, GTDB-Tk: a toolkit to classify genomes with the Genome Taxonomy Database. Bioinforma. Oxf. Engl., btz848 (2019).
(82) D. H. Parks, M. Chuvochina, D. W. Waite, C. Rinke, A. Skarshewski, P.-A. Chaumeil, P. Hugenholtz, A standardized bacterial taxonomy based on genome phylogeny substantially revises the tree of life. Nat. Biotechnol. 36, 996-1004 (2018).
(83) M. N. Price, P. S. Dehal, A. P. Arkin, FastTree 2—Approximately maximum-likelihood trees for large alignments. PLOS ONE. 5, e9490 (2010).
(84) I. Letunic, P. Bork, Interactive Tree Of Life (iTOL) v4: recent updates and new developments. Nucleic Acids Res. 47, W256-W259 (2019).
(85) K. Clark, I. Karsch-Mizrachi, D. J. Lipman, J. Ostell, E. W. Sayers, GenBank. Nucleic Acids Res. 44, D67-D72 (2016).
(86) I. Lee, Y. Ouk Kim, S.-C. Park, J. Chun, OrthoANI: An improved algorithm and software for calculating average nucleotide identity. Int. J. Syst. Evol. Microbiol. 66, 1100-1103 (2016).
(87) R. J. R. Molica, E. J. A. Oliveira, P. V. V. C. Carvalho, A. N. S. F. Costa, M. C. C. Cunha, G. L. Melo, S. M. F. O. Azevedo, Occurrence of saxitoxins and an anatoxin-a(s)-like anticholinesterase in a Brazilian drinking water supply. Harmful Algae. 4, 743-753 (2005).
(88) A. M. Giltrap, L. J. Dowman, G. Nagalingam, J. L. Ochoa, R. G. Linington, W. J. Britton, R. J. Payne, Total synthesis of teixobactin. Org. Lett. 18, 2788-2791 (2016).
(89) S. Matsunaga, R. E. Moore, W. P. Niemczura, W. W. Carmichael, Anatoxin-a(s), a potent anticholinesterase from Anabaena flos-aquae. J. Am. Chem. Soc. 111, 8021-8023 (1989).
(90) K. Levi, M. Rynge, E. Abeysinghe, and R. A. Edwards, “Searching the Sequence Read Archive using Jetstream and Wrangler,” in Proceedings of the Practice and Experience on Advanced Research Computing, Pittsburgh PA USA, July 2018, pp. 1-7. doi: 10.1145/3219104.3229278.
(91) Stella T. Lima et al., “Biosynthesis of Guanitoxin Enables Global Environmental Detection in Freshwater Cyanobacteria,” J. Am. Chem. Soc., vol. 144, no. 21, pp. 9372-9379, June 2022, doi: 10.1021/jacs.2c01424.
(92) A. Bankevich et al., “SPAdes: A New Genome Assembly Algorithm and Its Applications to Single-Cell Sequencing,” J. Comput. Biol., vol. 19, no. 5, pp. 455-477, April 2012, doi: 10.1089/cmb.2012.0021.
(93) E. Bushmanova, D. Antipov, A. Lapidus, and A. D. Przhibelskiy, “maSPAdes: a de novo transcriptome assembler and its application to RNA-Seq data,” September 2018, doi: 10.1101/420208.
(94) T. Seemann, “Prokka: rapid prokaryotic genome annotation,” Bioinformatics, vol. 30, no. 14, pp. 2068-2069, July 2014, doi: 10.1093/bioinformatics/btu153.
(95) A. Priyam et al., “Sequenceserver: A Modern Graphical User Interface for Custom BLAST Databases,” Mol. Biol. Evol., vol. 36, no. 12, pp. 2922-2924, December 2019, doi: 10.1093/molbev/msz185.
(96) J. Ye, G. Coulouris, I. Zaretskaya, I. Cutcutache, S. Rozen, and T. L. Madden, “Primer-BLAST: A tool to design target-specific primers for polymerase chain reaction,” BMC Bioinformatics, vol. 13, no. 1, p. 134, June 2012, doi: 10.1186/1471-2105-13-134.

1. A method of detecting guanitoxin-producing bacteria in an aqueous liquid, the method comprising detecting one or more guanitoxin biosynthetic genes in the aqueous liquid, wherein the one or more guanitoxin biosynthetic genes are GntA, GntB, GntC, GntD, GntE, GntF, GntG, GntH, GntI, GntJ, GntT, or a combination thereof.

2. The method of claim 1, wherein the one or more guanitoxin biosynthetic genes are GntA, GntJ, GntC, or a combination thereof.

3. The method of claim 1, comprising contacting the aqueous liquid with one or more nucleic acids, wherein each of the one or more nucleic acids are at least partially complementary to a portion of the one or more guanitoxin biosynthetic genes.

4. The method of claim 3, wherein the detecting comprises performing a PCR method, an isothermal amplification method, a sequencing method, or a combination thereof.

5. The method of claim 3, wherein the portion of the one or more guanitoxin biosynthetic genes comprises a coding sequence, a promoter region sequence, a terminator region sequence, or an intergene region sequence.

6. The method of claim 5, wherein the portion of the one or more guanitoxin biosynthetic genes comprises a coding sequence.

7. The method of 3, wherein the one or more nucleic acids each independently comprises a sequence having at least 80% identity to any one of SEQ ID NO:1 to SEQ ID NO:22, wherein each nucleic acid of the one or more nucleic acids is different.

8. (canceled)

9. The method of claim 7, wherein the one or more nucleic acids comprises a first nucleic acid comprising a sequence having at least 80% identity to SEQ ID NO:1 and a second nucleic acid comprising a sequence having at least 80% identity to SEQ ID NO:2.

10. The method of claim 7, wherein the one or more nucleic acids comprises a first nucleic acid comprising a sequence having at least 80% identity to SEQ ID NO:3 and a second nucleic acid comprising a sequence having at least 80% identity to SEQ ID NO:4.

11. The method of claim 1, wherein the guanitoxin-producing bacteria are cyanobacteria.

12. The method of claim 11, wherein the cyanobacteria are Sphaerospermopsis torques-reginae, Chrysosporum ovalisporum, Cuspidothrix, Cylindrospermopsis, Cylindrospermum, Dolichospermum, Microcystis, Oscillatoria, Planktothrix, Phormidium, Anabaena flos-aquae, A. lemmermannii Raphidiopsis mediterranea, Tychonema, or Woronichinia.

13. The method of claim 1, wherein the aqueous liquid is derived from a lake, river, or pond.

14. (canceled)

15. The method of claim 1, wherein the aqueous liquid is ingested by, inhaled by, or contacted with a subject.

16. The method of claim 15, wherein the subject is treated for guanitoxin-induced toxicity when the one or more guanitoxin biosynthetic genes are detected.

17. A kit for detecting guanitoxin-producing bacteria in an aqueous liquid, the kit comprising one or more nucleic acids each at least partially complementary to a portion of one or more guanitoxin biosynthetic genes, wherein the one or more guanitoxin biosynthetic genes are GntA, GntB, GntC, GntD, GntE, GntF, GntG, GntH, GntI, GntJ, GntT, or a combination thereof.

18. (canceled)

19. The kit of claim 17, wherein the portion of the one or more guanitoxin biosynthetic genes comprises a coding sequence, a promoter region sequence, a terminator region sequence, or an intergene region sequence.

20. (canceled)

21. The kit of claim 17, wherein the one or more nucleic acids each independently comprises a sequence having at least 80% identity to any one of SEQ ID NO:1 to SEQ ID NO:22, wherein each nucleic acid of the one or more nucleic acids is different.

22. (canceled)

23. (canceled)

24. (canceled)

25. The kit of claim 17, wherein the guanitoxin-producing bacteria are cyanobacteria.

26. (canceled)

27. (canceled)

28. (canceled)

29. (canceled)

30. The kit of claim 17, further comprising an enzyme, deoxynucleoside triphosphates (dNTPs), a control DNA, a detectable label, or a combination thereof.

31. The kit of claim 17, further comprising a therapeutic effective for treating guanitoxin-induced toxicity.


SEQUENCE LISTING (92 sequences)

SEQ ID NO: 1 - DNA, 25 bp, synthetic construct
  source 1..25: mol_type = other DNA
ttgctgtgtttgaaaaaccaatacc

SEQ ID NO: 2 - DNA, 24 bp, synthetic construct
  source 1..24: mol_type = other DNA
ggacgactatggtcatattttgct

SEQ ID NO: 3 - DNA, 20 bp, synthetic construct
  source 1..20: mol_type = other DNA
atatccaaacggcactggag

SEQ ID NO: 4 - DNA, 20 bp, synthetic construct
  source 1..20: mol_type = other DNA
ttgagttccatgaccagacc

SEQ ID NO: 5 - DNA, 22 bp, synthetic construct
  source 1..22: mol_type = other DNA
acaagtttgcaatttctcaagg

SEQ ID NO: 6 - DNA, 20 bp, synthetic construct
  source 1..20: mol_type = other DNA
ttaaagggtaatgctcggct

SEQ ID NO: 7 - DNA, 21 bp, synthetic construct
  source 1..21: mol_type = other DNA
gcctcatgaataacgacaccc

SEQ ID NO: 8 - DNA, 20 bp, synthetic construct
  source 1..20: mol_type = other DNA
ttggtactggaacaacgaca

SEQ ID NO: 9 - DNA, 21 bp, synthetic construct
  source 1..21: mol_type = other DNA
tgtttgttttgtgcagtgctt

SEQ ID NO: 10 - DNA, 20 bp, synthetic construct
  source 1..20: mol_type = other DNA
agtagtcaccacgagcagga

SEQ ID NO: 11 - DNA, 20 bp, synthetic construct
  source 1..20: mol_type = other DNA
accaggaggagcaggtgaat

SEQ ID NO: 12 - DNA, 21 bp, synthetic construct
  source 1..21: mol_type = other DNA
tcaacaacatcacgccaatct

SEQ ID NO: 13 - DNA, 20 bp, synthetic construct
  source 1..20: mol_type = other DNA
agagcaggttcagctttggc

SEQ ID NO: 14 - DNA, 21 bp, synthetic construct
  source 1..21: mol_type = other DNA
acagaaagaagaaaccaccgc

SEQ ID NO: 15 - DNA, 19 bp, synthetic construct
  source 1..19: mol_type = other DNA
ctgtgatggagcgcgtttg

SEQ ID NO: 16 - DNA, 20 bp, synthetic construct
  source 1..20: mol_type = other DNA
gggtaactgcgcgaattgtg

SEQ ID NO: 17 - DNA, 19 bp, synthetic construct
  source 1..19: mol_type = other DNA
gggtgtaccctattcgcgg

SEQ ID NO: 18 - DNA, 20 bp, synthetic construct
  source 1..20: mol_type = other DNA
ccgtgtgccagagacacata

SEQ ID NO: 19 - DNA, 20 bp, synthetic construct
  source 1..20: mol_type = other DNA
atcgattgggagtacgcagg

SEQ ID NO: 20 - DNA, 20 bp, synthetic construct
  source 1..20: mol_type = other DNA
tactgacgcactgtttgcca

SEQ ID NO: 21 - DNA, 20 bp, synthetic construct
  source 1..20: mol_type = other DNA
tacaggtcaaggtgcgaagg

SEQ ID NO: 22 - DNA, 20 bp, synthetic construct
  source 1..20: mol_type = other DNA
tccttccagtgcaagtgcta

SEQ ID NO: 23 - DNA, 804 bp, Sphaerospermopsis torques-reginae
  source 1..804: mol_type = other DNA
atgcgaaaaaatccttttgaaagtaatctagcactggaacacagttgttatgccaagtttgatggaacagaatttaaaatacttgagagtggtacaaaaatatcagagaaggtaactgaagtacacgctgaatttcgggagaaaatattgcatcgcactcctccttttacttgtatgggtgcaacatccgtatttaaccagaataactatcgtttcagtatgtacagtgatatgtgtacaaaagaaactaccttagcattggctcgaaatttattcacttttgtgaatgaacaagatagcatgaatagtcacttcacagcatttattgctgtgtttgaaaaaccaataccagaagatgaacaccactttgaaaaactggtgtggagtcaactacaaatgttacatgatgaagatagccgttaccatgattgggacacccaattcagcaacgatatcaataatcataaattttcatacagttttgctgaaagaagctttttcattattggaatgcaccctggaagttctcgaattgctaggcaattttcctacccacttctagtatttaatgccagtcgtcaatttgatcatttagttgaaactaaccaattccataacttcgtcagaattattcgggagcgtgacgtagctctgcaaggtagtattaatcctaatctgccagcaaaatatgaccatagtcgtcctgaagaacctgaagtcagacaatattctggacgtgcagtagaaaaagattggcaatgtcctttagttgtacaccagaagaaacataagcataatcaataa

SEQ ID NO: 24 - DNA, 804 bp, Sphaerospermopsis torques-reginae
  source 1..804: mol_type = other DNA
atgccaggcaatccttttgacagtaatctagcaatggaacacagttgttatgccaaatttgatggaacagaatttaaaatcattgagagtggtacaaaaatatcaaagaaagtaactgaagtacacgctcaatttcgggagaaaatactgcatcgcactccgccttttacttgtatgggtgcaacatccgtatttaaccaaaataactatcgtttcagtatgtaccgtgagatgtgtacaaaagaaactaccttagcattggctagaaatttattcacttttgtggatgaacaagatatcatgaatagtcactttacagcatttattgctgtgtttgaaaaaccaataccacaagatgaacacgaatttgaaaaactggtgtggactcaactgcaaatgttacatgatgaagatatccgttaccatgattgggacataaaattcagcaacgatatcaataatcataagttttcatacagctttgctgaaagaagctttttcattattggaatgcaccccggaagttctcgcattgccagacaattttcctacccacttctagtatttaatgccagtcgccaatttgaccatttagttgaaactaaccaattccataacttcgtcagaattattcgggagcgtgacatggctttgcaaggtagtattaatcccaatctgccagcaaaatatgaccatagtcgtccagaagaacctgaagtcagacaatattctggacgtgcagtagaaaaagattggcagtgtcctttagttgtacaccagaagaaacatgagcataatcaataa

SEQ ID NO: 25 - DNA, 804 bp, Sphaerospermopsis torques-reginae
  source 1..804: mol_type = other DNA
atgccaggcaatccttttgacagtaatctagcaatggaacacagttgttatgccaaatttgatggaacagaatttaaaatcattgagagtggtacaaaaatatcaaagaaagtaactgaagtacacgctcaatttcgggagaaaatactgcatcgcactccgccttttacttgtatgggtgcaacatccgtatttaaccaaaataactatcgtttcagtatgtaccgtgagatgtgtacaaaagaaacaaccttagctttggctcgaaatttattcacttttgtggatgaacaagatagcatgaatagtcacttcacagcatttattgctgtgtttgaaaaaccaataccacaagatgaacacgaatttgaaaaactggtgtggactcaactgcaaatgttacatgatgaagatatccgttaccatgattgggacataaaattcagcaacgatatcaataatcatcagttttcatacagctttgctgaaagaagctttttcattattggaatgcaccccggaagttctcgcattgccagacaattttcctacccacttctagtatttaatgccagtcgccaatttgaccatttagttgaaactaaccaattccataacttcgtcagaattattcgggagcgtgacatggctttgcaaggtagtattaatcccaatttgccagcaaaatatgaccatagtcgtccagaagaacctgaagtcagacaatattctggacgtgcagtagaaaaagattggcagtgtcctttagttgtacaccagaagaaacatgagcataatcaataa

SEQ ID NO: 26 - DNA, 804 bp, Sphaerospermopsis torques-reginae
  source 1..804: mol_type = other DNA
atgcgaaaaaatccttttgaaagtaatctagcactggaacacagttgttatgccaagtttgatggaacagaatttaaaatacttgagagtggtggaaaaatatcagataaggtaactgaagtacacgctgaatttcggaagaaaatactgcatcgcactccgccttttacttgtatgggtgcaacgtcggtatttaaccagaataactatcgtttcagtatgtacagtgatatgtgtactaaagaaactaccttagcattggcgagaaatttattcacttttgtggatgaacaagatagcatgaatagtcacttcacagcatttattgctgtgtttgaaaaaccaataccagaagatgaacaccactttgaaaaactggtgtggagtcaactacaaatgttgcatgacgaagatagccgttaccatgattgggacacccaattcagcaacgatatcaataatcataaattttcatacagttttgctcaaagaagctttttcattattggaatgcaccctggaagttctcgaattgctaggcaattttcctacccacttctagtatttaatgccagtcgtcaatttgatcatttagttgaaactaaccaattccataacttcgtcagaattattcgggaacgtgatatggccttgcaaggtagtattaatcccaatctgccagcaaaatatgaccatagtcgtcccgaagaacctgaagttagacaatattctggacgtgcagtagaaaaagattggcaatgtcccctaattgtacaccagaagaaagataggcataatgaatag

SEQ ID NO: 27 - DNA, 804 bp, Sphaerospermopsis torques-reginae; identical to SEQ ID NO: 24
  source 1..804: mol_type = other DNA

SEQ ID NO: 28 - DNA, 957 bp, Sphaerospermopsis torques-reginae
  source 1..957: mol_type = other DNA
atgaaaaaaaatattaaaaaatatcgcttcagcaattctatttatcatcaaataaaaccgcttttaaaacttgatcagtggcattgttggttggctttagcagaagactggatagttatttgttttgctatttccattagctattatttgtcttggtacttttatcctgttaccatactactcatcggttcacgtcagagagcattagccacattgctccatgaagcagcccatcaagtaattgccaaaaatcgcaccttaaactttatgatgggtacttttttctcaggatatttcattctgcaaacaatgtcatcttatcggaaatctcatgttgaaaaacatcatagatattttggcaatccggacgaagatcctgactacaagtttgcaatttctcaaggattatatcaacagagtttggatgataaaacctttcagatgctttatattttttctcctttgctacttgctcaagtacctaaatatataaaatctttattaattcaccgtttcctagaagatcaaaattatctagaactggttgctattggattgtggtggctaacaattattagcctcagcattttatttaacctatggcaatatttaattctcttctggttaattccttatttgacggtttttcaaatcattggctggtttattgaattagccgagcattaccctttaatgaataatgatatcaatttgtatatgagtcgtaaccgcaacagccattttattgagaaatttctaacaggtatgcataatgaaaactaccatttagtacatcacctatttcccaatattcctttctggaatgtgcctaaagcacacgaaatattgatgcaagatccaaattatgcccaacatcacagtcaaagtggaggaatagtctggtcaaataataatgcacctagtattttatcaggacttacacaagtgtcatcataa

SEQ ID NO: 29 - DNA, 1011 bp, Sphaerospermopsis torques-reginae
  source 1..1011: mol_type = other DNA
atgaaatacaattttcagttgaatcctaccatgaaaaaaatttttaaaaaatatcgcttcagtaattctgtttatcatcaaattaagccgctttttcaacttgataactggcattgctttttagctttagtagaaaactggataattatttgttttgtgatttctattagttattttttatcttggtacttttatcctttaaccatactaattattggttcacgtcagagggcattagccacactgctccacgaagctgcccatcaaataattgccaaaaatccgaccttaaactttatgatgggtacttgtttttcaggatattttattttccaaacaatgtcatcttatcgaaaatctcatgtggaaaaccatcatgggaatttcggcaatccagataaagatcctgactacaagtttgcaatttctcaaggattatatcaacaagatataaatgataaaatctttattaaacgttctatttttttaactttactatttattggagtacctaaatatataaaatctctattagttcaccgtttcctacaaaagcaaaattacctagaagtgtttgctattagcttatggtggctaataattattagcctcagtattctatgtaacctatggcaatatttaattctattttgggtaattccttatttgacggtttttcaaatcattggttggtttattgaattagccgagcattaccctttaatgaataatcatatcaatttgtatatgagtcgtaaccgtaacagtcattatattgaacgatttctaacgggtatgcataatgaaaactaccatttagtacatcacctatttccaaatattcctttctggaatgtacctaaagcacacgaaatattgatgcaagaccaaaattatgcccaacatcatagtcaaagtggcggaatattctggtcaaataatgatgcacccagtattttatatctaaagtctgaacatagctgcacaattccaatgggggaaaataaatga

SEQ ID NO: 30 - DNA, 978 bp, Sphaerospermopsis torques-reginae
  source 1..978: mol_type = other DNA
atgaaaaaaaatattaaaaaatatcacttcagtaattctgtttatcatcaaattaagccgctttttcaacttgataactggcattgcttcttagctttagcagaagactggataattatttgttttgcgatttctattagttattttttatcttggtacttttatcctttaaccatactaattattggttcacgtcagagggcattagccacactgctccacgaaactgcccatcaaataattgccaaaaatccgaccttaaactttatgatgggtacttgtttttcaggatattttattttccaaacaatgtcatcttatcgaaaatctcatgtggaaaaccatcatggtaatttcggcaatccagataaagatcctgactacaagtttgcaatttctcaaggattatatcaacaagatataaatgataaaatctttattaaacgttctattcttttaactttactatttattggagtacctaaatatataaaatctctattagttcaccgtttcctacaaaagcaaaattacctagaagtgtttgctattagcttatggtggctaataattattagcctcagtattctatgtaacctatggcaatatttaattctattttgggtaattccttatttgacggtttttcaaatcattggttggtttattgaattagccgagcattaccctttaatgaataatcatatcaatttgtatatgagtcgtaaccgcaacagccattttattgagcgatttttaacgggtatgcataatgaaaactaccatctaatacatcacctattcccaagtattcctttctggaatatacctaaagcacacgaaatcttgatgcaagacccaaattatgcccaacatcatcgtcaaagtggcggaatattctggtctaataatgatgcacccagtatttttctaaagtctaaacatagctgcacaattccaatggaggaaaataaatga

SEQ ID NO: 31 - DNA, 942 bp, Sphaerospermopsis torques-reginae
  source 1..942: mol_type = other DNA
atggaaaaaaagattcaaaattatcgcttcaataattctgtctatcgtcaaattaagccactttttgaacttgataactggcattgttttggagctttagcagaagactggataattatttgttttgcgatttatattagttgttttttatcttggtacttttatcctttaagtatactaattattggttcacgtcagagggcattagccacaatgctccacgaatccgcccatagaatactggcaaaaaatcccaacttaaacttgataatgggtacttttttttcaggatattttattttgcaaataatgtcgtcttatcggaaatctcatgtggaaaaccatcatggaaattttggacatccagataaagatcccgactacaaatttgccatttctcaaggattatatccaacaaatatagatgatttaagatttgcttcactactgaggcgattacctaaatatattaaatctttattagttcaccgtttcctagaagatcaaaaatccttagaaattctatctattagcttatggtggctagtaattattagcgtgagtattttatgtaacctatggcaatatttaattctattttggttaattccttatttaaccttttttcaaatcataggttggtttattgaattagccgaacattaccctttaatgaaccaccagataaatttgtatatgagtcgtaaccgtaacagtcatgacattgagctatttttaacgggtatgcataatgaaaactaccatttggtacatcacctatttccaaatattcctttctggaatgtacctaaagcacacgcaatattgatgcaagatccaaattatgcccaacatcacaatcaaagtggcggaatagtgtggtcaaataatcatgcagccagtattttatcaggactgtacaattgcaatgagggaaaataa

SEQ ID NO: 32 - DNA, 1011 bp, Sphaerospermopsis torques-reginae; identical to SEQ ID NO: 29
  source 1..1011: mol_type = other DNA

SEQ ID NO: 33 - DNA, 1113 bp, Sphaerospermopsis torques-reginae
  source 1..1113: mol_type = other DNA
atgaaaattcaacctgctttattagaaatgtggctgtctgaatatgaacgagtaccttataacttaggagaaagcagtgttgataattttaccttgggagaattactaaatcttacaggagatcgtgatgctcttgatcaactcagcctcatgaataacgacacccacggctccctgagattacgtgaagcgatcgcctcattagataaatcagtatccccagatgatattttagtgactgctgggacaacagaagccatattaatttattttaaagtacgttaccgttccggtgctaatgtcgttgttccagtaccaaccttccatgtactttatgaaactccagcttttttaggttacgaggtacgctatctgcaattacgagcagaaaatggttttcgcattgatcctcaagaattagccaagttagttgatgataatactgaagtaatagttttgaatactccacaaaatcctagtggtgttgtttgctcagaaacagaaattcagtccataattgagattgcagaaaagcataatgcagaaattttagctgatgagcattaccgttttttaccccatgatcaagacacagaaattctgccttcgttgtatggactttcacccaaaatcatcagtttaggttctacaggtaaatgctttggttgtattgggttacgcattggttggctgattggcaacccagaaataattaaagcttgtcactttttcaaagactacacaacccacactgtctgcgtccttaatgattacatcgctgctggagtcttattacacaaaggcaaaattttaccccgttatcgtcagatgattcagcacaatattcagcagtttgaaacttttatcaaacaacaacggggattaattgactgggttaaacctgaagcgggtactatcgcttttccgttttttaccgatccaaatatcaacagcaaaatagtagccaaacgtctggtagaagatcatggagtgttgttgctacctggtgaagcatttgatcgccccagtcatttccgcatagctttgggtgttgaaccaagcctttttcagtacgcattggaaaaacttgctattgtgatagaaacttcatga

SEQ ID NO: 34 - DNA, 1116 bp, Sphaerospermopsis torques-reginae
  source 1..1116: mol_type = other DNA
atgaaaattcaacctgctttattagaaatgtggctgtctgaatatgaacgagtaccttataacttaggagaaagcagtgttaataattttacccttggagaattactaagccttacaggagatcgtgacgctcttgataaactcagcctcatgaataacgacacccacggctctcggagattgcgtgaagcgatcgcctcattagatgaatcagtatccccagatgatattctagttacggctgggacaacagaagctattttaatttattttaaaatacgttaccgttctggtgctaatgtcgttgttccagtaccaacattccatgtactttatgaaactccagccttctttggttacgaggtacgctatctgcaattgcgagcagaaaatggttttcgcattgatcctgaagaattagccaagttagttgatgataatactgaaataattgttttaaatactccacaaaatcctagtggtgttgtttgctcagaaacagaaattcattccataattgagattgcagaaaagcataacgcagaaattttagctgatgagcattatcgttttttaccccatgatcaagacacagaaattctgccttcgttgtatggactttcacccaaaattatcagtttaagttctacaggtaaatgctttggttgtattggtttacgcattggttggctgattggcaacccagaagtaattaaagcttgtcactttttcaaagactacacaacccacactgtttgcgttctcaacgattacattgcatctggagtcttattacacagagacaaaattttaccccgttatcgtcagatgattcagcataatattcagcagtttgaaacttttatcaaacaacaacggggattaattgactgggttaaacctgaagcgggtactattgcttttccgttttttacaaatccaaatatcaacagcaaaacactagccaaacgtctggtggaagatcatggagtgttgttgcttcctggtgaagcgtttgatcgacccagtcatttccgcatagctttgggtgttgaaccaagcctttttcagtacgcattggaaaaacttgctattgtgataaaaacttcattatga

SEQ ID NO: 35 - DNA, 1116 bp, Sphaerospermopsis torques-reginae
  source 1..1116: mol_type = other DNA
atgaaaattcaacctgctttattagaaatctggctatctgaatatgaacgagtaccttataacttaggagaaagcagtgttaataattttaccctgggagaattactaagccttacaggagatcgtgatgctcttgataaactcagcctcatgaataacgacacccacggctctctgagattgcgtgaagcgatcgcctcattagataaatcagtatccccagataatattttagtgactgctgggacaacagaagctatattaatttattttaaagtacgttaccgttctggagctaatgtcgttgttccagtaccaacctttcatgtactttatgaaactccggccttctttggttacgaggtacgctatctgcaattgcgagcagaaaatggttttcgcattgatcctgaagaattagccaagttagttgatgataatactgaagtaatagttttgaatactccacaaaatcctagtggtgttgtctgctcagaaacagaaattcagtccataattgagattgcagaaaagcataacgcagaaattttagctgatgagcattatcgttttttaccccatgatctagacacagaaattctgccttcgttgtatggactttcacccaaaatcatcagtttaggttctacaggtaaatgctttggttgtattgggttacgcattggttggttgattggcaacccagaaataattaaagcttgtcactttttcaaagactacacaacccacactgtctgcgtccttaatgattacatcgctgctggagttttattacacaaagacaaaattttaccacgttatcgtcagatgattcagtataatattcagcagtttgaaacttttatcaaacaacaacggggattaattgactgggtaaaacctgaagcgggtactatcgcttttccgttttttacagatccaaatatcaacagcaaaacagtagccaaacttctggtagaagatcatggagtattgttgctacctggtgaagcatttgatcgacccagtcatttccgcatagctttgggtgttgaaccaagcctttttcagtacgcattggaaaaacttgctattgtgataaaaacttcattatga

SEQ ID NO: 36 - DNA, 1116 bp, Sphaerospermopsis torques-reginae
  source 1..1116: mol_type = other DNA
atgaaaattcaacctgctttattagaaatgtggctgtctgaatatgaacgagtaccttataacttaggagaaagcagtgttaataattttacgtttggagaattactaagccttaccggagatcgtgatgctcttgataaacttagcctcatgaataacgacacccacggatctttgagattgcgtgaagcgatcgcctcattagatgaatcagtatccccagatgatattctagttacggctgggacaacagaagctattttaatttattttaaaatacgttaccgtcccggagctaatgtcgttgttccagtaccaacctttcatgtactttatgaaactccagctttctgtggttatgagatacgctatctgcaattgcgagcagaaaatggatttcacattgatcctgaagaattagccaagttagttgatgataatactgaaataattgttttaaatactccacaaaatcctagtggtgttgtttgctcagaaacagaaattcactccataattgaaattgcacaaaagcataatgcagaaattttagctgatgagcattatcgttttttaccccatgatcaaaacacagaaattctcccttctttgtatggactttcaccccaaataaccagtttaggttctacaggtaaatgctttggttgtattggtttacgcattggttggctgattggcaacccagaaataataaaagcttgtcactttttcaaagactacacaacccacacggtttgtgttctcaacgattacattgcatctggagtcttattacacagagacaaaattttaccccgttatcgtcagatgattcagcataatattcagcagtttgaaacttttatcaaacaacaacggggattgattgactgggttaaacctgaagcgggtacggtcgcttttccgttttttacagatccgagtatcaacagcaaaacactagccaaacggctggtggaagatcacggagtgttgttgcttcctggtgaagcgtttgatcgtcccagtcatttccggatagctttaggtgttgaatcaaacttttttcaatatgcattggaaaaacttactattgtgatggcagattgtttatga

SEQ ID NO: 37 - DNA, 1116 bp, Sphaerospermopsis torques-reginae; identical to SEQ ID NO: 34
  source 1..1116: mol_type = other DNA

SEQ ID NO: 38 - DNA, 1044 bp, Sphaerospermopsis torques-reginae
  source 1..1044: mol_type = other DNA
atgatgagtattcaaacgttaaattttactcaatctgaacttgaagaaatagacattcttttagatcaagttactgctaactatagccatgcagaacaaccggaattgattgacactgctgcggtattagcccaaagtttgcccctacgcctgcgtcaatttttacatgagttcaaattgcgagaaaccgctcctgcttgtctcatcaagggttatccagttaatgataatactattggcgctactcctgaacattgggcaggtcatgaagatgccaagcgcactaaacgagagcaaattgtgtttgttttgtgcagtgctttactgggagaaatatttggttggctaacacagcaaaatgcctatctgatccacgatgtttttccaattaaaggctatgaacaggatcaaattggttctggtagtgaagcactactgctttggcatactgaagatgcttatcatcctgctcgtggtgactacttggggctaatgtgtttgcgtaatccttgccagagtataaccactattggttcagtggattcattaaaaaaactgactcagcagcaattagatatcttgtttcaaccacgttttattattcgcccagataatgcccatcatgccgataataattatcaaacagaaactgatttagatactcatcttcagaatgcttacaatcaaataaagcataaaattatctcccaagataatgctatggctttgctatatggagaacctagcgcaccctatttgtgtgttgatccagcttatatggaagcaacagatgaagaagctaaacagactttgcaagccttgattgatgctattgacattgaaatcatggatgtaattttacaacccggtgatatttgtttcgtagataatttccgtgctgtgcatgggagaaaaccatttaaagccaattatgatggtaatgatcggtggttaaagcgaattaatatcacacgcgatcttcgcaaatctaaaacatatcgttctcatcctacatctcgaattattgataaagtaccttcataa

SEQ ID NO: 39 - DNA, 1041 bp, Sphaerospermopsis torques-reginae
  source 1..1041: mol_type = other DNA
atgagtattcaaacgttaaatttgactgaagccgaacttgaagaaatagacattcttttagatcaagttactgctaactatagccatgcagaacaaccagaattgattgacactgctgcggtattagcccaaagtttgcccctacgcctacgtcaatttttacatgagttcaaattgcgagaaaccgctccttcttgtctcatcaagggttatccagttaatgataatactattggcactactcctaaacattgggcaggtcatgaagatgccaaccgcaccaaacgagagcaaattgtgtttgttttgtgcagtgctttactgggagaaatatttggttggctaacacagcaaaatgcctatctgatccacgatgtttttccaattaaaggctatgaacaggatcaaataggttctggtagtgaagcactactgctttggcatactgaagatgcttatcatcctgctcgtggtgactacttcgggctaatgtgtttacgtaatcctgctcaggctgtaaccactattggttcagttgattctttaaaaaagttgactcagcagcaattagatatcttgtttcaaccacgttttattattcacccagataatgcccatcacgcagataataattatcaaacagaaactcatttagatgcccatattaagaatgcttacaatcaaatcaagcagaaaattatctcccaagataacgctgtatctttgttatatggagagcctagcgcaccctatttatgcgtcgatcctgcttatatggaagcaacagatgaagaagctaaacagacgttacaagcattgattaatgctattgacattgaaatcatggatgtaattttacaacccggtgatatttatttcatagataacttccgtgctgtccatgggagaaaaccatttaaagccaattatgatggtaatgatcggtggttaaagcggattaatatcacccgtgatcttcgcaaatctagaacatatcgttctaatcctacatctcgaattattgataaagtaccttcataa

SEQ ID NO: 40 - DNA, 1041 bp, Sphaerospermopsis torques-reginae; identical to SEQ ID NO: 39
  source 1..1041: mol_type = other DNA

SEQ ID NO: 41 - DNA, 1041 bp, Sphaerospermopsis torques-reginae
  source 1..1041: mol_type = other DNA
atgactattcaaatcttaaaccttacgcaaactgaacttcaacaaatagacattcttttggatcaagttacagctaactataaccatgcggaacagccagaactgattgatactgctgcggtattagctcaaagtcttcccctgcgcttgtgtcaatttttatatgagttcaaattaagggaaaccgcccccgcttgtctcattaagggttatccagttagtgataatactattggtgttactcctaaacattgggcaggacatgaagatgccaaccgcaccaaacgagagcaaattctgtttgttttgtgcagtgctttactcggagaaatatttggttgggtaacacagcaaaatgcttatttgatccatgatgtttttcctattcccggctatgaacaggatcaaattggttctggtagtgaagcattactactttggcatactgaagatgcttatcatcctgctcgtggtgactacttgggactaatgtgcttacgtaattatgctcaggctgtaaccactattggttcagttgattctttaaaaaagttaactcagcagcaattagatattttgtttcaaccacgttttattattcgcccagataatgcacaccatgcaaataataattatcaaacagaaactcatttggatgctaatctagagaatgcttacaatcaaatcaagcagaaaattatttcacaggataacgctgtacctttgttatatggagagcctagcgcaccctatttatgtgtcgatcctgcttatatggtagcgactgatcaagaagctaaagaaactttacaagcattgattgatgccattgacattgaaatcatggatgtaattttagaacctggtgatatttgctttgtggataatttccgtgctgtccatggcagaaaaccatttaaagccaactatgatggctccgaccgttggttaaagcgaattaatatcacccgtgatcttcgcaaatctagaatatatcgctctaatcccacatctcgaattattgataaagtaccttcataa

SEQ ID NO: 42 - DNA, 1041 bp, Sphaerospermopsis torques-reginae; identical to SEQ ID NO: 39
  source 1..1041: mol_type = other DNA

SEQ ID NO: 43 - DNA, 1311 bp, Sphaerospermopsis torques-reginae
  source 1..1311: mol_type = other DNA
atgttaaaagattccccaaaatcccatgctttctttgaacgcgcacaaaaagttatcccctttggtgttaattccagccaccgctactggggcgaaaatacacctgtaattcagcgtggtgaaggagcgtatgtttatgactttgacggaaaaagctatattgattaccgtctaggttttggtccagttattcttggtcatgctcatccttttgttaatcagcgtgtcattgcagcaatacaaaatggtgtaagtttttctgcaactcaagaatatgaagtccgggtagctgaacatatcatcaagatgtgtccttctgttgacatggtacgtctggataatagtggttctgatgctacccgccatgcaattcgtctggccagaggttacactggacgagatctagtgttgaaatttgaaggtgcttatcacggtgattacgattatatgctttggacaacaccagatagtcttaaaagccgtgtgggaaaacgaggcgagccaattggacataaaaccagtcaaggtgtaccagacttagtatctcaattgttattaatagcagtttggaatgatcgtgagggaatagaaaaactgttaaaggaaaaaggagatagtattgctgccattattgtcgagccgattcttgctaatgcaggtggcttgatgccggaacctggatttttgcaattcctccgtaatcaatgtgatatttatggtattgttttaattttcgatgaagttaaaactggttttcgcatcgcaccaggaggagcaggtgaatattttggcgtacaggctgacttgagtgcctatgctaaagctatgggtaatggctatccgatttctgctattggtggcaagcgtgacattatgatgaccatagcaccaggtaaggtggtacatgcaggaacttatacaggaaatgtagtagttactgctgcggccgatgctacccttgaatatatgcaacagcaggatgtatttgggcatctcaaccacgttggacaacaactgatggacggtttagatgaaattctgactcgccatcaaattaatcatcatattcatggtttaccctcattgtttggtttaactttgtcagaatctaatcccaaagattggcgtgatgttgttgataccaatttggaattatctgaacaactactgattgaattggtcaatgctggtattatgactgattctgatccacaacaaacttggtatgtgtgtgagtctcacacgtctgaagatattgcagaaacactaaataggtttgaaactgccctgcgtaaagctttacagaaataa

SEQ ID NO: 44 - DNA, 1311 bp, Sphaerospermopsis torques-reginae
  source 1..1311: mol_type = other DNA
atgttaaaagattccccaaaatcccatgctttctttgaacgcgcacaaaaagttatcccctttggtgttaattccagccaccgctactggggtgaaaatacacctgtaattcagcgtggtgaaggagcttatgtttatgactttgacgggaaacgctatattgattaccgtctaggttttggtccagttattcttggtcattctcatccttttgttaatcagcgtgtcattgcagcgatacaaaatggtgtatgtttttctgcaactcaagaatatgaagtccgggtagctgaacatatcatcaagatgtgtccttctgttgacatggtacgtctggacaatagtggttctgatgctacccgccatgcaattcgtctggccagaggttacactggacgagatatagtgttgaaatttgaaggtgcttatcacggtgattacgattatatgctttggacaacaccagatagtctgaaaagccgtgtgggaaaacgaggcgagccaattggacataaaaccagccaaggtgtaccagacttagtatctcaattgttattaatagcagtttggaatgatcgtgagggaatagaaaaactgttaaaggaaaaaggagatagtattgctgccattattgtcgagccaattcttgctaatgcaggtggcttgatgccggaaccaggatttttgcaatttctccgtaatcaatgtgatatttatggtattgttttaattttcgatgaagttaaaactggttttcgcatcgcaccaggaggagcaggtgaatactttggcgtacaggctgatttgaatgcctatgctaaagctatggggaatggttatccaatttctgctattggtggcaagcgtgaaattatgatgactatagcaccaggtaaggtagtacacgcaggaacttatacaggaaatgtagtagttactgccgcagccgatgcgacccttgaatatatgcaacagcatgatgtatttgggcatctcaactgtgttggacaacaactgatggacggtttagatgaaattctgactcgccatcaaattgatcatcatattcatggtttaccctcattgtttggtttaactttgtcaacaactagtcccaaagattggcgtgatgttgttgataccaatttggaattatctgaacaattactaattgaattagtcaatgctggcattatgactgattctgacccacaacaaacttggtatgtatctgattctcacacctctgaagatattgcacaaacattagataagtttgaaactgccttgcataaagctttacgtgcataa

SEQ ID NO: 45 - DNA, 1311 bp, Sphaerospermopsis torques-reginae
  source 1..1311: mol_type = other DNA
atgttaaaagattccccaaaatcccatgctttctttgaacgcgcacaaaaagttatcccctttggtgttaattccagccaccgctactggggtgaaaatacacctgtaattcagcgtggtgaaggagcttatgtttatgactttgacgggaaacgctatattgattaccgtctaggttttggtccagttattcttggtcatgctcatccttttgttaatcagcgtgtcattgcagcgatacaaaatggtgtatgtttttctgcaactcaagaatatgaagtccgggtagctgaacatatcatcaagatgtgtccttctgttgacatggtacgtctggacaatagtggttctgatgctacccgccatgcaattcgtctggccagaggttacactggacgagatatagtgttgaaatttgaaggtgcttatcacggtgattacgattatatgctttggacaacaccagatagtctgaaaagccgtgtgggaaaacgaggcgagccaattagacataaaaccagccaaggtgtaccagacttagtatctcaattgttattaatggcagtttggaatgatcgtgagggaatagaaaaactgttaaaggaaaaaggagatagtattgctgccattattgtcgagccaattcttgctaatgcaggtggcttgatgccggaaccaggatttttgcaatttctccgtaatcaatgtgatatttatggtattgttttaattttcgatgaagttaaaactggttttcgcatcgcaccaggaggagcaggtgaatactttggcgtacaggctgatttgagtgcctatgctaaagctatggggaatggttatccaatttctgctattggtggcaagcgtgaaattatgatgactatagcaccaggtaaggtagtacacgcaggaacttatacaggaaatgtagtagttactgccgcagccgatgcgacccttgaatatatgcaacagcatgatgtatttgggcatctcaactgtgttggacaacaactgatggacggtttagatgaaattctgactcgccatcaaattgatcatcatattcatggtttaccctcattgtttggtttaactttgtcaacaactagtcccaaagattggcgtgatgttgttgataccaatttggaattatctgaacaattactaattgaattagtcaatgctggcattatgactgattctgacccacaacaaacttggtatgtatctgattctcacacctctgaagatattgcacaaacattagataagtttgaaactgccttgcataaagctttacgtgcataa

SEQ ID NO: 46 - DNA, 1311 bp, Sphaerospermopsis torques-reginae
  source 1..1311: mol_type = other DNA
atgttaaaagattccccaaaatcccgaactttttttgaacgcgcacaaaaagttatcccctttggtgttaattccagtcatcgttactggggaaaaaatacacctgttattgagcgtggcaaaggagcttatgtttatgactttgatggtaaacgctatattgattaccgtctaggttttggtccagtcatcctgggtcatgctcatccttttgtcaatcagcgtgtaattgcagcaatcgaaaatggtgtgactttttctgcaactcaagaatatgaagtccgagtagctgaacacatcatcaaaatgtgtccttctgttgacatggtacgtctagataatagtggttctgatgctacgcgccatgcaatacgcctggcaagaggttacactgggcgagatttagtgttgaaatttgaaggtgcttatcacggtgattatgattatatgctgtggacaacaccagatagtctgaaaaactgtgtgggaaaacgaggtaagccaattggacataaaaccagtcacggtgtaccagacttagtatctcaacttttattaatagcaatttggaatgatcgtgagggaatagaaaagctattaaaggaaaaaggagatagcattgctgcgattattgttgagccgattcttgctaatgctggtggcttgatgccagaaccaggatttttacaatttctccgtagtcaatgtgatatttatggtattgttttgatttttgatgaagttaaaactggttttcgcattgcaccaggaggagcaggtgaatactttggcgtgcaggctgatttgagtgcttatgctaaagctatgggaaatggttatccaatttctgccattggtggcaagcgtgaaattatgatgaatatagcaccaggtaaggtagtacaagcaggaacttatacaggaaatgtagtagttaccgccgccgccgatgcgaccctggaatatatgcaaaagcaggatgtatttgggcatctcaactgtgttggaacacaactgatggagggtttagatgaaattctaactcgccatcaaattgatcatcatattcatggtttaccctcattgtttggtttaactttgtcaaaaactaatcccacagattggcgtgatgttgttgatacaaatttggaattatctgaaaaattactcattgaattggtcaatgctggtgttatgactgattctgatccacaacaaacttggtatgtatctgattctcacacctctgaagatattgcacaaacattagataagtttgaaactgctttacataaagctgtacgtatataa

SEQ ID NO: 47 - DNA, 1311 bp, Sphaerospermopsis torques-reginae; identical to SEQ ID NO: 44
  source 1..1311: mol_type = other DNA

SEQ ID NO: 48 - DNA, 825 bp, Sphaerospermopsis torques-reginae
  source 1..825: mol_type = other DNA
atggaactcaagtttcaagaaaatgattttcaatcttttgaaccaataccttatctgaatgaatactttaattatccttcagattattatgggggtgtaggatttgagaatgaacaattcctacaattctttgctgaagttgcccatgaatatcatctcaataactatttgctattagatttcggttgtggtcctactatttattctatcctcagcttaggacggaattgccgtgaaatacatatgagtgattatttacaacaaaatttagagcaggttcagctttggcaacaaggaaagccccaagctttcgattggtatccttatctgcgtcgcgccttgcaaatggaaactgcgttaaatcagaatcagcctttagatagttttttagatgttacagatgaagaaatagaagaacgggcaagattgctacgggaaaaaataacttctatcagaaaaggcaatgctcgtgctactaatcctgtgggagaagaaggaaaggcattatatgaagcggtggtttcttctttctgtttagaaggtgtagcacaagatttagctgaatggaaaaatttgataactaatttgtcttccacaattaaaccagggggtttattgatatttgctactcaaattgaagctgatagttatcgaattggagagggttacggcacagtggttaatctcactgaacaagatattgtgcagacattattgaattgtaatttcgagtcaggttcaatcaaaacaaaggaacttaaaggcggtccgaatcacactgagtacgataaattcctgatgttgacagccagacttgctgtgtga

SEQ ID NO: 49 - DNA, 713 bp, Sphaerospermopsis torques-reginae
  source 1..713: mol_type = other DNA
aacaattcctacaattctttgctgaagttgcccatgaacatgatctcaaaaattgtttgctactagattttggttgtggtcctactatttattctatcctcagcttaggactgaactgctgtgaaatacacatgagtgattatttacaacaaaatttagagcaggttcagctttggcaacaaggaaagccccaagcttttgattggcatccttatctgcgtcgcgccctgcaaatggaaactgcgttaaatcagcatcagcctctagatagttttttagatgttacagatgaagaaatagaacaacgggcgagattgctacggggaaaaataacttctatcagacaaggcaatgctcgtgctattaatccagtgggagaagaaggaaaggcattatatgaagcggtggtttcttctttctgtttagaaggtgtagcacaagatttagctgaatggaaaaagttgatgactaatttgtcttccaccattaaaccagggggtttattgatatttgctactcaaattgaagctgatagttatcgaattggcgatggttacggcacagtggttaatctcagtgaacaagatattgtggaaacattattggattgtaatttcgagtcaagctcaatcaaaacgaaggaacttgaaggcggtccgaatcacacagagtacgagaaattccttatgctgacagcaagacttgctttgtga

SEQ ID NO: 50 - DNA, 825 bp, Sphaerospermopsis torques-reginae
  source 1..825: mol_type = other DNA
atgcaactgaaatttaaagaaaatgattttcaatcttttgagccaataccctatctgaatgaatactttaattatccttcagattattacggaggtgtaggatttgagaacgaacaattcctacaattctttgctgaagttgcccatgaacatgatctcaaaaattgtttgctactagattttggttgtggtcctactatttattctatcctcagcttaggactgaactgctgtgaaatacacatgagtgattatttacaacaaaatttagagcaggttcagctttggcaacaaggaaagccccaagcttttgattggcatccttatctgcgtcgcgccctgcaaatggaaactgcgttaaatcagcatcagcctctagatagttttttagatgttacagatgaagaaatagaacaacgggcgagattgctacggggaaaaataacttctatcagacaaggcaatgctcgtgctattaatccagtgggagaagaaggaaaggcattatatgaagcggtggtttcttctttctgtttagaaggtgtagcacaagatttagctgaatggaaaaagttgatgactaatttgtcttccaccattaaaccagggggtttattgatatttgctactcaaattgaagctgatagttatcgaattggcgatggttacggcacagtggttaatctcagtgaacaagatattgtggaaacattattggattgtaatttcgagtcaagctcaatcaaaacgaaggaacttgaaggcggtccgaatcacacagagtacgagaaattccttatgctgacagcaagacttgctttgtga

SEQ ID NO: 51 - DNA, 816 bp, Sphaerospermopsis torques-reginae
  source 1..816: mol_type = other DNA
atggaactgaaatttaaagaaaatgattttcaatcttttgagccaataccctatctgaatgaatactttaattatccttcagattattacggaggtgtaggatttgagaatgaacaattcctacaattctttgccgaagttgcccatgaacatgatctcaaaaattgtttgctactagattttggttgtggacctactatttactctatccttagcttaggacggaactgccgtgaaatacacatgagtgattatttacagcaaaatttagagcaggttcagctttggcaacagggaaaacctcaagcttttgattggcatccgtatctgcgtcgcgccctgcaaatggaaaccttgttaaatcagaatcagcctctagagagttttatggatcaacagatagaacaacgggcgagattgctacggggaaaaataacttctatcagacaagggaatgctcgtgctattaatccagtgggagaagaaggcaagggattatatgaagcggtggtttcttctttctgtttagaaggcgtagcacaagatttagctgagtggaaaagcttaataactaatttgtcttcgacaattaaaccagggggtttattgatatttgctactcaaattgaagctgatagttatcgaattggcgatggttacggcacagtggttaatctgagtgaacaagatattgtgcaaacattattagattgtaatttccagtcaaactcaatcaaaaccagggaacttcaaggcggtccgaatcacactgagtacgagaaattcctaatgttgacagcaagactcgctttgtaa

SEQ ID NO: 52 - DNA, 825 bp, Sphaerospermopsis torques-reginae
  source 1..825: mol_type = other DNA
atggaactgaaatttaaagaaaatgattttcaatcttttgagccaataccctatctgaatgaatactttaattatccttcagattattacggaggtgtaggatttgagaacgaacaattcctacaattctttgctgaagttgcccatgaacatgatctcaaaaattgtttgctactagattttggttgtggtcctactatttattctatcctcagcttaggactgaactgctgtgaaatacacatgagtgattatttacaacaaaatttagagcaggttcagctttggcaacaaggaaagccccaagcttttgattggcatccttatctgcgtcgcgccctgcaaatggaaactgcgttaaatcagcatcagcctctagatagttttttagatgttacagatgaagaaatagaacaacgggcgagattgctacggggaaaaataacttctatcagacaaggcaatgctcgtgctattaatccagtgggagaagaaggaaaggcattatatgaagcggtggtttcttctttctgtttagaaggtgtagcacaagatttagctgaatggaaaaagttgatgactaatttgtcttccaccattaaaccagggggtttattgatatttgctactcaaattgaagctgatagttatcgaattggcgatggttacggcacagtggttaatctcagtgaacaagatattgtggaaacattattggattgtaatttcgagtcaagctcaatcaaaacgaaggaacttgaaggcggtccgaatcacacagagtacgagaaattccttatgctgacagcaagacttgctttgtga

SEQ ID NO: 53 - DNA, 1035 bp, Sphaerospermopsis torques-reginae
  source 1..1035: mol_type = other DNA
atgagtgagcattttcccattgatttacgcagtgatacagttacccaaccgacaccatctatgcgtcaagctatggctgaagcgatagtaggtgacgatgtttatggagaagatccaacggttaaccgactggaacgagtagcagcagaacttttaggtaaagagtctgctatgtttgtacctagtggcacgatgggtaatctcatctgtatgttaactcattgtggacgtggagaagaagtgattattggtgatcaagctcatctgtttttgtatgaacgtggtggcagtgctgtattggggggtattcatccttacactgtcagcacagcaacagatggtactcttcctttatcagaaatccgcgctgctgtaagacctgatgatattcattgtccgagaacaaagctattatgtgtggaaaatacacaagcacaacgaggcggtttgcctttatcattaacttatttacacagcttgcgtgatttggcgaatgagttaggattaagattgcactgtgatggagcgcgtttgtggaacgcatctgtggcaattggggttgaacctagtgttttatcttcaccttttgattctttatctgtctgtttatcaaagggtctggctgcacctattggttctcttgtgatcggaacacagtcatttattgatgaagcacggcggttacgtaaactattgggtggtggtatgcgtcaagttggagcgatcgcctctgctggtttaattgccctagaacaaatgagggaaagactggctattgatcatgaaaacgcccaatttttagctgaaggtttacgtgaatgtggttatgaaatcactcatcctgtgcggactaatatcatattttttcgccagcatgagcaagataccttatctctccaggaaagattagctgtttggaaaaactctggcttgttagtctcagattttggcgatcgcacaattcgcgcagttacccattatggtattgaacgcaaagatattaatcaagcaatttctattttacaacaagtctag

SEQ ID NO: 54 - DNA, 1035 bp, Sphaerospermopsis torques-reginae
  source 1..1035: mol_type = other DNA
atgactcagcattttaccattgatttacgcagtgatacagtcacccaacccacaccctctatgcgtaaagctatggctgaagcgatagtaggtgacgatgtttatggagaagatccaacggttaaccgcctggaacgagtaacagcagaacttttaggtaaggagtctgcaatgtttgtacctagtggcacgatgggtaatctcatctgtgtgttaactcattgtggacgtggggaagaagtgattattggtgatcaagctcatctgtttttgtatgaacgtggtggcagtgctgtattggggggtattcatccttacactgtcagcacagcaacagatggttctcttcctttatcagaaatccgcgctgctgtacgacctgatgatattcattgtccgagaacaaagctattatgtgtggaaaatacacaagcacaacgaggcggtttgcctttatcattaacttatttacagagcttgcgtgatttggcaaatgagttaggatttgcattgcactgtgatggagcgcgtttgtggaatgcatctgtagcaactgggattgaaccaagtgttttatcttcaccttttgattctttatctgtctgtctatcaaaagggctggctgcacctattggttcccttgtgatcggaacacagtcatttattgatgaagcacggcggttacgtaaactattgggtggcggtatgcgtcaagttggagcgatcgcctctgctggtttaattgccatacaagaaatgagggaaagactggctattgatcatgaaaacgcccaatttttagctgaaggtctacgtgaatgtggttatgaaatcacccatccggtgcggactaatatcatattttttcgccagcatgagcaagataccttatctctccaggaaagattagctgtttggaaaaactctggattgttagtctcagattttggcgattgcacaattcgcgcagttacccattacggtattgaacgcaaagatattgatcaagtaatttctattttacaacaagtctag

SEQ ID NO: 55 - DNA, 1035 bp, Sphaerospermopsis torques-reginae; identical to SEQ ID NO: 54
  source 1..1035: mol_type = other DNA

SEQ ID NO: 56 - DNA, 1035 bp, Sphaerospermopsis torques-reginae
  source 1..1035: mol_type = other DNA
atgaccgagtattttaccattgatttacgcagtgatacagtgaccctacccacaccttctatgcgtaaagccatggctgaagcgatagtaggtgacgatgtttatggagaagatccaacggttaaccgcctagaacgagtagcagcagaacttttaggtaaagagtctgcaatgtttgtacctagtggtacgatgggtaatctcatctgtgttttaactcattgtggacgtggggaagaagtgattattggcgatcaagctcatctgtttttgtacgaacgtgggggcagtgcagtgttggggggtattcatccgtacactgttaagacagcaacagatggctctctccctttatcagcaatccgtgaagctgtacgaccggatgatattcattgcccgagaacaaaactattatgtctagaaaatacacaagcacaacgaggaggtgcgcctttatcattaacttatttacaaagtctgcgtgatttggcaaatgagttaggactggcattacactgtgatggagcgcgtttgtggaatgcatccgtcgcaactggagttgagccaagtgttttatcttcaccttttgattctttatctgtctgtttatcaaaggggttgggtgcgcctattggttcccttgtaatcggaacacagtcatttattgatgaagcacggcgattacgcaaactactgggtgggggtatgcgtcaagctggagcgatcgcggctgctggtttaattgccatacaagaaatgagggaaagactggctaccgatcatgaaaatgctcaatttttagccgcaggtttacgtgaatgtggttatgaaatcacccatccggtgcggactaatatcatattttttcgccagcataaggaagataccttatctgtccgggaaagattagctgtttggaaaaactctggcttgttagtctcagattttggtgattgcacaattcgcgcagttacccattacggtattgaacgcaaagatattgatcaagtaatttctattgtacaaaaagtctag

SEQ ID NO: 57 - RNA, 1035 nt, Sphaerospermopsis torques-reginae
  source 1..1035: mol_type = other RNA
atgactcagcattttaccattgatttacgcagtgatacagtcacccaacccacaccctctatgcgtaaagctatggctgaagcgatagtaggtgacgatgtttatggagaagatccaacggttaaccgcctggaacgagtaacagcagaacttttaggtaaggagtctgcaatgtttgtacctagtggcacgatgggtaatctcatctgtgtgttaactcattgtggacgtggggaagaagtgattattggtgatcaagctcatctgtttttgtatgaacgtggtggcagtgctgtattggggggtattcatccttacactgtcagcacagcaacagatggttctcttcctttatcagaaatccgcgctgctgtacgacctgatgatattcattgtccgagaacaaagctattatgtgtggaaaatacacaagcacaacgaggcggtttgcctttatcattaacttatttacagagcttgcgtgatttggcaaatgagttaggatttgcattgcactgtgatggagcgcgtttgtggaatgcatctgtagcaactgggattgaaccaagtgttttatcttcaccttttgattctttatctgtctgtctatcaaaagggctggctgcacctattggttcccttgtgatcggaacacagtcatttattgatgaagcacggcggttacgtaaactattgggtggcggtatgcgtcaagttggagcgatcgcctctgctggtttaattgccatacaagaaatgagggaaagactggctattgatcatgaaaacgcccaatttttagctgaaggtctacgtgaatgtggttatgaaatcacccatccggtgcggactaatatcatattttttcgccagcatgagcaagataccttatctctccaggaaagattagctgtttggaaaaactctggattgttagtctcagactttggcgattgcacaattcgcgcagttacccattacggtattgaacgcaaagatattgatcaagtaatttctattttacaacaagtctag

SEQ ID NO: 58 - DNA, 1242 bp, Sphaerospermopsis torques-reginae
  source 1..1242: mol_type = other DNA
atgagtaacaacaaacaagacatcaatcacacagaattaaatcccatggctcatagccgcaggggattttttactgtgtttattgcaggaatttctacatctgtatctagtattttcaggaaagatgttgctgtagctgagaaccataaagccttcaccgccgaaaacaaatcaatgctctctaccaataaccagtcatttttttttccaggtgaattgttagcatctgacgaaatgcggatcactattgtagggtcttcttccgtcgcacgcaaaacccaatctggaacaagcatctttctcgaactgggaaacggggacagtttcatctttgatgccggacctggtgtagctgccaactatacagcgttgggtgtaccctattcgcggatggatagaattttcctaagtcaccttcattgcgatcacataacggatcttgcctacatatatggtcaaggatcaatgtatgatcgcaaaacacccctacgtatctggggaccgagtggcaaggagtcgaagtttggcacaaatgctttcgtcgagtcaatgaaggctatgtgtctctggcacacggagagtctttcctaccaccagaacggtaatggttatgaaacagaggtacatgaatttgactatgctcaaaatccaggtgttgcatacaacgagaaaggagttgtgattaagcactttcccgcagttcacctgaaagacggtgcggtaagctaccgtgtggattggaatggtctcagttttgtcttctctggcgactcgcaaccgaatacatttatggttgagaacgcacaaaatgttgatatcttgattcaccaaacaattcctccagcacaggtttgggcaaaagaatacaatatgcccctcgaacaagttatagaactgcaaaacagatttgattctcctccccatgctgttggtgcgattttcgaccaaactagaccacgcttgggtgtactaactcatttatatctaaatgaaaatataaaagttgccattcttgactcgtgccaagaggtttacgacggacctattgaaattgctcaggatttgatggttttcaatatctccaagaatgaaatcaagcagcgcatagcgatcgtccctgaattagcgttgcaaaacaccgtcaagaagtacgaaaaccttccaccacctaagtataaaccaagggagcagttgtcccagtggcttctggacgcggtaattccgcaagaacaatggaaatag

SEQ ID NO: 59 - DNA, 1199 bp, Sphaerospermopsis torques-reginae
  source 1..1199: mol_type = other DNA
atgagtaacaacaagcaagacattaataacagaaaattaaatccaatggctcatagccgcaggggatttttaactgtgtctattgcaggaatttctacgactgtatctagtattctcaggaaagatgttgctgcggctgagaacaataaagctttcactgccgaaaacaaatcaatgctctccacaaataagcagtttttttttccaggtgaattgctagcatctgacgaaatgcggatcactattgtagggtcttcctcagtcgcacgcaaaacgcaatctggcacaagcatctttctcgaactgggaaacggggacaacttcatctttgatgccggacctggtgccgctgccaactatacggcgttgggtgtaccctattcgcggatggatagaatttttctgagccacctccattgcgatcacataacagatattgcctacatatatggtcaaggtccaatgtatgatcgcaaaacgcccctacgtatctggggaccgagtggcaaggagtcgaagttcggcacaaatgctttcatcgagtccatgaaggctatgtgtctctggcacacggagagtctttcctaccaccagaacgggaatggttacgaaacagaggtacatgaatttgactatactcaaaatccaggtgttgcatacaacgagaaaggagttgtgatcaagcacttccccgcagttcacctgaaagacggtgccgtaagctaccgtgtggattggaatggtctcagttttgtcttttctggtgactcgcaaccgaatacatttatggttgagaacgcacaaaatgttgacatcttgattcaccagacaattcccccagcacaggtttgggcgaaagaatacaatatgccgctaaaacaagttatagaactgcaaaacagatttgattctcctcccgatgctgttggtgcgattttcgcccgaactagaccacgcttaggtgtactaactcatatacatctgaatgagaatataaaagtttctattcttgactcctgccaagaggtttacgacggacctattgaaattgctcaggatttgatggtgttcaatatctccaagaatgaaatcaagcagcgcatagcgatcgtccctgaattagcgttgcaaaacaccgtcaagaagtacgaaaaccttccgccaccgaagtataaaccaagagagcagttgtcccagtg

SEQ ID NO: 60 - DNA, 1245 bp, Sphaerospermopsis torques-reginae
  source 1..1245: mol_type = other DNA
atgagtaacaacaagcaagacattaataacagaaaattaaatccaatggctcatagccgcaggggatttttaactgtgtctattgcaggaatttctacgactgtatctagtattctcaggaaagatgttgctgcggctgagaacaataaagctttcactgccgaaaacaaatcaatgctctccacaaataagcagtttttttttccaggtgaattgctagcatctgacgaaatgcggatcactattgtagggtcttcctcagtcgcacgcaaaacgcaatctggcacaagcatctttctcgaactgggaaacggggacaacttcatctttgatgccggacctggtgccgctgccaactatacggcgttgggtgtaccctattcgcggatggatagaatttttctgagccacctccattgcgatcacataacagatattgcctacatatatggtcaaggtccaatgtatgatcgcaaaacgcccctacgtatctggggaccgagtggcaaggagtcgaagttcggcacaaatgctttcatcgagtccatgaaggctatgtgtctctggcacacggagagtctttcctaccaccagaacgggaatggttacgaaacagaggtacatgaatttgactatactcaaaatccaggtgttgcatacaacgagaaaggagttgtgatcaagcacttccccgcagttcacctgaaagacggtgccgtaagctaccgtgtggattggaatggtctcagttttgtcttttctggtgactcgcaaccgaatacatttatggttgagaacgcacaaaatgttgacatcttgattcaccagacaattcccccagcacaggtttgggcgaaagaatacaatatgccgctaaaacaagttatagaactgcaaaacagatttgattctcctcccgatgctgttggtgcgattttcgcccgaactagaccacgcttaggtgtactaactcatatacatctgaatgagaatataaaagtttctattcttgactcctgccaagaggtttacgacggacctattgaaattgctcaggatttgatggtgttcaatatctccaagaatgaaatcaagcagcgcatagcgatcgtccctgaattagcgttgcaaaacaccgtcaagaagtacgaaaaccttccgccaccgaagtataaaccaagagagcagttgtcccagtggcttttggacgcggtaattccgcaagaagaatggaagaaagtctaa

SEQ ID NO: 61 - DNA, 1236 bp, Sphaerospermopsis torques-reginae
  source 1..1236: mol_type = other DNA
atgagtaacgacatcaataaaacagaattaaatccaatggcttctagccgcaggggattttttagtctgtcggttgcaggagttgtaacggctgtatctagtattctcagaaaagatgttactgcggctcagaaccataaagctttcactgttgaaaataaatcaatgctctcaaaaaataaccagttatttttttttcccggtgaattgttagcacccgacgaaatgcggatcactattatcgggtcttctccagtcgcacgcaaaacccaatctggtacaagcatctttctggaactgggaaacggtgacaacttcatctttgatgctggacctggtactgccgccaactatactgccttgggtgtaccctattcgcggatggatagaatttttctgactcacctccattgtgatcacatcaccgatcttccctacatctatggtcaaggtgcgatgtacgatcgcaaaacccccctacatatctggggtccgagtggtaaggagtcgaagttcggcacaaatgcttttgtcgagtcaatgaaggctatgtgtctctggcacacggaaagtctttcctaccaccagaacggtaatggttacgaaacggaagtacatgaatttgactatactcaaaatccaggtgttgcatataacaagaaaggagttgtgatcaagcacttccccgcagtccacctgaaagacggtgccgtaagctaccgtgtagattggaatggtctcagttttgtcttctctggtgactcacaaccgaatacatttatggttgagaatgcacaaaatgttgatatcttgattcaccagacaattcctccagcaacggtttgggcgaaagaatacaatatgccgctcgaaaaagttatagaactgcaaaacagatttgattctcctcccaatgctgttggtcagattttcgcccgcactaaaccacgtttgggtatactaacccatatatatattaataaaaatattaaactttctcttctcaaatcctgccaagaggtttacgacggatatattgaaattgctcaggatttgatggtattcaatatctccaaaaatcaaatcaagcggcgcatagcgatcgtccctgaattagtcctacaaaatattgtcaagaagtacgaaaacctgccgccaccgaagtataaaccaagggagcagttgtcccagtggcttctcgacgcggtaattccacccgaagaatggaagatgtaa

SEQ ID NO: 62 - DNA, 1245 bp, Sphaerospermopsis torques-reginae; identical to SEQ ID NO: 60
  source 1..1245: mol_type = other DNA

SEQ ID NO: 63 - DNA, 918 bp, Sphaerospermopsis torques-reginae
  source 1..918: mol_type = other DNA
ttgaaaaaatccatcgacaagatcattaccaacttaccgctatgggatacacaaggtgctctgaaaattgtgccaatcggtgaccaaaacttcaccaatcagaattaccgagttgagagcaacggacaggtattcaaggttcgcatcagtgaaggaaatcaaaacctcatagggattgccaaagaagaagaactgattgtgcttaaggcagttgctaaaatgggtataggaccagaggtcattgcatacataccaccagaaggtcatctggtaacgcgatttatagagggtaagcatttttctctacaggaaatcacgcaaccggaaaacatacatcgaattgcccagatcctaaagcaagttcatagtattgaggggattgacgctgcaccgccaccatttgagcgaattgaggggctgatgaggaacgctgaacgcaaaaatggcatatttcctgatgattttgaacagttactcgctgacctacaagctataaaggctgtactgactaaagtccacagaaaaccatgtctgtgtcataacgacttggctggctcgaatatcattgaggccgatggttcaattttcctgatcgattgggagtacgcaggtgtagcaaatccgatgtttgacctagctaacttttctatgaatcagaacctggatagagaaggtgataaaattctcattgagagctactttgggaaagttagtcaggtggagttagcagaaatcaatctttggaagatagttgctatttttattgaaggtgtttggggtgttttgcaggcaaaaatttctcaatttgatcgggactatcaaggttttgctaacgaaaattggcaaacagtgcgtcagtacacgcaaaaggaaagcttttcagaatggctgtccatgatatcgttatcaaaccactga

SEQ ID NO: 64 - DNA, 918 bp, Sphaerospermopsis torques-reginae
  source 1..918: mol_type = other DNA
ttgaaaaaatccatagacaagataattaccaacttaccgctatgggatacacaagatgctctgaaaattatgccaattggtgatcaaaacttcaccaatcagaattaccgagttgagggcaacggacaggtattcaagattcgcatcagcgaaggaaatcaaaacctaatagggattgctagggaagaagaactaaatgttcttaaggcagttgctaaaatgggtataggatcagaagtgattgcatacataccaccagaagggcatctggtgacgcgatttatagagggtaggcatttttctctagaggaaattacacaaccggaaaacatacatcgaattgcccagatcctaaagcaagttcatagtattgaggggattgacgctgcaccgccaccatttgagcgaattgaggggctgatgaggaacgctgaacgcaaaaatggcatatttcctgacgattttgaacagctactcgctaatctacaagctataaaggctgtactgactaaagtccacagaaaaccatgtctgtgtcataacgacttggctggatcgaatatcatcgagtccgatggttcaattttcctaatcgattgggagtacgcaggtctagcaaatccgatgtttgatctagctaacttttctatgaatcagaacctggatagagaaggtgatcaaattctcattgagagctactttgggaaagttagtcagatggagttagcagaaatcaatctttggaagatagttgctatcttcattgaaggtgtttggggtattttacaggcgaaaatttctcaatttgatcgggactatcaaggctttgctaacgaaaattggcaaacagtgcgtcagtacacgcaaaaggaaagcttttcacaatggctctctatgatatcgtcatcaaaccactga

SEQ ID NO: 65 - DNA, 918 bp, Sphaerospermopsis torques-reginae
  source 1..918: mol_type = other DNA
ttgaaaaaatccatagacaagataattaccaacttaccgctatgggatacacaagatgctctgaaaattatgccaattggtgatcaaaacttcaccaatcagaattaccgagttgagggcaacggacaggtattcaagattcgcatcagcgaaggaaatcaaaacctaatagggattgctagggaagaagaactaaatgttcttaaggcagttgctcaaatgggactgggatcagaagtgattacatacataccaccagaagggcatctggtgacgcgatttatagagggtaggcatttttctctagaggaaattacacaaccggaaaacatacatcgaattgcccagatcctaaagcaagttcatagtattgaggggattgacgctgcaccgccaccatttgagcgaattgaggggctgatgaggaacgctgaacgcaaaaatggcatatttcctgacgattttgaacagctactcgctaatctacaagctataaaggctgtactgactaaagtccacagaaaaccatgtctgtgtcataacgacttggctggatcgaatatcatcgagtccgatggttcaattttcctaatcgattgggagtacgcaggtctagcaaatccgatgtttgatctagctaacttttctatgaatcagaacctggatagagaaggtgatcaaattctcattgagagctactttgggaaagttagtcagatggagttagcagaaatcaatctttggaagatagttgctatctttattgaaggtgtttggggtattttacaggcgaaaatttctcaatttgatcgggactatcaaggctttgctaacgaaaattggcaaacagtgcgtcagtacacgcaaaaggaaagcttttcacaatggctctctatgatatcgtcatcaaaccactga

SEQ ID NO: 66 - DNA, 918 bp, Sphaerospermopsis torques-reginae; identical to SEQ ID NO: 65
  source 1..918: mol_type = other DNA

SEQ ID NO: 67 - DNA, 457 bp, Sphaerospermopsis torques-reginae
  source 1..457: mol_type = other DNA
ttgaaaaaatccatagacaagataattaccaacttaccgctatgggatacacaagatgctctgaaaattatgccaattggtgatcaaaacttcaccaatcagaattaccgagttgagggcaacggacaggtattcaagattcgcatcagcgaaggaaatcaaaacctaatagggattgctagggaagaagaactaaatgttcttaaggcagttgctaaaatgggtataggatcagaagtgattgcatacataccaccagaagggcatctggtgacgcgatttatagagggtaggcatttttctctagaggaaattacacaaccggaaaacatacatcgaattgcccagatcctaaagcaagttcatagtattgaggggattgacgctgcaccgccaccatttgagcgaattgaggggctgatgaggaacgctgaacgcaaaaatggcatatttcctg

SEQ ID NO: 68 - DNA, 750 bp, Sphaerospermopsis torques-reginae
  source 1..750: mol_type = other DNA
atggtaaaatcaaatattttaagtcgtaatgttgtaggttataattccactgatttaagttcagatcacattcaggcagaagcacgtgatttttatgcaagacaagcaggagtatttgctaagattctcaacgaatcttggtcagaatacacgaatatttctcatcctggcttcaaaaatgatctcgacctcattcgtcaagtaaaagaacttgttgctcctggtgcaaaaggaattgatgctggttgtggtcctcttgcccgggaggtattctgcttttatgaagatggttatgatatgtatggtatggatgtagttccagaaaatatccaaacggcactggagtatcatccttcattggctgatcggctttttttggctgatttgacgcagccacttgatgtccctgatcattcctttgattttattatttgcaatacagtaattcaacacattaatctcaattctttaactaatgtaacctttccagagtggacacgaatacttaagcctggtggaatcattcagttaatgtttaggtctggtcatggaactcaaattattcatgatgtagaatttaacgatcaacgtagtttttatctgtatcaaccagaggaaattctagaaatactggcgagatatggattgtctttaattccagaagagaatggcaatttaggaggaattatttatgttactgatgtcagaccaatggaaaattgtattttctgtgtgcggaagacagattaa

SEQ ID NO: 69 - DNA, 753 bp, Sphaerospermopsis torques-reginae
  source 1..753: mol_type = other DNA
atggtacaaccagagattttaaatcgtaatgttgtaggtcataagtctactgatttaagtttggatcaaattcaggcagaagcgcgtgatttttatgcaagacaagcaggagtatttgctaagattctaaacgaatcttggtctgaatacacgaatatttctcatcctggattcaaaaatgatcttgacctcattcgtcaagtaaaacaatttattgctcctggtgcaaaaggaattgatgctggttgtggtcctcttgcccgtgaggtattctgcttttatgaagatggttatgatatgtatggcatggatgtagtgccagaaaatatccaaacggcactggagtatcatccttcattggctgatcggctttttttggctgatttgacgcaaccacttgatttccctgataattcctttgattttattatttgcaatacagtcattcaacacattaatctcaattctttaactaatgtaacctttccagagtggacacgaatacttaagcctggtggaatcattcagttaatgtttaggtctggtcatggaactcaaactattcaggatgtagaatttaacgatcaacgtagtttttatctgtatcaaccacaggaaattctagaaatacttgcgcgatatggattgtctttaattccagaagagaatggcaatttaggaggaattatttatgttactgatgtcagaccaatggaaaattgtattttctgtgtgcggaagacacatgattga

SEQ ID NO: 70 - DNA, 753 bp, Sphaerospermopsis torques-reginae
  source 1..753: mol_type = other DNA
atggtacaaccagagattttaaatcgtaatgttgtaggtcataagtctactgatttaagtttggatcaaattcaggcagaagcgcgtgatttttatgcaagacaagcaggagtatttgctaagattctaaacgaatcttggtctgaatacacgaatatttctcatcctggattcaaaaatgatcttgacctcattcgtcaagtaaaacaatttattgctcctggtgcaaaaggaattgatgctggttgtggtcctcttgcccgtgaggtattctgcttttatgaagatggttatgatatgtatggcatggatgtagtgccagaaaatatccaaacggcactggagtatcatccttcattggctgatcggctttttttggctgatttgacgcaaccacttgatttccctgataattcctttgattttattatttgcaatacagtcattcaacacattaatctcaattctttaactaatgtaacctttccagagtggacacgaatacttaagcctggtggaatcattcagttaatgtttaggtctggtcatggaactcaaactattcaggatgtagaatttaacgatcaacgtagtttttatctgtatcaaccacaggaaattctagaaatacttgcgcgatatggattgtctttaattccagaagaaaatggcaatttaggaggaattatttatgttactgatgtcagaccaatggaaaattgtattttctgtgtgcggaagacacatgattga

SEQ ID NO: 71 - DNA, 753 bp, Sphaerospermopsis torques-reginae
  source 1..753: mol_type = other DNA
atggtcaaatcaaatattttaagtcgtaatgttgtaggttgtaattccactgatttaagttcggatcaaattcaggcagaagcgcgtgatttttatgcaagacaagcgggagtatttgctaagattctcaaggaatcttggtcagaatacacgaatatttctcatcctggattcaaaaatgatctcgacctgattcgtcaagtaaaagaacttgttgctcctggtgcaaaaggaattgatgctggttgtggtcctcttgcccgtgaggtattctggttttatgaagatggttatgatatgtatggtatggatgtagttccagaaaatatccaaacggcactggagtatcatccttcattgactgatcggctttttttagctgatttgactcagccacttgatttccctgatcattcctttgattttattatttgcaatacggtaattcaacacattaatctcaatactttaactaatgtaacctttccagagtggacacgaatacttaagcctggtggaatcattcagctaatgtttaggtctggtcatggaactcaaattattcaggatgtagaatttaatgatcaacgtagtttctatctgtatcaaccagaggaaattctagaaatactggcgagatatggattgtctttaattccagaagagaatggcaatttaggaggaattatttatgttactgatgtcagaccaatggaaaattgtattttctgtgtgcggaagatagatgattga

SEQ ID NO: 72 - DNA, 753 bp, Sphaerospermopsis torques-reginae; identical to SEQ ID NO: 69
  source 1..753: mol_type = other DNA

SEQ ID NO: 73 - DNA, 1359 bp, Sphaerospermopsis torques-reginae
  source 1..1359: mol_type = other DNA
atgtatttgacactcttcaaacagtatgactttctgaaccgatttattcggttatcggttgccaatatagtctccaatctcatgatccctctagctggttccatcagtctagtttttttgggtcatctctcaggagtccatgatttggccggatcatcttttgcagtagtattatttgattttatctactacggtttttactttttacgacagagtaccactggcgttaccgctcaggcaataggaagagaagaccaggaggagatgttactgataggactgcgaaatatcttaattgctttagtcttaggcatactgcttttgattttgcagtacccatttagggagatagcattttccatgttaaatgatactccagaggttaaagcttcggcggttgcctatttcaatgctcggattattggtgcgcctgcggttttcatcaattacgttctgattggttgtttgctaggaagggaagagagcggtaaggtgttactaatatcagtaatgggaaatgctgtcaacatattactagactatttgctgattatccattggggttggggagctacaggagctggactctctatggctattagtcagtatttaatgttattggtaggattaatatttttttgccttcaaatccagtggcaaatgttaaaagctataactggaaaacttttggattggtcagctttgaaatctgcctttgtcctgaatggcaatatatttgttagtaccttgatcctgatctccaccactgatatattcagtttagagagcaatacaatggggacaatcatctttactcaaaataccttgatattacaaatcattttcctagctctctattttgttgaaggattaggattagctacagagagtttagtaggctactttacaggtcaaggtgcgaaggaaaagttagcttctttggtagggctttctgcacaagctagtcttgttttgggactcgcctttgctcttgtacctgcgctttttcctcaatgggtatttgggttattcaccgaccatattgaaataattagtcaaattgatacatatatttggtggttaccactttttttgacatttagttcagtagcacttgcactggaaggatactttctaggtttagcagaaggacgcactcttcgcaatattaacttaactgccattattttaggatttatgcctttaagtatagccgcttggcagtttcacaataaccatttgttgtggcttgcttattctatgtttatgtgcatcagaatggtgatgcttagtatacaagtaccaagaacttttgaaagtgattttggagagcgaaaaattttgggggagatggagtag

SEQ ID NO: 74 - DNA, 540 bp, Sphaerospermopsis torques-reginae
  source 1..540: mol_type = other DNA
ttgatattacaaatcattttcctagctctctatgttgttgaaggattaggattagctacagagagtttagtaggctactttacaggtcaaggtgcgaaggaaaagttagcttctttggtaaggctttctggacaagctagtctttttttgggacttgcctttgctcttgtacctgcactttttcctcaaaggatatttgggttgttcaccgaccacgttgaaataattagtcaaattgatacatatatttggtggttaccactttttttgacgtttagttcagtagcacttgcactggaaggatactttctgggtttagcagaaggacgtactcttcgcaacattaacttaactgccattactttaggatttgcgcctttaagtatagtcgcttggcagtttcacaataaccatttgttgtggctggcttattctatgttcatgtgcatcagaatggtgatgcttggtatacaagtcccaaaaacttttgaaagtgattttgacactctccaggctgaagcaacggagattcttaagtag

SEQ ID NO: 75 - DNA, 473 bp, Sphaerospermopsis torques-reginae
  source 1..473: mol_type = other DNA
atgcatttgacactcttcaaacagtatgactttcttaaccgatttattcggttatcgcttgccaatataatctccaatctcatgatcccgctagctggttcaatcagtctagtttttttaggtcatctgtcagaagtccatgatttggcaggaccatcttttgcagtagtattatttgatttcatttactacgggttttactttctacgacagagtaccactggagttatcgctcaggcaataggacgagaagaccaaagggaagtgttgctgatagggctacgaaatatcttaattgctttagttttaggcatactgcttctgattttgcagtacccattaagggagataggattttccatgttaagtgacactccagaggtaaaagcttcggcggttgcccatttcaatgctcggattattggtgcgcctgcggttttcgccaattacgttctgattggttggttactagg

SEQ ID NO: 76 - protein, 267 aa, Sphaerospermopsis torques-reginae
MRKNPFESNLALEHSCYAKFDGTEFKILESGTKISEKVTEVHAEFREKILHRTPPFTCMGATSVFNQNNYRFSMYSDMCTKETTLALARNLFTFVNEQDSMNSHFTAFIAVFEKPIPEDEHHFEKLVWSQLQMLHDEDSRYHDWDTQFSNDINNHKFSYSFAERSFFIIGMHPGSSRIARQFSYPLLVFNASRQFDHLVETNQFHNFVRIIRERDVALQGSINPNLPAKYDHSRPEEPEVRQYSGRAVEKDWQCPLVVHQKKHKHNQ

SEQ ID NO: 77 - protein, 318 aa, Sphaerospermopsis torques-reginae
MKKNIKKYRFSNSIYHQIKPLLKLDQWHCWLALAEDWIVICFAISISYYLSWYFYPVTILLIGSRQRALATLLHEAAHQVIAKNRTLNFMMGTFFSGYFILQTMSSYRKSHVEKHHRYFGNPDEDPDYKFAISQGLYQQSLDDKTFQMLYIFSPLLLAQVPKYIKSLLIHRFLEDQNYLELVAIGLWWLTIISLSILFNLWQYLILFWLIPYLTVFQIIGWFIELAEHYPLMNNDINLYMSRNRNSHFIEKFLTGMHNENYHLVHHLFPNIPFWNVPKAHEILMQDPNYAQHHSQSGGIVWSNNNAPSILSGLTQVSS

SEQ ID NO: 78 - protein, 370 aa, Sphaerospermopsis torques-reginae
MKIQPALLEMWLSEYERVPYNLGESSVDNFTLGELLNLTGDRDALDQLSLMNNDTHGSLRLREAIASLDKSVSPDDILVTAGTTEAILIYFKVRYRSGANVVVPVPTFHVLYETPAFLGYEVRYLQLRAENGFRIDPQELAKLVDDNTEVIVLNTPQNPSGVVCSETEIQSIIEIAEKHNAEILADEHYRFLPHDQDTEILPSLYGLSPKIISLGSTGKCFGCIGLRIGWLIGNPEIIKACHFFKDYTTHTVCVLNDYIAAGVLLHKGKILPRYRQMIQHNIQQFETFIKQQRGLIDWVKPEAGTIAFPFFTDPNINSKIVAKRLVEDHGVLLLPGEAFDRPSHFRIALGVEPSLFQYALEKLAIVIETS

SEQ ID NO: 79 - protein, 347 aa, Sphaerospermopsis torques-reginae
MMSIQTLNFTQSELEEIDILLDQVTANYSHAEQPELIDTAAVLAQSLPLRLRQFLHEFKLRETAPACLIKGYPVNDNTIGATPEHWAGHEDAKRTKREQIVFVLCSALLGEIFGWLTQQNAYLIHDVFPIKGYEQDQIGSGSEALLLWHTEDAYHPARGDYLGLMCLRNPCQSITTIGSVDSLKKLTQQQLDILFQPRFIIRPDNAHHADNNYQTETDLDTHLQNAYNQIKHKIISQDNAMALLYGEPSAPYLCVDPAYMEATDEEAKQTLQALIDAIDIEIMDVILQPGDICFVDNFRAVHGRKPFKANYDGNDRWLKRINITRDLRKSKTYRSHPTSRIIDKVPS

SEQ ID NO: 80 - protein, 436 aa, Sphaerospermopsis torques-reginae
MLKDSPKSHAFFERAQKVIPFGVNSSHRYWGENTPVIQRGEGAYVYDFDGKSYIDYRLGFGPVILGHAHPFVNQRVIAAIQNGVSFSATQEYEVRVAEHIIKMCPSVDMVRLDNSGSDATRHAIRLARGYTGRDLVLKFEGAYHGDYDYMLWTTPDSLKSRVGKRGEPIGHKTSQGVPDLVSQLLLIAVWNDREGIEKLLKEKGDSIAAIIVEPILANAGGLMPEPGFLQFLRNQCDIYGIVLIFDEVKTGFRIAPGGAGEYFGVQADLSAYAKAMGNGYPISAIGGKRDIMMTIAPGKVVHAGTYTGNVVVTAAADATLEYMQQQDVFGHLNHVGQQLMDGLDEILTRHQINHHIHGLPSLFGLTLSESNPKDWRDVVDTNLELSEQLLIELVNAGIMTDSDPQQTWYVCESHTSEDIAETLNRFETALRKALQK

SEQ ID NO: 81 - protein, 274 aa, Sphaerospermopsis torques-reginae
MELKFQENDFQSFEPIPYLNEYFNYPSDYYGGVGFENEQFLQFFAEVAHEYHLNNYLLLDFGCGPTIYSILSLGRNCREIHMSDYLQQNLEQVQLWQQGKPQAFDWYPYLRRALQMETALNQNQPLDSFLDVTDEEIEERARLLREKITSIRKGNARATNPVGEEGKALYEAVVSSFCLEGVAQDLAEWKNLITNLSSTIKPGGLLIFATQIEADSYRIGEGYGTVVNLTEQDIVQTLLNCNFESGSIKTKELKGGPNHTEYDKFLMLTARLAV

SEQ ID NO: 82 - protein, 344 aa, Sphaerospermopsis torques-reginae
MSEHFPIDLRSDTVTQPTPSMRQAMAEAIVGDDVYGEDPTVNRLERVAAELLGKESAMFVPSGTMGNLICMLTHCGRGEEVIIGDQAHLFLYERGGSAVLGGIHPYTVSTATDGTLPLSEIRAAVRPDDIHCPRTKLLCVENTQAQRGGLPLSLTYLHSLRDLANELGLRLHCDGARLWNASVAIGVEPSVLSSPFDSLSVCLSKGLAAPIGSLVIGTQSFIDEARRLRKLLGGGMRQVGAIASAGLIALEQMRERLAIDHENAQFLAEGLRECGYEITHPVRTNIIFFRQHEQDTLSLQERLAVWKNSGLLVSDFGDRTIRAVTHYGIERKDINQAISILQQV

SEQ ID NO: 83 - protein, 413 aa, Sphaerospermopsis torques-reginae
MSNNKQDINHTELNPMAHSRRGFFTVFIAGISTSVSSIFRKDVAVAENHKAFTAENKSMLSTNNQSFFFPGELLASDEMRITIVGSSSVARKTQSGTSIFLELGNGDSFIFDAGPGVAANYTALGVPYSRMDRIFLSHLHCDHITDLAYIYGQGSMYDRKTPLRIWGPSGKESKFGTNAFVESMKAMCLWHTESLSYHQNGNGYETEVHEFDYAQNPGVAYNEKGVVIKHFPAVHLKDGAVSYRVDWNGLSFVFSGDSQPNTFMVENAQNVDILIHQTIPPAQVWAKEYNMPLEQVIELQNRFDSPPHAVGAIFDQTRPRLGVLTHLYLNENIKVAILDSCQEVYDGPIEIAQDLMVFNISKNEIKQRIAIVPELALQNTVKKYENLPPPKYKPREQLSQWLLDAVIPQEQWK

SEQ ID NO: 84 - protein, 305 aa, Sphaerospermopsis torques-reginae
MKKSIDKIITNLPLWDTQGALKIVPIGDQNFTNQNYRVESNGQVFKVRISEGNQNLIGIAKEEELIVLKAVAKMGIGPEVIAYIPPEGHLVTRFIEGKHFSLQEITQPENIHRIAQILKQVHSIEGIDAAPPPFERIEGLMRNAERKNGIFPDDFEQLLADLQAIKAVLTKVHRKPCLCHNDLAGSNIIEADGSIFLIDWEYAGVANPMFDLANFSMNQNLDREGDKILIESYFGKVSQVELAEINLWKIVAIFIEGVWGVLQAKISQFDRDYQGFANENWQTVRQYTQKESFSEWLSMISLSNH

SEQ ID NO: 85 - protein, 249 aa, Sphaerospermopsis torques-reginae
MVKSNILSRNVVGYNSTDLSSDHIQAEARDFYARQAGVFAKILNESWSEYTNISHPGFKNDLDLIRQVKELVAPGAKGIDAGCGPLAREVFCFYEDGYDMYGMDVVPENIQTALEYHPSLADRLFLADLTQPLDVPDHSFDFIICNTVIQHINLNSLTNVTFPEWTRILKPGGIIQLMFRSGHGTQIIHDVEFNDQRSFYLYQPEEILEILARYGLSLIPEENGNLGGIIYVTDVRPMENCIFCVRKTD

SEQ ID NO: 86 - protein, 452 aa, Sphaerospermopsis torques-reginae
MYLTLFKQYDFLNRFIRLSVANIVSNLMIPLAGSISLVFLGHLSGVHDLAGSSFAVVLFDFIYYGFYFLRQSTTGVTAQAIGREDQEEMLLIGLRNILIALVLGILLLILQYPFREIAFSMLNDTPEVKASAVAYFNARIIGAPAVFINYVLIGCLLGREESGKVLLISVMGNAVNILLDYLLIIHWGWGATGAGLSMAISQYLMLLVGLIFFCLQIQWQMLKAITGKLLDWSALKSAFVLNGNIFVSTLILISTTDIFSLESNTMGTIIFTQNTLILQIIFLALYFVEGLGLATESLVGYFTGQGAKEKLASLVGLSAQASLVLGLAFALVPALFPQWVFGLFTDHIEIISQIDTYIWWLPLFLTFSSVALALEGYFLGLAEGRTLRNINLTAIILGFMPLSIAAWQFHNNHLLWLAYSMFMCIRMVMLSIQVPRTFESDFGERKILGEME

SEQ ID NO: 87 - DNA, 58 bp, synthetic construct
  source 1..58: mol_type = other DNA
ttgtttaactttaataaggagatataccatgaagaaaaacatcaagaaataccgtttc

SEQ ID NO: 88 - DNA, 44 bp, synthetic construct
  source 1..44: mol_type = other DNA
gcattatgcggccgcaagcttgttagctgctaacttgggtcaga

SEQ ID NO: 89 - DNA, 47 bp, synthetic construct
  source 1..47: mol_type = other DNA
gttaagtataagaaggagatatacatatgaagatccagccggcgctg

SEQ ID NO: 90 - DNA, 55 bp, synthetic construct
  source 1..55: mol_type = other DNA
ccgatatccaattgagatctgccatatgttagctggtttcaataacgatcgccag

SEQ ID NO: 91 - DNA, 22 bp, synthetic construct
  source 1..22: mol_type = other DNA
caagcttgcggccgcataatgc

SEQ ID NO: 92 - DNA, 31 bp, synthetic construct
  source 1..31: mol_type = other DNA
catggtatatctccttattaaagttaaacaa